(19) United States
(12) Patent Application Publication
(10) Pub. No.: US 2021/0298276 A1
(43) Pub. Date: Sep. 30, 2021

(54) A METHOD OF GENERATING STERILE AND MONOSEX PROGENY

(71) Applicant: Center for Aquaculture Technologies, Inc., San Diego, CA (US)

(72) Inventors: Xavier Christophe Lauth, San Diego, CA (US); John Terrell Buschanan, San Diego, CA (US)

(73) Assignee: Center for Aquaculture Technologies, Inc., San Diego, CA (US)

(21) Appl. No.: 17/264,029

(22) PCT Filed: Aug. 12, 2019
(86) PCT No.: PCT/US19/46088

§ 371 Date: Jan. 28, 2021

Related U.S. Application Data
(60) Provisional application No. 62/717,201, filed on Aug. 10, 2018.

(51) Int. Cl. A01K 67/027; A01K 67/033

(52) U.S. Cl. CPC A01K 67/027; A01K 2227/40; A01K 67/033

(57) ABSTRACT

The disclosure provides a method of generating a sterile sex-determined fish, crustacean, or mollusk. The method comprises breeding (i) a fertile homozygous mutated female fish, crustacean, or mollusk having at least a first mutation and a second mutation with (ii) a fertile homozygous mutated male fish, crustacean, or mollusk having at least the first mutation and the second mutation to produce the sterile sex-determined fish, crustacean, or mollusk. The first mutation disrupts one or more genes that specify sexual differentiation, the second mutation disrupts one or more genes that specify gamete function, and the fertility of the fertile homozygous female fish, crustacean, or mollusk and the fertile homozygous mutated male fish, crustacean, or mollusk has been rescued. The disclosure also provides methods of making broodstock for use in producing sterile sex-determined fish, crustacean, or mollusks, as well as the broodstock itself.

### STATEMENT OF GOVERNMENT RIGHTS

Aspects of the work described herein were supported by grant award #2018-33522-28745 from the USDA-National Institute of Food and Agriculture. The United States Government may have certain rights in these inventions.

### FIELD

The present disclosure relates generally to methods of sterilizing and sex-determining freshwater and seawater organisms.

### BACKGROUND

The following paragraphs are not an admission that anything discussed in them is prior art or part of the knowledge of persons skilled in the art.

Fish species have been genetically engineered (GE) to produce valuable pharmaceutical proteins or to incorporate advantageous traits for aquaculture. A variety of fish with improved growth rates, food conversion ratios, resistance to disease, and enhanced nutritional benefits, have been developed to address the future demand for seafood and the need to improve sustainability in the aquaculture industry. However, worldwide adoption of these GE fish is hampered by concerns over their accidental release into natural ecosystems. Cultured fish have been shown to reproduce and survive in natural environments, resulting in feral populations. Similarly, GE fish may have native relatives, raising the possibility that the genetic modifications will spread throughout the wild population and alter the native gene pool. Commercial GE fish therefore represent a potential threat to the environment and a challenge to policy makers and regulatory agencies tasked with risk-benefit evaluations.

One approach to address one or more of the aforementioned issues is to sterilize fish. The induction of triploidy is the most used and best studied approach for producing sterile fish. Generally, triploid fish are produced by applying temperature or pressure shock to fertilized eggs, forcing the incorporation of the second polar body and producing cells with three chromosome sets (3N). Triploid fish do not develop normal gonads as the extra chromosome set disrupts meiosis. At the industrial scale, the logistics of reliably applying pressure or temperature shocks to batches of eggs is complicated and carries significant costs. An alternative to triploid induced by physical treatments is triploid induced by genetics, which results from crossing a tetraploid with a diploid fish. Tetraploid fish, however, are difficulty to generate due to poor embryonic survival and slow growth. In some examples, triploid males produce some normal haploid sperm cells thus allowing males to fertilize eggs, though at a reduced efficiency. Also, in some species, negative performance characteristics have been associated with triploid phenotype, including reduced growth and sensitivity to disease.

Another approach for sterilizing fish is by hormone treatment extending over several weeks. However, in many cases, including these intensive long-term treatment processes do not have a desirable efficacy of sterility, and/or have been associated with decreased fish growth performance. Furthermore, treatments involving a synthetic steroid may result in higher mortality rates.

Another approach for sterilizing fish is by using transgenic-based technologies, which include a step of integrating a transgene that induce germ cell death or disrupts their migration patterns resulting in their ablation in developing embryos. However, transgenes are subject to position effect as well as silencing. Consequently, such approaches are subject to extended regulatory review processes before being considered acceptable for commercial use.

An alternative approach for sterilizing fish is by knockdown or knockout of genes governing primordial germ cell (PGC) development. Such approaches have been reported to cause PGC loss and sterility. However, the sterile trait in these fish is not heritable. Accordingly, utilizing an approach of knockdown or knockout of genes governing PGC development may be logistically challenging and costly and thus impractical to efficiently mass produce sterile fish at commercial scale.

Mechanisms governing sexual or gonadal differentiation in teleost fish are complex processes influenced by internal (genetic and endocrine factors) and external factors, including social interaction and environmental conditions (water temperature, pH and oxygen), whose relative contributions can vary significantly depending on the species.

Improvements in generating sterile, sex-determined fish, crustaceans, or mollusks is desirable.

### INTRODUCTION

The following introduction is intended to introduce the reader to this specification but not to define any invention. One or more inventions may reside in a combination or sub-combination of the instrument elements or method steps described below or in other parts of this document. The inventors do not waive or disclaim their rights to any invention or inventions disclosed in this specification merely by not describing such other invention or inventions in the claims.

One or more of the previously proposed methods used for sterilizing freshwater and seawater organisms may result in: (1) an insufficient efficacy; (2) increased difficulty to propagate the sterility trait by, for example, having to perform genetic selection to identify a subpopulation of sterile individual, and/or repeating treatment at each generation; (3) an increase in operating costs by, for example, incorporating significant changes in husbandry practices, being untransferable across multiple species, increasing production times, increasing the percentage of sterile organisms with reduced growth and increased sensitivity to disease, increasing mortality rates of sterile organisms, or a combination thereof; (4) gene flow to wild populations and colonization of new habitats by cultured, non-native species; or (4) a combination thereof.

The present disclosure provides methods of producing sex-determined sterilized freshwater and seawater organisms by disrupting their sexual differentiation and gametogenesis pathways. One or more examples of the present disclosure may: (1) increase efficacy of sterilization, by for example, allowing mass production of sterile individuals and ensuring that all individuals are completely sterile; (2) decrease operating costs by, for example, decreasing the amount of costly equipment or treatments, being commercially scalable, being transferable across multiple species, decreasing feed, decreasing production times, decreasing the percentage of organisms that attain sexually maturity, increasing the physical size of sexually mature organisms, or a combination thereof; (3) decrease gene flow to wild populations and colonization of new habitats by cultured non-native species; (4) increase culture performance by decreasing loss of energy to gonad development; or (5) a combination thereof, compared to one or more previously proposed methods used for sterilizing freshwater and seawater organisms.

One or more examples of the present disclosure may yield at least a 10% improvement in food conversion rates (FCR=amount of weight gained per quantity of food fed) and about 20% faster growth rates, compared to other lines currently used in production systems (Methyltestosterone treatment). These performance benefits may only impact feed costs (direct reduction in feed costs) and labor (reduced labor due to shortened culture times). Based on averaged itemized costs of a U.S. tilapia farming operation producing 1000 lbs of product, savings of about $0.23 per market sized fish (1.5 pounds) using all male sterile-Tilapia may be realized, suggesting that an operation choosing to retain its savings in production costs may experience an increase in profit margin approaching about 130%.

The present disclosure also discusses methods of making broodstock freshwater and seawater organisms for use in producing sex-determined sterilized freshwater and seawater organisms, as well as the broodstock itself.

The present disclosure provides a method of generating a sterile sex-determined fish, crustacean, or mollusk, comprising the steps of: breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation; and selecting a progenitor that is homozygous by genotypic selection, the homozygous mutated progenitor being the sterile sex-determined fish, crustacean, or mollusk, wherein the first mutation disrupts one or more genes that specify sexual differentiation, and wherein the second mutation disrupts one or more genes that specify gamete function.

The present disclosure also provides a method of generating a sterile sex-determined fish, crustacean, or mollusk, comprising the step of: breeding (i) a fertile homozygous mutated female fish, crustacean, or mollusk having at least a first mutation and a second mutation with (ii) a fertile homozygous mutated male fish, crustacean, or mollusk having at least the first mutation and the second mutation to produce the sterile sex-determined fish, crustacean, or mollusk, wherein the first mutation disrupts one or more genes that specify sexual differentiation, wherein the second mutation disrupts one or more genes that specify gamete function, and wherein the fertility of the fertile homozygous female fish, crustacean, or mollusk and the fertile homozygous mutated male fish, crustacean, or mollusk has been rescued.

The fertility rescue may comprise germline stem cell transplantation. The fertility rescue may further comprise sex steroid alteration. The alteration of sex steroid may be an alteration of estrogen, or an alteration of an aromatase inhibitor.

The germline stem cell transplantation may comprise the steps of: obtaining a germline stem cell from a sterile homozygous male fish, crustacean, or mollusk having at least the first mutation and the second mutation or a germline stem cell from a sterile homozygous female fish, crustacean, or mollusk having at least the first mutation and the second mutation; and transplanting the germline stem cell into a germ cell-less recipient male fish, crustacean, or mollusk, or into a germ cell-less recipient female fish, crustacean, or mollusk. The germ cell-less recipient male fish, crustacean, or mollusk and the germ cell-less recipient female fish, crustacean, or mollusk may be homozygous for a null mutation of the dnd, Elavl2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The germline stem cell transplantation may comprise the steps of: obtaining a spermatogonial stem cell from a sterile homozygous male fish, crustacean, or mollusk having at least the first mutation and the second mutation or a oogonial stem cell from a sterile homozygous female fish, crustacean, or mollusk having at least the first mutation and the second mutation; and transplanting the spermatogonial stem cell into a testis of a germ cell-less fertile male fish, crustacean, or mollusk or the oogonial stem cell into an ovary of a germ cell-less fertile female fish, crustacean, or mollusk. The germ cell-less fertile male fish, crustacean, or mollusk and the germ cell-less fertile female fish, crustacean, or mollusk may be homozygous for the mutation of the dnd, ElavI2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The sterile sex-determined sterile fish, crustacean, or mollusk may be a sterile male fish, crustacean, or mollusk. The first mutation may comprise a mutation in one or more genes that modulates the synthesis of androgen and/or estrogen. The first mutation may comprise a mutation in one or more genes that modulate the expression of aromatase Cyp19a1a, Cyp17, or a combination thereof. The one or more genes that modulate the expression of aromatase Cyp19a1a may be one or more genes selected from the group consisting of cyp19a1a, FoxL2, and an ortholog thereof. The one or more genes that modulate the expression of Cyp17 may be cyp17l or an ortholog thereof. The second mutation may comprise a mutation in one or more genes that modulate spermiogenesis. The second mutation may comprise a mutation in one or more genes that cause globozoospermia. The second mutation in one or more genes that cause globozoospermia may cause sperm with round-headed, round nucleus, disorganized midpiece, partially coiled tails, or a combination thereof. The second mutation may comprise a mutation in one or more genes selected from the group consisting of Gopc, Hiat1, Tjp1a, Smap2, Csnk2a2, and an ortholog thereof.

The sterile sex-determined sterile fish, crustacean, or mollusk may be a sterile female fish, crustacean, or mollusk. The first mutation may comprise a mutation in one or more genes that modulate the expression of an aromatase Cyp19a1a inhibitor. The one or more genes that modulate the expression of an aromatase Cyp19a1a inhibitor may be one or more genes selected from the group consisting of Gsdf, dmrt1, Amh, Amhr, and an ortholog thereof. The second mutation may comprise a mutation in one or more genes that modulate oogenesis, folliculogenesis, or a combination. The one or more genes that modulate oogenesis may modulate the synthesis of estrogen. The one or more genes that modulate the synthesis of estrogen may be FSHR or an ortholog thereof. The one or more genes that modulate folliculogenesis may modulate the expression of vitellogenins. The one or more genes that modulate the expression of vitellogenins may be vtgs or an ortholog thereof. The one or more genes that modulate the expression of vitellogenins may be a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof.

The present disclosure also provides a method of generating a sterile sex-determined fish, crustacean, or mollusk, comprising the step of: breeding (i) a fertile female fish, crustacean, or mollusk having a homozygous mutation with (ii) a fertile male fish, crustacean, or mollusk having a homozygous mutation to produce the sterile sex-determined fish, crustacean, or mollusk, wherein the mutation directly or indirectly disrupts spermiogenesis, and/or directly disrupts vitellogenesis, and wherein the fertility of the fertile female fish, crustacean, or mollusk and the fertile male fish, crustacean, or mollusk have been rescued.

The mutation that directly or indirectly disrupts spermiogenesis may be a mutation in Gopc, Hiat1, Tjp1a, Smap2, Csnk2a2, or an ortholog thereof. The mutation that directly disrupts vitellogenesis may be a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof. The fertile female fish, crustacean, or mollusk and the fertile male fish, crustacean, or mollusk may have a plurality of homozygous mutations that, in combination: directly or indirectly disrupt spermiogenesis; directly disrupt vitellogenesis; or both.

The fertility rescue may comprise germline stem cell transplantation. The fertility rescue may further comprise sex steroid alteration. The alteration of sex steroid may be an alteration of estrogen, or an alteration of an aromatase inhibitor.

The germline stem cell transplantation may comprise the steps of: obtaining a germline stem cell from a sterile homozygous male fish, crustacean, or mollusk having at least the homozygous mutation or a germline stem cell from a sterile homozygous female fish, crustacean, or mollusk having at least the homozygous mutation; and transplanting the germline stem cell into a germ cell-less recipient male fish, crustacean, or mollusk, or into a germ cell-less recipient female fish, crustacean, or mollusk. The germ cell-less recipient male fish, crustacean, or mollusk and the germ cell-less recipient female fish, crustacean, or mollusk may be homozygous for a null mutation of the dnd, Elavl2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The fertile female fish, crustacean, or mollusk and the fertile male fish, crustacean, or mollusk may have an additional homozygous mutation that specifies sexual differentiation. The mutation that specifies sexual differentiation may modulate the expression of aromatase Cyp19a1a, Cyp17, an inhibitor to aromatase Cyp19a1a, or a combination thereof. The mutation that modulates the expression of Cyp17 may be a mutation in cyp17l or an ortholog thereof. The mutation that modulates the expression of aromatase Cyp19a1a inhibitor may be a mutation in Gsdf, dmrt1, Amh, Amhr, or an ortholog thereof.

The breeding step of the herein disclosed methods may comprise hybridization or hormonal manipulation and breeding strategies, to specify sexual differentiation.

The fish, crustacean, or mollusk of the herein disclosed methods may be a fish.

The present disclosure also provides a fertile homozygous mutated fish, crustacean, or mollusk for producing a sterile sex-determined fish, crustacean, or mollusk, the fertile homozygous mutated fish, crustacean, or mollusk having at least a first mutation and a second mutation, wherein the first mutation disrupts one or more genes that specify sexual differentiation, wherein the second mutation disrupts one or more genes that specify gamete function, and wherein the fertility of the fertile homozygous mutated fish, crustacean, or mollusk has been rescued. The fertility rescue may comprise germline stem cell transplantation. The fertility rescue may further comprise sex steroid alteration. The alteration of sex steroid may be an alteration of estrogen, or an alteration of an aromatase inhibitor.

The germline stem cell transplantation may comprise the steps of: obtaining a germline stem cell from a sterile homozygous male fish, crustacean, or mollusk having at least the first mutation and the second mutation or a germline stem cell from a sterile homozygous female fish, crustacean, or mollusk having at least the first mutation and the second mutation; and transplanting the germline stem cell into a germ cell-less recipient male fish, crustacean, or mollusk, or into a germ cell-less recipient female fish, crustacean, or mollusk. The germ cell-less recipient male fish, crustacean, or mollusk and the germ cell-less recipient female fish, crustacean, or mollusk may be homozygous for a null mutation of the dnd, Elavl2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The germline stem cell transplantation may comprise the steps of: obtaining a spermatogonial stem cell from a sterile homozygous male fish, crustacean, or mollusk having at least the first mutation and the second mutation or a oogonial stem cell from a sterile homozygous female fish, crustacean, or mollusk having at least the first mutation and the second mutation; and transplanting the spermatogonial stem cell into a testis of a germ cell-less fertile male fish, crustacean, or mollusk or the oogonial stem cell into an ovary of a germ cell-less fertile female fish, crustacean, or mollusk. The germ cell-less fertile male fish, crustacean, or mollusk and the germ cell-less fertile female fish, crustacean, or mollusk may be homozygous for the mutation of the dnd, Elavl2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The sterile sex-determined sterile fish, crustacean, or mollusk may be a sterile male fish, crustacean, or mollusk. The first mutation may comprise a mutation in one or more genes that modulates the synthesis of androgen and/or estrogen. The first mutation may comprise a mutation in one or more genes that modulate the expression of aromatase Cyp19a1a, Cyp17, or a combination thereof. The one or more genes that modulate the expression of aromatase Cyp19a1a may be one or more genes selected from the group consisting of cyp19a1a, FoxL2, and an ortholog thereof. The one or more genes that modulate the expression of Cyp17 may be cyp17l or an ortholog thereof. The second mutation may comprise a mutation in one or more genes that modulate spermiogenesis. The second mutation may comprise a mutation in one or more genes that cause globozoospermia. The second mutation in one or more genes that cause globozoospermia may cause sperm with round-headed, round nucleus, disorganized midpiece, partially coiled tails, or a combination thereof. The second mutation may comprise a mutation in one or more genes selected from the group consisting of Gopc, Hiat1, Tjp1a, Smap2, Csnk2a2, and an ortholog thereof.

The sterile sex-determined sterile fish, crustacean, or mollusk may be a sterile female fish, crustacean, or mollusk. The first mutation may comprise a mutation in one or more genes that modulate the expression of an aromatase Cyp19a1a inhibitor. The one or more genes that modulate the expression of an aromatase Cyp19a1a inhibitor may be one or more genes selected from the group consisting of Gsdf, dmrt1, Amh, Amhr, and an ortholog thereof. The second mutation may comprise a mutation in one or more genes that modulate oogenesis, folliculogenesis, or a combination. The one or more genes that modulate oogenesis may modulate the synthesis of estrogen. The one or more genes that modulate the synthesis of estrogen may be FSHR or an ortholog thereof. The one or more genes that modulate folliculogenesis may modulate the expression of vitellogenins. The one or more genes that modulate the expression of vitellogenins may be vtgs or an ortholog thereof. The one or more genes that modulate the expression of vitellogenins may be a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof.

The present disclosure also provides a fertile fish, crustacean, or mollusk having a homozygous mutation for producing a sterile sex-determined fish, crustacean, or mollusk, wherein the mutation directly or indirectly disrupts spermiogenesis, and/or directly disrupts vitellogenesis, and wherein the fertility of the fertile fish, crustacean, or mollusk has been rescued.

The mutation that directly or indirectly disrupts spermiogenesis may be a mutation in Gopc, Hiat1, Tjp1a, Smap2, Csnk2a2, or an ortholog thereof. The mutation that directly disrupts vitellogenesis may be a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof. The fertile fish, crustacean, or mollusk may have a plurality of homozygous mutations that, in combination: directly or indirectly disrupt spermiogenesis; directly disrupt vitellogenesis; or both. The fertility rescue may comprise germline stem cell transplantation. The fertility rescue may further comprise sex steroid alteration. The alteration of sex steroid may be an alteration of estrogen, or an alteration of an aromatase inhibitor.

The germline stem cell transplantation may comprise the steps of: obtaining a germline stem cell from a sterile homozygous male fish, crustacean, or mollusk having at least the homozygous mutation or a germline stem cell from a sterile homozygous female fish, crustacean, or mollusk having at least the homozygous mutation; and transplanting the germline stem cell into a germ cell-less recipient male fish, crustacean, or mollusk, or into a germ cell-less recipient female fish, crustacean, or mollusk. The germ cell-less recipient male fish, crustacean, or mollusk and the germ cell-less recipient female fish, crustacean, or mollusk may be homozygous for a null mutation of the dnd, Elavl2, vasa, nanos3, or piwi-like gene. The germ cell-less recipient male fish, crustacean, or mollusk and the germ cell-less recipient female fish crustacean, or mollusk may be created using ploidy manipulation. The germ cell-less recipient male fish, crustacean, or mollusk and the germ cell-less recipient female fish crustacean, or mollusk may be created by hybridization. The germ cell-less recipient male fish, crustacean, or mollusk and the germ cell-less recipient female fish crustacean, or mollusk may be created using exposure to high levels of sex hormones.

The fertile fish, crustacean, or mollusk may have an additional homozygous mutation that specifies sexual differentiation. The mutation that specifies sexual differentiation may modulate the expression of aromatase Cyp19a1a, Cyp17, an inhibitor to aromatase Cyp19a1a, or a combination thereof. The one or more genes that modulate the expression of aromatase Cyp19a1a may be one or more genes selected from the group consisting of cyp19a1a, FoxL2, and an ortholog thereof. The one or more genes that modulate the expression of aromatase Cyp19a1a inhibitor may be one or more genes selected from the group consisting of Gsdf, dmrt1, Amh, Amhr, and an ortholog thereof.

Producing a sterile sex-determined fish, crustacean, or mollusk may comprise a breeding step comprising hybridization or hormonal manipulation and breeding strategies, to specify sexual differentiation.

The herein disclosed fertile fish, crustacean, or mollusk may be a fish.

The present disclosure also provides a method of making a fertile homozygous mutated fish, crustacean, or mollusk that generates a sterile sex-determined fish, crustacean, or mollusk, comprising the steps of: breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation; selecting a progenitor that is homozygous by genotypic selection; and rescuing the fertility of the homozygous progenitor, wherein the first mutation disrupts one or more genes that specify sexual differentiation, and wherein the second mutation disrupts one or more genes that specify gamete function.

Other aspects and features of the present disclosure will become apparent to those ordinarily skilled in the art upon review of the following description of specific examples in conjunction with the accompanying figures.

### BRIEF DESCRIPTION OF THE DRAWINGS

Examples of the presently disclosed methods and organisms will now be described, by way of example only, with reference to the attached Figures.

FIG. 1 is a flowchart showing an example of a method of generating a sterile sex-determined fish, crustacean, or mollusk and propagating a mutated line.

FIG. 2 is illustrations and graphs showing an example of F0 mosaic founder mutant identification and selection strategy. Mutant alleles were identified by fluorescence PCR with genes specific primers designed to amplify the regions around the targeted loci (120-300 bp). For fluorescent PCR, both combination of gene specific primers and two forward oligos with the fluorophore 6-FAM or NED attached were added to the reaction. A control reaction using wild type DNA is used to confirm the presence of single Peak amplification at each loci. The resulting amplicon were resolved via capillary electrophoresis (CE) with an added LIZ labeled size standard to determine the amplicon sizes accurate to base-pair resolution (Retrogen). The raw trace files were analyzed on Peak Scanner software (ThermoFisher). The size of the peak relative to the wild-type peak control determines the nature (insertion or deletion) and length of the mutation. The number of peaks indicate the level of mosaicism. We selected F0 mosaic founder carrying the fewest number of mutant alleles (2-4 peak preferentially).

FIG. 3 is a graph illustrating an example Melt Curve plot visualizing the genotypes of heterozygous, homozygous mutant and wild type samples. The negative change in fluorescence is plotted versus temperature (−dF/dT). Each trace represents a sample. The melting temperature of the wild-type allele in this example is ˜81° C. (wild type peak), the melting temperature of the homozygous mutant product (homozygous deletion peak) is ˜79° C. The remaining trace represents a heterozygote.

FIG. 4 panels A to D are photographs of different stages of growth of a Tilapia F0 generation comprising double-allelic knockout of pigmentation genes.

FIG. 5 panels A to B are photographs of Tilapia after multi-gene targeting comprising dead end1 (dnd) and tyrosinase (Tyr). FIG. 5 panel A is an F0 Tyr deficient albino. FIG. 5 panel B shows dissected testis from control (WT) and sterile (F0 dnd KO) tilapia.

FIG. 6 panels A to B are photographs of germ cell depleted testis and ovary (arrowheads point toward the gonads) from Elavl2-Knockout tilapia (ElavI2^{Δ8/Δ8}). Small photo inserts show the urogenital papillae. Elavl2 mutants were produced by microinjecting engineered nucleases targeting Elavl2 coding sequence into one cell stage tilapia embryos. One of the resulting founder males was mated with a wild-type female and produced heterozygous mutants in the F1 generation. Mating of these F1 mutants ElavI2^{Δ8/+} produced an F2 generation with approximately 25% of the clutch being sterile homozygous mutant of both sexes.

FIG. 7 panels A to C are illustrations of selected mutant alleles at the tilapia cyp17loci. FIG. 7 panel A is a schematic of the cyp17gene. Exons (E1-8) are shown as shaded boxes; translational start and stop sites as ATG and TAA, respectively. Arrows point to targeted sites in the first exon. FIG. 7 panel B is the wild-type reference sequence (SEQ ID NO: 60) with the selected germ-line mutant allele (SEQ ID NO: 61) from an offspring of Cyp17 F0 mutated tilapia. This 11nt+5 nt deletion is predicted to create a truncated protein that terminates at amino acid 44 rather than position 521. FIG. 7 panel C is the predicted protein sequences of WT (SEQ ID NO: 62) and mutant cyp17allele (SEQ ID NO: 63) in which the first 16 amino acids are identical to those of the wild-type Cyp17 protein and the 44 amino acids are miscoded. Altered amino acids are highlighted.

FIG. 8 panels A to C are graphs, illustrations, and photographs showing cyp17 loss of function produces all-male offspring with no secondary sex characteristics. FIG. 8 panel A is a graph showing Cyp17 mutant fish exhibiting complete male biased. A founder male with germline mutations at the cyp17loci was bred with a wild type female, and the male and female F1 progeny carrying the null Δ16-cyp17allele were selected and crossed to produce F2 generation of wild type (WT) homozygous (−/−) and hemizygous mutants (+/−). The graph shows the count of males and females for a given genotype. FIG. 8 panel B shows an undetectable level of testosterone in cyp17 loss of function mutants. Blood was collected from the caudal vein and centrifuged at 3000 rpm for 10 min. Plasma was separated and frozen at −80° C. and free plasmatic testosterone level was measured by enzyme linked immunosorbent assay (ELISA) (Cayman Chemical, Michigan, USA). Plasma samples were analyzed in triplicate. FIG. 8 panel C shows photographs of two cyp17 F0 KO (−/−) males with underdeveloped UGP compared to an age matched non-treated male (right image).

FIG. 9 panels A to E are illustrations showing Cyp17 loss of function mutants are sexually delayed with smaller testes and oligospermia. F2 progeny from hemizygous cyp17 mutants were raised to 5 months of age, weighted (FIG. 9 panel C), and genotyped. FIG. 9 panel A shows males were sacrificed, and their testes exposed (FIG. 9 panel A) and dissected (FIG. 9 panel B) revealing a gradient of color and size (FIG. 9 panel D) with WT being the most mature gonad and homozygous appearing as sexually delayed. FIG. 9 panel E shows volume of strippable milt from 8 homozygous and WT males and FIG. 9 panel F shows spectrophotometric comparison of sperm concentration (absorbance at 600 nm).

FIG. 10 panels A to C are illustrations of selected mutant alleles at the tilapia Tight junction protein 1 (Tjp1a) loci. FIG. 10 panel A is a schematic of the Tjp1a gene. Exons (E1-32) are shown as shaded boxes; translational start and stop sites as ATG and TAA, respectively. Arrows point to targeted exons 15 and 17. FIG. 10 panel B is the wild-type reference sequence (SEQ ID NO: 71) with the selected germ-line mutant allele (SEQ ID NO: 72) from an offspring of Tjp1a F0 mutated tilapia. This 7 nt deletion is predicted to create a truncated protein that terminates at amino acid 439 rather than position 1652. FIG. 10 panel C is the predicted protein sequences of WT (SEQ ID NO: 73) and mutant Tjp1a allele (SEQ ID NO: 74) in which the first 439 amino acids are identical to those of the wild-type Tjp1a protein.

FIG. 11 panels A to C are illustrations of selected mutations at the tilapia Hippocampus abundant transcript 1 a (Hiat1) loci. FIG. 11 panel A is a schematic of the tilapia Hiat1 gene. Exons (E1-12) are shown as shaded boxes; 5′ and 3′ untranslated regions are shown as open boxes. Arrows point to targeted exons 4 and 6. FIG. 11 panel B is the wild-type reference sequence (SEQ ID NO: 75) with the sequence of the selected germ-line mutant allele (SEQ ID NO: 76) from an offspring of Hiat1 F0 mutated tilapia. Location of the 17 nucleotides deletion is shown by dashes. This frameshift mutation is predicted to create a truncated protein that terminates at amino acid 234 rather than position 491. FIG. 11 panel C shows the predicted protein sequences of WT (SEQ ID NO: 77) and truncated mutant Hiat1 protein (SEQ ID NO: 78) in which the first 218 amino acids are identical to those of the wild-type and the following 16 amino acids are miscoded.

FIG. 12 panels A to C are illustrations of selected mutations at the tilapia Small ArfGAP2 (Smap2) loci. FIG. 12 panel A is a schematic of the tilapia Smap2 gene. Exons (E1-12) are shown as shaded boxes, and 3′ untranslated region is shown as open box. Arrows point to targeted exons 2 and 9. FIG. 12 panel B is the wild-type reference sequence (SEQ ID NO: 79) with the sequence of the selected germ-line mutant allele (SEQ ID NO: 80) from an offspring of Smap2 F0 mutated tilapia. Location of the 17 nucleotides deletion is shown by dashes. This frameshift mutation is predicted to create a truncated protein that terminates at amino acid 118 rather than position 429. FIG. 12 panel C shows the predicted protein sequences of WT (SEQ ID NO: 81) and truncated mutant Smap2 protein (SEQ ID NO: 82) in which the first 53 amino acids are identical to those of the wild-type and the following 63 amino acids are miscoded.

FIG. 13 panels A to C are illustrations of selected mutant alleles at the tilapia Casein kinase 2, alpha prime polypeptide a (Csnk2a2) loci. FIG. 13 panel A is a schematic of the Csnk2a2 gene. Exons (E1-11) are shown as shaded boxes; translational start and stop sites as ATG and TGA, respectively. Arrows point to targeted exons 1 and 2. FIG. 13 panel B is the wild-type reference sequence (SEQ ID NO: 83) with the selected germ-line mutant allele (SEQ ID NO: 84) from an offspring of Csnk2a2 F0 mutated tilapia. This 22 nt deletion is predicted to create a truncated protein that terminates at amino acid 31 rather than position 350. FIG. 13 panel C is the predicted protein sequences of WT (SEQ ID NO: 85) and mutant Csnk2a2 allele (SEQ ID NO: 86) in which the first 31 amino acids are miscoded.

FIG. 14 panels A to C are illustrations of selected mutant alleles at the tilapia Golgi-associated PDZ and coiled-coil motif (Gopc) loci. FIG. 14 panel A is a schematic of the Gopc gene. Exons (E1-9) are shown as shaded boxes; translational start and stop sites as ATG and TAA, respectively. Arrows point to targeted exons 1 and 2. FIG. 14 panel B is the wild-type reference sequence (SEQ ID NO: 87) with the selected germ-line mutant allele (SEQ ID NO: 88) from an offspring of Gopc F0 mutated tilapia. This 8 nt deletion is predicted to create a truncated protein that terminates at amino acid 30 rather than position 444. FIG. 14 panel C is the predicted protein sequences of WT (SEQ ID NO: 89) and mutant Gopc allele (SEQ ID NO: 90) in which the first 9 amino acids are identical to those of the wild-type Gopc protein and the following 21 amino acids are miscoded.

FIG. 15 panels A and B are photographs and graphs showing tilapia spermiogenesis specific gene knockouts phenocopy human and mice deficiencies. FIG. 15 panel A shows malformation of spermatozoa in F0 deficient tilapia for the five candidate genes. Microscopic images of spermatozoa collected from wild-type (WT) and from Tjp1a, Gopc, Smap2, Hiat1 and Csnk2a2 F0 mutant fish respectively. Black arrowheads point to WT size sperm head and yellow arrowheads indicate enlarged round spermatozoa head. Scale bars: 100 μm. FIG. 15 panel B shows the fertilization success rate from hand-stripped gametes, followed by in vitro fertilization in which dry gametes (200 eggs and stripped milt) were mixed together and immediately activated with 2 mL of hatching water. Data are means+/−SD, n=3 replicates.

FIG. 16 panels A to C are images and graphs showing expression levels of SMS genes in fertile and germ cell free testes. FIG. 16 panel A shows testes dissected from 4 months old dnd1 Knockout and wild type aged match control. FIG. 16 panel B illustrates that the relative expression level of vasa, a germ cell specific gene is reduced to undetectable level in testis from dnd1 KO fish but strongly expressed in wild type testis, while the Sertoli specific gene Dmrt1 is expressed at the same level in testes from wild-type and sterile tilapia. β-actin was used as the reference gene to normalize expression level of vasa and Dmrt1. FIG. 16 panel C illustrates the relative expression level of SMS genes Tjp1a, Hiat1, Gopc and Csnk2a2 in testes from wild type and sterile tilapia. Dmrt1 was used as the reference gene to normalize expression level of SMS genes. In all cases, value represent average of 3 biological replicates, +/−SD.

FIG. 17 panels A to C are illustrations of the selected mutation at the Cyp9a1a loci. FIG. 17 panel A is a schematic of the tilapia Cyp9a1a gene. Exons (E1-9) are shown as shaded boxes. Arrows point to targeted exons 1 and 9. FIG. 17 panel B is the wild-type reference sequence (SEQ ID NO: 65) with the sequences of the selected germ-line mutant alleles from Cyp19a1a F0 mutated tilapia (SEQ ID NOs: 66 and 67). The 7 nt (del 8 and ins1) and 10 nt deletions are indicated by dashes. These frameshift mutations are predicted to create truncated proteins that terminate at amino acid 12 and 11 rather than position 511. FIG. 17 panel C is the predicted protein sequences of WT (SEQ ID NO: 68) and truncated mutant proteins (SEQ ID NOs: 69 and 70), in which the first 7 and 5 amino acids are identical to those of the wild-type Cyp19a1a protein and the following 5 and 6 amino acids are miscoded. Altered amino acids are highlighted.

FIG. 18 is an illustration and table showing an example of the breeding scheme and anticipated genotypes of mutant progeny from double heterozygote parents. m1, 2, 3 symbols indicate different mutations at the Tjp1a locus in F0 mosaic female. Each column in the table shows the frequency of an expected F2 progeny for each combination of cyp17 and Tjp1a alleles, as well as the projected sex ratio and fertility status. The progeny anticipated to be all-male and sterile is circled.

FIG. 19 panels A to C are illustrations of the selected mutation at the Dmrt1 loci. FIG. 19 panel A is a schematic of the tilapia Dmrt1 gene. Exons (E1-9) are shown as shaded boxes. Arrows point to targeted exons 1 and 3. FIG. 19 panel B is the wild-type reference sequence (SEQ ID NO: 91) with the sequences of the selected germ-line mutant alleles from Dmrt1 F0 mutated tilapia (SEQ ID NOs: 92 and 93). The 7 nt and 13 nt deletions are indicated by dashes. These frameshift mutations are predicted to create truncated proteins that terminate at amino acid 40 and 38 rather than position 293. FIG. 19 panel C is the predicted protein sequences of WT (SEQ ID NO: 94) and truncated mutant proteins (SEQ ID NOs: 95 and 96), in which the first 16 amino acids are identical to those of the wild-type Dmrt1 protein and the following 24 and 22 amino acids are miscoded. Altered amino acids are highlighted.

FIG. 20 panels A to C are illustrations of the selected mutation at the growth/differentiation factor 6-B-like loci (Gsdf). FIG. 20 panel A is a schematic of the tilapia Gsdf gene. Exons (E1-5) are shown as shaded boxes. Arrows point to targeted exons 2 and 4. FIG. 20 panel B is the wild-type reference sequence (SEQ ID NO: 97) with the sequences of the selected germ-line mutant alleles from Gsdf F0 mutated tilapia (SEQ ID NOs: 98 and 99). The 5 nt and 22 nt deletions are indicated by dashes. These frameshift mutations are predicted to create truncated proteins that terminate at amino acid 56 and 46 rather than position 213. FIG. 20 panel C is the predicted protein sequences of WT (SEQ ID NO: 100) and truncated mutant proteins (SEQ ID NOs: 101 and 102), in which the first 52 and 46 amino acids are identical to those of the wild-type Gsdf protein and the following 4 and 0 amino acids are miscoded. Altered amino acids are highlighted.

FIG. 21 panels A to C are illustrations of selected mutations at the tilapia Folliculogenesis stimulating hormone receptor (FSHR) loci. FIG. 21 panel A is a schematic of the tilapia FSHR gene. Exons (E1-15) are shown as shaded boxes; 5′ and 3′ untranslated regions are shown as open boxes. Arrows point to targeted exons 11 and 15. FIG. 21 panel B is the wild-type reference sequence (SEQ ID NO: 103) with the sequence of the selected germ-line mutant allele (SEQ ID NO: 104) from an offspring of FSHR F0 mutated tilapia. Location of the 5 nucleotides deletion is shown by dashes. This frameshift mutation is predicted to create a truncated protein that terminates at amino acid 264 rather than position 689. FIG. 21 panel C shows the predicted protein sequences of WT (SEQ ID NO: 105) and truncated mutant FSHR protein (SEQ ID NO: 106) in which the first 258 amino acids are identical to those of the wild-type and the following 6 amino acids are miscoded.

FIG. 22 panels A to C are illustrations of the selected mutations at the Vitellogenin Aa (VtgAa) loci. FIG. 22 panel A is a schematic of the tilapia VtgAa gene. Exons (E1-35) are shown as shaded boxes. Arrows point to targeted exons 7 and 22. FIG. 22 panel B is the wild-type reference sequence (SEQ ID NO: 107) with the sequences of the selected germ-line mutant alleles from Gsdf F0 mutated tilapia (SEQ ID NOs: 108 and 109). The 5 nt and 25 nt deletions are indicated by dashes. These frameshift mutations are predicted to create truncated proteins that terminate at amino acid 279 and 301 rather than position 1657. FIG. 22 panel C is the predicted protein sequences of WT (SEQ ID NO: 110) and truncated mutant proteins (SEQ ID NOs: 111 and 112), in which the first 278 and 269 amino acids are identical to those of the wild-type VtgAa protein and the following 1 and 32 amino acids are miscoded. Altered amino acids are highlighted.

FIG. 23 panels A to C are illustrations of selected mutations at the tilapia Vitellogenin Ab (VtgAb) loci. FIG. 23 panel A is a schematic of the tilapia VtgAb gene. Exons (E1-35) are shown as shaded boxes; 5′ untranslated region is shown as open boxes. Arrows point to targeted exons 5 and 22. FIG. 23 panel B is the wild-type reference sequence (SEQ ID NO: 113) with the sequence of the selected germ-line mutant allele (SEQ ID NO: 114) from an offspring of VtgAb F0 mutated tilapia. Location of the 8 nucleotides deletion is shown by dashes. This frameshift mutation is predicted to create a truncated protein that terminates at amino acid 202 rather than position 1747. FIG. 23 panel C shows the predicted protein sequences of WT (SEQ ID NO: 115) and truncated mutant VtgAb protein (SEQ ID NO: 116) in which the first 270 amino acids are identical to those of the wild-type VtgAb protein and the following 32 amino acids are miscoded. Altered amino acids are highlighted.

FIG. 24 panels A and B is a photograph and graph showing that females deficient for VtgAa fail to produce viable progeny. FIG. 24 panel A is a photograph of 8 hours post fertilization embryos incubation in hatching water containing methylene blue (Roth, 0.01% of stock solution in hatching water). Blue staining indicates unfertilized eggs and dead embryos. Embryos were inspected daily under a light stereomicroscope and dead embryos counted and removed. FIG. 24 panel B shows survival percentage in the progeny from F0 VtgAa males and females outcrossed with wild type fish. Data are means+/−SD, n=2×3 replicates.

FIG. 25 is an illustration that shows breeding scheme and genotype of mutant progeny from double heterozygous parents. m1−n and m1 symbols indicate mosaic mutations in F0 and one specific mutation selected for each targeted loci. F1 genotypes shown correspond to one of the four combinations of alleles we plan to establish. Each column in the table indicates the relative frequency of expected F2 progeny for each combination of alleles, as well as the projected sex ratio and fertility status. The progeny anticipated to be all-female and sterile is circled in red.

FIG. 26 are photographs showing the impact of FSHR deficiency on ovarian development. Siblings 12 months old fertile control (WT body color-bottom panel) and albino F0 FSHR mutant female (FSHR−/−, tyr−/−; top panel) of similar body size were dissected for morphological analysis of their gonads. Left images show dissected ovaries in the peritoneal cavity of control and mutant females. The white arrows point to the gonads and the black arrows point to the urogenital papillae. Mutation of FSHR resulted in complete folliculogenesis arrest and atrophic string like gonad. Wild type female displays a large and prominent urogenital papilla while albino F0 FSHR−/−female show a significantly smaller papilla.

FIG. 27 is an illustration showing a germ cell transplantation strategy to allow mass production of donor derived gametes carrying mutations in FEM (cyp17, Cyp19a1a), SMS (TjP1a, Csnk2a2, Gopc, Smap2, Hiat1), MA (Dmrt1, Gsdt and FLS genes (Vtgs, FSHR). In the mutant donor, the defective gene causes the development of monosex male (FEM genes) or female (MA genes) populations or render spermatozoa (SMS genes) or oocytes (FLS genes) non-functional. As such, mass production of these homozygous mutant is not possible. To circumvent this limitation, we only targeted genes whose mutant phenotypes is caused by defective function in the soma and not in germ cells and produced chimeric embryos using the “germ cell transplantation” techniques. To produce chimera, ovarian or testicular cell suspension obtained from juvenile homozygous mutant fish were transplanted into the peritoneal cavity of germ cell-free recipient embryos that are wild type for the targeted gene(s). With this strategy, the wild type host chimeric embryo has normal somatic cells but a mutant germline. These chimeric recipients restore the normal sex ratio and/or sterility as they possess functional somatic gene(s). These recipient fish can be used as commercial broodstock for mass production of monosex and/or sterile fish.

FIG. 28 is an illustration showing a germ cell transplantation method to mass produce functional sperm carrying a spermiogenesis deficient gene (SMS (−)). No defects are found during the generation of primordial germ cells (PGCs) and spermatogonia in SMS-null fish progenies obtained from heterozygous SMS mutant parents. At maturity however, SMS mutant males only produce round headed, immotile sperm and are infertile. Female SMS-mutants are fertile. The SMS gene is expressed in somatic cells surrounding the germ cells (Sertoli and Leydig cells) where it exerts its activity. The lack of SMS protein causes a defective microenvironment where sperm maturation is impaired. To restore spermiogenesis, a germline stem cell can be isolated from juvenile SMS mutant and transplanted into recipient embryos depleted of their own PGCs but carrying a functional SMS gene. Transplanted SMS−/−spermatogonial stem cell will colonize the recipient gonad and since SMS is dispensable for their continued development, the recipient somatic cells will nurse transplanted germ cell, restore spermiogenesis and allow production of functional spermatozoa, all of which carrying the mutant SMS gene.

FIG. 29 is an illustration showing a germ cell transplantation method for production of functional eggs carrying a Vitellogenin deficient gene (Vtg (−)). No defects are found during the generation of primordial germ cells (PGCs) and oogonia in Vtg-null fish progenies obtained from heterozygous Vtg mutant parents. At maturity however, Vtg mutant female only produce oocyte lacking Vtg protein resulting in female sterility. Vtg deficient male develop normally and are fertile. The Vtg gene(s) are normally expressed in liver cells and Vtg protein(s) transported to the oocyte through the blood stream. The lack of Vtg protein cause the eggs to lack critical nutrient necessary to sustain early embryo or larvae development, resulting in developmental arrest. As such, Vtg−/− female are child-less. To restore vitellogenesis, a germline stem cell can be isolated from juvenile Vtg null-mutant and transplanted into recipient embryos depleted of their own PGCs but carrying a functional Vtg gene. Transplanted Vtg−/− germline stem cell will colonize the recipient gonad and the liver cells of the surrogate mother will ensure that nutrients supporting early development are properly loaded into the eggs. These recipient females crossed with Vtg−/− male will produce viable Vtg−/− offspring.

FIG. 30 is an illustration showing a germ cell transplantation method for production of viable FSHR-mutant eggs (FSHR (−)). No defects are found during the generation of primordial germ cells (PGCs) and oogonia in FSHR-null fish progenies obtained from heterozygous FSHR mutant parents. At maturity however, FSHR mutant female fail to respond to FSH-mediated signaling, resulting in folliculogenesis arrest and female. FSHR knock-out males develop normally and are fertile. Since FSHR is solely expressed in somatic follicular cells, transplantation of germline stem cells from juvenile FSHR null-mutant into recipient embryos depleted of their own PGCs but carrying a functional FSHR gene will restore normal oocyte development and allow production of viable eggs. These recipient females crossed with FSHR (−/−) males will only produce FSHR (−/−) offspring.

FIG. 31 is an illustration showing a germ cell transplantation method for production of functional FEM-mutant eggs (FEM: Cyp19a1a, and cyp17). We found no defects during the generation of primordial germ cells (PGCs) and oogonia in FEM-null fish progenies obtained from heterozygous FEM mutant parents. At maturity however, FEM mutant female do not convert androgen into estrogen resulting in reprograming of ovarian somatic supporting cells (Thecal and granulosa cells) into testicular somatic supporting cells (Leydig and Sertoli cells) and reversion of genetic female into phenotypic male. FEM deficient male develop normally and are fertile. The FEM gene(s) are normally expressed in ovarian somatic cells. To allow mass production of oocytes carrying FEM deficient gene, a germline stem cell can be isolated from juvenile FEM null-mutant and transplanted into recipient embryos depleted of their own PGCs but carrying a functional FEM gene. Transplanted FEM germline cells will colonize the recipient gonad. The somatic cells surrounding the donor oocyte will produce normal amount of estrogen allowing progression of folliculogenesis and maintenance of female fate. These recipient females crossed with FEM (−/−) males will produce only FEM−/− offspring.

FIG. 32 is a schematic representation of a strategy to mass-produce all male sterile fish population. Double KO parents (e.g. SMS and cyp17) can be propagated by germ cell transplantation technique as described in FIGS. 27-32. These broodstock parents only produce donor derived gametes carrying the mutated genes. Natural or artificial mating of this broodstock only produce an all-male sterile population.

FIG. 33 panels A and B show a germ cell transplantation experiment demonstrating successful colonization and production of donor derived tilapia gametes. FIG. 33 panel A show a graphical illustration of germ cell transplantation into newly hatched germ cell free tilapia larvae. Donor spermatogonial stem cells (SSCs) carrying mutations were transplanted into the peritoneal cavity of the hatchling depleted of endogenous germ cells. Two groups of SSCs were transplanted simultaneously, one carrying an in frame Δ3nt deletion in the reference gene and a 6 nt insertion in the pigment gene (tyr^{i6/i6}) and the other carrying an out of frame 4 nt deletion in the reference gene and a 22 deletion in the pigment gene (tyr^{Δ22/Δ22}). The 3 nt deletion is not expected to alter the gene function and thus, served as positive control. The transplanted cells migrate and colonize the genital ridges of the recipient. After attaining sexual maturation, the recipient fish gametes were collected, and their DNA analyzed by PCR fragment sizing assay utilizing PCR primers that flank the mutation region of donor derived gamete. The amplification products were sized and detected using capillary electrophoresis. The percentage of female and male recipients producing functional eggs and sperm derived from donor cells after the transplantation of spermatogonial stem cells were provided. FIG. 33 panel B shows capillary fragment length analysis of sperm DNA from a wild type control and from a transplanted fertile tilapia. The bottom trace show only donor derived Δ3nt and Δ4nt deletion fragments from the reference gene, together with a 6nt insertion and Δ22nt deletion fragment in the pigment gene. A negative control with wild-type sized gene specific fragments (268 bp) for the test gene and 467nt for the tyr gene is shown for reference.

FIG. 34 panels A to D are illustrations showing different methods for propagating monosex sterile populations. FEM−/− and MA−/− represent femaleness and maleness null genes. SMS−/− and FLS−/− represent spermiogenesis and folliculogenesis null genes. Males and females Seedstock are produced thru steroid hormone manipulation and by germ cell transplantations (FIG. 34 panels A and B) of thru gem cell transplantation only (FIG. 34 panels C and D). A limited number of seedstock can be crossed to mass-produce millions of all-male sterile embryos (FIG. 34 panels A and C) or all-female sterile embryos (FIG. 34 panels B and D) for use in aquaculture systems.

### DETAILED DESCRIPTION

Generally, the present disclosure provides a method of generating a sterile sex-determined fish, crustacean, or mollusk. The method comprises the steps of: breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation; and selecting a progenitor that is homozygous by genotypic selection, the homozygous mutated progenitor being the sterile sex-determined fish, crustacean, or mollusk. The first mutation disrupts one or more genes that specify sexual differentiation. The second mutation disrupts one or more genes that specify gamete function.

The present disclosure also provides a method of generating a sterile sex-determined fish, crustacean, or mollusk. The method comprises the step of: breeding (i) a fertile homozygous mutated female fish, crustacean, or mollusk having at least a first mutation and a second mutation with (ii) a fertile homozygous mutated male fish, crustacean, or mollusk having at least the first mutation and the second mutation to produce the sterile sex-determined fish, crustacean, or mollusk. The first mutation disrupts one or more genes that specify sexual differentiation. The second mutation disrupts one or more genes that specify gamete function. The fertility of the fertile homozygous female fish, crustacean, or mollusk and the fertile homozygous mutated male fish, crustacean, or mollusk having been rescued.

The present disclosure also provides a method of generating a sterile sex-determined fish, crustacean, or mollusk. The method comprises the step of: breeding (i) a fertile female fish, crustacean, or mollusk having a homozygous mutation with (ii) a fertile male fish, crustacean, or mollusk having a homozygous mutation to produce the sterile sex-determined fish, crustacean, or mollusk. The mutation directly or indirectly disrupts spermiogenesis, and/or that directly disrupts vitellogenesis. The fertility of the fertile female fish, crustacean, or mollusk and the fertile male fish, crustacean, or mollusk have been rescued.

The present disclosure also provides method of making a fertile homozygous mutated fish, crustacean, or mollusk that generates a sterile sex-determined fish, crustacean, or mollusk. The method comprises the steps of: breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation; selecting a progenitor that is homozygous by genotypic selection; and rescuing the fertility of the homozygous progenitor. The first mutation disrupts one or more genes that specify sexual differentiation. The second mutation disrupts one or more genes that specify gamete function.

The present disclosure further provides a fertile homozygous mutated fish, crustacean, or mollusk for producing a sterile sex-determined fish, crustacean, or mollusk. The fertile homozygous mutated fish, crustacean, or mollusk having at least a first mutation and a second mutation, where the first mutation disrupts one or more genes that specify sexual differentiation, and the second mutation disrupts one or more genes that specify gamete function. The fertility of the fertile homozygous mutated fish, crustacean, or mollusk having been rescued.

The present disclosure further provides a fertile fish, crustacean, or mollusk having a homozygous mutation for producing a sterile sex-determined fish, crustacean, or mollusk, wherein the mutation directly or indirectly disrupts spermiogenesis, and/or directly disrupts vitellogenesis, and wherein the fertility of the fertile fish, crustacean, or mollusk has been rescued.

In the context of the present disclosure, a fish refers to any gill-bearing craniate animal that lacks limbs with digits. Examples of fish are carp, tilapia, salmon, trout, and catfish. In the context of the present disclosure, a crustacean refers to any arthropod taxon. Examples of crustaceans are crabs, lobsters, crayfish, and shrimp. In the context of the present disclosure, a mollusk refers to any invertebrate animal with a soft unsegmented body usually enclosed in a calcareous shell. Examples of mollusks are clams, scallops, oysters, octopus, squid and chitons.

A sterile fish, crustacean, or mollusk refers to any fish, crustacean, or mollusk with a diminished ability to generate progeny through breeding or crossing as compared to its wild-type counterpart; for example, a sterile fish, crustacean, or mollusk may have an about 50%, about 75%, about 90%, about 95%, or 100% reduced likelihood of producing viable progeny. In contrast, a fertile fish, crustacean, or mollusk refers to any fish, crustacean, or mollusk that possesses the ability to produce progeny through breeding or crossing. Breeding and crossing refer to any process in which a male species and a female species mate to produce progeny or offspring.

A sex-determined fish, crustacean, or mollusk refers to any fish, crustacean, or mollusk progenitor in which the sex of the progenitor has been pre-determined by disrupting the progenitor's sexual differentiation pathway. In some examples, sex-determined progenitor of the same generation are monosex.

Gamete function refers to the process in which a gamete fuses with another gamete during fertilization in organisms that sexually reproduce.

A mutation that disrupts one or more genes that specify sexual differentiation refers to any genetic mutation that directly or indirectly modulates gonadal function. Directly or indirectly affecting gonadal function refers to: (1) mutating the coding sequence of one or more gonadal genes; (2) mutating a non-coding sequence that has at least some control over the transcription of one or more gonadal genes; (3) mutating the coding sequence of another gene that is involved in post-transcriptional regulation of one or more gonadal genes; or (4) a combination thereof, to modulate gonadal function. Modulating gonadal function refers to specifying that the gonad produces female gametes or produces male gametes. Examples for when masculinization is preferred include modulating one or more genes that modulate the synthesis of androgen and/or estrogen, for example, modulating the expression of aromatase Cyp19a1a, Cyp17, or a combination thereof. Genes involved in modulating the expression of aromatase Cyp19a1a include cyp19ala, FoxL2, sf1 (steroidogenic factor 1), and an ortholog thereof. Genes involved in modulating the expression of Cyp17 include cyp17l or an ortholog thereof. Examples for when feminization is preferred include modulating one or more genes that modulate the expression of an aromatase Cyp19a1a inhibitor. Genes involved in modulating the expression of an aromatase Cyp19a1a inhibitor include Gsdf, dmrt1, Amh, Amhr, and an ortholog thereof.

Alternatively, sexual differentiation may be specified without one or more genetic mutations. Examples of non-genetic mutational methods of specifying sexual differentiation include utilizing sex reversal (hormonal manipulation) and breeding, progeny testing, androgenesis, and gynogenesis, which can produce monosex male or female populations that are homozygous XX, YY or ZZ (see for example [21]; Dunham 2004, which is incorporated by reference). In some examples according to the present disclosure, the step of breeding (i) a fertile female fish, crustacean, or mollusk having a homozygous mutation with (ii) a fertile male fish, crustacean, or mollusk having a homozygous mutation to produce the sterile sex-determined fish, crustacean, or mollusk comprises a non-genetic mutational method of specifying sexual differentiation. In some examples according to the present disclosure using Atlantic salmon, creating and crossing a neomale (XX) with a female produces a monosex progeny of females. In another example according to the present disclosure, specifying sexual differentiation can be achieved by interspecific hybridization (see for example Pruginin, Rothbard et al. 1975, Wolters and DeMay 1996, which is incorporated by reference).

A mutation that disrupts one or more genes that specify gamete function refers to any genetic mutation that directly or indirectly modulates spermiogenesis, oogenesis, and/or folliculogenesis to produce a sterile fish, crustacean, or mollusk. Directly or indirectly modulating spermiogenesis, oogenesis, and/or folliculogenesis refers to: (1) mutating the coding sequence of one or more gamete genes; (2) mutating a non-coding sequence that has at least some control over the transcription of one or more gamete genes; (3) mutating the coding sequence of another gene that is involved in post-transcriptional regulation of one or more gamete genes; or (4) a combination thereof, to produce a sterile fish, crustacean, or mollusk.

A mutation that directly or indirectly disrupts spermiogenesis, and/or directly disrupts vitellogenesis refers to any genetic mutation that directly or indirectly modulates spermiogenesis, and/or directly disrupts vitellogenesis to produce a sterile fish, crustacean, or mollusk. Directly or indirectly modulating spermiogenesis refers to: (1) mutating the coding sequence of one or more gamete genes involved in spermiogenesis; (2) mutating a non-coding sequence that has at least some control over the transcription of one or more gamete genes involved in spermiogenesis; (3) mutating the coding sequence of another gene that is involved in post-transcriptional regulation of one or more gamete genes involved in spermiogenesis; or (4) a combination thereof, to produce a sterile fish, crustacean, or mollusk. Directly modulating vitellogenesis refers to: (1) mutating the coding sequence of one or more gamete genes involved in vitellogenesis; (2) mutating a non-coding sequence that has at least some control over the transcription of one or more gamete genes involved in vitellogenesis; or (3) a combination thereof, to produce a sterile fish, crustacean, or mollusk.

Examples for when producing a sterile male fish, crustacean, or mollusk is preferred include modulating one or more genes that modulate spermiogenesis. Examples of one or more genes that modulate spermiogenesis may cause globozoospermia, sperm with round-headed, round nucleus, disorganized midpiece, partially coiled tails, or a combination thereof. Examples of genes that cause globozoospermia include Gopc, Hiat1, Tjp1a, Smap2, Csnk2a2, and an ortholog thereof. Examples for when producing a sterile female fish, crustacean, or mollusk is preferred include modulating one or more genes that modulate oogenesis, folliculogenesis, or a combination. Examples of one or more genes that modulate oogenesis include one or more genes that modulate the synthesis of estrogen. Examples of one or more genes that modulate the synthesis of estrogen include FSHR or an ortholog thereof. Examples of one or more genes that modulate folliculogenesis include one or more genes that modulate the expression of vitellogenins. Examples of one or more genes that modulate the expression of vitellogenins include vtgs or an ortholog thereof. Examples of mutations that directly or indirectly disrupt spermiogenesis are mutations in Gopc, Hiat1, Tjp1a, Smap2, Csnk2a2, or an ortholog thereof. Examples of mutations that directly disrupts vitellogenesis are mutations in a gene encoding or regulating: Vitellogenin; Estrogen receptor1; cytochrome p450, family 1, subfamily a; Zona pellucida glycoprotein; Choriogenin H; Peroxisome proliferator-activated receptor; Steroidogenic acute regulatory protein, or an ortholog thereof.

A mutation may be any type of alteration of a nucleotide sequence of interest, for example, nucleotide insertions, nucleotide deletions, and nucleotide substitutions.

Rescuing sterility or fertility refers to any process in which a sterile fish, crustacean, or mollusk is converted into a fertile fish, crustacean, or mollusk. In some examples, an aromatase inhibitor is provided to the sterile fish, crustacean, or mollusk to restore fertility. In other examples, germline stem cell transplantation of the sterile fish, crustacean, or mollusk restores fertility. Germline stem cell transplantation refers to any process in which reproductive stem cells from a sterile fish, crustacean, or mollusk is transplanted into a fertile fish, crustacean, or mollusk and restores fertility. In some examples according to the present disclosure, the germline stem cell transplantation is a process comprising: obtaining a germline stem cell from a sterile homozygous male fish, crustacean, or mollusk having at least the first mutation and the second mutation or a germline stem cell from a sterile homozygous female fish, crustacean, or mollusk having at least the first mutation and the second mutation; and transplanting the germline stem cell into a germ cell-less recipient male fish, crustacean, or mollusk, or into a germ cell-less recipient female fish, crustacean, or mollusk. A recipient male or female fish, crustacean, or mollusk is any embryo depleted of their own germ cells but carrying functional copies of genes targeted that specify sexual differentiation and gamete function. Alternatively, the germ cell depleted recipient can be a juvenile or adult fish carrying functional copies of genes targeted. Preferably, the recipient species is the same as the donor species (allogenic recipient) but other species may be used (Xenogeneic recipient). The recipient after transplantation is a chimeric fish, crustacean or mollusk with normal somatic cells but a mutant germline. These chimeric recipients restore the normal sex ratio and/or sterility as they possess functional somatic gene(s). A germ cell-less recipient may be created using ploidy manipulation, hybridization strategies, or exposure to high levels of sex hormones. Exposure of juvenile aquatic species to high levels of sex hormones may result in sterility in the exposed animals. This technique has been demonstrated (Hunter et al, 1982; Solar et al, 1984; Piferrer et al, 1994), but has not been used at a commercial scale. While the technique may be effective in creating sterile fish, it has never been demonstrated effective at inducing sterility in 100% of the treated fish. Treated fish may be suitable for research, or as recipients for germ cell transfer, but the technique may not be adequate for creating sterile fish for commercial farming (see also Hunter, G. A., E. M. Donaldson, F. W. Goetz, and P. R. Edgell. 1982. Production of all-female and sterile Coho salmon, and experimental evidence for male heterogamety. Transactions of the American Fisheries Society 111: 367-372; Piferrer, F, M Carillo, S. Zanuy, I. I. Solar, and E. M. Donaldson. 1994. Induction of sterility in Coho salmon (Oncorhynchus kisutch) by androgen immersion before first feeding. Aquaculture 119: 409-423; and Solar, I., E. M. Donaldson, and G. A. Hunter. 1984. Optimization of treatment regimes for controlled sex differentiation and sterilization in wild rainbow trout (Salmo gairdeneri Richardson) by oral administration of 17α-methyltestosterone. Aquaculture 42: 129-139.

In some examples, the germline stem cell transplantation is a process comprising: obtaining a spermatogonial stem cell from a sterile homozygous male fish, crustacean, or mollusk or a oogonial stem cell from a sterile homozygous female fish, crustacean, or mollusk, and transplanting the spermatogonial stem cell into the peritoneal cavity of a germ cell-less embryo or into a germ cell-less differentiated testis or ovary of a fish, crustacean, or mollusk. Optionally, in addition to germline stem cell transplantation, an exogenous sex steroid is provided to the sterile fish, crustacean, or mollusk, for example, estrogen to restore fertility. In other examples, an aromatase inhibitor is provided to the sterile fish, crustacean, or mollusk to restore fertility.

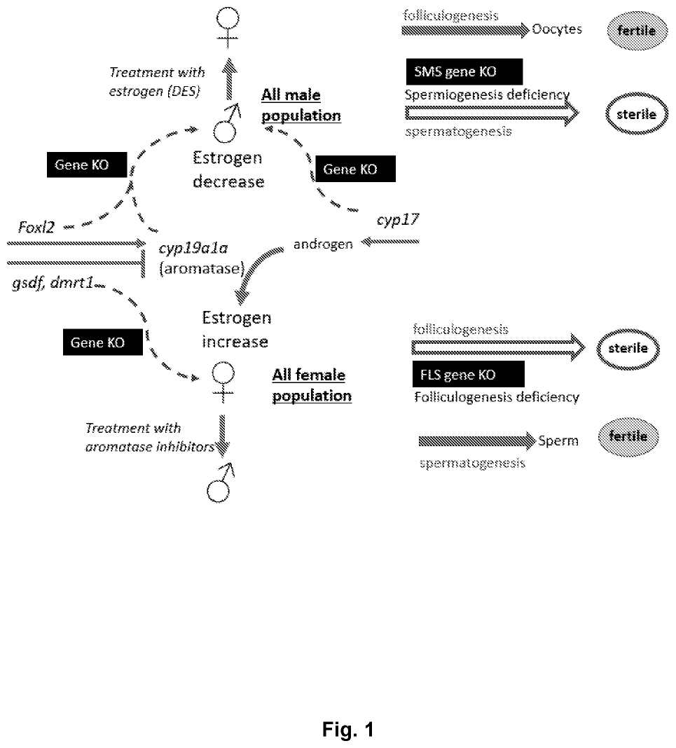

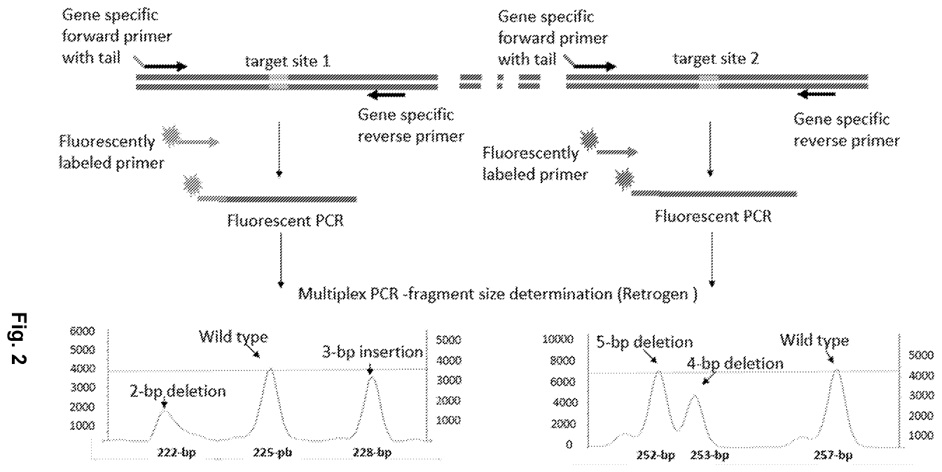

FIG. 1 illustrates a flowchart according to the present disclosure of how to make a male and female broodstock, i.e. a fertile homozygous mutated male and female fish, crustacean, or mollusk for use in producing a sterile sex-determined fish, crustacean, or mollusk.

FIG. 1 illustrates genetic pathways governing sex differentiation and gametogenesis and gene KO strategies to produce monosex sterile populations.

One or more mutations in the gene cyp19a1a, Foxl2, or a combination thereof, results in low or decreased estrogen expression causing testis formation and the production of a male fish, crustacean, or mollusk. Similarly, one or more mutations in the gene cyp17 results in low or decreased estrogen and androgen expression producing a male fish, crustacean, or mollusk. One or more additional mutations in a gene that disrupts spermiogenesis (SMS) causes the male fish, crustacean, or mollusk to be sterile. Accordingly, a sterile homozygous mutated male fish, crustacean, or mollusk is produced.

In an additional step used to propagate the line, the fertility of the sterile homozygous mutated male fish, crustacean, or mollusk may be rescued with treatment of estrogen. Following treatment, a fertile homozygous mutated female fish, crustacean, or mollusk is generated. In this sex reversal process, the phenotypic female is carrying the one or more mutations disrupting spermiogenesis and should be fertile, and oocytes carrying the one more mutations disrupting spermiogenesis should be produced and allow for propagation of the line. Alternatively, and as described in Example 10, the fertility of the sterile homozygous mutated male fish, crustacean, or mollusk may be rescued by implanting a germ cell from the sterile homozygous mutated male fish, crustacean, or mollusk into a fertile wild-type male testis cell to generate a fertile homozygous mutated male fish, crustacean, or mollusk, which allows for propagation of the line.

On the flip side of FIG. 1, one or more mutations in the gene Gsdf, Dmrt1, or a combination thereof, results in inactivation of Cyp19a1a inhibitors and causes high or increased estrogen expression resulting in ovarian formation and the production of a female fish, crustacean, or mollusk. One or more additional mutations in a gene that modulates oogenesis, folliculogenesis (FLS), or a combination thereof causes the female fish, crustacean, or mollusk to be sterile. Accordingly, a sterile homozygous mutated female fish, crustacean, or mollusk is produced.

In an additional step used to propagate the line, the fertility of the sterile homozygous mutated female fish, crustacean, or mollusk may be rescued with treatment of an aromatase inhibitor. Following treatment, a fertile homozygous mutated male fish, crustacean, or mollusk is generated. In this sex reversal process, the phenotypic male is carrying the one or more mutations disrupting oogenesis, folliculogenesis, or a combination and should be fertile, and sperm carrying the one more mutations disrupting oogenesis, folliculogenesis, or a combination should be produced and allow for propagation of the line. Alternatively, and as described in Example 10, the fertility of the sterile homozygous mutated female fish, crustacean, or mollusk may be rescued by implanting a germ cell from the sterile homozygous mutated female fish, crustacean, or mollusk into a fertile wild-type female ovary cell to generate a fertile homozygous mutated female fish, crustacean, or mollusk, which allows for propagation of the line.

### EXAMPLES

### Example 1—Materials and Methods

Animal used and ethical statement: All experiments complied with US regulations ensuring animal welfare and animal husbandry procedures were performed according to IACUC-approved animal protocol CAT-004. Tilapia (Oreochromis niloticus) lines used in this study are derived from a Brazilian strain obtained from a US commercial producer.

Generation of nucleases and strategies: Generation of F0 mutants: Tilapia orthologs of the cyp17, Cyp19a1a, Tjp1a, Csnk2a2, Hiat1, Smap2, Gopc, Gsdf, Dmrt1, FSHR and vitellogenin genes (VtgAa and VtgAb) were identified in silico from genomic databases.

To create DNA double strand breaks (DSBs) at specific genomic site, we used engineered nucleases. In most applications, a single DSB was produced in the absence of a repair template, leading to the activation of the non-homologous end joining (NHEJ) repair pathway. The NHEJ can be an imperfect repair process, generating insertions or deletions (indels) at the target site. Introduction of an indel can create a frameshift within the coding region of the gene resulting in abnormal protein products with an incorrect amino acid sequence. To enhance the frequency of generating null mutations in the gene of interest, we targeted 2 separate exons simultaneously apart from those targeting cyp17. Alongside the gene of interest, we co-targeted a pigmentation gene to serve as a mutagenesis selection marker. Typically, mutagenic frequency between the pigment gene and the gene of interest are correlated. Thus, embryos showing complete lack of pigmentation (albino phenotype) were preferentially selected compare to mosaic pigment phenotype (partial gene inactivation). To confirm functionality of the newly designed nuclease, five albino embryos from each treated batch were quantitatively assayed for genome modifications at the loci of interest by PCR fragment analysis. Treated embryos of the same batch were eliminated if all five embryos tested showed no indels at the targeted loci. Furthermore, we preferentially raised batches of embryos in which mutations are produced at the one or two cell stage, (i.e. detection of 2 or 4 mutant alleles per targeted loci by fragment analysis assay).

The template DNA coding for the engineered nuclease were linearized and purified using a DNA Clean & concentrator-5 column (Zymo Resarch). One microgram of linearized template was used to synthesize capped RNA using the mMESSAGE mMACHINE T3 kit (Invitrogen), purified using Qiaquick (Qiagen) columns and stored at −80° in RNase-free water at a final concentration of 800 ng/μl.

Embryo injections: Embryos were produced from in vitro fertilization. Approximately 10 nL total volume of solution containing the programmed nucleases were co-injected into the cytoplasm of one-cell stage embryos. Injection of 200 embryos typically produce 10-60 embryos with complete pigmentation defect (albino phenotype). Embryo/larvae survival was monitored for the first 10-12 days post injection.

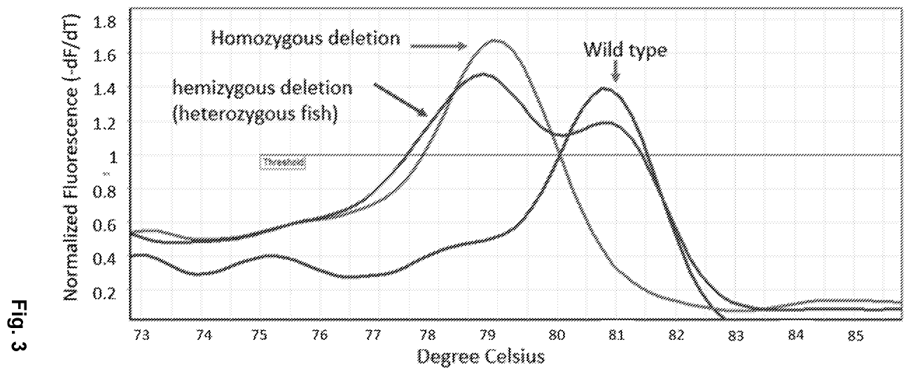

Selection of founders: A minimum of 10 albino embryos were raised to 3 months of age and quantitatively assayed for genome modifications by fluorescence PCR fragment analysis (see Table 1 for gene specific genotyping primers columns 8 and 11). We preferentially selected founders in which mutations were produced at the one or two cell stage (detection of 2 or 4 mutant alleles per target loci by fragment analysis (FIG. 2).

F1 genotyping: The selected founders were outcrossed with wild-type lines. Their F1 progeny were raised to 2 months of age, anesthetized by immersion in 200 mg/L MS-222 (tricaine) and transferred onto a clean surface using a plastic spoon. Their fin was clipped with a razor blade, and place onto a well (96 well plate with caps). Fin clipped fish were then placed in individual jars while their fin DNA was analyzed by fluorescence PCR. In brief, 60 μl of a solution containing 9.4% Chelex and 0.625 mg/ml proteinase K was added to each well for overnight tissue digestion and gDNA extraction in a 55° C. incubator. The plate was then vortexed and centrifuged. gDNA extraction solution was then diluted 10× with ultra-clean water to remove any PCR inhibitors in the mixture. Typically, we analyzed 80 juveniles/founder to select and raised batches of approximately 20 juveniles carrying identical size mutations.

Fluorescence PCR (see FIG. 2): PCR reactions used 3.8 μL of water, 0.2 μL of fin-DNA and 5 μL of PCR master mix (Quiagen Multiplex PCR) with 1 ul of primer mix consisting of the following three primers: the Labeled tail primer with fluorescent tag (6-FAM, NED), amplicon-specific forward primer with forward tail (SEQ ID NO: 117: 5′-TGTAAAACGACGGCCAGT-3′ and SEQ ID NO: 118: 5′-TAGGAGTGCAGCAAGCAT-3′) amplicon-specific reverse primer (Fluorescent PCR gene-specific primers are listed in Table 1). PCR conditions were as follows: denaturation at 95° C. for 15 min, followed by 30 cycles of amplification (94° C. for 30 sec, 57° C. for 45 sec, and 72° C. for 45 sec), followed by 8 cycles of amplification (94° C. for 30 sec, 53° C. for 45 sec, and 72° C. for 45 sec) and final extension at 72° C. for 10 min, and an indefinite hold at 4° C.

One-two microliters of 1:10 dilution of the resulting amplicons were resolved via capillary electrophoresis (CE) with an added LIZ labeled size standard to determine the amplicon sizes accurate to base-pair resolution (Retrogen Inc., San Diego). The raw trace files were analyzed on Peak Scanner software (ThermoFisher). The size of the peak relative to the wild-type peak control determines the nature (insertion or deletion) and length of the mutation. The number of peak(s) indicate the level of mosaicism. We selected F0 mosaic founder carrying the fewest number of mutant alleles (2-4 peak preferentially).

The allele sizes were used to calculate the observed indel mutations. Mutations that are not in multiples of 3 bp and thus predicted to be frameshift mutations were selected for further confirmation by sequencing. Mutations of size greater than 8 bp but smaller than 30 bp were preferentially selected to ease genotyping by QPCR melt analysis for subsequent generations. For sequence confirmation, the PCR product of the selected indel was further submitted to sequencing. Sequencing chromatography of PCR showing two simultaneous reads are indicative of the presence of indels. The start of the deletion or insertion typically begins when the sequence read become divergent. The dual sequences were carefully analyzed to detect unique nucleotide reads. The pattern of unique nucleotide read is then analyzed against series of artificial single read patterns generated from shifting the wild type sequence over itself incrementally.

QPCR genotyping of F1 and F2 generations: Real-time qPCR was performed on a ROTOR-GENE RG-3000 REAL TIME PCR SYSTEM (Corbett Research). 1-μL genomic DNA (gDNA) template (diluted at 5-20 ng/μl) was used in a total volume of 10 μL containing 0.15 μM concentrations each of the forward and reverse primers and 5 μL of QPCR 2× Master Mix (Apex Bio-research products). qPCR primers used are presented in Table 2 (Genotyping RT-PCR primers columns 11-14). The qPCR was performed using 40 cycles of 15 seconds at 95° C., 60 seconds at 60° C., followed by melting curve analysis to confirm the specificity of the assay (67° C. to 97° C.). In this approach, short PCR amplicons (approx 120-200 bp) that include the region of interest are generated from a gDNA sample, subjected to temperature-dependent dissociation (melting curve). When induced indels are present in hemizygous gDNA, heteroduplex as well as different homoduplex molecules are formed. The presence of multiple forms of duplex molecules is detected by Melt profile, showing whether duplex melting acts as a single species or more than one species. Generally, the symmetry of the melting curve and melting temperature infers on the homogeneity of the dsDNA sequence and its length. Thus, homozygous and wild type (WT) show symmetric melt curved that are distinguishable by varied melting temperature. The Melt analysis was performed by comparison with reference DNA sample (from control wild type DNA) amplified in parallel with the same master mix reaction. In short, variation in melt profile distinguishes amplicons generated from homozygous, hemizygous and WT gDNA (see FIG. 3).

Assessment of sterility in males: The volume of strippable sperm and sperm density was measured from 10 males (5 months of age) for each genotype. Sperm were counted using a Neubauer hemocytometer slide, as well as by spectrophotometry (optical density (O.D) at 600 nm) of serially diluted samples. Sperm motility was measured in terms of percent motile spermatozoa in field of view [4]. Morphology of the sperm cells stained with eosin-nigrosin was analyzed under light microscopy at 400×. Fertilization capacity of sperm was assayed by in vitro fertilization of wild type eggs from 3 different females at the optimal sperm to egg ratio (100 eggs for 5·10⁶ spermatozoa). Wild type egg quality was tested in parallel using sperm from WT males. Fertilization rates was expressed as a percentage of surviving embryos to total eggs collected at 24 hrs post fertilization. The mean values obtained from these studies was compared across mutant genotypes using an unpaired t-test.

Assessment of sterility in females: We recorded the body weight of all fish sampled. A minimum of six females for each genotype was dissected at 4 and 6 months of age and their gonads photographed in situ before dissection. The mean total gonadosomatic index was statistically compared across all genotypes (unpaired T-test). Survival of eggs, embryos and larvae produced from a minimum of six mutant females outcrossed with wild-type males were statistically analyzed (unpaired T-test) and compared to controls (wild-type females crossed with mutant males).

Donor cell isolation and germ cell transplantation: Germ cell stem cells were harvested from the gonads of 3-4 months old fish (˜50-70 g) through enzymatic digestion as described by Lacerda [5]. In brief, the freshly isolated gonads were minced and incubated in 1 ml of 0.5% trypsin (Worthington Biochemical Corp., Lakewood, N.J.) in PBS (pH 8.2) containing 5% fetal bovine serum (Gibco Invitrogen Co., Grand Island, N.Y.) and 0.05% DNase I (Roche Diagnostics, Mannheim, Germany) for 3-4 h at 25° C. During incubation, gentle pipetting was applied to physically disrupt any remaining intact portions of the gonads. The resulting cell suspension was filtered through a nylon screen with a pore size of 42 μm (N-No. 330T; Tokyo Screen Co. Ltd., Tokyo, Japan) to remove any undissociated cell clumps and then resuspended in L-15 medium (Gibco Invitrogen Co.) before storage on ice until transplantation.

Germ cell-free recipient larvae (5-7dpf) were anesthetized with 0.0075% ethyl 3-aminobenzoate methanesulfonate salt (Sigma-Aldrich Inc.) and transferred to a Petri dish coated with 2% agar. Cell transplantation was performed by injecting approximately 15,000 testicular cells into the peritoneal cavity of approximately 80 larvae progeny from Elavl2 hemizygous mutant parents. Alternatively, PGC-free embryos were obtained from a cross between MSC homozygous female and wild type male [6]. After transplantation, recipient larvae were transferred back to aerated embryo hatching water and raised to adulthood.

TABLE 1
Primers

| | Tilapia | | | | | | | | | | |
|---|---|---|---|---|---|---|---|---|---|---|---|
| | homolog | | | | Forward | SEQ | | Reverse | SEQ | | Amplicon |
| | gene | NCBI & Ensembl | Targeted | | primer | ID | | primer | ID | | size |
| full gene name | (alias) | Accession # | exon | Label | exon | NO | forward primer | exon | NO | Reverse primer | (bp) |
| cytochrome | Cyp17 | Acc: ZDB-GENE- | 1 | FAM | 5′UTR | SEQ | ttgaagttgctacataaaag | 1 | SEQ | TGGTTGATGACAATCAC | 357 |
| P450, family 17, | | 040213-2 | | | | 1 | | | 2 | ACTGT | |
| subfamily A, | | ENSONIG00000 | 1 | FAM | 5′UTR | SEQ | ttgaagttgctacataaaag | 1 | SEQ | TGGTTGATGACAATCAC | 357 |
| polypeptide 1 | | 009168 | | | | 1 | | | 2 | ACTGT | |
| cytochrome | Cyp19 | Acc: NM_001279 | 1 | NED | 5′UTR | SEQ | tgttctacatcatcacccttctc | 1 | SEQ | AGCAGACAGACGAGCA | 169 |
| P450, family 19, | a1a | 586 | | | | 3 | | | 4 | GTATCAG | |
| subfamily A, | | ENSONIG00000 | 9 | FAM | 9 | SEQ | TGATGGAGAGCTTCATC | 9 | SEQ | GTTCCAGGTTAAATTGA | 186 |
| polypeptide 1a | | 000155 | | | | 5 | TACGAA | | 6 | TTG | |
| gene | | | | | | | | | | | |
| Tight junction | Tjp1a | Acc: ZDB-GENE- | 15 | NED | Intron | SEQ | gcgtgatttgctgacctttttac | 15 | SEQ | acacttacCCTGAGAATCT | 216 |
| protein 1a | | 031001-2 | | | 14-15 | 7 | | | 8 | GG | |
| | | ENSONIG00000 | 17 | FAM | 16 | SEQ | GAAAAAGGATGgtgaggg | 17 | SEQ | GAGTGTGTCTACCACAC | 239 |
| | | 006221 | | | | 9 | atgac | | 10 | GGAAAA | |
| Casein Kinase II | Csnk2a | Acc: 100690588 | 1 | FAM | 5′UTR | SEQ | gtatttagaaggcggtgaaggt | 1 | SEQ | CAGTTTGGCACATGAGC | 153 |
| subunit alpha | 2 | | | | | 11 | c | | 12 | ATCGTA | |
| | | ENSONIG00000 | 2 | NED | 1 | SEQ | ATGCTCATGTGCCAAAC | 2 | SEQ | cCTTCAGGATTTTCACCA | 222 |
| | | 015598 | | | | 13 | TG | | 14 | CCACT | |
| Hippocampus | Hiat1 | Acc: 100705862 | 4 | FAM | Intron | SEQ | tactgacacatccagcagcgtc | Intron | SEQ | cagcactgagccgtcagtattc | 211 |
| abundant | | | | | 3-4 | 15 | t | 4-5 | 16 | t | |
| transcript 1a | | ENSONIG00000 | 6 | NED | 6 | SEQ | TGGAGCCTACCTGTCTG | 6 | SEQ | tactcacAGCGAAGGGGT | 182 |
| | | 018605 | | | | 17 | AG | | 18 | CT | |
| small ArfGAP2 | Smap2 | Acc: ZDB-GENE- | 2 | NED | Intron | SEQ | gctcctctgcgaagactctc | 2 | SEQ | aagacctccgacCTGGACT | 211 |
| | | 060503-374 | | | 1-2 | 19 | | | 20 | TGCT | |
| | | ENSONIG00000 | 9 | FAM | 9 | SEQ | AGAGGAGGGCACAGTC | 10 | SEQ | TTGGATATCCCATTTGG | 226 |
| | | 004622 | | | | 21 | AAGAAAC | | 22 | TTCAT | |
| Golgi- | Gopc | Acc: 100692751 | 1 | NED | 5′UTR | SEQ | tttaacggtgttggcagagatt | 1 | SEQ | AGATCCACATCCACGAA | 207 |
| associated PDZ | | | | | | 23 | | | 24 | AGCCT | |
| and coiled-coil | | ENSONIG00000 | 2 | FAM | Intron | SEQ | tgcccctttaaaccaccta | 2 | SEQ | CTCAGCTTGGCCTTGCT | 207 |
| motif | | 001688 | | | 1-2 | 25 | | | 26 | TGACAT | |
| containing gene | | | | | | | | | | | |
| doublesex and | Dmrt1 | Acc: ZDB-GENE- | 1 | NED | 5′UTR- | SEQ | ttgccaggacccATGAGCC | 1 | SEQ | AGACACGTATCCGTGAT | 135 |
| mab-3 related | | 050511-1 | | | 1 | 27 | AG | | 28 | TTCTAC | |
| transcription | | ENSONIG00000 | 3 | FAM | Intron | SEQ | ctcttcatcctctgtgtctcatc | 3 | SEQ | GGGTTTCCAGCAGGAG | 140 |
| factor 1 | | 014201 | | | 2-3 | 29 | | | 30 | GTCAGA | |
| growth/differen- | Gsdf | Acc: 100710262 | 2 | NED | 2 | SEQ | ttatgttcagGTGCCAAGG | 2 | SEQ | TGGCTGTGTGAGAAAC | 156 |
| tiation factor 6- | | | | | | 31 | TG | | 32 | GATGCTG | |
| B-like | | ENSONIG00000 | 4 | FAM | 4 | SEQ | agATCTGGGCTGGGACA | 4 | SEQ | tgttaactatacCTGTGTGT | 145 |
| | | 007633 | | | | 33 | | | 34 | TGG | |
| Follicle | FSHR | Acc: ZDB-GENE- | 11 | NED | Intron | SEQ | ttttctccgcttgcttctgc | 11 | SEQ | AAAGAGCTGAATAGGA | 137 |
| stimulating | | 020423-5 | | | 10-11 | 35 | | | 36 | GGAAGTT | |
| hormone | | ENSONIG00000 | 15 | FAM | 15 | SEQ | CATCTTGGCGTTCTTCTG | 15 | SEQ | CTTGAGGGCAGCTGAG | 181 |
| receptor | | 015917 | | | | 37 | TGT | | 38 | ATGGC | |
| Vitellogenin Aa | VtgAa | Acc: ZDB-GENE- | 7 | NED | 7 | SEQ | GCAATCCTTGATGCTCC | 7 | SEQ | CTGAGACTCTATGTCGT | 163 |
| | | 001201-1 | | | | 39 | TTGAC | | 40 | TGATA | |
| | | ENSONIG00000 | 22 | FAM | 22 | SEQ | AGAAGATCATCAAACAC | 22 | SEQ | GACTTGTTGAGCAGTTG | 227 |
| | | 007355 | | | | 41 | ATCACG | | 42 | CATCAA | |
| Vitellogenin Ab | VtgAb | Acc: ZDB-GENE- | 5 | NED | 5 | SEQ | ttttgtgatctagTCTGGAG | 5 | SEQ | gctcttacAGCTTCACAAT | 183 |
| | | 001201-1 | | | | 43 | | | 44 | CAT | |
| | | ENSONIG00000 | 22 | FAM | 22 | SEQ | CTTCTGGACCAGTCATT | 22 | SEQ | AGACTTGTTGGAGCTA | 227 |
| | | 007369 | | | | 45 | GAG | | 46 | GAG | |

TABLE 2
Primers

| | | Genotyping RT-PCR primers | | | | | | |
|---|---|---|---|---|---|---|---|---|
| | Tilapia | | | | | | | Ampli- |
| | homolog | Forward | SEQ | | Reverse | SEQ | | con |
| | gene | primer | ID | | primer | ID | | size |
| full gene name | (alias) | exon | NO | Forward primer sequence | exon | NO | Reverse Primer sequence | (bp) |
| cytochrome P450, | Cyp17 | 1 | SEQ | GAACCAAACCCCTCTGTCAC | 1 | SEQ | GTAATTCACTCCGCAGGCTCA | 184 |
| family 17, | | | 47 | TG | | 48 | G | |
| subfamily A, | | 1 | SEQ | GAACCAAACCCCTCTGTCAC | 1 | SEQ | GTAATTCACTCCGCAGGCTCA | 184 |
| polypeptide 1 | | | 47 | TG | | 48 | G | |
| cytochrome P450, | Cyp19a1 | 1 | SEQ | ggcgATGAATCCTGTAG | 1 | SEQ | ATGGCATTTGAGGTCACAGAG | 63 |
| family 19, | a | | 49 | | | 50 | A | |
| subfamily A, | | 9 | SEQ | TGATGGAGAGCTTCATCTAC | 9 | SEQ | GTTCCAGGTTAAATTGATTG | 186 |
| polypeptide 1a | | | 5 | GAA | | 6 | | |
| gene | | | | | | | | |
| Tight junction | Tjp1a | 15 | SEQ | GTTCAAGAAGGGAGAGAGT | 15 | SEQ | AAAAATTCCCACATCGTT | 61 |
| protein 1a | | | 51 | | | 52 | | |
| | | 17 | SEQ | tgctttggcttcagTGTATC | 17 | SEQ | AATGCGTTCGAATGTAGAA | 71 |
| | | | 53 | | | 54 | | |
| Casein Kinase II | Csnk2a2 | 5′UTR | SEQ | gtatttagaaggcggtgaaggtc | 1 | SEQ | CAGTTTGGCACATGAGCATCG | 153 |
| subunit alpha | | | 11 | | | 12 | TA | |
| | | 1 | SEQ | ATGCTCATGTGCCAAACTG | 2 | SEQ | cCTTCAGGATTTTCACCACCAC | 222 |
| | | | 13 | | | 14 | T | |
| Hippocampus | Hiat1 | Intron | SEQ | tactgacacatccagcagcgtct | Intron | SEQ | cagcactgagccgtcagtattct | 211 |
| abunda nt | | 3-4 | 15 | | 4-5 | 16 | | |
| transcript 1a | | 6 | SEQ | CATCTGCTTCATCCTGGTGG | 6 | SEQ | tactcacAGCGAAGGGGTCT | 110 |
| | | | 55 | CTG | | 18 | | |
| small ArfGAP2 | Smap2 | 2 | SEQ | AATTTGGGCATCTTCATCTG | 2 | SEQ | GACAGACTTGACCTTGGAGAT | 81 |
| | | | 56 | TAT | | 57 | G | |
| | | 9 | SEQ | AGAGGAGGGCACAGTCAAG | 10 | SEQ | TTGGATATCCCATTTGGTTCAT | 226 |
| | | | 21 | AAAC | | 22 | | |
| Golgi-associated | Gopc | 1 | SEQ | ATGTCTGCTTCGACTGGATG | 1 | SEQ | GCCATCGAAACATGGACATAC | 76 |
| PDZ and coiled-coil | | | 58 | C | | 59 | TG | |
| motif containing | | Intron | SEQ | tgcccctttaaaccaccta | 2 | SEQ | CTCAGCTTGGCCTTGCTTGACA | 207 |
| gene | | 1-2 | 25 | | | 26 | T | |
| doublesex and | Dmrt1 | 5′UTR-1 | SEQ | ttgccaggacccATGAGCCAG | 1 | SEQ | AGACACGTATCCGTGATTTCTA | 135 |
| mab-3 related | | | 27 | | | 28 | C | |
| transcription | | Intron | SEQ | ctcttcatcctctgtgtctcatc | 3 | SEQ | GGGTTTCCAGCAGGAGGTCAG | 140 |
| factor | | 2-3 | 29 | | | 30 | A | |
| growth/differentia- | Gsdf | 2 | SEQ | ttatgttcagGTGCCAAGGTG | 2 | SEQ | TGGCTGTGTGAGAAACGATGC | 156 |
| tion | | | 31 | | | 32 | TG | |
| factor 6-8-like | | 4 | SEQ | agATCTGGGCTGGGACA | 4 | SEQ | tgttaactatacCTGTGTGTTGG | 145 |
| | | | 33 | | | 34 | | |
| Follicle | FSHR | Intron | SEQ | ttttctccgcttgcttctgc | 11 | SEQ | AAAGAGCTGAATAGGAGGAA | 137 |
| stimulating | | 10-11 | 35 | | | 36 | GTT | |
| hormone receptor | | 15 | SEQ | CATCTTGGCGTTCTTCTGTG | 15 | SEQ | CTTGAGGGCAGCTGAGATGGC | 181 |
| | | | 37 | T | | 38 | | |
| Vitellogenin Aa | | 7 | SEQ | GCAATCCTTGATGCTCCTTG | 7 | SEQ | CTGAGACTCTATGTCGTTGATA | 163 |
| VtgAa | | | 39 | AC | | 40 | | |
| | | 22 | SEQ | AGAAGATCATCAAACACATC | 22 | SEQ | GACTTGTTGAGCAGTTGCATC | 227 |
| | | | 41 | ACG | | 42 | AA | |
| Vitellogenin Ab | VtgAb | 5 | SEQ | ttttgtgatctagTCTGGAG | 5 | SEQ | gctcttacAGCTTCACAATCAT | 183 |
| | | | 43 | | | 44 | | |
| | | 22 | SEQ | CTTCTGGACCAGTCATTGAG | 22 | SEQ | AGACTTGTTGGAGCTAGAG | 227 |
| | | | 45 | | | 46 | | |

### Example 2—Use of a Gene Editing Tool to Induce Double-Allelic Knockout in Tilapia F0 Generation

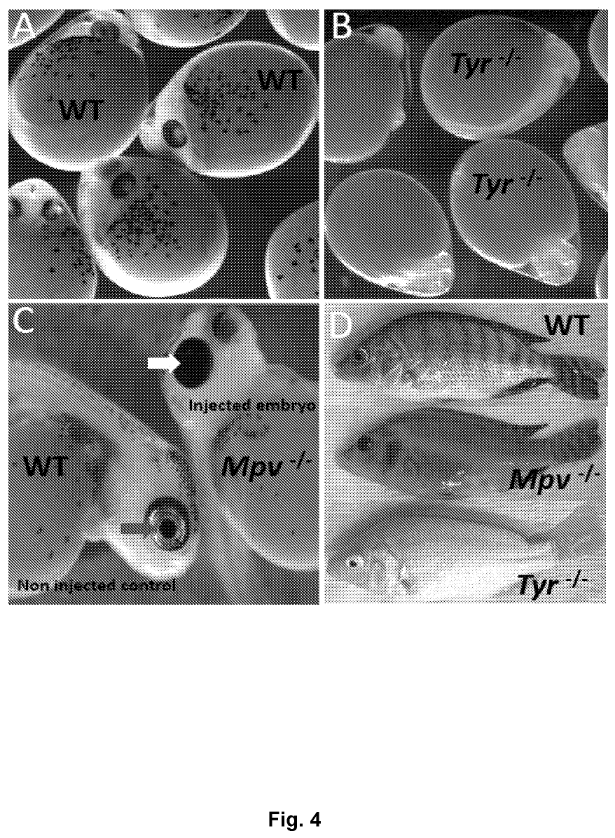

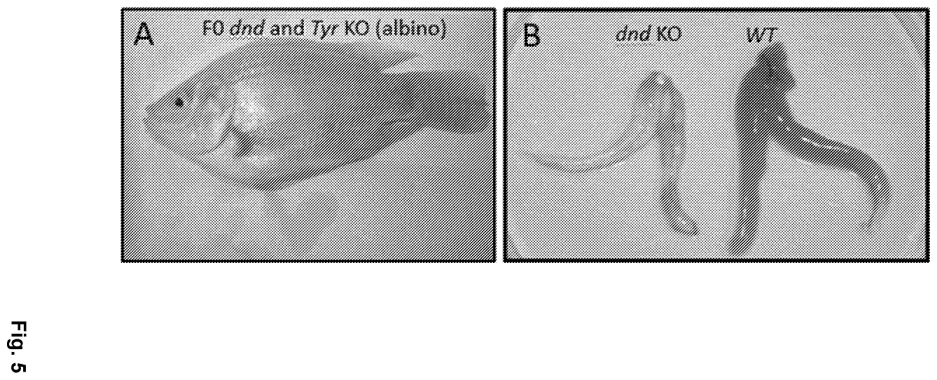

We have independently targeted two genes involved in pigmentation, namely the genes encoding tyrosinase (tyr) [2] and the mitochondrial inner membrane protein MpV17 (mpv17) (Krauss, Astrinides et al. 2013) [8]. We found that 50% and 46% of all injected embryos showed a high degree of mutation at the tyr and mpv17loci respectively (FIG. 4). Loss-of-function alleles cell-autonomously lead to unpigmented melanophores in the embryo body (FIG. 4 panel B) and in the retinal pigment epithelium (FIG. 4 panel C), producing embryonic phenotypes ranging from complete to partial loss of melanine and iridophore pigmentation that are readily identifiable against wild type phenotype (FIG. 4 panels A and C). Embryos showing a complete lack of pigmentation (10-30% of treated fish) were raised to 3 months of age and all lacked wild type tyr and mpv17 sequences. These fish display transparent and albino phenotypes (FIG. 4 panel D), indicating that functional studies can be performed in F0 tilapia.

### Example 3—Multi-Gene Targeting in Tilapia

We tested whether multiple genomic loci can be targeted simultaneously and whether mutagenic efficiency measured at one loci is predictable of mutation at other loci in the tilapia genome. To test our hypothesis, we co-targeted tyr and Dead-end1 (dnd). Dnd is a PGC-specific RNA binding protein (RBP) that maintains germ cell fate and migration ability [3]. Following injection of programmed nucleases, we found that mutations in both gene targets tyr and dnd were highly correlated. Approximately 95% of abino (tyr) mutants also carried mutations at the dnd loci, demonstrating the suitability of the pigmentation defect as a selection marker (FIG. 5 panel A). Upon further analysis of the gonads from 10 albino fish, 6 were translucid germ cell-free testes (FIG. 5 panel B). Expression of vasa, a germ cell specific marker strongly expressed in wild type testes, was strikingly not detected in dnd mutant testes. This result indicates that zygotic dnd expression is necessary for the maintenance of germ cells and that maternally contributed dnd mRNA and/or protein cannot rescue the zygotic loss of this gene.

### Example 4—Producing Germ Cell Free Gonads

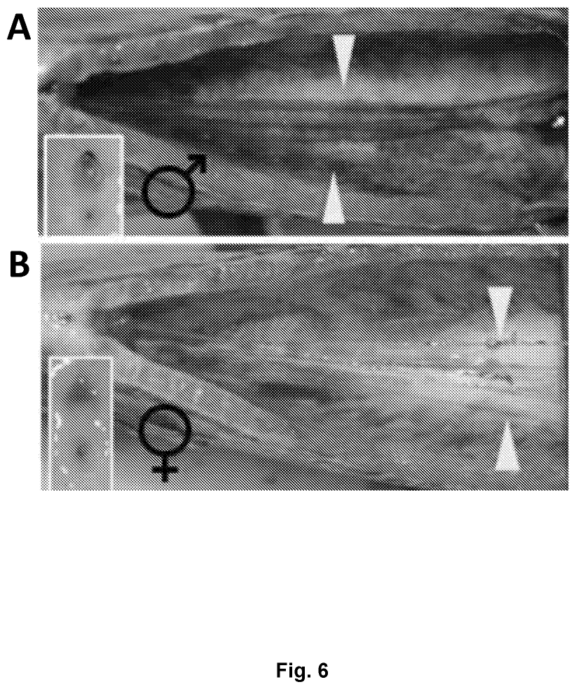

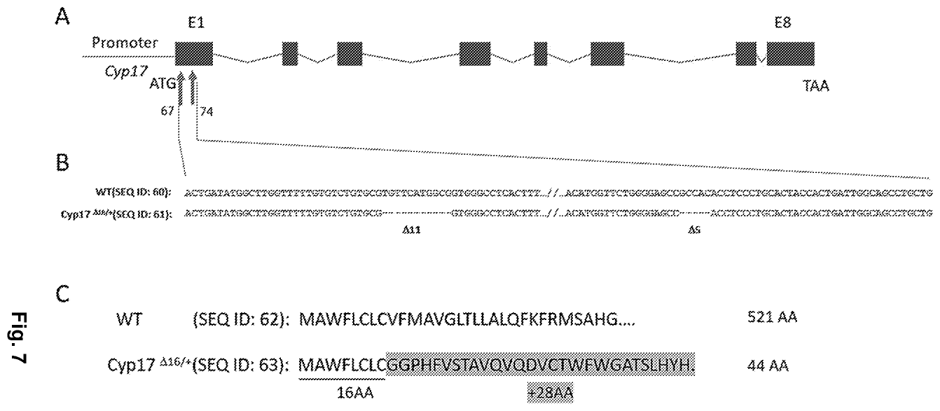

We produced sterile tilapia by implementing transient silencing of the dnd gene in embryos via microinjection of antisense modified oligonucleotides (dnd-Morpholino as well as dnd-AUM oligos). We produced sterile tilapia following bath-immersion of embryos exposed to a small molecule initially discovered in a screen to ablate PGCs in zebrafish [10]. We further generated sterile tilapia using gene knockout strategies as describe for dnd in the section above (Example 3). We also found that breeding Elavl2 heterozygous mutant lines and selecting the homozygous-mutant progeny allow production of germ cell-free adult of both sexes (FIG. 6). These gene KO approaches, along with others mentioned above, produce infertile tilapia, displaying either female urogenital papillae (UGP) and a string-like gonad or male UGP and a translucid tube-like gonad (FIG. 6). These methodologies, however, are not viable solutions for commercial production of sterile fish because only a 25% of progeny from heterozygous mutant parent are sterile and other knock down approaches are insufficiently robust and reliable to ensure complete sterilization of each fish in every batch treatment. In the present invention mass-production of sterile fish rely on broodstock surrogate parents that start as germ cell free fish, then receive germline stem cell transplant and ultimately produce donor derived sperm or eggs. Sterilization of these recipient broodstock in our approach preferentially use knockout strategies (e.g. elavl2-null progeny from heterozygous parents; see Example 11). Knockout strategies other than Elavl2 may be used to produce sterile recipient, including a null mutant for dead-end1, vasa, nanos3 or piwi-like genes. Such a knockout recipient ensures that only donor derived gametes are produced after transplantation. Depending on the species of fish, crustacean or mollusk, alternative strategies to produce sterile recipient can be used, including hybridization and triploidization (Benfey et al., 1984; Felip et al., 2001).

### Example 5—Cyp17l is Necessary for Female Development in Nile Tilapia

The balance of steroidogenic hormones may govern sex differentiation and maturation of the gonads in teleost fish, with estrogen playing an essential role for female differentiation. However, gonadal differentiation and gametogenesis in the absence of both androgen and estrogen has not been investigated. To this end, we produced an in vivo tilapia model lacking the cyp17l gene (hereafter referred to as cyp17).

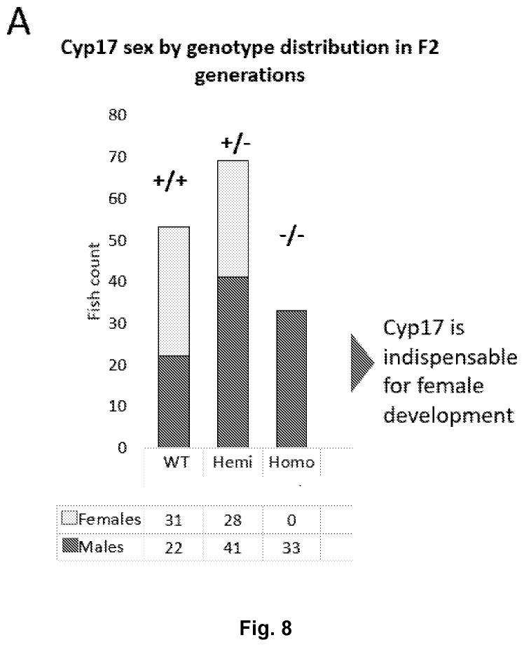

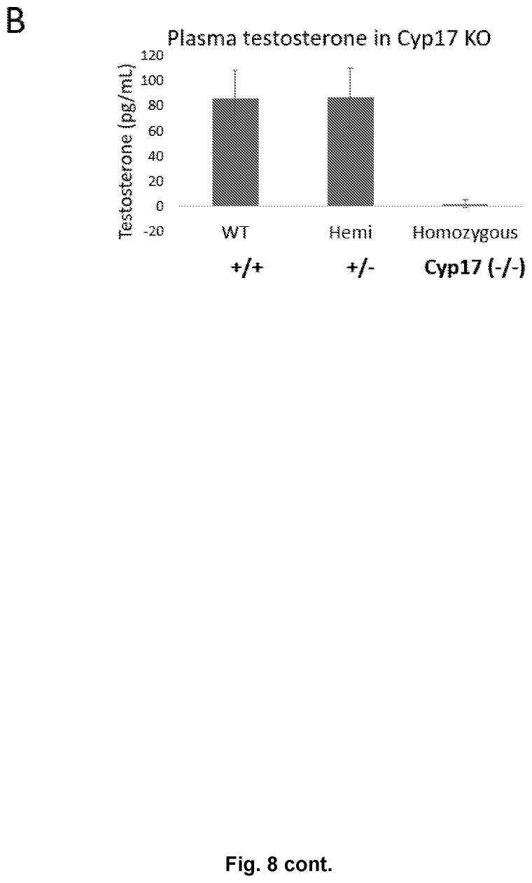

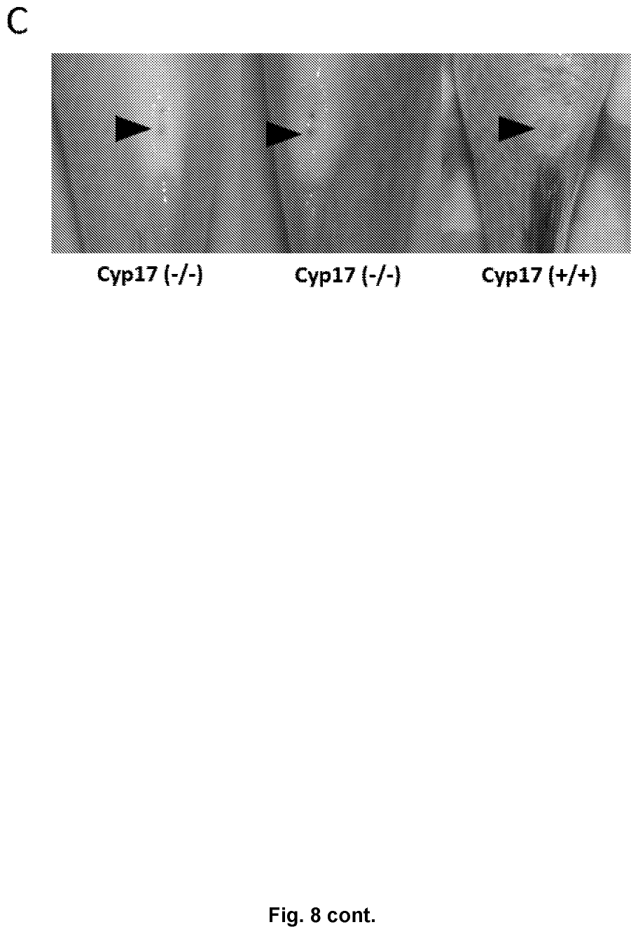

In Nile tilapia, this enzyme is exclusively expressed in Theca cells and produces androgens in response to luteinizing hormone (LH) [13]. Androgens are then converted into estrogen by follicle stimulating hormone (FSH)-induced aromatase (cyp19a1a) in the neighboring granulosa cells of growing follicles. Accordingly, cyp17 loss of function (via gene editing knockout) should simultaneously block androgen and estrogen synthesis. Consistent with this model, we found that 20 of the 22 selected F0 albino/cyp17 mutants developed as phenotypic males, which all displayed minuscule UGP (FIG. 8 panel C). The atrophy of the genitalia is not unexpected given the relationship between androgens and genital papilla [14]. These F0 males remained fertile however, possibly due to a partial loss of function phenotype in the mosaic F0 context. For a complete phenotypic analysis, we generated individuals carrying the same null Δ16-cyp17 mutation in all cells of their body by selective breeding of F1 progeny (FIG. 7). Intercrossing between F1 heterozygotes (cyp17+/−) produced ˜360 F2 progeny and a typical Mendelian segregation of wildtype (n=110; cyp17+/+), hemizygous (n=159; cyp17+/−) and homozygous animals (n=91; cyp17−/−). A total of 155 F2 progeny were sexed at 6 months of age, based on the morphological characteristics of their urogenital papillae (UGP). We found that all 33 homozygotes fish developed as phenotypic males, with atrophic UGP (FIG. 8 panel A). Our results indicate that Cyp17 is indispensable for female development.

We then quantified the amount of free plasma testosterone by ELISA in wild-type and cyp17-mutant tilapia. A mean of 86 pg/mL of testosterone was measured in wild-type (cyp17+/+) and heterozygous mutant tilapia (cyp17+/−) whereas no detectable level of testosterone was found in homozygous mutant (cyp17−/−) (FIG. 8 panel B). This confirm the essential role of this enzyme in androgens production.

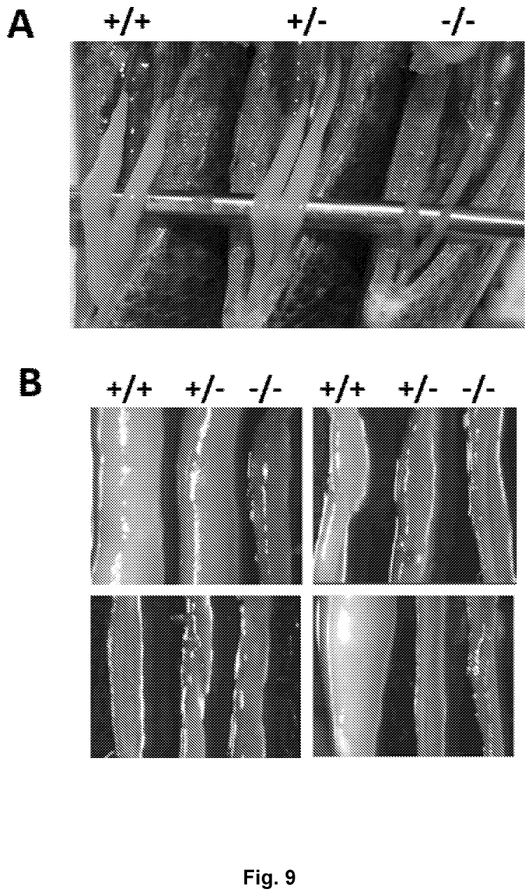

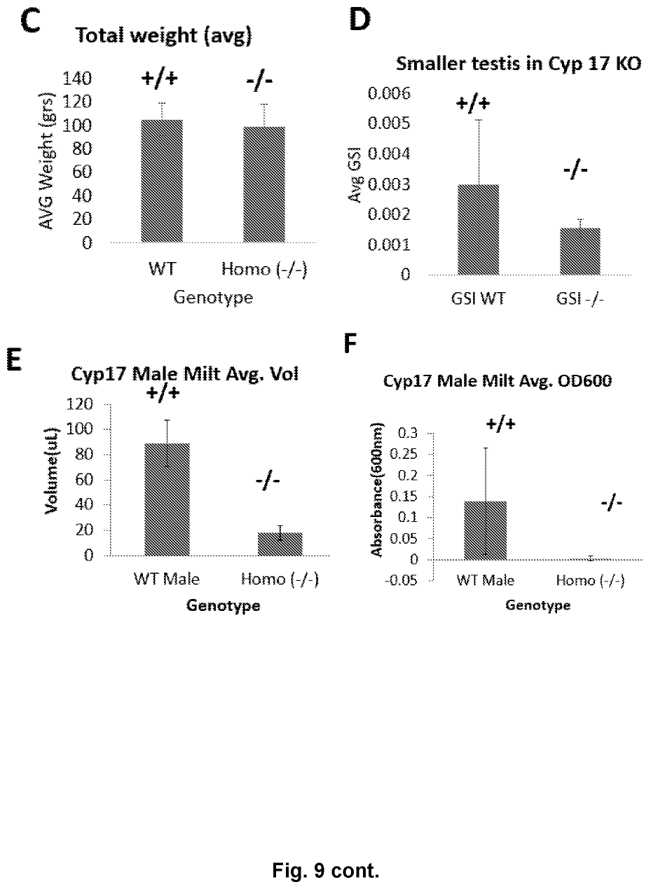

We further examined the morphology and functionality of the gonads in Cyp17 deficient fish. Sibling 5-month-old males cyp17+/+, cyp17+/− and cyp17−/−, of identical size were dissected and all organs except the gonads were removed from their body cavity (FIG. 9 panel A). WT and hemizygous mutants showed pink colored testes typically found in sexually mature fish, while homozygous mutants exhibited translucid testes (FIG. 9 panels A and B). Furthermore, mutant testes were 50% smaller than controls (FIG. 9 panel D) and strippable milt volume was less than 20% of WT (FIG. 9 panel E). In addition, sperm concentration in homozygous cyp17 mutants was reduced 20 and 6-fold at 5 and 6 months of age respectively (FIG. 9 panel F). We found no defect in sperm morphology, motility or functionality, as evidenced by the successful fertilization of WT eggs with milt collected from 10 null mutants.

The fact that cyp17 null mutants can undergo spermatogenesis suggests that androgens are not strictly necessary for this process in Nile tilapia. Thus, a loss of function mutation in this gene may not be sufficient to produce all-sterile male populations. To identify the regulatory mechanism responsible for the formation of functional spermatozoa, we investigated additional genes associated with male infertility in mammals.

### Example 6—Gene Candidates for Targeting Spermiogenesis

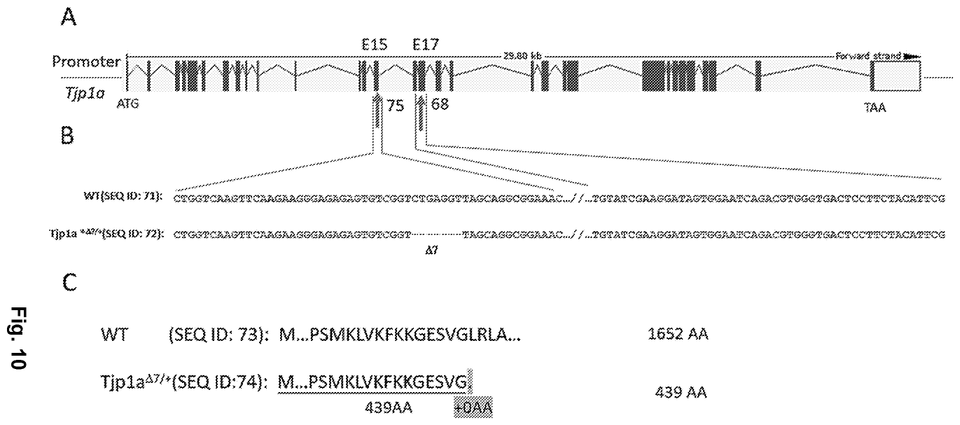

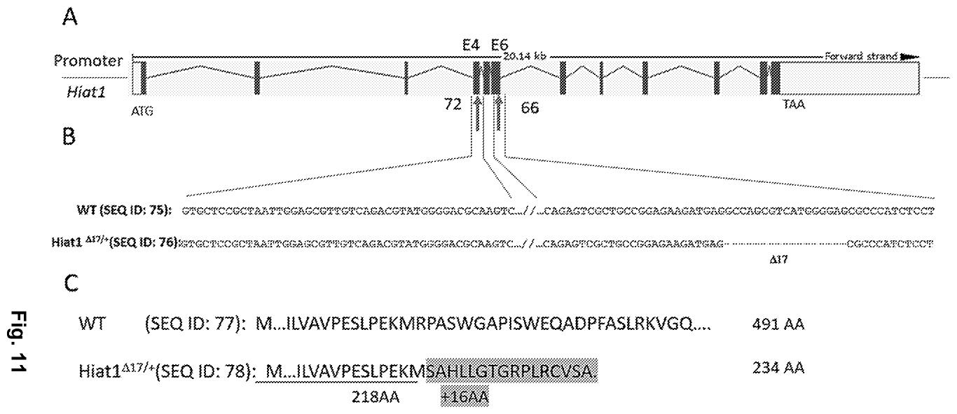

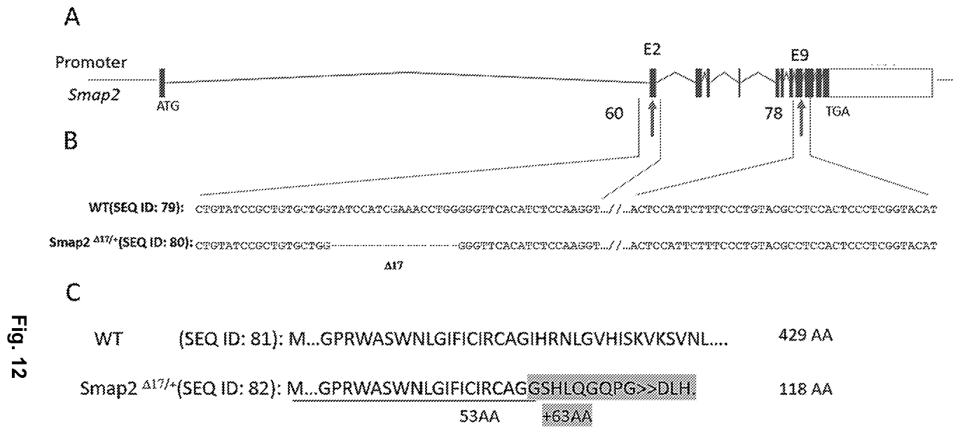

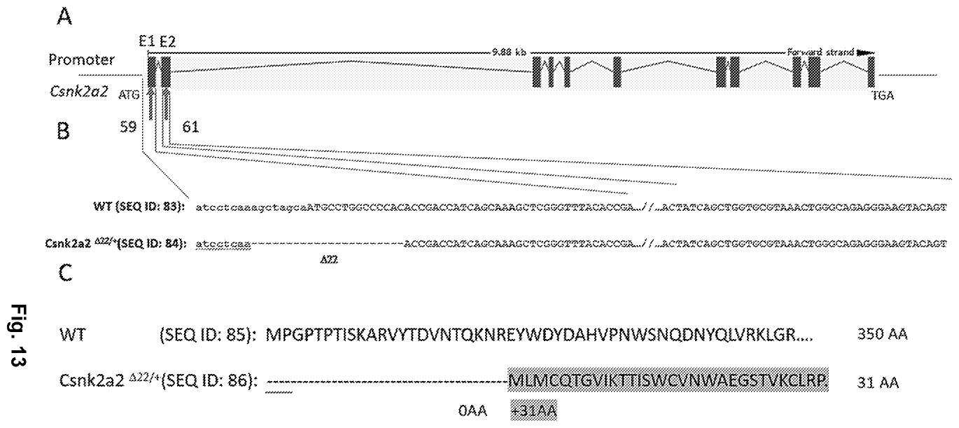

There are significant differences in the morphology and function of mammalian and fish sperm. In particular, fish sperm lack an acrosome and are immotile in seminal fluid, while mammalian spermatozoa possess an acrosome (a key organelle necessary to penetrate the egg chorion) and is mobile in seminal fluid. Globozoospermia is a rare and severe form of human infertility characterized by sperm defective in both morphology and function. Fish models of this disease, however, have not been developed, likely because fish sperm lack an acrosome. Using genomic databases, we identified in silico the tilapia orthologs of the following mammalian genes: Csnk2a2 [15] Gopc [16, 17], Hiat1 [18], Tjp1a, Smap2 [21]. To explore their function in tilapia, we targeted 2 separate exons for each gene (see FIGS. 10 to 14). A pigmentation gene (tyrosinase) was co-targeted and used as a mutagenesis selection marker.

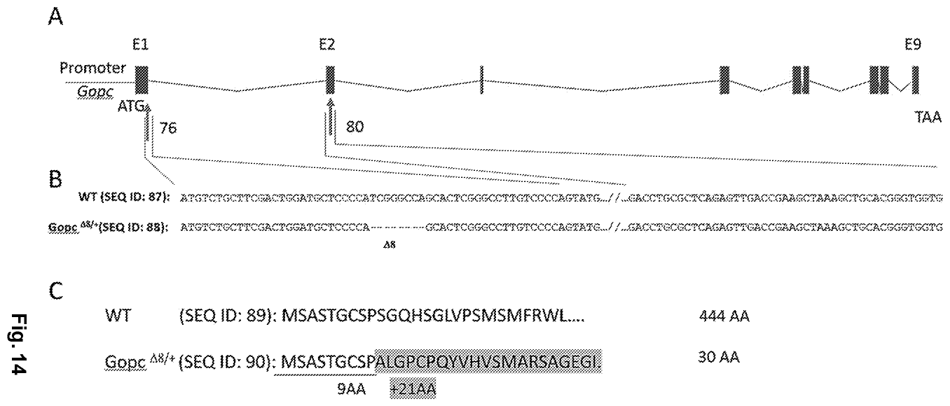

In conjunction with non-treated controls, approximately 20 embryos per candidate gene displaying pigmentation defects were raised to adulthood. At 5 months of age, milt from F0 males and WT controls were stripped to assay sperm density, motility and morphology. Compared to controls, all F0 mutant males produced diluted sperm. Under microscopy, mutant spermatozoa largely produced only a trembling movement and we found wide-ranging frequencies (25%-95%) of abnormally shaped sperm heads, characteristic of the defects seen in human and mice with globozoospermia (FIG. 15 panel A). These mutations caused significant decreases in fertilization rates (FIG. 15 panel B). Furthermore, we found a positive correlation between the severity of the sterility phenotype and the observed frequency of the sperm deformities, with the lowest fertilization rate found in Tjp1a mutants where 95% of sperm were deformed (FIG. 15 panels A and B). We found that all females in these F0 mutant lines are fertile.

Our results point to the existence of an evolutionarily conserved pathway controlling spermiogenesis in fish and mammals. These results support the idea that the targeted disruption of these corresponding genes will cause a sterility phenotype in many other teleost species, and possibly more broadly in other taxa as well.

### Example 7—Sterile all-Male Fish in Cyp17 KO Background

To engineer male sterility, we first evaluated the effect of null mutations in the cyp17gene, which controls an important branch point in steroid hormone synthesis, regulating both androgen and estrogen production. We found that all cyp17−/− fish develop as male. Surprisingly, milt produced by cyp17−/− contained a small number of mature spermatozoa that could fertilize oocytes by in vitro fertilization. We than investigated the possibility of blocking spermiogenesis. Our preliminary screens focused on five genes associated with globozoospermia (collectively termed spermiogenesis specific genes or SMS-genes: Smap2, Cnsk2a2, Gopc, Hiat1 and Tjp1a), whose mutations caused subfertility in F0 males with severe oligo-astheno-teratozoospermia, while F0 mutant females were fully fertile. Previous genetic characterizations of F0 KO fish indicate that they typically carry mosaic mutations at the corresponding targeted loci, some of which are often in-frame causing partial rescue of the phenotype. Thus, to measure the full loss-of function phenotype, we performed additional phenotypic characterization on homozygous SMS-null-mutants. We further established lines of tilapia carrying double homozygous mutations to interrogate the effect of simultaneously impairing spermiogenesis and steroid hormone synthesis.

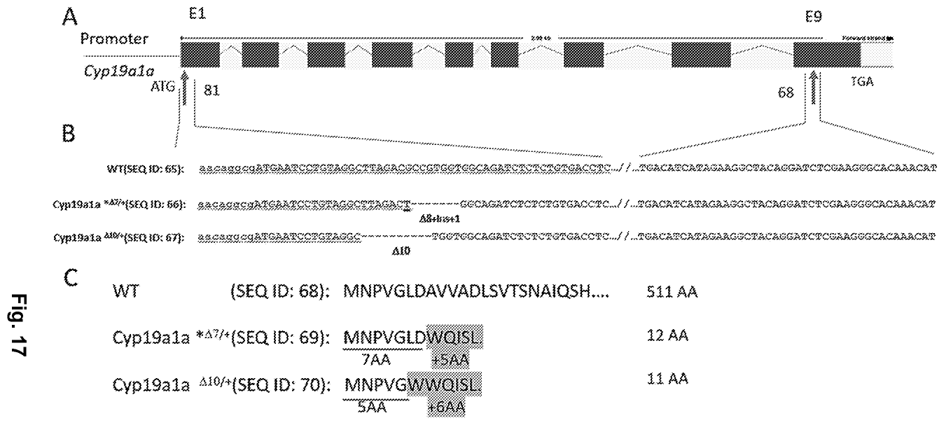

Experiment: To assess in vivo function of double gene knockouts in cyp17 and one of the 5 SMS gene, we outcrossed F0 SMS mutant females with cyp17^{Δ16/+} males. Offspring (120 to 180 fish) were genotyped at each target locus by PCR fragment analysis (as described in FIG. 2) [22]. We only raised individuals carrying an identical mutant allele, hereafter referred to as m1 (FIG. 18), at the selected SMS locus (typically 12-50% of the F1 progeny population share the same genotype). A minimum of 10 double heterozygotes (e.g. cyp17^{Δ16/+}; SMS^{m1/+}) were raised to adulthood. These double heterozygotes were inter-crossed, and their progeny genotyped at 1 month of age by QPCR melt analysis. For each of the 9 ensuing possible F2 genotypes (see FIG. 9), a minimum of 30 fish are currently being raised to adulthood and will be assayed for fertility. Females cyp17+/+; SMS+/m1 (e.g. cyp17+/+; Tjp1a+/m1) were set aside for further studies described in section 2 below. FIG. 9 summarizes this experimental scheme, using Tjp1a as an example of an SMS gene target.

Without being bound by theory, we believe that in finfish, as in mammals, null mutations in all 5 conserved spermiogenesis specific genes will result in oligo-astheno-teratozoospermia and cause infertility. We expect that all double homozygous mutants (cyp17−/−; SMS−/−) will develop as sterile males with even lower sperm counts than any single KO male defective in spermiogenesis (SMS−/−). Indeed, cyp17−/− fish should be deficient in 11-ketotestosterone, a positive regulator of spermatogenesis. Consistent with the idea that androgen plays an intra-testicular paracrine role in spermatogenesis, cyp17−/− tilapia have previously been shown to display low sperm counts. FIG. 9 shows the nine genotypes along with four different corresponding phenotypes with the expected percentages: 1) ˜56% fertile for both sexes, 2) ˜19% fertile female and sterile male, 3) ˜19% all fertile male; and 4)˜6% all-sterile male. Looking at each trait individually, we expect a progeny population of 62% male with 25% of these males being sterile.

### Example 8—Sterile all-Male Fish in Cypnala KO Background

An alternative strategy to generate all-male population is to inactivate the Cyp19a1a aromatase (hereafter referred to as Cyp19). We created out of frame mutations in the coding sequence of the tilapia cyp19 gene (FIG. 17). This enzyme is produced by the somatic gonad and convert testosterone into estrogen. Consistent with this model, we found a strong male bias amongst the 25 F0 Cyp19 mutants selected, with 20 mutants developing as phenotypic males (Table 3). Notably these mutant males displayed normally appearing male urogenital papillae, indicating that androgen production is not impaired and secondary male sexual characteristics develop normally. This stand in contrast to cyp17 KO males, which lack androgen and accordingly develop atrophic urogenital papillae. The generation of all-male sterile tilapia populations, which either express or do not express androgens (as in cyp19 KO and cyp17 KO backgrounds respectively), will allow us to interrogate the influence of male sex steroid hormone on tilapia growth performance. The stimulatory action of testosterone on GH secretion and responsiveness is well documented in mammals. For a complete phenotypic analysis, we generated individuals carrying the same null mutations in all cells of their body. Heterozygous cyp19 F1 offspring with a Δ10-cyp19 deletions in the first exon were selected to breed the F2 generation. This frame-shift mutation is expected to create a truncated protein lacking >98% of its wild type amino acid sequence (FIG. 17). This F2 generation was genotyped and sexed. As expected, we found that homozygous A10-cyp19 tilapia all develop as males (n=38) while hemizygous (n=97) and wild-type (n=40) had a normal sex ratio. We further established lines of tilapia carrying double homozygous mutations to interrogate the effect of simultaneously impairing spermiogenesis and steroid hormone synthesis.

Experiment: We first outcrossed heterozygous F1 males Δ10-cyp19a1a with the heterozgygous mutant females from Example 7 (Gopc^{Δ8/+}; Smap2^{Δ17/+}; TjP1a^{Δ7/+}; Csnk2a2^{Δ22/+}; Hiat1^{Δ17/+}. Only SMS genes that cause male sterility when disrupted in a Cyp17 null background (results from Example 7) will be selected. The progeny will be genotyped and at least 10 double heterozygous will be raised to adulthood, sexed, and inter-crossed. The resulting progeny will be assayed for fertility as described in Example 7. A maximum of 5 different double KO males will be generated. Without being bound by theory, we expect double KO cyp19−/−; SMS−/− fish to develop as sterile males and anticipate a progeny population of 62% male, with 25% of them being sterile.

TABLE 3
Description of single gene mutant alleles, double hemizygous mutant alleles and homozygous mutant alleles generated
in this study. Genes names are listed based on their specific role in feminization (FEM), spermiogenesis (SMS),
masculinization (MA) and folliculogenesis (FLS). Phenotypes observed in selected F0 mutant are described.

| Gene | Gene | phenotype in F0 | Selected F1 genotypes | Selected double | |
|---|---|---|---|---|---|
| categories | names | albino | (hemizygous) | hemizygous mutant | F2 homozygous |
| FEM | Cyp17 | 80% male (n = 20) | Cyp17^{Δ16/+} | | Cyp17^{Δ16/Δ16} |
| | Cyp19a1a | 75% male (n = 23) | Cyp19^{Δ7/+}, Cyp19^{Δ10/+} | | Cyp19^{Δ10/Δ10} |
| SMS | Tjp1a | Reduced fertility | Tjp1a^{Δ7/+} | Tjp1a^{Δ7/+} and Cyp19^{Δ10/+} | Tjp1a^{Δ7/Δ7} |
| | Csnk2a2 | in males (70-90%) | Csnk2a2^{Δ22/+} | Csnk2a2^{Δ22/+} and Cyp17^{Δ16/+} | Csnk2a2^{Δ22/Δ22} |
| | Hiat1 | | Hiat1^{Δ17/+} | Hiat1^{Δ17/+} and Cyp17^{Δ16/+} | Hiat1^{Δ17/Δ17} |
| | Smap2 | | Smap2^{Δ17/+} | Smap2^{Δ17/+} and Cyp19^{Δ10/+} | Smap2^{Δ17/Δ17} |
| | Gopc | | Gopc^{Δ8/+} | | Gopc^{Δ8/Δ8} |
| MA | Dmrt1 | 50% female (n = 24) | Dmrt1^{Δ7/+}, Dmrt1^{Δ13/+} | | |
| | Gsdf | 95% female (n = 20) | Gsdf^{Δ5/+}, Gsdf^{Δ22/+} | | |
| FLS | FSHR | Sterile females | FSHR^{Δ5/+} | FSHR^{Δ5/+} and Dmrt1^{Δ7/+} | |
| | VtgAa | (2 in 6 tested) | VtgAa^{Δ5/+}, VtgAa^{Δ25/+} | VtgAa^{Δ25/+} and Gsdf^{Δ22/+} | VtgAa^{Δ25/Δ25} |
| | VtgAb | Sterile females | VtgAb^{Δ8/+} | VtgAb^{Δ8/+} and VtgAa^{Δ25/+} | VtgAb^{Δ8/Δ8} |
| | | (3 in 5 tested) | | | |
| | | Reduced fertility | | | |
| | | in females (70-90%) | | | |

### Example 9—Evaluate Two Genes Targeting Male Differentiation in Conjunction with Two Other Genes Controlling Oogenesis to Produce a Sterile all-Female Population

The transcriptional inhibitor Gonadal soma-derived factor (Gsdf) is a TGF-b superfamily member expressed only in the gonads of fish, predominantly in the Sertoli cells. Similarly, the transcription factor Dmrt1 is preferentially expressed in pre Sertoli and Sertoli cells as well as in epithelial cells of the testis. Both genes are necessary for normal testis development ([23, 24]).

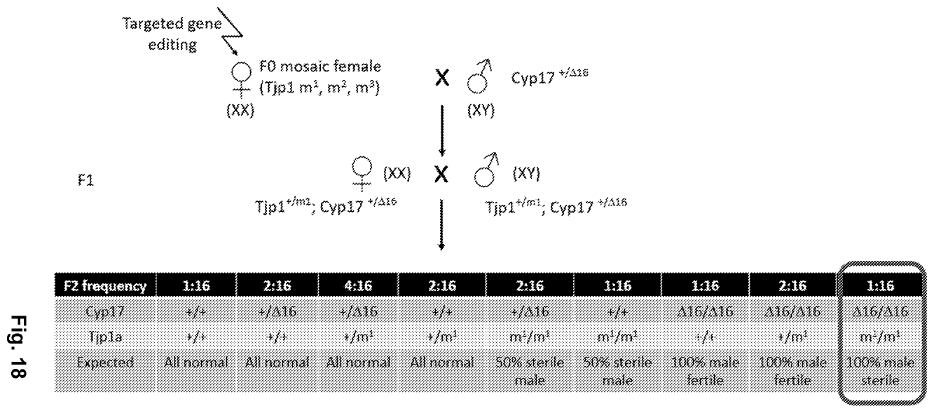

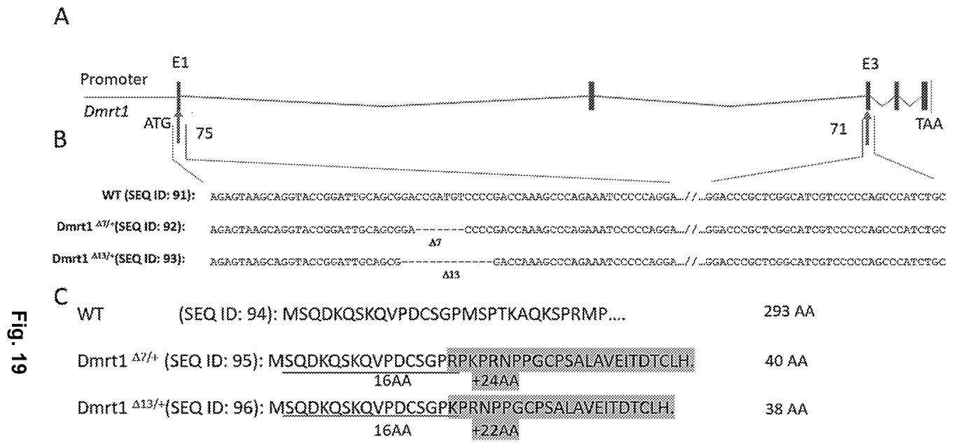

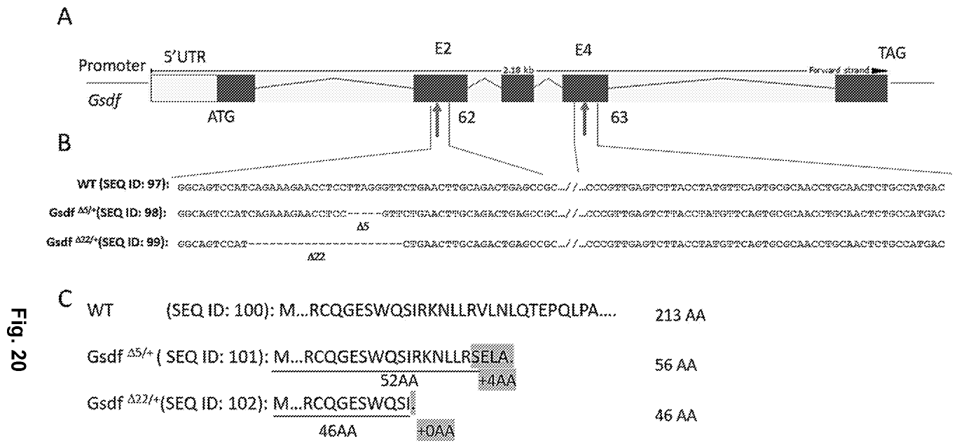

To produce all-female tilapia populations, we generated null mutations in either Dmrt1 or Gsdf genes (maleness genes or MA) (FIG. 19 and FIG. 20). We found that 19 out of 20 Gsdf mutated albino tilapia developed as females (Table 3). In contrast, F0 mutant showing mosaic pigment defect had normal sex ratio. Postulating a positive correlation of mutagenic frequency between co-targeted tyrosinase and Gsdf genes, our result suggests that high-mutation-rate in Gsdf cause XY male to sex reverse into female. Surprisingly we did not observe a female sex bias amongst selected F0 Dmrt1 mutant (Table 3).

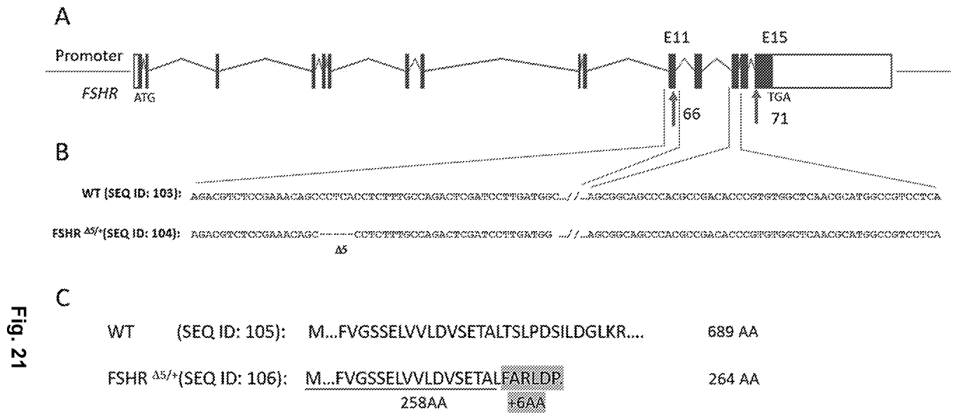

To engineer sterility in females, we targeted genes involved in the maturation of ovarian follicle. We have identified two genes in the molecular pathway controlling folliculogenesis: 1) FSHR which acts upstream of ovarian estrogen synthesis and. 2) vitellogenins (Vtgs) which act downstream of ovarian estrogen synthesis. Vitellogenins are preferentially produced by the liver while FSHR, the follicle-stimulating hormone (FSH) receptor is expressed in Theca cells surrounding the developing oocytes. To test the necessity of FSHR and Vtgs in normal ovarian development (folliculogenesis specific genes or FLS) we produced loss-of-function mutations in those genes in independent F0 lines (FIGS. 22-24).

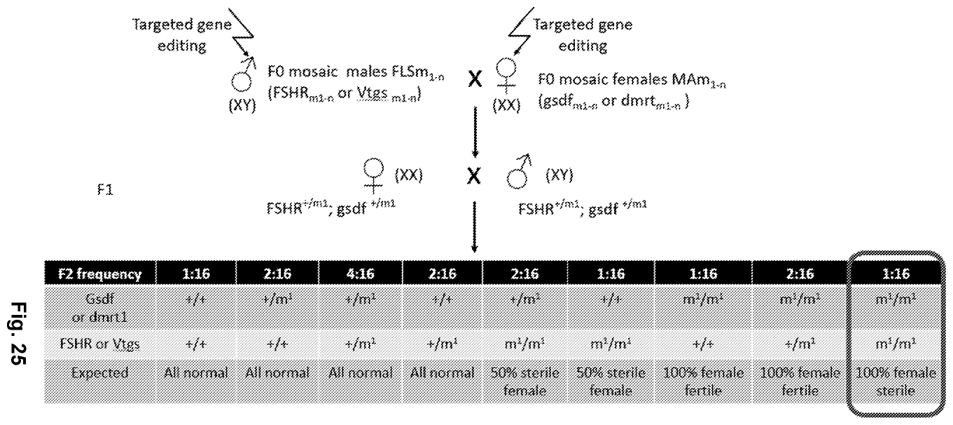

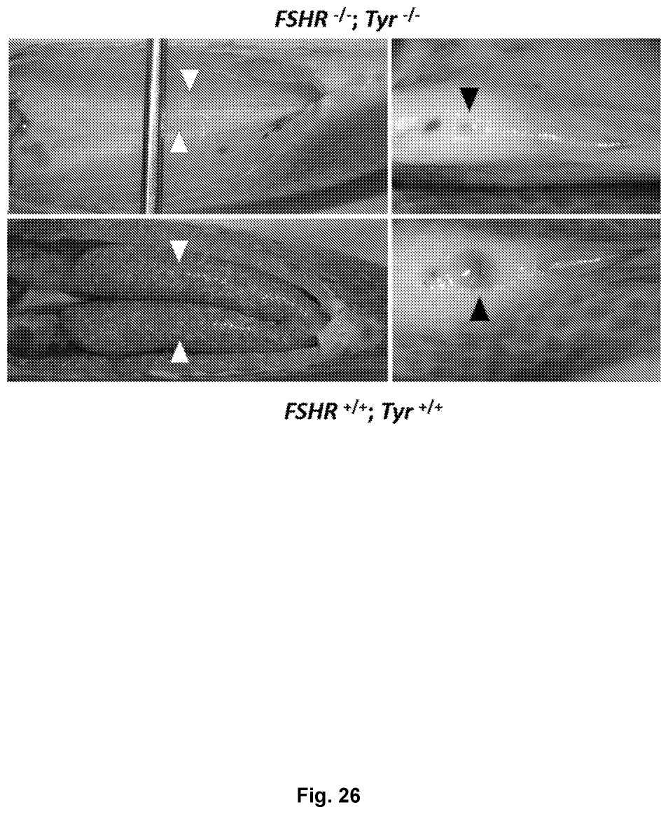

We found that FSHR is indispensable to folliculogenesis and the disruption of the FSHR gene resulted in a complete failure of follicle activation and female sterility (FIG. 26 and Table 3). In tilapia, FSHR mutation was not followed by masculinization of genetic females into males, as previously described in zebrafish [29]. However, we found that F0 FSHR mutant females had significantly smaller urogenital papillae when compared to control female. This observation likely reflects a reduced level of estrogen in FSHR mutant, consistent with a role of FSHR in locally up-regulating aromatase expression and estrogen production. We found no significant reproductive phenotype in F0 FSHR mutant male.

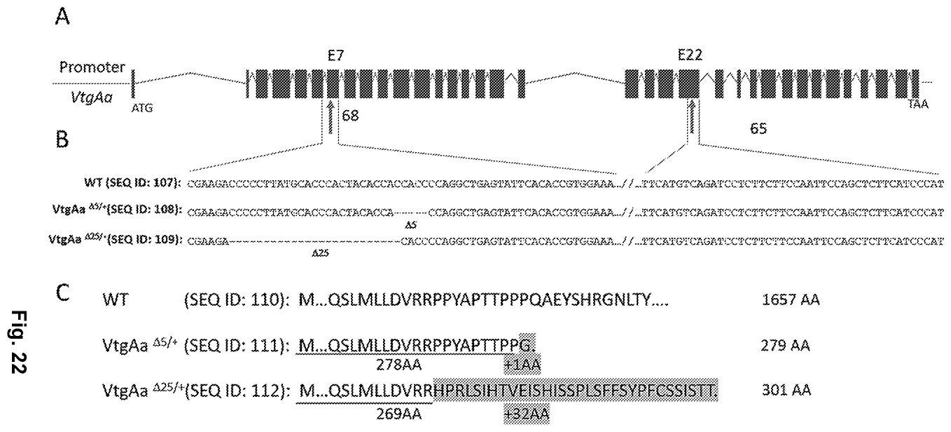

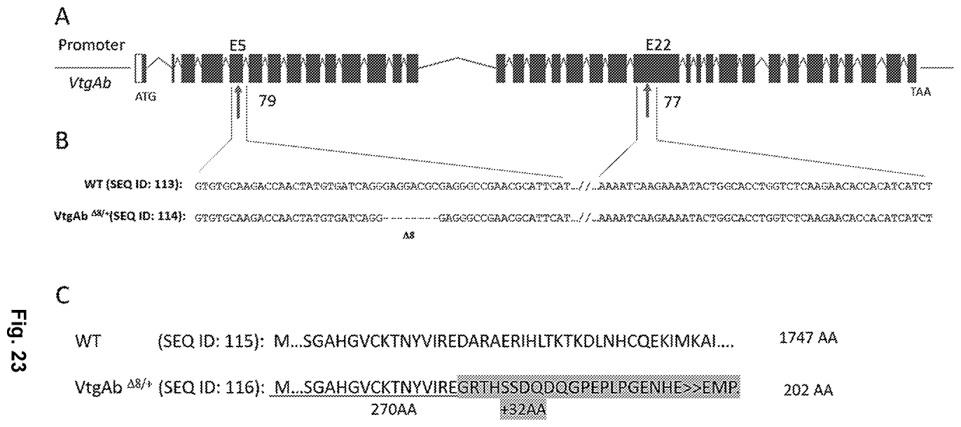

Nile tilapia only possess 3 Vtg genes [25], two forms of complete Vtgs (VtgAa and VtgAb) and one form of incomplete C-type teleost vitellogenin, lacking three protein domains (VtgC). Since VtgAa and VtgAb are expressed at higher level than VtgC and assumed to be critical to early embryo development, we targeted those two genes individually as well as jointly (FIGS. 22, 23, and Table 3). Consistent with functions in oocyte maturation and nutritional support for embryogenesis, we found that 3 F0 females mutated in VtgAa out of 4 tested failed repeatedly to produce viable progeny (FIG. 24). We also found that one F0 female carrying mutations in VtgAb out of 5 produced embryos progeny that died before hatch (data not shown).

For a complete phenotypic characterization, it is essential to generate identical mutations in every cell of the animal. Thus, we will establish and characterize 4 lines of tilapia deficient in both masculinization and vitellogenesis.

At 6 months of age, mosaic F0 XX MA m₁₋ₙ female (e.g. Dmrt1 m₁₋ₙ or Gsdf m₁₋ₙ) were outcrossed to mosaic F0 FLS m₁₋ₙ males (FSHRm₁₋ₙ or Vtgs m₁₋ₙ) and their F1 progeny genotyped to identify double heterozygous mutants (e.g. Dmrt1^{Δ7/+}-FSHR^{Δ5/+}) carrying the same gene specific indel at each locus (Table 3).

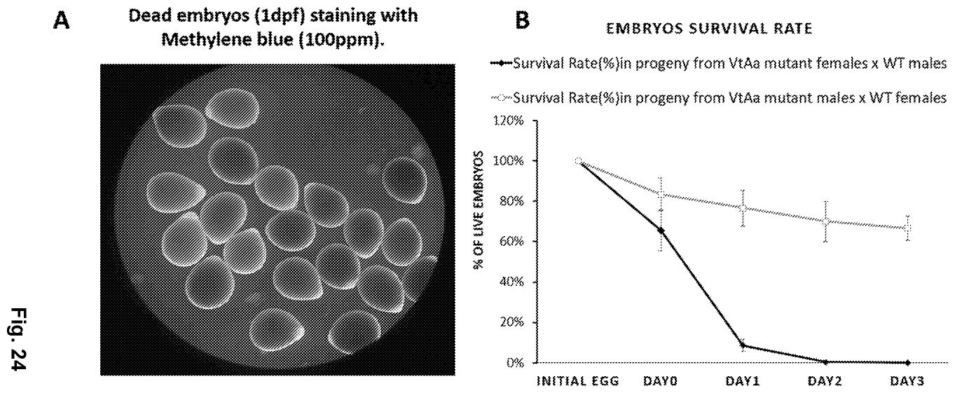

Experiment: A minimum of 10 double heterozygotes (for each of the four gene combinations) are currently being raised to adulthood. The WT alleles should ensure that these F1 fish develop as both fertile males and females. These double heterozygous mutants will then be incrossed, and their progeny genotyped at 1 month of age by QPCR melt analysis. For each of the 9 ensuing possible genotypes (see FIG. 25), a minimum of 30 fish will be raised to adulthood, then sexed, and assayed for fertility.

FIG. 25 shows nine genotypes and the corresponding four different phenotypes we expect with the following fractional ratios: 1) ˜56% fertile for both sexes, 2) ˜19% fertile female and sterile male, 3) ˜19% all fertile male; and 4) ˜6% all-sterile female. Looking at each trait individually, we expect a progeny population of 62% female with 25% of these females being sterile.

Our phenotypic investigations in F0 mutant lines (Table 3) mostly agree with our initial hypothesis and we fully expect corroborating genotype-phenotype relationships in subsequent generations. We found that Gsdf deficiency caused feminization while FSHR and Vtgs inactivation resulted in female sterility. These results strongly suggest that double FSHR-Gsdf KOs will develop into monosex sterile female populations characterized by atrophic ovaries containing follicles arrested at the previtellogenic stage. The lack of a sex differentiation phenotype in F0 Dmrt1 mutant likely reflects incomplete editing, regional mosaicism and compensation by non-mutated cells. Without being bound by theory however, we believe that double FSHR-Dmrt1 KOs in which the mutations have been inherited through the germline, will develop into all female sterile populations. In our F0 mutagenesis screen we observed that blocking the precursor of major yolk proteins (as in Vtg KOs), compromises egg quality and impairs the development and survival of embryos. As such, we expect that double KOs Gsdf-Vtgs and Dmrt1-Vtgs will develop into monosex sterile female populations.

### Example 10—Propagation of all-Male and all-Female Sterile Lines by Germline Transplantation into Sterile Surrogate Adults

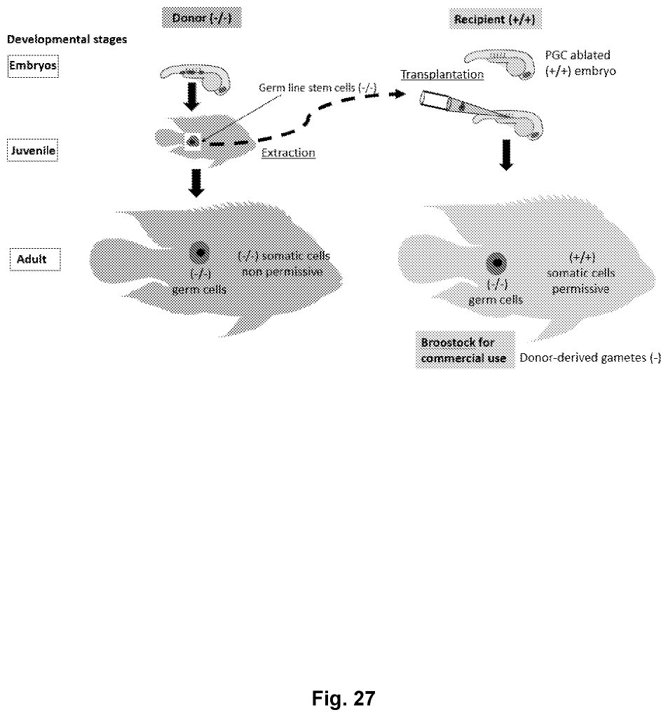

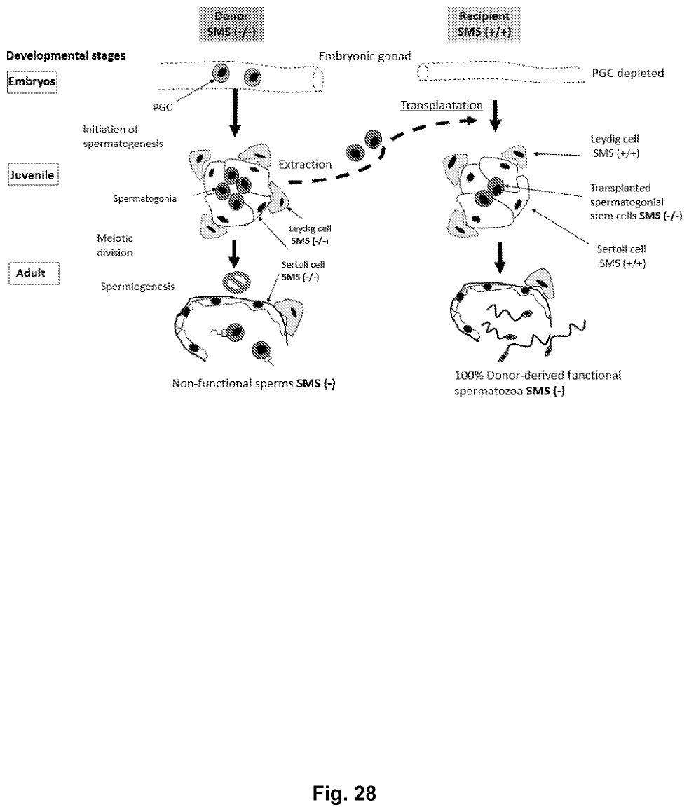

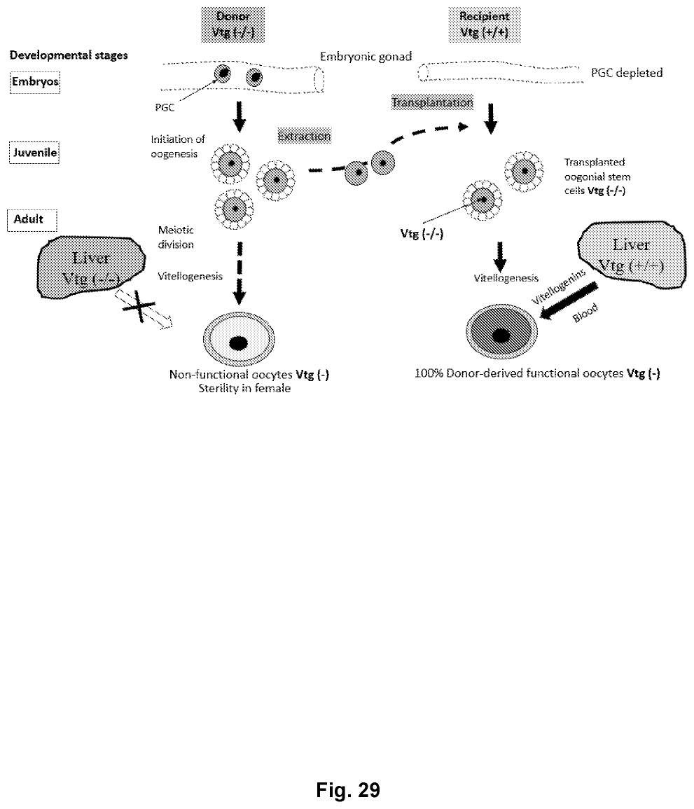

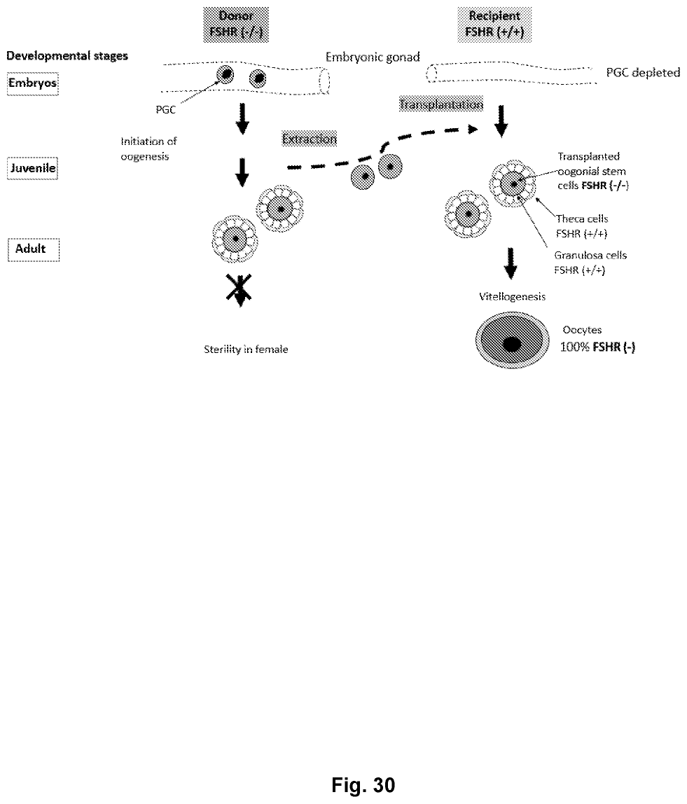

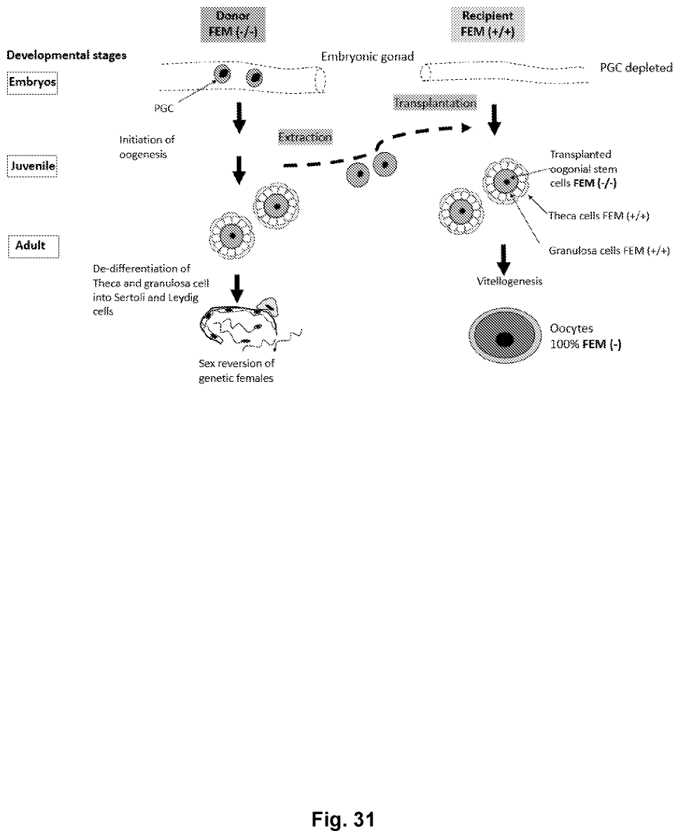

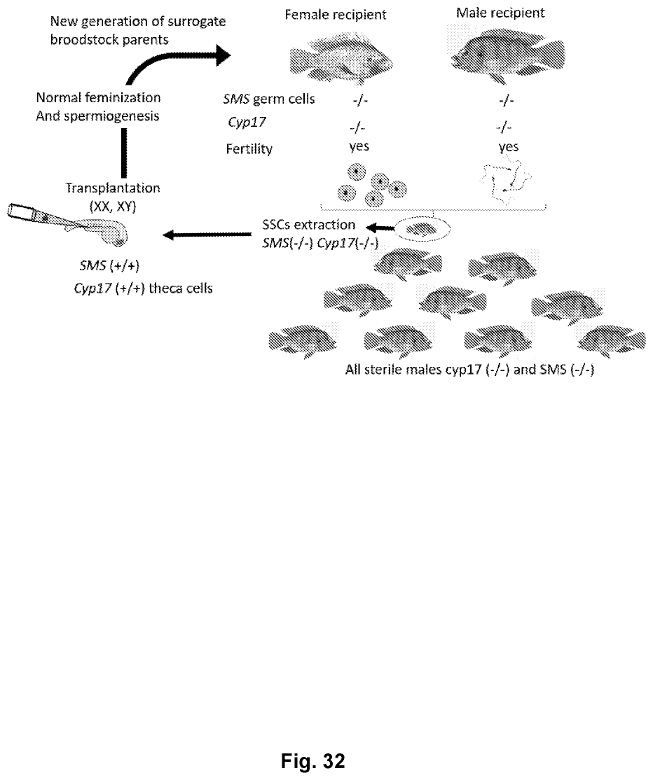

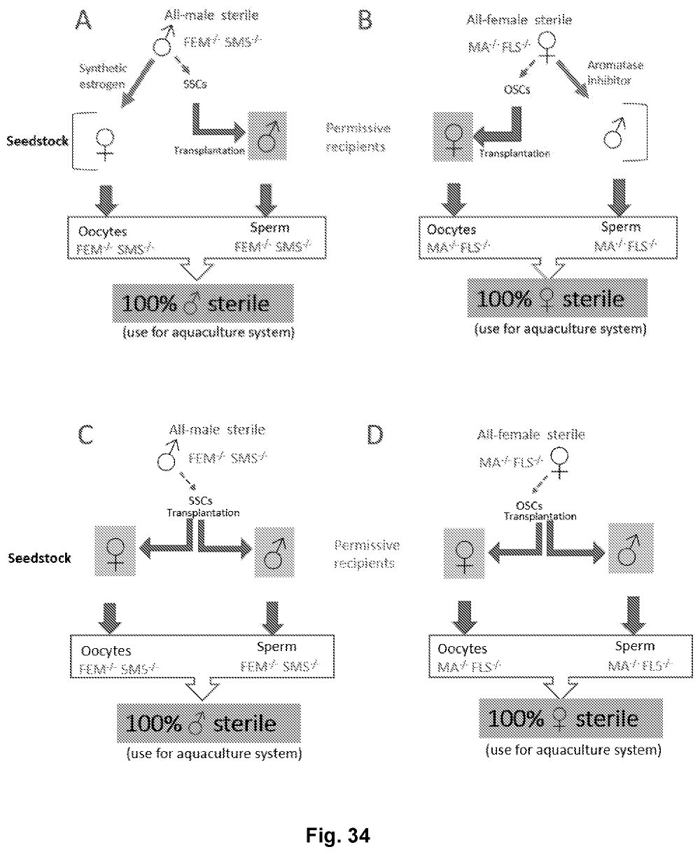

Examples 8 and 9 above illustrate how to generate monosex sterile fish by breeding double hemizygous mutant and by individually selecting the subpopulation of double KO progeny. This approach however may not be sufficiently efficient and may be too expensive to be used in industrial settings. Intracytoplasmic sperm injection in assisted reproduction offers a solution to propagate male broodstock that are defective in spermiogenesis. However, this approach is also not scalable for mass production of commercial stocks (as it requires conducting methods on ‘one fish at a time). The key to larger scale production is to generate male and female broodstock that only produce mutant gametes so that no selection is needed to identify the double KO progeny. Importantly, those mutant gametes should also be functional so that natural mating of these broodstock can be used to produce a viable population of monosex sterile progeny. This is only possible if sex ratio and gamete functionality are rescued in the broodstock. We speculated that this can be achieved by germline stem cell transplantation from a double KO mutant fish to a germ cell free recipient not mutated for the same genes. Such transplanted broodstock have normal somatic cells but a mutant germline (see FIGS. 27-32). These chimeric recipients possess functional MA or FEM somatic gene(s) that ensure normal sex ratio (FIG. 34 panels C and D) and functional SMS or FLS somatic genes to rescue spermiogenesis (FIG. 28) or oogenesis (FIGS. 29 and 30) assuming the mutated genes do not function in germ cells.

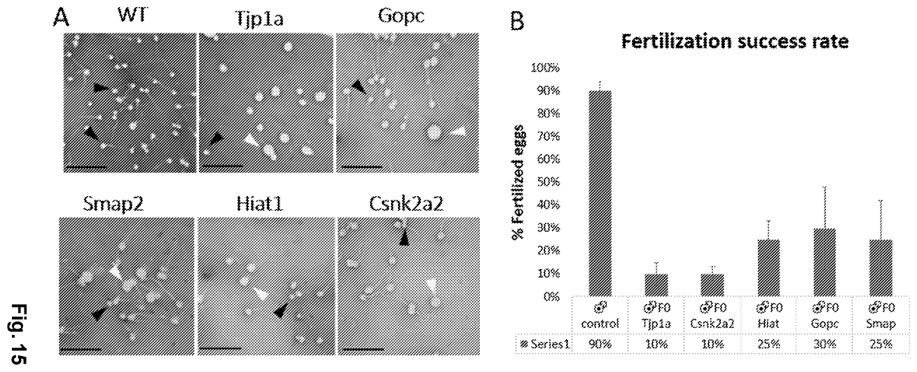

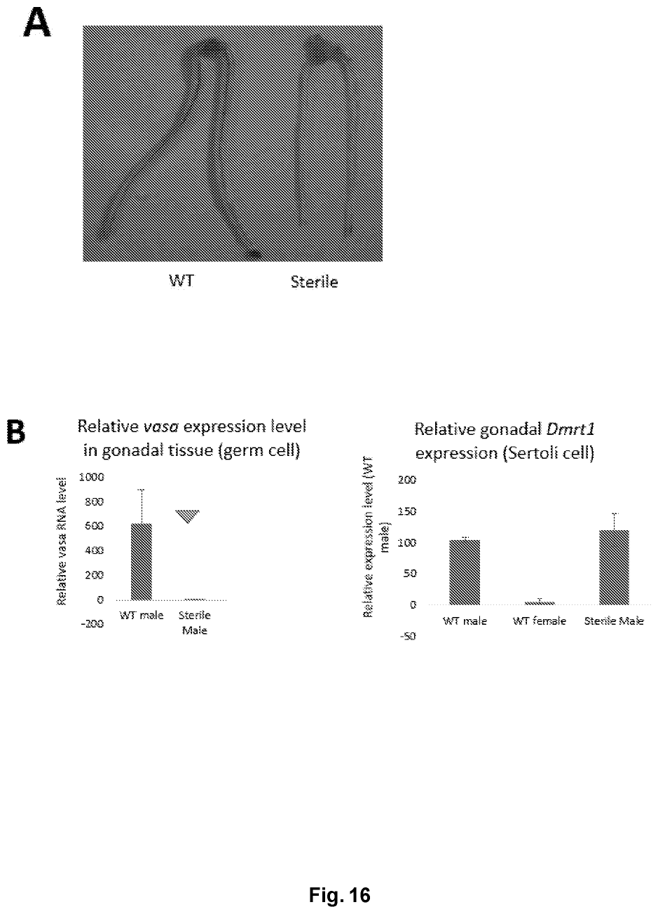

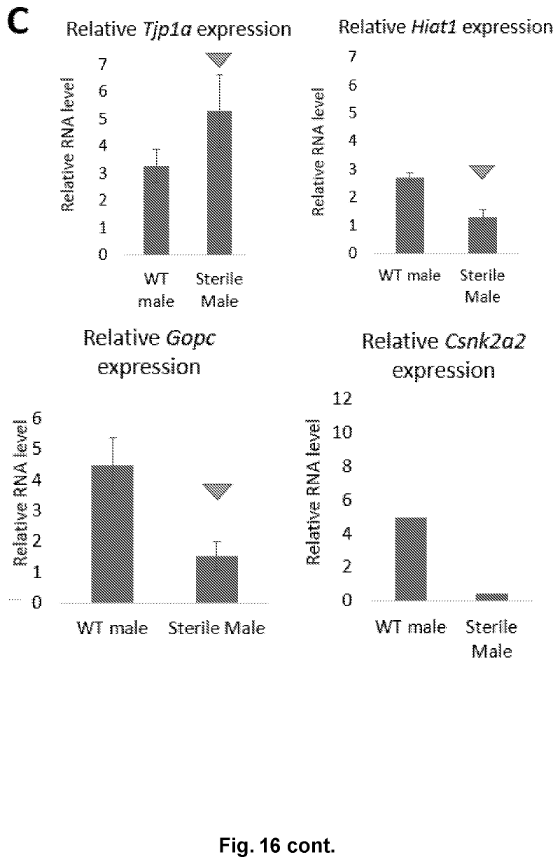

Since spermatogenic failure can result from defects in germ cells or in their somatic environment we analyzed the SMS genes expressions to identify those preferentially not expressed in germ cells (FIG. 16). Our SMS gene expression study in sterile testes point to a role of gonad somatic cells in supporting germ cell development. For example, we found that Tjp1a is a highly expressed in sterile testes at level above wild type testes, while Hiat1 and Gopc expression levels are only slightly reduced compare to fertile testes (FIG. 16).

These results suggest that mutant of those genes develop a testicular microenvironment, where spermiogenesis is impaired due to Sertoli and/or Leydig-specific defects (FIG. 28). Consequently, we expect that transplantation of spermatogonial stem cells from the male knockout infertile donors to a permissive wild type testicular environment will restore sperm functionality and fertility (FIG. 28).

Likewise, FSHR and Vtgs, are strictly expressed in somatic cells (Theca and liver cells respectively). Thus, oocytes carrying null alleles of these genes should retain their intrinsic capacity to proliferate and differentiate, ensuring that oogonial stem cells from a sterile female mutant donor can re-populate the ovaries and differentiate into functional eggs upon transplantation into a WT/permissive recipient (FIGS. 29 and 30). Thus, we believe that recipient males or females can produce gametes that carry the donor genotype.

### Example 11—Elavl2 KO Recipients can Produce Functional Gametes

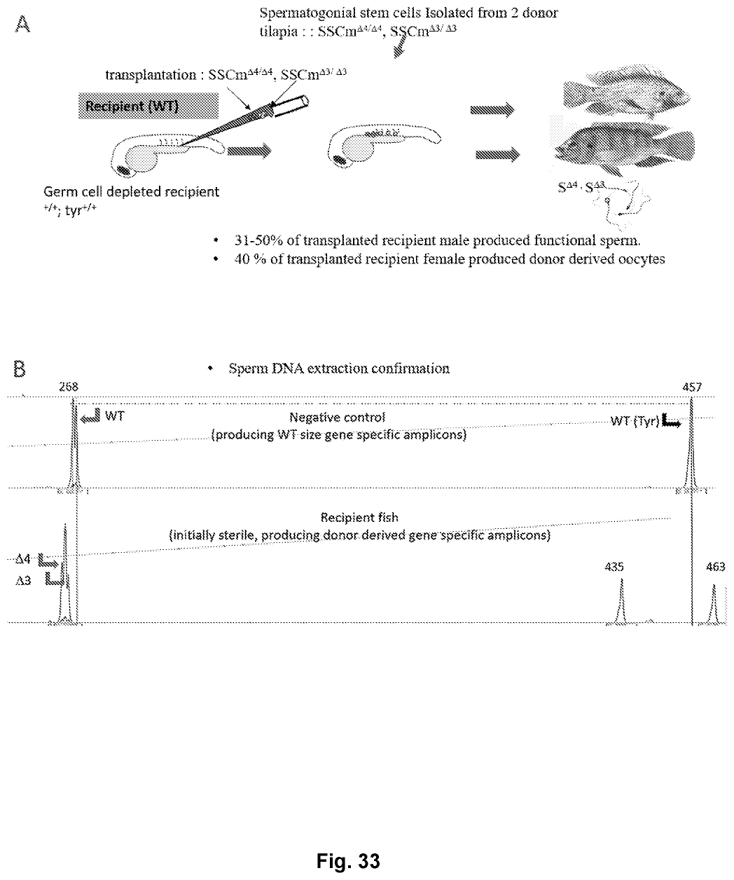

To confirm that sterile Elavl2 KO recipients can produce functional gametes from donor-derived germ cells, we harvested spermatogonial stem from the testes of albino (tyr−/−) male tilapia carrying mutations (in-frame and out-of-frame) in a reference gene (FIG. 33 panel A). We transplanted the testicular cell suspension from both mutant lines, into germ cell depleted recipient embryos progeny from Elavl2−/+, tyr+/+ parents. We genotyped transplanted fish to select homozygous Elavl2−/− mutant and raised them to adulthood. At 5 months of age, between 31-50% of transplanted Elavl2−/− male and 40% of six months old transplanted Elavl2−/− female produced exclusively albino progeny when outcrossed with albino male and female. Non-transplanted Elavl2−/− controls were sterile. Thus, Elavl2−/− recipients can produce donor-derived gametes after germline stem cell transplantation illustrating the feasibility to create a tilapia that produced only donor derived gametes. Using albinism to assay for gametes carrying tyr alleles provided an easy quantifiable high-throughput assay for germline transmission efficacy of mutant alleles, but these experiments do not demonstrate that the null mutations was successfully propagated. To this end, we extracted and analyzed the sperm DNA from one fertile recipient by PCR fragment sizing assay. The amplification products were sized using capillary electrophoresis (FIG. 33 panel B). Results reveal that the recipient fish only produces sperm containing donor derived in-frame and out-of-frame (3 nt and 4 nt) deletions fragments suggesting that the null allele (4 nt deletion) can colonize the gonad and proliferate as efficiently as the positive control mutation (3 nt deletion) (FIG. 33 panel B).

Experiment: Spermatogonial and oogonial stem cells (SSCs, OSCs) will be isolated from all-male and all-female juvenile tilapia lines (developed as per Examples 7, 8, and 9). After harvest, these germline stem cells will be transplanted into Elavl2 KO recipient hatchlings as described above. Without being bound by theory, we expect production of functional spermatozoa and oocytes carrying the donor genotypes. To evaluate the functionality of donor-derived gametes produced after transplantation, in vitro fertilization assays will be performed. Moreover, we expect only albino progeny to arise from a cross between the naturally pigmented recipient carrying albino donor gametes and albino lines. We will genotype 10 progenies for mutations in donor-derived spermatogenesis and vitellogenesis specific genes.

As illustrated in FIG. 34 panel B, crossing surrogate mothers with double KO sex reversed males, obtained from treatment with aromatase inhibitors, will produce all-female sterile progeny. Alternatively, crossing surrogate fathers with double KO sex reversed female mutants rescued after estrogen treatment, will produce all-male sterile populations (FIG. 34 panel A). Sex reversal of double KO with estrogen (as in FIG. 34 panel A) or androgen inhibitor (as in FIG. 34 panel B) can otherwise be substituted by germ line transplantation method to produce the female broodstock (FIG. 34 panel C) or male broodstock (FIG. 34 panel D).

### Example 12—Tank Grow-Out Trials

There is a direct trade-off between growth and reproduction, as energy channeled into the gonads detracts from somatic growth. Nile tilapia mature precociously and can reproduce throughout the year, with short vitellogenic periods [26], and a physiological process that demands a high metabolic rate. Furthermore, Tilapia species can suppress growth to maintain their reproductive capacity [27], and in other fish species the onset of puberty can have a major impact on important production parameters in fish farming such as appetite, growth rate, feed conversion efficiency, flesh quality traits, external appearance, health, welfare and survival rates. Thus, delaying or blocking sexual maturation is likely to confer significant benefits to commercial aquaculture producers. In our efforts to develop sterile monosex populations, we have targeted genes whose mutations block or delay the onset of puberty. However, genes targeted for these effects might also have pleiotropic effects, detrimental to the line, acting via unknown hormonal, physiological or behavioral changes.

Experiment: To generate groups used for growth performance trials, embryos from single paired crossings (at least three separate crosses) will be produced for each line of interest. Treatment and control embryos will be reared separately using established hatchery procedures. At the feeding stage, half of the control animals will be sex reversed using appropriate exogenous hormone treatment protocols (i.e. feeding methyl testosterone or DES). When fish within a group (treatment and control) reach a mean weight of 60 g, they will be PIT tagged and divided into six 1000 L tanks (3 control and 3 treatment tanks, with 50 fish/tank). All fish will be fed three times daily, to satiation.

Each fish will be individually weighed, and the length of each fish measured at 4-week intervals over a period necessary to reach market size (680 g Sdv: 77 g, 8 months). At the end of the experiment, fish will be sacrificed and sexed based on the structure of the urogenital orifice. We will record the individual weights of dissected gonads and carcass for calculation of gonadosomatic index (GSI) and carcass index (n=60 per group). Specific growth rate (G) will be calculated according to the formula of Houde & Scheckter [28]

Without being bound by theory, we believe that most, if not all, double KO fish created in Examples 7, 8, and 9 will develop as monosex and be sterile with no other biological processes impaired. Thus, selected mutations should not negatively impact the overall fish performance. On the contrary, we expect to find an improved growth rate and feed conversion ratios inversely correlated to gonad weight. Mutant lines should be sexually delayed (male sterile) or immature (female arrested at the previtellogenic stage). In the unlikely event that we achieve only partial sterilization of monosex populations, we expect improvement in productivity in tilapia to be proportional to the fraction of sterile fish in the population, as a result of reduced energy expenditure. In all cases, we anticipate sterile fish and fish with atrophic gonads to out-perform their fully fertile counterparts (e.g. monosex populations derived from exogenous hormone treatments) in regard to growth characteristics.

### REFERENCES

1. Dunham, R., Aquaculture and Fisheries Biotechnology, CABI Publishing. 2004.
2. Pruginin, Y., et al., All-male broods of Tilapia niloticax T. aurea hybrids. Aquaculture, 1975. 6(1): p. 11-21.
3. Wolters, W. R. and R. DeMay, Production characteristics of striped bassx white bass and striped bassx yellow bass hybrids. Journal of the World Aquaculture Society, 1996. 27(2): p. 202-207.
4. McMaster, M., et al., Milt characteristics, reproductive performance, and larval survival and development of white sucker exposed to bleached kraft mill effluent. Ecotoxicology and environmental safety, 1992. 23(1): p. 103-117.
5. Lacerda, S. M., et al., A new and fast technique to generate offspring after germ cells transplantation in adult fish: the Nile tilapia (Oreochromis niloticus) model. PLoS One, 2010. 5(5): p. e10740.
6. Lauth, X. and J. T. Buchanan, Maternally induced sterility in animals. 2015, Google Patents.
7. Koga, A., et al., Insertion of a novel transposable element in the tyrosinase gene is responsible for an albino mutation in the medaka fish, Oryzias latipes. Molecular and General Genetics MGG, 1995. 249(4): p. 400-405.
8. Krauss, J., et al., transparent, a gene affecting stripe formation in Zebrafish, encodes the mitochondrial protein Mpv17 that is required for iridophore survival. Biology open, 2013. 2(7): p. 703-710.
9. Weidinger, G., et al., dead end, a Novel Vertebrate Germ Plasm Component, Is Required for Zebrafish Primordial Germ Cell Migration and Survival. Current Biology, 2003. 13(16): p. 1429-1434.
10. Peterson, R. T. and P. J. Schlueter, Germ cell ablation compounds and uses thereof. 2017, Google Patents.
11. Benfey, T. J. and A. M. Sutterlin, Growth and gonadal development in triploid landlocked Atlantic salmon (Salmo salar). Canadian Journal of Fisheries and Aquatic Sciences, 1984. 41(9): p. 1387-1392.
12. Felip, A., et al., Induction of triploidy and gynogenesis in teleost fish with emphasis on marine species. Genetica, 2001. 111(1-3): p. 175-195.
13. Zhou, L.-Y., et al., A novel type of P450c17 lacking the lyase activity is responsible for C21-steroid biosynthesis in the fish ovary and head kidney. Endocrinology, 2007. 148(9): p. 4282-4291.
14. Carlisle, S., et al., Carneiro, L. & Grober, M S (2000). Effects of 11-ketotestosterone on genital papilla morphology in the sex changing fish Lythrypnus dalli. Journal of Fish Biology 57, 445-456. The resolution of FIG. 2 when originally printed was unsatisfactory. The correct. Journal of Fish Biology, 2001. 58: p. 299.
15. Xu, X., et al., Globozoospermia in mice lacking the casein kinase II α′ catalytic subunit. Nature genetics, 1999. 23(1): p. 118.
16. Yao, R., et al., Lack of acrosome formation in mice lacking a Golgi protein, GOPC. Proceedings of the National Academy of Sciences, 2002. 99(17): p. 11211-11216.
17. Suzuki-Toyota, F., et al., Factors maintaining normal sperm tail structure during epididymal maturation studied in Gopc−/− mice. Biology of reproduction, 2007. 77(1): p. 71-82.
18. Doran, J., et al., Mfsd14a (Hiat1) gene disruption causes globozoospermia and infertility in male mice. Reproduction, 2016. 152(1): p. 91-99.
19. Rocha, D. and N. Affara, The genetic basis of impaired spermatogenesis and male infertility. Current Obstetrics & Gynaecology, 2000. 10(3): p. 139-145.
20. Truong, B., et al., Searching for candidate genes for male infertility.

Asian journal of andrology, 2003. 5(2): p. 137-147.
21. Funaki, T., et al., The Arf GAP SMAP2 is necessary for organized vesicle budding from the trans-Golgi network and subsequent acrosome formation in spermiogenesis. Molecular biology of the cell, 2013. 24(17): p. 2633-2644.
22. Oka, K., et al., Genotyping of 38 insertion/deletion polymorphisms for human identification using universal fluorescent PCR. Molecular and cellular probes, 2014. 28(1): p. 13-18.
23. Jiang, D. N., et al., gsdf is a downstream gene of dmrt1 that functions in the male sex determination pathway of the Nile tilapia. Molecular reproduction and development, 2016. 83(6): p. 497-508.
24. Li, M., et al., Efficient and heritable gene targeting in tilapia by CRISPR/Cas9. Genetics, 2014. 197(2): p. 591-599.
25. Davis, L. K., et al., Gender-specific expression of multiple estrogen receptors, growth hormone receptors, insulin-like growth factors and vitellogenins, and effects of 17β-estradiol in the male tilapia (Oreochromis mossambicus). General and comparative endocrinology, 2008. 156(3): p. 544-551.
26. Naylor, R. L., et al., Effect of aquaculture on world fish supplies. Nature, 2000. 405(6790): p. 1017.
27. Coward, K. and N. R. Bromage, Spawning frequency, fecundity, egg size and ovarian histology in groups of Tilapia zillii maintained upon two distinct food ration sizes from first-feeding to sexual maturity. Aquatic Living Resources, 1999. 12(1): p. 11-22.
28. Houde, E. D., Growth rates, rations and cohort consumption of marine fish larvae in relation to prey concentrations. Rapp. P.-V. Reun. Cons. Int. Explor. Mer, 1981. 178: p. 441-453.
29. Zhang, Z., et al., Disruption of zebrafish follicle-stimulating hormone receptor (fshr) but not luteinizing hormone receptor (Ihcgr) gene by TALEN leads to failed follicle activation in females followed by sexual reversal to males. Endocrinology, 2015. 156(10): p. 3747-3762.

SEQUENCE LISTING

SEQ ID NO 1
LENGTH: 38
TYPE: DNA
ORGANISM: Artificial Sequence
OTHER INFORMATION: Description of Artificial Sequence: Forward tailed Primer (FAM)
SEQUENCE: 1
TGTAAAACGACGGCCAGTttgaagttgctacataaaag
SEQ ID NO 2
LENGTH: 22
TYPE: DNA
ORGANISM: Artificial Sequence
OTHER INFORMATION: Description of Artificial Sequence: Primer
SEQUENCE: 2
TGGTTGATGACAATCACACTGT
SEQ ID NO 3
LENGTH: 41
TYPE: DNA
ORGANISM: Artificial Sequence
OTHER INFORMATION: Description of Artificial Sequence: Forward tailed Primer (NED)
SEQUENCE: 3
TAGGAGTGCAGCAAGCATtgttctacatcatcacccttctc
SEQ ID NO 4
LENGTH: 23
TYPE: DNA
ORGANISM: Artificial Sequence
OTHER INFORMATION: Description of Artificial Sequence: Primer
SEQUENCE: 4
AGCAGACAGACGAGCAGTATCAG
SEQ ID NO 5
LENGTH: 41
TYPE: DNA
ORGANISM: Artificial Sequence
OTHER INFORMATION: Description of Artificial Sequence: Forward tailed Primer (FAM)
SEQUENCE: 5
TGTAAAACGACGGCCAGTTGATGGAGAGCTTCATCTACGAA
SEQ ID NO 6
LENGTH: 20
TYPE: DNA
ORGANISM: Artificial Sequence
OTHER INFORMATION: Description of Artificial Sequence: Primer
SEQUENCE: 6
GTTCCAGGTTAAATTGATTG
SEQ ID NO 7
LENGTH: 41
TYPE: DNA
ORGANISM: Artificial Sequence
OTHER INFORMATION: Description of Artificial Sequence: Forward tailed Primer (NED)
SEQUENCE: 7
TAGGAGTGCAGCAAGCATgcgtgatttgctgacctttttac
SEQ ID NO 8
LENGTH: 22
TYPE: DNA
ORGANISM: Artificial Sequence
OTHER INFORMATION: Description of Artificial Sequence: Primer
SEQUENCE: 8
acacttacCCTGAGAATCTGG
SEQ ID NO 9
LENGTH: 41
TYPE: DNA
ORGANISM: Artificial Sequence
OTHER INFORMATION: Description of Artificial Sequence: Forward tailed Primer (FAM)
SEQUENCE: 9
TGTAAAACGACGGCCAGTGAAAAAGGATGgtgagggatgac
SEQ ID NO 10
LENGTH: 23
TYPE: DNA
ORGANISM: Artificial Sequence
OTHER INFORMATION: Description of Artificial Sequence: Primer
SEQUENCE: 10
GAGTGTGTCTACCACACGGAAAA
SEQ ID NO 11
LENGTH: 41
TYPE: DNA
ORGANISM: Artificial Sequence
OTHER INFORMATION: Description of Artificial Sequence: Forward tailed Primer (FAM)
SEQUENCE: 11
TGTAAAACGACGGCCAGTgtatttagaaggcggtgaaggtc
SEQ ID NO 12
LENGTH: 23
TYPE: DNA
ORGANISM: Artificial Sequence
OTHER INFORMATION: Description of Artificial Sequence: Primer
SEQUENCE: 12
CAGTTTGGCACATGAGCATCGTA
SEQ ID NO 13
LENGTH: 37
TYPE: DNA
ORGANISM: Artificial Sequence
OTHER INFORMATION: Description of Artificial Sequence: Forward tailed Primer (NED)
SEQUENCE: 13
TAGGAGTGCAGCAAGCATATGCTCATGTGCCAAACTG
SEQ ID NO 14
LENGTH: 23
TYPE: DNA
ORGANISM: Artificial Sequence
OTHER INFORMATION: Description of Artificial Sequence: Primer
SEQUENCE: 14
cCTTCAGGATTTTCACCACCACT
SEQ ID NO 15
LENGTH: 41
TYPE: DNA
ORGANISM: Artificial Sequence
OTHER INFORMATION: Description of Artificial Sequence: Forward tailed Primer (FAM)
SEQUENCE: 15
TGTAAAACGACGGCCAGTtactgacacatccagcagcgtct
SEQ ID NO 16
LENGTH: 23
TYPE: DNA
ORGANISM: Artificial Sequence
OTHER INFORMATION: Description of Artificial Sequence: Primer
SEQUENCE: 16
cagcactgagccgtcagtattct
SEQ ID NO 17
LENGTH: 37
TYPE: DNA
ORGANISM: Artificial Sequence
OTHER INFORMATION: Description of Artificial Sequence: Forward tailed Primer (NED)
SEQUENCE: 17
TAGGAGTGCAGCAAGCATTGGAGCCTACCTGTCTGAG
SEQ ID NO 18
LENGTH: 20
TYPE: DNA
ORGANISM: Artificial Sequence
OTHER INFORMATION: Description of Artificial Sequence: Primer
SEQUENCE: 18
tactcacAGCGAAGGGGTCT
SEQ ID NO 19
LENGTH: 38
TYPE: DNA
ORGANISM: Artificial Sequence
OTHER INFORMATION: Description of Artificial Sequence: Forward tailed Primer (NED)
SEQUENCE: 19
TAGGAGTGCAGCAAGCATgctcctctgcgaagactctc
SEQ ID NO 20
LENGTH: 23
TYPE: DNA
ORGANISM: Artificial Sequence
OTHER INFORMATION: Description of Artificial Sequence: Primer
SEQUENCE: 20
aagacctccgacCTGGACTTGCT
SEQ ID NO 21
LENGTH: 41
TYPE: DNA
ORGANISM: Artificial Sequence
OTHER INFORMATION: Description of Artificial Sequence: Forward tailed Primer (FAM)
SEQUENCE: 21
TGTAAAACGACGGCCAGTAGAGGAGGGCACAGTCAAGAAAC
SEQ ID NO 22
LENGTH: 22
TYPE: DNA
ORGANISM: Artificial Sequence
OTHER INFORMATION: Description of Artificial Sequence: Primer
SEQUENCE: 22
TTGGATATCCCATTTGGTTCAT
SEQ ID NO 23
LENGTH: 40
TYPE: DNA
ORGANISM: Artificial Sequence
OTHER INFORMATION: Description of Artificial Sequence: Forward tailed Primer (NED)
SEQUENCE: 23
TAGGAGTGCAGCAAGCATtttaacggtgttggcagagatt
SEQ ID NO 24
LENGTH: 22
TYPE: DNA
ORGANISM: Artificial Sequence
OTHER INFORMATION: Description of Artificial Sequence: Primer
SEQUENCE: 24
AGATCCACATCCACGAAAGCCT
SEQ ID NO 25
LENGTH: 37
TYPE: DNA
ORGANISM: Artificial Sequence
OTHER INFORMATION: Description of Artificial Sequence: Forward tailed Primer (FAM)
SEQUENCE: 25
TGTAAAACGACGGCCAGTtgcccctttaaaccaccta
SEQ ID NO 26
LENGTH: 23
TYPE: DNA
ORGANISM: Artificial Sequence
OTHER INFORMATION: Description of Artificial Sequence: Primer
SEQUENCE: 26
CTCAGCTTGGCCTTGCTTGACAT
SEQ ID NO 27
LENGTH: 39
TYPE: DNA
ORGANISM: Artificial Sequence
OTHER INFORMATION: Description of Artificial Sequence: Forward tailed Primer (NED)
SEQUENCE: 27
TAGGAGTGCAGCAAGCATttgccaggacccATGAGCCAG
SEQ ID NO 28
LENGTH: 23
TYPE: DNA
ORGANISM: Artificial Sequence
OTHER INFORMATION: Description of Artificial Sequence: Primer
SEQUENCE: 28
AGACACGTATCCGTGATTTCTAC
SEQ ID NO 29
LENGTH: 41
TYPE: DNA
ORGANISM: Artificial Sequence
OTHER INFORMATION: Description of Artificial Sequence: Forward tailed Primer (FAM)
SEQUENCE: 29
TGTAAAACGACGGCCAGTctcttcatcctctgtgtctcatc
SEQ ID NO 30
LENGTH: 22
TYPE: DNA
ORGANISM: Artificial Sequence
OTHER INFORMATION: Description of Artificial Sequence: Primer
SEQUENCE: 30
GGGTTTCCAGCAGGAGGTCAGA
SEQ ID NO 31
LENGTH: 39
TYPE: DNA
ORGANISM: Artificial Sequence
OTHER INFORMATION: Description of Artificial Sequence: Forward tailed Primer (NED)
SEQUENCE: 31
TAGGAGTGCAGCAAGCATttatgttcagGTGCCAAGGTG
SEQ ID NO 32
LENGTH: 23
TYPE: DNA
ORGANISM: Artificial Sequence
OTHER INFORMATION: Description of Artificial Sequence: Primer
SEQUENCE: 32
TGGCTGTGTGAGAAACGATGCTG
SEQ ID NO 33
LENGTH: 35
TYPE: DNA
ORGANISM: Artificial Sequence
OTHER INFORMATION: Description of Artificial Sequence: Forward tailed Primer (FAM)
SEQUENCE: 33
TGTAAAACGACGGCCAGTagATCTGGGCTGGGACA
SEQ ID NO 34
LENGTH: 23
TYPE: DNA
ORGANISM: Artificial Sequence
OTHER INFORMATION: Description of Artificial Sequence: Primer
SEQUENCE: 34
tgttaactatacCTGTGTGTTGG
SEQ ID NO 35
LENGTH: 38
TYPE: DNA
ORGANISM: Artificial Sequence
OTHER INFORMATION: Description of Artificial Sequence: Forward tailed Primer (NED)
SEQUENCE: 35
TAGGAGTGCAGCAAGCATttttctccgcttgcttctgc
SEQ ID NO 36
LENGTH: 23
TYPE: DNA
ORGANISM: Artificial Sequence
OTHER INFORMATION: Description of Artificial Sequence: Primer
SEQUENCE: 36
AAAGAGCTGAATAGGAGGAAGTT
SEQ ID NO 37
LENGTH: 39
TYPE: DNA
ORGANISM: Artificial Sequence
OTHER INFORMATION: Description of Artificial Sequence: Forward tailed Primer (FAM)
SEQUENCE: 37
TGTAAAACGACGGCCAGTCATCTTGGCGTTCTTCTGTGT
SEQ ID NO 38
LENGTH: 21
TYPE: DNA
ORGANISM: Artificial Sequence
OTHER INFORMATION: Description of Artificial Sequence: Primer
SEQUENCE: 38
CTTGAGGGCAGCTGAGATGGC
SEQ ID NO 39
LENGTH: 40
TYPE: DNA
ORGANISM: Artificial Sequence
OTHER INFORMATION: Description of Artificial Sequence: Forward tailed Primer (NED)
SEQUENCE: 39
TAGGAGTGCAGCAAGCATGCAATCCTTGATGCTCCTTGAC
SEQ ID NO 40
LENGTH: 22
TYPE: DNA
ORGANISM: Artificial Sequence
OTHER INFORMATION: Description of Artificial Sequence: Primer
SEQUENCE: 40
CTGAGACTCTATGTCGTTGATA
SEQ ID NO 41
LENGTH: 41
TYPE: DNA
ORGANISM: Artificial Sequence
OTHER INFORMATION: Description of Artificial Sequence: Forward tailed Primer (FAM)
SEQUENCE: 41
TGTAAAACGACGGCCAGTAGAAGATCATCAAACACATCACG
SEQ ID NO 42
LENGTH: 23
TYPE: DNA
ORGANISM: Artificial Sequence
OTHER INFORMATION: Description of Artificial Sequence: Primer
SEQUENCE: 42
GACTTGTTGAGCAGTTGCATCAA
SEQ ID NO 43
LENGTH: 38
TYPE: DNA
ORGANISM: Artificial Sequence
OTHER INFORMATION: Description of Artificial Sequence: Forward tailed Primer (NED)
SEQUENCE: 43
TAGGAGTGCAGCAAGCATttttgtgatctagTCTGGAG
SEQ ID NO 44
LENGTH: 22
TYPE: DNA
ORGANISM: Artificial Sequence
OTHER INFORMATION: Description of Artificial Sequence: Primer
SEQUENCE: 44
gctcttacAGCTTCACAATCAT
SEQ ID NO 45
LENGTH: 41
TYPE: DNA
ORGANISM: Artificial Sequence
OTHER INFORMATION: Description of Artificial Sequence: Forward tailed Primer (FAM)
SEQUENCE: 45
TGTAAAACGACGGCCAGTAGAAGATCATCAAACACATCACG
SEQ ID NO 46
LENGTH: 23
TYPE: DNA
ORGANISM: Artificial Sequence
OTHER INFORMATION: Description of Artificial Sequence: Primer
SEQUENCE: 46
GACTTGTTGAGCAGTTGCATCAA
SEQ ID NO 47
LENGTH: 22
TYPE: DNA
ORGANISM: Artificial Sequence
OTHER INFORMATION: Description of Artificial Sequence: Primer
SEQUENCE: 47
GAACCAAACCCCTCTGTCACTG
SEQ ID NO 48
LENGTH: 22
TYPE: DNA
ORGANISM: Artificial Sequence
OTHER INFORMATION: Description of Artificial Sequence: Primer
SEQUENCE: 48
GTAATTCACTCCGCAGGCTCAG
SEQ ID NO 49
LENGTH: 17
TYPE: DNA
ORGANISM: Artificial Sequence
OTHER INFORMATION: Description of Artificial Sequence: Primer
SEQUENCE: 49
ggcgATGAATCCTGTAG
SEQ ID NO 50
LENGTH: 22
TYPE: DNA
ORGANISM: Artificial Sequence
OTHER INFORMATION: Description of Artificial Sequence: Primer
SEQUENCE: 50
ATGGCATTTGAGGTCACAGAGA
SEQ ID NO 51
LENGTH: 19
TYPE: DNA
ORGANISM: Artificial Sequence
OTHER INFORMATION: Description of Artificial Sequence: Primer
SEQUENCE: 51
GTTCAAGAAGGGAGAGAGT
SEQ ID NO 52
LENGTH: 18
TYPE: DNA
ORGANISM: Artificial Sequence
OTHER INFORMATION: Description of Artificial Sequence: Primer
SEQUENCE: 52
AAAAATTCCCACATCGTT
SEQ ID NO 53
LENGTH: 20
TYPE: DNA
ORGANISM: Artificial Sequence
OTHER INFORMATION: Description of Artificial Sequence: Primer
SEQUENCE: 53
tgctttggcttcagTGTATC
SEQ ID NO 54
LENGTH: 19
TYPE: DNA
ORGANISM: Artificial Sequence
OTHER INFORMATION: Description of Artificial Sequence: Primer
SEQUENCE: 54
AATGCGTTCGAATGTAGAA
SEQ ID NO 55
LENGTH: 23
TYPE: DNA
ORGANISM: Artificial Sequence
OTHER INFORMATION: Description of Artificial Sequence: Primer
SEQUENCE: 55
CATCTGCTTCATCCTGGTGGCTG
SEQ ID NO 56
LENGTH: 23
TYPE: DNA
ORGANISM: Artificial Sequence
OTHER INFORMATION: Description of Artificial Sequence: Primer
SEQUENCE: 56
AATTTGGGCATCTTCATCTGTAT
SEQ ID NO 57
LENGTH: 22
TYPE: DNA
ORGANISM: Artificial Sequence
OTHER INFORMATION: Description of Artificial Sequence: Primer
SEQUENCE: 57
GACAGACTTGACCTTGGAGATG
SEQ ID NO 58
LENGTH: 21
TYPE: DNA
ORGANISM: Artificial Sequence
OTHER INFORMATION: Description of Artificial Sequence: Primer
SEQUENCE: 58
ATGTCTGCTTCGACTGGATGC
SEQ ID NO 59
LENGTH: 23
TYPE: DNA
ORGANISM: Artificial Sequence
OTHER INFORMATION: Description of Artificial Sequence: Primer
SEQUENCE: 59
GCCATCGAAACATGGACATACTG
SEQ ID NOs 60 and 62 (wild-type Cypl7a1)
LENGTH: 1563 bp and 521 aa
TYPE: cDNA (SEQ ID NO: 60) and Protein (SEQ ID NO: 62)
ORGANISM: Nile tilapia

| | | |
|---|---|---|
| 1 | GAACCAAACCCCTCTGTCACTGATATGGCTTGGTTTTTGTGTCTGTGCGTGTTCATGGCG | 60 |
| 1 | -E--P--N--P--S--V--T--D--M--A--W--F--L--C--L--C--V--F--M--A- | 20 |
| 61 | GTGGGCCTCACTTTGTTAGCACTGCAGTTCAAGTTCAGGATGTCTGCACATGGTTCTGGG | 120 |
| 21 | -V--G--L--T--L--L--A--L--Q--F--K--F--R--M--S--A--H--G--S--G- | 40 |
| 121 | GAGCCGCCACACCTCCCTGCACTACCACTGATTGGCAGCCTGCTGAGCCTGCGGAGTGAA | 180 |
| 41 | -E--P--P--H--L--P--A--L--P--L--I--G--S--L--L--S--L--R--S--E- | 60 |
| 181 | TTACCACCGCATGTGCTTTTCAAAGAACTGCAGGTAAAATACGGACATACATACTCGCTG | 240 |
| 61 | -L--P--P--H--V--L--F--K--E--L--Q--V--K--Y--G--H--T--Y--S--L- | 80 |
| 241 | ATGATGGGCTCCCACAGTGTGATTGTCATCAACCAGCATGTGCACGCCAAAGAAGTCTTG | 300 |
| 81 | -M--M--G--S--H--S--V--I--V--I--N--Q--H--V--H--A--K--E--V--L- | 100 |
| 301 | CTCAAGAAGGGAAAGACGTTTGCAGGAAGACCTAGAACTGTAACCACAGATATTCTGACT | 360 |
| 101 | -L--K--K--G--K--T--F--A--G--R--P--R--T--V--T--T--D--I--L--T- | 120 |
| 361 | AGAGATGGGAAGGACATTGCATTTGGAGACTACAGTGCTACGTGGAAGTTCCACAGGAAG | 420 |
| 121 | -R--D--G--K--D--I--A--F--G--D--Y--S--A--T--W--K--F--H--R--K- | 140 |
| 421 | ATAGTCCATGGAGCCCTGTGCATGTTTGGAGAAGGTTCTGCCTCTATTGAGAAGACCATT | 480 |
| 141 | -I--V--H--G--A--L--C--M--F--G--E--G--S--A--S--I--E--K--T--I- | 160 |
| 481 | TGTGCAGAGGCCCAGTCTCTGTGCTCCGTGCTGTCTGAGGCAGCAGATGTGGGACTGGCC | 540 |
| 161 | -C--A--E--A--Q--S--L--C--S--V--L--S--E--A--A--D--V--G--L--A- | 180 |
| 541 | CTGGATCTTGCTCCTGAGCTGACTCGCGCTGTCACCAACGTTATCTGTTCTCTCTGCTTC | 600 |
| 181 | -L--D--L--A--P--E--L--T--R--A--V--T--N--V--I--C--S--L--C--F- | 200 |
| 601 | AACTCGTCCTACTGCCGAGGCGACTCAGAGTTTGAGACAATGCTGCAGTACAGCCAGGGC | 660 |
| 201 | -N--S--S--Y--C--R--G--D--S--E--F--E--T--M--L--Q--Y--S--Q--G- | 220 |
| 661 | ATTGTGGACACTGTGGCTAAAGACAGCCTGGTAGACATTTTCCCCTGGCTTCAGATCTTT | 720 |
| 221 | -I--V--D--T--V--A--K--D--S--L--V--D--I--F--P--W--L--Q--I--F- | 240 |
| 721 | CCTAATGCGGACCTACGTCTCCTAAAACATTGTGTTTCCATCAGAGACAAACTTCTACAG | 780 |
| 241 | -P--N--A--D--L--R--L--L--K--H--C--V--S--I--R--D--K--L--L--Q- | 260 |
| 781 | AGGAAATTTGATGAACACAAGGTGAATTACAATGATCACGTGCAGAGAGACTTGATAGAC | 840 |
| 261 | -R--K--F--D--E--H--K--V--N--Y--N--D--H--V--Q--R--D--L--I--D- | 280 |
| 841 | GCTCTGCTAAGAGCCAAGCGCAGTGCGGAGAACAACAACACATCAGAGATAAGTGCAGAG | 900 |
| 281 | -A--L--L--R--A--K--R--S--A--E--N--N--N--T--S--E--I--S--A--E- | 300 |
| 901 | TCTGTGGGCCTGAGTGATGACCACATTCTCATGACAGTGGGAGACATCTTTGGCGCTGGC | 960 |
| 301 | -S--V--G--L--S--D--D--H--I--L--M--T--V--G--D--I--F--G--A--G- | 320 |
| 961 | GTGGAAACCACTACCACTGTGCTCAAATGGGCCATAACGTACCTCATTCATCACCCAGAG | 1020 |
| 321 | -V--E--T--T--T--T--V--L--K--W--A--I--T--Y--L--I--H--H--P--E- | 340 |
| 1021 | GTGCAAAGACGTATCCAGGATGAGCTGGACAGGACGGTGGGTGACAGCCGCTCTCCTAAA | 1080 |
| 341 | -V--Q--R--R--I--Q--D--E--L--D--R--T--V--G--D--S--R--S--P--K- | 360 |
| 1081 | CTCACCGACAGAGGCAGTCTGCCTTATCTGGAGGCCACCATTAGGGAAGTATTGCGGATT | 1140 |
| 361 | -L--T--D--R--G--S--L--P--Y--L--E--A--T--I--R--E--V--L--R--I- | 380 |
| 1141 | CGCCCCGTGGCACCACTACTCATCCCCCATGTGGCTCTCTGTGACACCAGCATTGGAGAT | 1200 |
| 381 | -R--P--V--A--P--L--L--I--P--H--V--A--L--C--D--T--S--I--G--D- | 400 |
| 1201 | TTCACAGTGAGAAAAGGAACTCGAGTCATTATCAACCTGTGGGCTCTGCACCATGATGAG | 1260 |
| 401 | -F--T--V--R--K--G--T--R--V--I--I--N--L--W--A--L--H--H--D--E- | 420 |
| 1261 | AAGGAGTGGAAGAACCCAGAGCGGTTTGACCCTGGCCGGTTCTTGAAAAGTGAAGGCACA | 1320 |
| 421 | -K--E--W--K--N--P--E--R--F--D--P--G--R--F--L--K--S--E--G--T- | 440 |
| 1321 | GGACTGACAATCCCATCACCCAGCTACCTGCCCTTTGGTGCTGGGCTGAGAGTATGTTTA | 1380 |
| 441 | -G--L--T--I--P--S--P--S--Y--L--P--F--G--A--G--L--R--V--C--L- | 460 |
| 1381 | GGTGAGGCCTTGGCCAAGATGGAGCTCTTTCTCTTCCTGTCCTGGATCCTGCAGCGCTTC | 1440 |
| 461 | -G--E--A--L--A--K--M--E--L--F--L--F--L--S--W--I--L--Q--R--F- | 480 |
| 1441 | ACTCTGTCTGTCCCACCAGGCCACAGTCTGCCCAGTCTGGAGGGAAAGTTTGGAGTGGTC | 1500 |
| 481 | -T--L--S--V--P--P--G--H--S--L--P--S--L--E--G--K--F--G--V--V- | 500 |
| 1501 | CTGCAGACAGCCAAGTACAAGGTGAATGCCACAATCAGACCAGACTGGGCAAGACATAAG | 1560 |
| 501 | -L--Q--T--A--K--Y--K--V--N--A--T--I--R--P--D--W--A--R--H--K- | 520 |
| 1561 | TGC | 1563 |
| 521 | -C- | 521 |

SEQ ID NOs 61 and 63 (Cyp17a1 mutant allele- 16 nt deletion)
LENGTH: 1563 bp and 44 aa
TYPE: cDNA (SEQ ID NO: 61) and Protein (SEQ ID NO: 63)
ORGANISM: Nile tilapia

| | | |
|---|---|---|
| 1 | GAACCAAACCCCTCTGTCACTGATATGGCTTGGTTTTTGTGTCTGTGCGGTGGGCCTCAC | 60 |
| 1 | -E--P--N--P--S--V--T--D--M--A--W--F--L--C--L--C--G--G--P--H- | 20 |
| 61 | TTTGTTAGCACTGCAGTTCAAGTTCAGGATGTCTGCACATGGTTCTGGGGAGCCACCTCC | 120 |
| 21 | -F--V--S--T--A--V--Q--V--Q--D--V--C--T--W--F--W--G--A--T--S- | 40 |
| 121 | CTGCACTACCACTGATTGGCAGCCTGCTGAGCCTGCGGAGTGAATTACCACCGCATGTGC | 180 |
| 41 | -L--H--Y--H--*- | 44 |

SEQ ID NOs 65 and 68 (wild-type Cyp19a1a)
LENGTH: 1707 bp and 511 aa
TYPE: cDNA (SEQ ID NO: 65) and Protein (SEQ ID NO: 68)
ORGANISM: Nile tilapia

| | | |
|---|---|---|
| 1 | GCGATGAATCCTGTAGGCTTAGACGCCGTGGTGGCAGATCTCTCTGTGACCTCAAATGCC | 60 |
| | ...-M--N--P--V--G--L--D--A--V--V--A--D--L--S--V--T--S--N--A- | 19 |
| 61 | ATCCAATCGCATGGGATATCAATGGCAACCAGAACGCTGATACTGCTCGTCTGTCTGCTG | 120 |
| 20 | -I--Q--S--H--G--I--S--M--A--T--R--T--L--I--L--L--V--C--L--L- | 39 |
| 121 | TTGGTTGCCTGGAGTCACACGGACAAGAAAATTGTGCCAGGTCCTTCTTTCTGTTTGGGT | 180 |
| 40 | -L--V--A--W--S--H--T--D--K--K--I--V--P--G--P--S--F--C--L--G- | 59 |
| 181 | TTGGGCCCACTTCTGTCATATCTGAGATTTATCTGGACTGGCATAGGCACAGCCAGCAAC | 240 |
| 60 | -L--G--P--L--L--S--Y--L--R--F--I--W--T--G--I--G--T--A--S--N- | 79 |
| 241 | TACTACAATAACAAGTATGGAGACATTGTTAGAGTCTGGATCAACGGAGAAGAGACGCTC | 300 |
| 80 | -Y--Y--N--N--K--Y--G--D--I--V--R--V--W--I--N--G--E--E--T--L- | 99 |
| 301 | ATACTAAGCAGATCTTCAGCAGTGCACCATGTGCTGAAGAACGGAAACTATACTTCACGT | 360 |
| 100 | -I--L--S--R--S--S--A--V--H--H--V--L--K--N--G--N--Y--T--S--R- | 119 |
| 361 | TTTGGGAGCATCCAGGGACTCAGCTGCCTCGGCATGAACGAGAGAGGCATCATATTCAAC | 420 |
| 120 | -F--G--S--I--Q--G--L--S--C--L--G--M--N--E--R--G--I--I--F--N- | 139 |
| 421 | AACAACGTAACTCTGTGGAAAAAGATACGCACCTATTTTGCTAAAGCTCTGACAGGCCCA | 480 |
| 140 | -N--N--V--T--L--W--K--K--I--R--T--Y--F--A--K--A--L--T--G--P- | 159 |
| 481 | AATTTGCAGCAGACGGCGGATGTTTGCGTCTCCTCCATACAGGCTCACCTGGACCACCTG | 540 |
| 160 | -N--L--Q--Q--T--A--D--V--C--V--S--S--I--Q--A--H--L--D--H--L- | 179 |
| 541 | GACAGCCTGGGACACGTTGATGTCCTCAATTTGCTGCGCTGCACCGTGCTGGACATCTCT | 600 |
| 180 | -D--S--L--G--H--V--D--V--L--N--L--L--R--C--T--V--L--D--I--S- | 199 |
| 601 | AACCGACTCTTCCTGGACGTACCTCTCAATGAGAAAGAGCTGATGCTGAAGATTCAAAAG | 660 |
| 200 | -N--R--L--F--L--D--V--P--L--N--E--K--E--L--M--L--K--I--Q--K- | 219 |
| 661 | TATTTTCACACATGGCAGGATGTGCTTATCAAACCTGACATCTACTTCAAGTTCGGCTGG | 720 |
| 220 | -Y--F--H--T--W--Q--D--V--L--I--K--P--D--I--Y--F--K--F--G--W- | 239 |
| 721 | ATTCACCACAGGCACAAGACAGCAACCCAGGAGTTACAAGATGCCATTAAACGTCTTGTA | 780 |
| 240 | -I--H--H--R--H--K--T--A--T--Q--E--L--Q--D--A--I--K--R--L--V- | 259 |
| 781 | GATCAAAAGAGGAAAAATATGGAGCAGGCAGATACGCTGGACAACATCAACTTCACGGCA | 840 |
| 260 | -D--Q--K--R--K--N--M--E--Q--A--D--T--L--D--N--I--N--F--T--A- | 279 |
| 841 | GAGCTCATATTTGCACAAAACCACGGTGAGCTGTCTGCTGAGAATGTGACGCAGTGCGTG | 900 |
| 280 | -E--L--I--F--A--Q--N--H--G--E--L--S--A--E--N--V--T--Q--C--V- | 299 |
| 901 | CTGGAGATGGTGATTGCAGCTCCGGACACTCTGTCCCTCAGTCTCTTCTTCATGCTTCTG | 960 |
| 300 | -L--E--M--V--I--A--A--P--D--T--L--S--L--S--L--F--F--M--L--L- | 319 |
| 961 | CTCCTCAAACAAAACCCGCACGTGGAGCCGCAGCTGCTACAGGAGATAGACGCTGTTGTG | 1020 |
| 320 | -L--L--K--Q--N--P--H--V--E--P--Q--L--L--Q--E--I--D--A--V--V- | 339 |
| 1021 | GGTGAGAGACAGCTTCAGAACCAGGATCTTCACAAGCTGCAGGTGATGGAGAGCTTCATC | 1080 |
| 340 | -G--E--R--Q--L--Q--N--Q--D--L--H--K--L--Q--V--M--E--S--F--I- | 359 |
| 1081 | TACGAATGCTTGCGCTTCCACCCAGTGGTGGACTTCACCATGCGTCGAGCCCTGTCTGAT | 1140 |
| 360 | -Y--E--C--L--R--F--H--P--V--V--D--F--T--M--R--R--A--L--S--D- | 379 |
| 1141 | GACATCATAGAAGGCTACAGGATCTCGAAGGGCACAAACATCATTCTGAACACAGGCCGA | 1200 |
| 380 | -D--I--I--E--G--Y--R--I--S--K--G--T--N--I--I--L--N--T--G--R- | 399 |
| 1201 | ATGCACCGCACCGAGTTTTTCCTCAAAGCCAATCAATTTAACCTGGAACACTTTGAAAAC | 1260 |
| 400 | -M--H--R--T--E--F--F--L--K--A--N--Q--F--N--L--E--H--F--E--N- | 419 |
| 1261 | AATGTTCCTCGGCGCTACTTTCAGCCGTTCGGTTCAGGCCCTCGCGCATGCATTGGCAAG | 1320 |
| 420 | -N--V--P--R--R--Y--F--Q--P--F--G--S--G--P--R--A--C--I--G--K- | 439 |
| 1321 | CACATCGCCATGGTGATGATGAAATCCATTTTGGTGACACTGCTGTCTCAGTACTCTGTT | 1380 |
| 440 | -H--I--A--M--V--M--M--K--S--I--L--V--T--L--L--S--Q--Y--S--V- | 459 |
| 1381 | TGTACTCACGAGGGCCCGATCCTGGACTGCCTCCCACAAACCAACAACCTTTCCCAGCAG | 1440 |
| 460 | -C--T--H--E--G--P--I--L--D--C--L--P--Q--T--N--N--L--S--Q--Q- | 479 |
| 1441 | CCTGTAGAGCACCAGCAGGCGGAGACTGAACATCTCCACATGAGGTTCTTACCCAGGCAG | 1500 |
| 480 | -P--V--E--H--Q--Q--A--E--T--E--H--L--H--M--R--F--L--P--R--Q- | 499 |
| 1501 | AGAAGCAGCTGTCAAACCCTCCGAGACCCGAACCTTTAGCTGTACCTGCACTTTTGTATA | 1560 |
| 500 | -R--S--S--C--Q--T--L--R--D--P--N--L--*-..................... | 511 |
| 1561 | CTTAATTTGTATAATCTTATAACGACACACAGCTAGCCTTTATATTTTGATAGACGCAAA | 1620 |
| | ............................................................ | |
| 1621 | GATTGTATTTGTACTCAAACTGTATGCATGATGTGAAATGTACATTTAAACCTGCTAACA | 1680 |
| | ............................................................ | |
| 1681 | CTGAAATAAATGTAAGTTATTGTGTCA | 1707 |
| | ............................................................ | |

SEQ ID NOs 66 and 69 (Cvp19ala mutant allele- 7 nt deletion)
LENGTH: 1707 bp and 12 aa
TYPE: cDNA (SEQ ID NO: 66) and Protein (SEQ ID NO: 69)
ORGANISM: Nile tilapia

| | | |
|---|---|---|
| 1 | GCGATGAATCCTGTAGGCTTAGACTGGCAGATCTCTCTGTGACCTCAAATGCCATCCAAT | 60 |
| | ...-M--N--P--V--G--L--D--W--Q--I--S--L--*- | 12 |

SEQ ID NOs 67 and 70 (Cvp19ala mutant allele- 10 nt deletion)
LENGTH: 1707 bp and 11 aa
TYPE: cDNA (SEQ ID NO: 67) and Protein (SEQ ID NO: 70)
ORGANISM: Nile tilapia

| | | |
|---|---|---|
| 1 | GCGATGAATCCTGTAGGCTGGTGGCAGATCTCTCTGTGACCTCAAATGCCATCCAATCGC | 60 |
| 11 | ...-M--N--P--V--G--W--W--Q--I--S--L--*- | |

SEQ ID NOs 71 and 73 (wild-type TiPla)
LENGTH: 6674 bp and 1652 aa
TYPE: cDNA (SEQ ID NO: 71) and Protein (SEQ ID NO: 73)
ORGANISM: Nile tilapia

| | | |
|---|---|---|
| 1 | AAAGAGGAAAACAATGCATCATATAACTTTATAAGTAAGAGTGCGGCGATGGAGGAAACC | 60 |
| 1 | -K--E--E--N--N--A--S--Y--N--F--I--S--K--S--A--A--M--E--E--T- | 20 |
| 61 | GTCATATGGGAACAGCACACAGTTACCCTTCACAGGGCCCCAGGATTTGGGTTTGGCATT | 120 |
| 21 | -V--I--W--E--Q--H--T--V--T--L--H--R--A--P--G--F--G--F--G--I- | 40 |
| 121 | GCCATCTCGGGTGGGCGAGACAACCCTCATTTCCAGAGTGGTGAAACATCTATTGTAATA | 180 |
| 41 | -A--I--S--G--G--R--D--N--P--H--F--Q--S--G--E--T--S--I--V--I- | 60 |
| 181 | TCGGATGTGCTGAAAGGAGGTCCTGCAGAGGGTCTGCTACAAGAAAATGATCGAGTAGTA | 240 |
| 61 | -S--D--V--L--K--G--G--P--A--E--G--L--L--Q--E--N--D--R--V--V- | 80 |
| 241 | ATGGTCAATGCAGTCTCTATGGACAATGTAGAGCATGCCTATGCTGTTCAACAACTTCGA | 300 |
| 81 | -M--V--N--A--V--S--M--D--N--V--E--H--A--Y--A--V--Q--Q--L--R- | 100 |
| 301 | AAGAGTGGCAAAAATGCAAAGATAACTATTCGCAGAAAAAGGAAAGTACAAATCCCAGCG | 360 |
| 101 | -K--S--G--K--N--A--K--I--T--I--R--R--K--R--K--V--Q--I--P--A- | 120 |
| 361 | TCACGGCACGGGGACAGGGAGACGATGTCTGAGCACGAGGAGGAGGACAGCGATGAGGCT | 420 |
| 121 | -S--R--H--G--D--R--E--T--M--S--E--H--E--E--E--D--S--D--E--A- | 140 |
| 421 | GATGCTTACGATCACCGCAGTGGACGTGGTGGACAAAACGATCGGGAGCGTAGCAGCAGT | 480 |
| 141 | -D--A--Y--D--H--R--S--G--R--G--G--Q--N--D--R--E--R--S--S--S- | 160 |
| 481 | GGGAGGCGGGATCACAGTGCCTCACAGGAGAGGAGCATCTCACCACGCTCCGATCGCCGA | 540 |
| 161 | -G--R--R--D--H--S--A--S--Q--E--R--S--I--S--P--R--S--D--R--R- | 180 |
| 541 | TCACAAGCCTCTTCTGCTCCACCCAGGCCCTCCAAGGTCACTCTTGTCAAGTCCCGCAAA | 600 |
| 181 | -S--Q--A--S--S--A--P--P--R--P--S--K--V--T--L--V--K--S--R--K- | 200 |
| 601 | AACGAAGAATATGGACTGCGGCTGGCCAGCCATATCTTTGTGAAGGACATCTCTCCAGAG | 660 |
| 201 | -N--E--E--Y--G--L--R--L--A--S--H--I--F--V--K--D--I--S--P--E- | 220 |
| 661 | AGCCTTGCAGCCAGAGATGGAAACATTCAGGAGGGAGATGTTGTACTTAAGATTAACGGC | 720 |
| 221 | -S--L--A--A--R--D--G--N--I--Q--E--G--D--V--V--L--K--I--N--G- | 240 |
| 721 | ACAGTTACAGAGAACCTATCACTGACAGATGCCAAGAAGCTGATTGAGAGGTCAAAGGGC | 780 |
| 241 | -T--V--T--E--N--L--S--L--T--D--A--K--K--L--I--E--R--S--K--G- | 260 |
| 781 | AAGCTGAAGATGGTAGTGCAGAGAGACGAGCGGGCCACGCTGCTCAATATTCCTGACCTT | 840 |
| 261 | -K--L--K--M--V--V--Q--R--D--E--R--A--T--L--L--N--I--P--D--L- | 280 |
| 841 | GACGACAGCATCCCATCAGCCAATAACTCAGACAGAGATGACATTTCAGAGATACATTCA | 900 |
| 281 | -D--D--S--I--P--S--A--N--N--S--D--R--D--D--I--S--E--I--H--S- | 300 |
| 901 | CTGACATCCGATCATTCCAATCGATCCCATGGGAGAGGAAGTCAATCCCATTCGCCTGAC | 960 |
| 301 | -L--T--S--D--H--S--N--R--S--H--G--R--G--S--Q--S--H--S--P--D- | 320 |
| 961 | AGGGTTGAAACATCCGAGCATCTCCGCCACTCACCGCGGCAGATCAGCAATGGCAGTAAT | 1020 |
| 321 | -R--V--E--T--S--E--H--L--R--H--S--P--R--Q--I--S--N--G--S--N- | 340 |
| 1021 | GGATTTCTCTGGGAAAGAGCTGAGGAATTAATCAAACAGGAATGGGTGGTGAAACAGGAA | 1080 |
| 341 | -G--F--L--W--E--R--A--E--E--L--I--K--Q--E--W--V--V--K--Q--E- | 360 |
| 1081 | TGTTATTTTGCCTGTGCCCATACTATAAAATGTGTAATAACCGTGACAGTTTGGGCAAAA | 1140 |
| 361 | -C--Y--F--A--C--A--H--T--I--K--C--V--I--T--V--T--V--W--A--K- | 380 |
| 1141 | AAACCCCAAAACAGTAACATGCCAGAACCAAAGCCAGTTTATGCACAGCCTGGTCAGCCT | 1200 |
| 381 | -K--P--Q--N--S--N--M--P--E--P--K--P--V--Y--A--Q--P--G--Q--P- | 400 |
| 1201 | GACGTGGACCTGCCTGTCAGCCCATCTGATGCCCCTGTACCCAGTGCTGCACATGATGAC | 1260 |
| 401 | -D--V--D--L--P--V--S--P--S--D--A--P--V--P--S--A--A--H--D--D- | 420 |
| 1261 | AGCATTCTCAGACCAAGTATGAAGCTGGTCAAGTTCAAGAAGGGAGAGAGTGTCGGTCTG | 1320 |
| 421 | -S--I--L--R--P--S--M--K--L--V--K--F--K--K--G--E--S--V--G--L- | 440 |
| 1321 | AGGTTAGCAGGCGGAAACGATGTGGGAATTTTTGTGGCAGGAGTTTTGGAAGACAGCCCC | 1380 |
| 441 | -R--L--A--G--G--N--D--V--G--I--F--V--A--G--V--L--E--D--S--P- | 460 |
| 1381 | GCAGCCAAGGAGGGGCTGGAAGAGGGAGACCAGATTCTCAGGGTGAACAACGTGGACTTT | 1440 |
| 461 | -A--A--K--E--G--L--E--E--G--D--Q--I--L--R--V--N--N--V--D--F- | 480 |
| 1441 | GCTAACATCATCCGGGAAGAGGCTGTGCTTTTTCTGCTCGATCTTCCAAAAGGAGATGAC | 1500 |
| 481 | -A--N--I--I--R--E--E--A--V--L--F--L--L--D--L--P--K--G--D--D- | 500 |
| 1501 | GTTACTATTCTGGCTCAGAAGAAAAAGGATGTGTATCGAAGGATAGTGGAATCAGACGTG | 1560 |
| 501 | -V--T--I--L--A--Q--K--K--K--D--V--Y--R--R--I--V--E--S--D--V- | 520 |
| 1561 | GGTGACTCCTTCTACATTCGAACGCATTTTGAATATGAAAAAGAGTCACCGTATGGGCTG | 1620 |
| 521 | -G--D--S--F--Y--I--R--T--H--F--E--Y--E--K--E--S--P--Y--G--L- | 540 |
| 1621 | AGCTTTAACAAGGGAGAGGTTTTCCGTGTGGTAGACACACTCTATAATGGCAAATTAGGC | 1680 |
| 541 | -S--F--N--K--G--E--V--F--R--V--V--D--T--L--Y--N--G--K--L--G- | 560 |
| 1681 | TCCTGGCTCGCTATCCGTATCGGCAAGAACCACCAGGAAGTGGAAAGAGGCATAATCCCC | 1740 |
| 561 | -S--W--L--A--I--R--I--G--K--N--H--Q--E--V--E--R--G--I--I--P- | 580 |
| 1741 | AACAAGAATAGAGCCGAGCAGCTATCCAGTGTGCAGTACACCCTTCCTAAAACGCCTGGG | 1800 |
| 581 | -N--K--N--R--A--E--Q--L--S--S--V--Q--Y--T--L--P--K--T--P--G- | 600 |
| 1801 | GGCGACAGAGCTGACTTCTGGAGGTTCAGAGGGCTGCGGAGTTCCAAGAGGAATTTGCGG | 1860 |
| 601 | -G--D--R--A--D--F--W--R--F--R--G--L--R--S--S--K--R--N--L--R- | 620 |
| 1861 | AAAAGCAGGGAGGACCTGTCGGCCCAGCCTGTTCAGACCAAGTTCCCTGCCTATGAGAGG | 1920 |
| 621 | -K--S--R--E--D--L--S--A--Q--P--V--Q--T--K--F--P--A--Y--E--R- | 640 |
| 1921 | GTGGTGCTGAGGGAAGCTGGGTTCCTGAGGCCTGTGGTTATCTTTGGGCCGATTGCAGAC | 1980 |
| 641 | -V--V--L--R--E--A--G--F--L--R--P--V--V--I--F--G--P--I--A--D- | 660 |
| 1981 | GTGGCCCGAGAGAAACTGGCCAGGGAGGTGCCCGAAGTGTTTGAGCTAGCCAAGAGTGAA | 2040 |
| 661 | -V--A--R--E--K--L--A--R--E--V--P--E--V--F--E--L--A--K--S--E- | 680 |
| 2041 | CCCAGGGATGCAGGAACAGACCAGAAGAGCTCTGGCATCATCCGCCTGCACACCATTAAG | 2100 |
| 681 | -P--R--D--A--G--T--D--Q--K--S--S--G--I--I--R--L--H--T--I--K- | 700 |
| 2101 | CAGATCATTGATCGAGACAAGCATGCAGTGCTGGATATAACCCCGAATGCAGTGGACCGA | 2160 |
| 701 | -Q--I--I--D--R--D--K--H--A--V--L--D--I--T--P--N--A--V--D--R- | 720 |
| 2161 | CTGAACTACGCTCAGTGGTATCCAATTGTGGTGTTTCTCAACCCGGACACCAAGCAGGGC | 2220 |
| 721 | -L--N--Y--A--Q--W--Y--P--I--V--V--F--L--N--P--D--T--K--Q--G- | 740 |
| 2221 | ATCAAGAACATGAGGACACGGCTCTGCCCCGAGTCTAGGAAGAGCGCGAGAAAGCTTTAT | 2280 |
| 741 | -I--K--N--M--R--T--R--L--C--P--E--S--R--K--S--A--R--K--L--Y- | 760 |
| 2281 | GATCGAGCCCTCAAGTTAAGAAAGAACAACCACCACCTCTTCACCACAACCATTAACTTG | 2340 |
| 761 | -D--R--A--L--K--L--R--K--N--N--H--H--L--F--T--T--T--I--N--L- | 780 |
| 2341 | AACAACATGAACGATGGTTGGTTTGGAGCACTGAAAGAAATCATCCATCAGCAGCAGAAC | 2400 |
| 781 | -N--N--M--N--D--G--W--F--G--A--L--K--E--I--I--H--Q--Q--Q--N- | 800 |
| 2401 | CAGCTGGTGTGGGTTTCAGAGGGCAAGGCTGATGGAGTTGGCGACGATGACCTGGACATC | 2460 |
| 801 | -Q--L--V--W--V--S--E--G--K--A--D--G--V--G--D--D--D--L--D--I- | 820 |
| 2461 | CACGACGACCGCCTTTCCTACCTGTCGGCGCCAGGCAGTGAGTATTCCATGTACAGCACC | 2520 |
| 821 | -H--D--D--R--L--S--Y--L--S--A--P--G--S--E--Y--S--M--Y--S--T- | 840 |
| 2521 | GACAGCCGCCACACCTCCGATTACGAGGACACGGACACAGAGGGAGGAGCCTACACCGAC | 2580 |
| 841 | -D--S--R--H--T--S--D--Y--E--D--T--D--T--E--G--G--A--Y--T--D- | 860 |
| 2581 | CAGGAGCTGGATGAAACGCTGAACGATGACGTGGGTCCACCCACGGAGCCTGCCATCACG | 2640 |
| 861 | -Q--E--L--D--E--T--L--N--D--D--V--G--P--P--T--E--P--A--I--T- | 880 |
| 2641 | CGGTCCTCTGAGCCTGTCCGTGAGGACCCGCCTGTCATCCAAGAGCCCCCTGGCTATGTC | 2700 |
| 881 | -R--S--S--E--P--V--R--E--D--P--P--V--I--Q--E--P--P--G--Y--V- | 900 |
| 2701 | AGCTACCCGCACACAGTGCAGCCGGACCCCCTGAACCGCATCGACCCGGCTGGTTTCAAG | 2760 |
| 901 | -S--Y--P--H--T--V--Q--P--D--P--L--N--R--I--D--P--A--G--F--K- | 920 |
| 2761 | GCACCAGCGCCGCAGCAGATGTTTCAGAAGGATCCGTACAGCACAGACAACATAGGCAGA | 2820 |
| 921 | -A--P--A--P--Q--Q--M--F--Q--K--D--P--Y--S--T--D--N--I--G--R- | 940 |
| 2821 | GGTGGTCACGGCATGAAGCCTGTGACGTACAACCCTCAGCAGGGGTATCACCCCGACGAG | 2880 |
| 941 | -G--G--H--G--M--K--P--V--T--Y--N--P--Q--Q--G--Y--H--P--D--E- | 960 |
| 2881 | CAGCCATACAGAGATTACGATCACCCACCCAGCCGGTATGACATCAGCAGCAGTGGTGTC | 2940 |
| 961 | -Q--P--Y--R--D--Y--D--H--P--P--S--R--Y--D--I--S--S--S--G--V- | 980 |
| 2941 | GGCGGTGGCTACCAGGAGCCAAAGTACCGTAACTATGAGAGCTATGAGAACAGCGTGCCT | 3000 |
| 981 | -G--G--G--Y--Q--E--P--K--Y--R--N--Y--E--S--Y--E--N--S--V--P- | 1000 |
| 3001 | CACTACGACCAGCAACCGTGGAACCCCTACAACCAGCCGTTCTCCACTGCCAACACCCAG | 3060 |
| 1001 | -H--Y--D--Q--Q--P--W--N--P--Y--N--Q--P--F--S--T--A--N--T--Q- | 1020 |
| 3061 | GCCTACGATCCCCGTCCTCCTTACGGTGAGGGCCCCGACTCTCATTACACCCCTCCCCTG | 3120 |
| 1021 | -A--Y--D--P--R--P--P--Y--G--E--G--P--D--S--H--Y--T--P--P--L- | 1040 |
| 3121 | CGCTACGACGAGCCGCCACCTCAGCAGGGATTTGACGGACGGCCTCGCTACGGCAAACCG | 3180 |
| 1041 | -R--Y--D--E--P--P--P--Q--Q--G--F--D--G--R--P--R--Y--G--K--P- | 1060 |
| 3181 | ACAGTTTCAGCACCTGTCCGTTACGATGATCTTCCGCCTCCCCCTCAGCCGTCTGAATTG | 3240 |
| 1061 | -T--V--S--A--P--V--R--Y--D--D--L--P--P--P--P--Q--P--S--E--L- | 1080 |
| 3241 | CACTATGACCCAAATTCTCACCTGAGCACATACCCCTCAGCTGCCCGCTCACCAGAACCC | 3300 |
| 1081 | -H--Y--D--P--N--S--H--L--S--T--Y--P--S--A--A--R--S--P--E--P- | 1100 |
| 3301 | GCTGCCCAGCGACCCGCCTATAACCAGGGACCAGCATCGCAGCAGAAAGGTTACAAACCT | 3360 |
| 1101 | -A--A--Q--R--P--A--Y--N--Q--G--P--A--S--Q--Q--K--G--Y--K--P- | 1120 |
| 3361 | CAGCAGTACGATCCTGCTCCTGTGAACTCTGAATCCAGCCCCAGCCTTCATAAAGTCGAG | 3420 |
| 1121 | -Q--Q--Y--D--P--A--P--V--N--S--E--S--S--P--S--L--H--K--V--E- | 1140 |
| 3421 | ACGCCCTCACCTTCACCTGCTGATGTTCCAAAAGCTGCACCTGCAAGAGATGAGCAGCAG | 3480 |
| 1141 | -T--P--S--P--S--P--A--D--V--P--K--A--A--P--A--R--D--E--Q--Q- | 1160 |
| 3481 | GAGGAGGATCCAGCCATGCGGCCTCAGTCAGTACTGACGAGGGTCAAAATGTTTGAGAAC | 3540 |
| 1161 | -E--E--D--P--A--M--R--P--Q--S--V--L--T--R--V--K--M--F--E--N- | 1180 |
| 3541 | AAACGCTCTGTGTCCATGGACCGAGCCAGAGATGCCGGGGATTCATTTGGGAATAAGGCA | 3600 |
| 1181 | -K--R--S--V--S--M--D--R--A--R--D--A--G--D--S--F--G--N--K--A- | 1200 |
| 3601 | GCCGATTTGCCCTTGAAAGCTGGTGGAGTAATCCCTAAAGCAAATTCTCTGAGCAACCTG | 3660 |
| 1201 | -A--D--L--P--L--K--A--G--G--V--I--P--K--A--N--S--L--S--N--L- | 1220 |
| 3661 | GATCAAGAGAAGACCTTTAGCAGAGGGCCAGAGCCTCAGAAGCCTCAGTCCAAGGGATCC | 3720 |
| 1221 | -D--Q--E--K--T--F--S--R--G--P--E--P--Q--K--P--Q--S--K--G--S- | 1240 |
| 3721 | GATGACATCGTGCGCTCCAACCATTATGACCCTGATGAGGATGAGGACTACTACAGGAAA | 3780 |
| 1241 | -D--D--I--V--R--S--N--H--Y--D--P--D--E--D--E--D--Y--Y--R--K- | 1260 |
| 3781 | CAGTTGTCTTACTTTGACAGACTGCAGACTGGCTCCAATAAACCCCAACCACAAGCACAG | 3840 |
| 1261 | -Q--L--S--Y--F--D--R--L--Q--T--G--S--N--K--P--Q--P--Q--A--Q- | 1280 |
| 3841 | TCCAGCCACAGCTTCCCCAGCCATTATACACATTTTGGATATTCAAGTGTCTTTCTTTTC | 3900 |
| 1281 | -S--S--H--S--F--P--S--H--Y--T--H--F--G--Y--S--S--V--F--L--F- | 1300 |
| 3901 | TTTTCCTTAATGATGGACTCTGTGGAGAAACCAAGCCCACTGGAGAAAAAATATGAACCA | 3960 |
| 1301 | -F--S--L--M--M--D--S--V--E--K--P--S--P--L--E--K--K--Y--E--P- | 1320 |
| 3961 | GTTCCCCAAGTGACACCAGCTGTGCCACCGGCCACGCTGCCCAAGCCCTCACCTGATGGT | 4020 |
| 1321 | -V--P--Q--V--T--P--A--V--P--P--A--T--L--P--K--P--S--P--D--G- | 1340 |
| 4021 | AAAATTGACTGTAGTCAGGATTTTTATCTCATCTCTTTGACTGATGTGCGTTGCTCTTCC | 4080 |
| 1341 | -K--I--D--C--S--Q--D--F--Y--L--I--S--L--T--D--V--R--C--S--S- | 1360 |
| 4081 | ACAGCCAAACCTCCTGCTCGAGAGGACACGGTCCAGACCAACTTTCTTCCTCACAAGAGC | 4140 |
| 1361 | -T--A--K--P--P--A--R--E--D--T--V--Q--T--N--F--L--P--H--K--S- | 1380 |
| 4141 | TTCCCTGAGAAGTCTCCAGTCAATGGCACCAGTGAACAGCCTCCAAAGACGGTCACTAGC | 4200 |
| 1381 | -F--P--E--K--S--P--V--N--G--T--S--E--Q--P--P--K--T--V--T--S- | 1400 |
| 4201 | ACCGGGGGTTTGCCCACATCCACCTACAACCGCTTTGCGCCCAAGCCCTACACCTCCTCT | 4260 |
| 1401 | -T--G--G--L--P--T--S--T--Y--N--R--F--A--P--K--P--Y--T--S--S- | 1420 |
| 4261 | GCCAAGCCTTTTTCGCGCAAGTTCGACAGTCCTAAATTCAACCACAACCTCCTGTCCAAT | 4320 |
| 1421 | -A--K--P--F--S--R--K--F--D--S--P--K--F--N--H--N--L--L--S--N- | 1440 |
| 4321 | GACAAGCCTGAGAGTGCTCCCAAGGGACGGAGCTCGAGTCCGGTAAAGCCTCAGGTACCC | 4380 |
| 1441 | -D--K--P--E--S--A--P--K--G--R--S--S--S--P--V--K--P--Q--V--P- | 1460 |
| 4381 | CCACAGCCCCAGAACGCAGACCAAGACAGTGGCCTGGACACTTTCACACGCACAACGGAC | 4440 |
| 1461 | -P--Q--P--Q--N--A--D--Q--D--S--G--L--D--T--F--T--R--T--T--D- | 1480 |
| 4441 | CCCCGATCCAAATACCAGCAGAACAACGTAAACGCCGTGCCCAAGGCCATCCCTGTGAGC | 4500 |
| 1481 | -P--R--S--K--Y--Q--Q--N--N--V--N--A--V--P--K--A--I--P--V--S- | 1500 |
| 4501 | CCCAGTGCCCTAGAGGATGATGAAGATGAAGACGAAGGTCACACTGTGGTGGCAACAGCT | 4560 |
| 1501 | -P--S--A--L--E--D--D--E--D--E--D--E--G--H--T--V--V--A--T--A- | 1520 |
| 4561 | CGTGGCATCTTCAACTCTAACGGTGGCGTTCTGAGCTCCATCGAGACAGGTGTCAGCATC | 4620 |
| 1521 | -R--G--I--F--N--S--N--G--G--V--L--S--S--I--E--T--G--V--S--I- | 1540 |
| 4621 | ATTATCCCACAGGGTGCCATCCCCGACGGCGTGGAGCAAGAGATTTACTTCAAGGTCTGT | 4680 |
| 1541 | -I--I--P--Q--G--A--I--P--D--G--V--E--Q--E--I--Y--F--K--V--C- | 1560 |
| 4681 | CGAGACAACAGCATCCTGCCGCCACTCGACAAGGAGAAAGGAGAGACTCTGCTCAGCCCT | 4740 |
| 1561 | -R--D--N--S--I--L--P--P--L--D--K--E--K--G--E--T--L--L--S--P- | 1580 |
| 4741 | CTGGTGATGTGTGGACCTCACGGCCTAAAGTTCCTGAAGCCTGTGGAGCTACGCTTACCT | 4800 |
| 1581 | -L--V--M--C--G--P--H--G--L--K--F--L--K--P--V--E--L--R--L--P- | 1600 |
| 4801 | CACTGTGCGTCAATGACCCCTGATGGTTGGTCTTTTGCTCTAAAATCCTCCGACTCCTCG | 4860 |
| 1601 | -H--C--A--S--M--T--P--D--G--W--S--F--A--L--K--S--S--D--S--S- | 1620 |
| 4861 | TCGGGTGATCCAAAAAGCTGGCAGAACAAGTCTCTCACCGGAGACCCCAACTACCTGGTG | 4920 |
| 1621 | -S--G--D--P--K--S--W--Q--N--K--S--L--T--G--D--P--N--Y--L--V- | 1640 |
| 4921 | GGAGCCAACTGTGTCTCTGTGCTCATTGACCACTTTTAAAGAAGAAGCAGCAGGTGTGAT | 4980 |
| 1641 | -G--A--N--C--V--S--V--L--I--D--H--F--*-..................... | 1652 |
| 4981 | GTTACTGAATGTGGAAGAATGGCGGATGAAATGAAGACGATGGAAACGCACGCACGCAAA | 5040 |
| | ............................................................ | |
| 5041 | CACACACATATACCACTACACACACACACACACTGACAGACGCACTCCAAGCAAACCAAC | 5100 |
| | ............................................................ | |
| 5101 | ACACAGCATAGAGTATGAAGAAGACCCAGACAGTGCTGGACGAAGGAGAGACACCAATGA | 5160 |
| | ............................................................ | |
| 5161 | TCGTTACGAGCTGTTCTTTAAACTCAATTTCAAAGTTTTGATGTAAAATGATGCATGCCC | 5220 |
| | ............................................................ | |
| 5221 | AACGTCACTGACGATTGACACTTATATATAAAGCAATGTTTAATGTAATTTTTCTTTTTT | 5280 |
| | ............................................................ | |
| 5281 | CTTTTTTTACAAAAGTATAGATGGATGTATGGCTTTTGAGGCAGCATACATGCTTGAAAA | 5340 |
| | ............................................................ | |
| 5341 | ATCTGTGTCAATGTATTTATGCTATATATGCCTACAGTATATATAGAAGAATAGAGAAGA | 5400 |
| | ............................................................ | |
| 5401 | AATTGGACTCGAATTCGATCGCCAGTCAACATCTTGTTGTTTTTTCAGTTCAGGGGACTG | 5460 |
| | ............................................................ | |
| 5461 | GATTTTTTGTTTGTTTGTTTGTTTGTTTTTTTCCCTTCCACATTGAAGGAATCTTACTGA | 5520 |
| | ............................................................ | |
| 5521 | AGGTTTGATACAGTTGGTTTAAGGAGGTGGCAAGACATGAGCTGGACATGAACCCAAGCA | 5580 |
| | ............................................................ | |
| 5581 | GCAGCAACAGCACACTTTTAGAGACGTTCTTCCTACACTTCTCACTTTGTTCTTCCTTTT | 5640 |
| | ............................................................ | |
| 5641 | CTTACCTTTTGTAGCTTCCTCTCTTACTGAGCACCACCTCTCTCCTTCCAGCCTGAGGGA | 5700 |
| | ............................................................ | |
| 5701 | GATCTATGCATGTTCTTTACTCAGGTCCAGTAGCCTCCTCGGTTCCTTCCTCACATCTAC | 5760 |
| | ............................................................ | |
| 5761 | TTAATATCTTTCCTTTCTCTGTGCACTCTTTGCACTCACACAAATAAGCAGTGATGCCTT | 5820 |
| | ............................................................ | |
| 5821 | ATCTGCAGATTATTCACTTTTCATTAAGAAAAAAAAGTAAGTTATGATAAATTATGGTAT | 5880 |
| | ............................................................ | |
| 5881 | AATGTCATTTGTTTTGCCATTTTTTTGTGAACCCTCTGTATAAATAAACTTGGGTTTAGC | 5940 |
| | ............................................................ | |
| 5941 | ACACGCAGAAACAGTCGATAAAAGATAACAAAGGTATGCTCTTCTTTTATCTGCTATGCA | 6000 |
| | ............................................................ | |
| 6001 | TTGCTTAAAAACAAAAAACCATCAGAGAAGAAGTGGCTGTAAATAAAGCTAGCATATTGC | 6060 |
| | ............................................................ | |
| 6061 | CTTGTTTCTTTTTTGTTGTAAATGAACTTTTTGTATGTCTTTCTTTTTTGTATAAAACTT | 6120 |
| | ............................................................ | |
| 6121 | AGAGAAAACATGTTTTAAAAAAGCAGAAGGAAGTGAAAGTGGTTATCTTTGTATTATGGG | 6180 |
| | ............................................................ | |
| 6181 | CATACCTTCAAGCCTTTGAATTGTAACCTAACAATAATACATCACACCTTTTCTACCGAT | 6240 |
| | ............................................................ | |
| 6241 | ATGTTGCCGCCGCTATTTTACCGTCTCAAAAAGGTCGTCTTTTTTTATTTTTATTTCTAT | 6300 |
| | ............................................................ | |
| 6301 | TTTTATTACTTTGTCCACGTAGGGTTAAGGAAAGTATTTGCGGCTCAGATGCATTTAAAA | 6360 |
| | ............................................................ | |
| 6361 | CATCTTCATTTGGAAGGGTGTGCTCTCAAAGTGTCCCTCTCACTGATTTCTGATACTGGA | 6420 |
| | ............................................................ | |
| 6421 | TGCTATATTGTGTGCCCTTAAATGTATTTTTGTACTAATAGACAATATATTACGTATGGC | 6480 |
| | ............................................................ | |
| 6481 | ACACCAGTACTGTTTTACTTTTAGATATAACACGGCTTTATTGGATATTAGCTCTTCACT | 6540 |
| | ............................................................ | |
| 6541 | TGTGGCTGACTTTTTTTTTTTTCCCCTCTGCAACACAATTTTAAGTATACCACTGTATTA | 6600 |
| | ............................................................ | |
| 6601 | ATAAATAAAATCATTTTTAAATTAAAGAGTGTGTAAGGATTTTTTATTTTTTTTTACCCT | 6660 |
| | ............................................................ | |
| 6661 | ACAGGGTTTTGTAT | 6674 |
| | .............. | |

SEQ ID NOs 72 and 74 (Tip1a mutant allele- 7 nt deletion)
LENGTH: 6674 bp and 439 aa
TYPE: cDNA (SEQ ID NO: 72) and Protein (SEQ ID NO: 74)
ORGANISM: Nile tilapia

| | | |
|---|---|---|
| 1 | AAAGAGGAAAACAATGCATCATATAACTTTATAAGTAAGAGTGCGGCGATGGAGGAAACC | 60 |
| 1 | -K--E--E--N--N--A--S--Y--N--F--I--S--K--S--A--A--M--E--E--T- | 20 |
| 61 | GTCATATGGGAACAGCACACAGTTACCCTTCACAGGGCCCCAGGATTTGGGTTTGGCATT | 120 |
| 21 | -V--I--W--E--Q--H--T--V--T--L--H--R--A--P--G--F--G--F--G--I- | 40 |
| 121 | GCCATCTCGGGTGGGCGAGACAACCCTCATTTCCAGAGTGGTGAAACATCTATTGTAATA | 180 |
| 41 | -A--I--S--G--G--R--D--N--P--H--F--Q--S--G--E--T--S--I--V--I- | 60 |
| 181 | TCGGATGTGCTGAAAGGAGGTCCTGCAGAGGGTCTGCTACAAGAAAATGATCGAGTAGTA | 240 |
| 61 | -S--D--V--L--K--G--G--P--A--E--G--L--L--Q--E--N--D--R--V--V- | 80 |
| 241 | ATGGTCAATGCAGTCTCTATGGACAATGTAGAGCATGCCTATGCTGTTCAACAACTTCGA | 300 |
| 81 | -M--V--N--A--V--S--M--D--N--V--E--H--A--Y--A--V--Q--Q--L--R- | 100 |
| 301 | AAGAGTGGCAAAAATGCAAAGATAACTATTCGCAGAAAAAGGAAAGTACAAATCCCAGCG | 360 |
| 101 | -K--S--G--K--N--A--K--I--T--I--R--R--K--R--K--V--Q--I--P--A- | 120 |
| 361 | TCACGGCACGGGGACAGGGAGACGATGTCTGAGCACGAGGAGGAGGACAGCGATGAGGCT | 420 |
| 121 | -S--R--H--G--D--R--E--T--M--S--E--H--E--E--E--D--S--D--E--A- | 140 |
| 421 | GATGCTTACGATCACCGCAGTGGACGTGGTGGACAAAACGATCGGGAGCGTAGCAGCAGT | 480 |
| 141 | -D--A--Y--D--H--R--S--G--R--G--G--Q--N--D--R--E--R--S--S--S- | 160 |
| 481 | GGGAGGCGGGATCACAGTGCCTCACAGGAGAGGAGCATCTCACCACGCTCCGATCGCCGA | 540 |
| 161 | -G--R--R--D--H--S--A--S--Q--E--R--S--I--S--P--R--S--D--R--R- | 180 |
| 541 | TCACAAGCCTCTTCTGCTCCACCCAGGCCCTCCAAGGTCACTCTTGTCAAGTCCCGCAAA | 600 |
| 181 | -S--Q--A--S--S--A--P--P--R--P--S--K--V--T--L--V--K--S--R--K- | 200 |
| 601 | AACGAAGAATATGGACTGCGGCTGGCCAGCCATATCTTTGTGAAGGACATCTCTCCAGAG | 660 |
| 201 | -N--E--E--Y--G--L--R--L--A--S--H--I--F--V--K--D--I--S--P--E- | 220 |
| 661 | AGCCTTGCAGCCAGAGATGGAAACATTCAGGAGGGAGATGTTGTACTTAAGATTAACGGC | 720 |
| 221 | -S--L--A--A--R--D--G--N--I--Q--E--G--D--V--V--L--K--I--N--G- | 240 |
| 721 | ACAGTTACAGAGAACCTATCACTGACAGATGCCAAGAAGCTGATTGAGAGGTCAAAGGGC | 780 |
| 241 | -T--V--T--E--N--L--S--L--T--D--A--K--K--L--I--E--R--S--K--G- | 260 |
| 781 | AAGCTGAAGATGGTAGTGCAGAGAGACGAGCGGGCCACGCTGCTCAATATTCCTGACCTT | 840 |
| 261 | -K--L--K--M--V--V--Q--R--D--E--R--A--T--L--L--N--I--P--D--L- | 280 |
| 841 | GACGACAGCATCCCATCAGCCAATAACTCAGACAGAGATGACATTTCAGAGATACATTCA | 900 |
| 281 | -D--D--S--I--P--S--A--N--N--S--D--R--D--D--I--S--E--I--H--S- | 300 |
| 901 | CTGACATCCGATCATTCCAATCGATCCCATGGGAGAGGAAGTCAATCCCATTCGCCTGAC | 960 |
| 301 | -L--T--S--D--H--S--N--R--S--H--G--R--G--S--Q--S--H--S--P--D- | 320 |
| 961 | AGGGTTGAAACATCCGAGCATCTCCGCCACTCACCGCGGCAGATCAGCAATGGCAGTAAT | 1020 |
| 321 | -R--V--E--T--S--E--H--L--R--H--S--P--R--Q--I--S--N--G--S--N- | 340 |
| 1021 | GGATTTCTCTGGGAAAGAGCTGAGGAATTAATCAAACAGGAATGGGTGGTGAAACAGGAA | 1080 |
| 341 | -G--F--L--W--E--R--A--E--E--L--I--K--Q--E--W--V--V--K--Q--E- | 360 |
| 1081 | TGTTATTTTGCCTGTGCCCATACTATAAAATGTGTAATAACCGTGACAGTTTGGGCAAAA | 1140 |
| 361 | -C--Y--F--A--C--A--H--T--I--K--C--V--I--T--V--T--V--W--A--K- | 380 |
| 1141 | AAACCCCAAAACAGTAACATGCCAGAACCAAAGCCAGTTTATGCACAGCCTGGTCAGCCT | 1200 |
| 381 | -K--P--Q--N--S--N--M--P--E--P--K--P--V--Y--A--Q--P--G--Q--P- | 400 |
| 1201 | GACGTGGACCTGCCTGTCAGCCCATCTGATGCCCCTGTACCCAGTGCTGCACATGATGAC | 1260 |
| 401 | -D--V--D--L--P--V--S--P--S--D--A--P--V--P--S--A--A--H--D--D- | 420 |
| 1261 | AGCATTCTCAGACCAAGTATGAAGCTGGTCAAGTTCAAGAAGGGAGAGAGTGTCGGTTAG | 1320 |
| 421 | -S--I--L--R--P--S--M--K--L--V--K--F--K--K--G--E--S--V--G--*- | 439 |

SEQ ID NOs 75 and 77 (wild-type Hiat1a)
LENGTH: 5281 bp and 491 aa
TYPE: cDNA (SEQ ID NO: 75) and Protein (SEQ ID NO: 77)
ORGANISM: Nile tilapia

| | | |
|---|---|---|
| 1 | TTCTGCTTCGCCCTTGTATTAGACAGCCAATCGCTGGACGTCACTCCGCCAGAAGGGGTG | 60 |
| | ............................................................ | |
| 61 | GGTTGACGTAGTACAGGAAGCCAGGCGAGGTGAGGTGGGGAGGAGAGATCACAAAATTGT | 120 |
| | ............................................................ | |
| 121 | TAGCTCGCTGCTAGCTGCCTCCTCCGATTTGCCCGAAGTGCGATGAGCCCAGGAGGCGAA | 180 |
| | ............................................................ | |
| 181 | ATTTGTGGGGTTTTTTGGTTTTGATTGGCGCGACGATGACCCTCTGACCCTAAGAATGGA | 240 |
| | ............................................................ | |
| 241 | CATAAGTTAATGATGACGGGGGAGAAGAAGAAGAAGAAGCGGCTGAACCGCAGCATTCTT | 300 |
| | .........-M--M--T--G--E--K--K--K--K--K--R--L--N--R--S--I--L- | 17 |
| 301 | CTTGCAAAGAAAATTATAATAAAAGATGGAGGAAGTCCTCAGGGAATCGGGGAGCCCAGT | 360 |
| 18 | -L--A--K--K--I--I--I--K--D--G--G--S--P--Q--G--I--G--E--P--S- | 37 |
| 361 | GTTTACCATGCTGTGGTGGTCATCTTCCTGGAGTTTTTTGCATGGGGTCTGCTCACTACC | 420 |
| 38 | -V--Y--H--A--V--V--V--I--F--L--E--F--F--A--W--G--L--L--T--T- | 57 |
| 421 | CCGATGCTCACGGTATTACACCAGACATTCCCCCAACACACATTCCTGATGAATGGGCTC | 480 |
| 58 | -P--M--L--T--V--L--H--Q--T--F--P--Q--H--T--F--L--M--N--G--L- | 77 |
| 481 | ATTCATGGTGTGAAGGGCCTGTTATCATTTCTCAGTGCTCCGCTAATTGGAGCGTTGTCA | 540 |
| 78 | -I--H--G--V--K--G--L--L--S--F--L--S--A--P--L--I--G--A--L--S- | 97 |
| 541 | GACGTATGGGGACGCAAGTCCTTCCTGCTGCTAACGGTCTTCTTCACTTGCGCGCCCATT | 600 |
| 98 | -D--V--W--G--R--K--S--F--L--L--L--T--V--F--F--T--C--A--P--I- | 117 |
| 601 | CCGCTGATGAAGATCAGTCCATGGTGGTACTTTGCAGTCATCTCGATGTCCGGTGTTTTT | 660 |
| 118 | -P--L--M--K--I--S--P--W--W--Y--F--A--V--I--S--M--S--G--V--F- | 137 |
| 661 | GCCGTCACCTTCTCTGTGATCTTTGCCTATGTGGCAGACATCACACAAGAGCATGAGAGG | 720 |
| 138 | -A--V--T--F--S--V--I--F--A--Y--V--A--D--I--T--Q--E--H--E--R- | 157 |
| 721 | AGCACAGCTTATGGTTTGGTATCAGCTACCTTTGCAGCAAGCCTGGTTACCAGCCCAGCC | 780 |
| 158 | -S--T--A--Y--G--L--V--S--A--T--F--A--A--S--L--V--T--S--P--A- | 177 |
| 781 | ATTGGAGCCTACCTGTCTGAGGCTTACAGTGACACCTTGGTTGTGATCCTGGCCACAGCC | 840 |
| 178 | -I--G--A--Y--L--S--E--A--Y--S--D--T--L--V--V--I--L--A--T--A- | 197 |
| 841 | ATCGCACTGCTCGACATCTGCTTCATCCTGGTGGCTGTACCAGAGTCGCTGCCGGAGAAG | 900 |
| 198 | -I--A--L--L--D--I--C--F--I--L--V--A--V--P--E--S--L--P--E--K- | 217 |
| 901 | ATGAGGCCAGCGTCATGGGGAGCGCCCATCTCCTGGGAACAGGCAGACCCCTTCGCTTCT | 960 |
| 218 | -M--R--P--A--S--W--G--A--P--I--S--W--E--Q--A--D--P--F--A--S- | 237 |
| 961 | CTGCGTAAAGTGGGCCAGGACTCTACGGTGCTCCTCATCTGTATCACAGTGTTCCTCTCC | 1020 |
| 238 | -L--R--K--V--G--Q--D--S--T--V--L--L--I--C--I--T--V--F--L--S- | 257 |
| 1021 | TACCTCCCTGAGGCCGGCCAGTACTCCAGCTTCTTCCTCTATCTCAGACAGGTCATAGGC | 1080 |
| 258 | -Y--L--P--E--A--G--Q--Y--S--S--F--F--L--Y--L--R--Q--V--I--G- | 277 |
| 1081 | TTCTCCTCAGAGACAGTGGCTGCCTTCATCGCTGTTGTGGGAATCCTCTCAATATTAGCT | 1140 |
| 278 | -F--S--S--E--T--V--A--A--F--I--A--V--V--G--I--L--S--I--L--A- | 297 |
| 1141 | CAGACGGTCGTGTTGGGGATCCTGATGCGCTCTATAGGAAATAAGAACACAATCCTGCTC | 1200 |
| 298 | -Q--T--V--V--L--G--I--L--M--R--S--I--G--N--K--N--T--I--L--L- | 317 |
| 1201 | GGCCTCGGCTTTCAGATCCTCCAGCTGGCCTGGTATGGCTTTGGATCTCAGCCCTGGATG | 1260 |
| 318 | -G--L--G--F--Q--I--L--Q--L--A--W--Y--G--F--G--S--Q--P--W--M- | 337 |
| 1261 | ATGTGGGCAGCTGGAGCCGTTGCTGCCATGTCCAGCATCACGTTCCCCGCCATCAGCGCC | 1320 |
| 338 | -M--W--A--A--G--A--V--A--A--M--S--S--I--T--F--P--A--I--S--A- | 357 |
| 1321 | ATTGTGTCCCGTAATGCGGATCCTGACCAGCAAGGTGTTGTTCAGGGCATGATCACTGGA | 1380 |
| 358 | -I--V--S--R--N--A--D--P--D--Q--Q--G--V--V--Q--G--M--I--T--G- | 377 |
| 1381 | ATTCGAGGCCTCTGTAACGGTTTGGGTCCTGCTCTTTACGGCTTCGTCTTCTACTTATTC | 1440 |
| 378 | -I--R--G--L--C--N--G--L--G--P--A--L--Y--G--F--V--F--Y--L--F- | 397 |
| 1441 | CACGTGGAGCTGACCGACACGGACGGCTCTGAGAAAGGTGCCAAAGGGAACATGGCCAAC | 1500 |
| 398 | -H--V--E--L--T--D--T--D--G--S--E--K--G--A--K--G--N--M--A--N- | 417 |
| 1501 | CCCACTGACGAGAGTGCCATCATCCCAGGTCCTCCCTTCCTCTTTGGTGCATGCTCAGTG | 1560 |
| 418 | -P--T--D--E--S--A--I--I--P--G--P--P--F--L--F--G--A--C--S--V- | 437 |
| 1561 | CTGCTGTCTCTGCTGGTGGCGCTGTTCATCCCGGAGCACACTGGGCCCGGTATGAGGCCC | 1620 |
| 438 | -L--L--S--L--L--V--A--L--F--I--P--E--H--T--G--P--G--M--R--P- | 457 |
| 1621 | GGCTCCTACAAGAAGCACAGCAACGGGGCACAGAGTCACTCCCACAGCCCGCAAGGCAGC | 1680 |
| 458 | -G--S--Y--K--K--H--S--N--G--A--Q--S--H--S--H--S--P--Q--G--S- | 477 |
| 1681 | GGGGCAGAGGGCAAGGAGCCGCTGCTGGAGGACAGCAGCGTATAACCTCAGCTCAGGGGG | 1740 |
| 478 | -G--A--E--G--K--E--P--L--L--E--D--S--S--V--*-............... | 491 |
| 1741 | GGCAGACTCCCTCGCTCCACCTCAAAATGCCCTGCACACATGGACAGATACACATAATTT | 1800 |
| | ............................................................ | |
| 1801 | ATCACAAGGACACACACGCACCTCAGGCACACGTCACACTCGAGTGCCGCAAAGAGATGT | 1860 |
| | ............................................................ | |
| 1861 | TTGTCTGTTTTGCTGTCCACAGCACAAAGTTGGGCGCTCCTTCCTTAGCAACCCTTTTCT | 1920 |
| | ............................................................ | |
| 1921 | TTATAATAGCTGGGTTATTGTGAGGACTTTCTAAAGACCCTGTGTGAAGAAAGTGTGTCG | 1980 |
| | ............................................................ | |
| 1981 | AGCATCATCAGGGCTGCAGTGGAAGACCGTGTATGTGTGTGTGTGTGTGTGTGTGTGTGT | 2040 |
| | ............................................................ | |
| 2041 | GTGTGTGTGGCTGAGCTGAGCTGAGCTGGACTCCAATCTTTGGTTTGTCTGAAGTTGTAA | 2100 |
| | ............................................................ | |
| 2101 | CAGTGGAGCACACAACAGCTTGTCCCCCTCCTGGCGCGAAACAGGACTGAAGTGACTTTG | 2160 |
| | ............................................................ | |
| 2161 | GTTTAATGTGCGAGTGGGGATATATCTCTGATACGTTACTAAATACCTGTGTGACTCTTG | 2220 |
| | ............................................................ | |
| 2221 | ATTATTCCTCTTTAGTTAGCCAAGTGGCACCTTCGTTTGTCAGAGGAGAGCGTGACGAAC | 2280 |
| | ............................................................ | |
| 2281 | GCCCTCTCACATGCTAATACTTCTGTTCTGATGCTTGTCTTTATGACTACAGCTCTGTTT | 2340 |
| | ............................................................ | |
| 2341 | AGGCGTCCAAGAAGGAAACATAGTTCTTCCTCTGTGTGGACAACAGGGGAGCGCAGCAGC | 2400 |
| | ............................................................ | |
| 2401 | TGTTAAACCTGTGAAAGGAGCCTGCAAACCAGTATTGGAGAGGCGCTGCCTAATTGCAGT | 2460 |
| | ............................................................ | |
| 2461 | CAGGGTTGGCAACCAGTTCAGATACAAAAAGCTTTGTTAGGACCAGGTTTTGTTCAAATA | 2520 |
| | ............................................................ | |
| 2521 | TCAAACTTCTTACAGAGAGATGACTAGAAGAGACCACTTTATTAGCTCAAAATGGTTTTT | 2580 |
| | ............................................................ | |
| 2581 | CAATGTTTACTTGCCATTCTCTAGATTAGTAGTACAGTTTGGGTTGTATATTTTTCTCTG | 2640 |
| | ............................................................ | |
| 2641 | TTCAAACTGAAGGCTAGTTGTGCTTCAAGTTTTTATTCAAGAAACAAATGTTGCCTTGAA | 2700 |
| | ............................................................ | |
| 2701 | GTGACTTAAGATATATATGGAGACATTACGTAACCTGTATGAAGACCGAGGTCTGAGAAG | 2760 |
| | ............................................................ | |
| 2761 | GCTCTGTAATCTTGCGCTATTGCTCCCATCGGAGCCGTTACACACTTTTTATTCCTTTGT | 2820 |
| | ............................................................ | |
| 2821 | ATTCATGCCCTTCCTGTTACTTTGTTTCCTGACATTTATCACCATCAAGTTGAGGCTTAC | 2880 |
| | ............................................................ | |
| 2881 | AGAGACACGGTTTTATTTTTAAAAAGCCTCTGGACCATTTGGAGCTGGAGCATTGCTATC | 2940 |
| | ............................................................ | |
| 2941 | AGGATGTCGGTGTCTGCACTGACTGTTTGAGTTGATATCATTAGGTTCAGCAGAATATCA | 3000 |
| | ............................................................ | |
| 3001 | GCCATGCTGCTGCAGTAGTAAATACAAAGGTTAATCAGTGTGGCGTAAAGTGGTGGATAA | 3060 |
| | ............................................................ | |
| 3061 | GAATTATAACTGTGTCTTGTAGTCCCTGACATTTAAGCTAACATGCGTACACTCAAAGAG | 3120 |
| | ............................................................ | |
| 3121 | GCAGGCCACACTTCTCCCAATGCCTAACATGAAGCACCTCACGGACGTGTCTGGCAACTT | 3180 |
| | ............................................................ | |
| 3181 | GTGTAGAAGCTCTGCAGATGCCAGCCTGCGCCACCTAAGAGGCAGAAACAAATAGCAGTA | 3240 |
| | ............................................................ | |
| 3241 | GTGGAGTAGATGGCTGGAAATGTTCATGTTATCCTCAAACAGTGAAGCAAAGTAAAAATC | 3300 |
| | ............................................................ | |
| 3301 | TGGAGGTTGTGTCAATGTGGAGAGTATTGCGAAATCTGCAATGATCCCAGATTTCATTAG | 3360 |
| | ............................................................ | |
| 3361 | TTTAAAAAAAAGAGAAAATAAGAAGAAGAAGAAAATCCACTTAAAAGTGTAAATCCTGAA | 3420 |
| | ............................................................ | |
| 3421 | TTTTTATTATCGTTCAGATCTGCAGATGTCTCTGGGTTTTTCTGCAGGTCTGAACTGCTG | 3480 |
| | ............................................................ | |
| 3481 | CTGCCACGTTTATTTTTATTTTCCCCGGTCAACAGGTGGCGCAGTCTGTACCTGGCATGC | 3540 |
| | ............................................................ | |
| 3541 | CTGTAAGGTGCTCGTGTGGTTTTTGTTTTCTTTTTTTCAGTCATGTGGATCAGCGATACT | 3600 |
| | ............................................................ | |
| 3601 | GCGTTCCCTTCATTCACATACTATGTCGCCACCTTTCCACATTGTAACTTTGATCTGTGA | 3660 |
| | ............................................................ | |
| 3661 | ATGCCTCTCGTAGCTAACAACTGGTTTCATGCTGTTTAACATCTGTATGAACTGAAACAT | 3720 |
| | ............................................................ | |
| 3721 | ACGTCACGTATTTAGTGCCATATCTTCTTGATTTGCTTTTTTCTTTTGTACTGTGTGTGT | 3780 |
| | ............................................................ | |
| 3781 | GAATGTACACTTGTGTGATTTGAGTGTTTTTGTTGTTCTTTTTATTTTCTCTTGTCTTAA | 3840 |
| | ............................................................ | |
| 3841 | TTTCTTTGACTGAAGATTTAAGTTTTAATGCTATTTTTTTAATAGCTTTTTAAAACTTCA | 3900 |
| | ............................................................ | |
| 3901 | GTCATTTTTTTAGGATTAATTGTCAAAATTGGATGGTAAATTATCAAATGTCCATCTGTC | 3960 |
| | ............................................................ | |
| 3961 | CCCTTTGTTATGTTGTTTGTTTTTGATTTCAGCCTCGGTCTTCATTTAATAACAAGCATT | 4020 |
| | ............................................................ | |
| 4021 | TCACCATGGTTTGTTAAGCTCATAATTTTTTCCCAGATTTCTCTGAATGTTTCCAATGAA | 4080 |
| | ............................................................ | |
| 4081 | ACTGAACATGTTGACCACACAGTACCCTCAATCTTTAGGTTTTTTTTGTTTTGTCTTTTA | 4140 |
| | ............................................................ | |
| 4141 | AGAGGGGATGTTACTACACAGGAGGCCATTATTCCCGTTTTTTTTTTTTTGTTTGTTTTT | 4200 |
| | ............................................................ | |
| 4201 | TTTAAATCATGTAATTGAACAACAGAAAATCGGATCCTGGTAAGATTCTGCACCAGCCCC | 4260 |
| | ............................................................ | |
| 4261 | CCACCACCACCACCCACGTGCACACCTACAGCCTCCAAGCAGACGACTGTAAATGTACAA | 4320 |
| | ............................................................ | |
| 4321 | AAATCACCTGTACCTAGAGAAAAATGTATATATTTATTCCTCAAGGAGATGGCCACCTCT | 4380 |
| | ............................................................ | |
| 4381 | TGGTCAATTTGGTTGTATGGTGCAATTATTATTATAATTATTATATATTTCTCCAGAATT | 4440 |
| | ............................................................ | |
| 4441 | ACCTGCTAGCCACTCCTGTTTTTAGTACAATGTGGTTTGTGGCCTGAACTCCCCTCTCTG | 4500 |
| | ............................................................ | |
| 4501 | TGTGCCTAAAATTAGCCAAGAAATGAGTATGGCAACCTAAGTAAGTAAAATGGTGGTTAT | 4560 |
| | ............................................................ | |
| 4561 | TAATGTAAATATGGGAAACTAATGATAAACTATTTATTAAAGGTTTATTGTACAATGAAA | 4620 |
| | ............................................................ | |
| 4621 | CGTTTCGGGTTGCCTCTGTGGTTTCTGGGTGGGTAACACAGGTGAAATCATGTTACTGTA | 4680 |
| | ............................................................ | |
| 4681 | GCAGTGAGTGAGCATCTGAGCAGCGATAATCATTTGGTCGTTGCATTTACGGCGATGATC | 4740 |
| | ............................................................ | |
| 4741 | CTATAGTTAATGGCTGCTAAATCCCAGTAAGTCTCACTATAAACTGGTAGCATTCCTGTT | 4800 |
| | ............................................................ | |
| 4801 | GGGCTTTACTTGCTGTTATATTACTGCACCCCCATTTTTTTTTTAATGTAATGCTCTGAC | 4860 |
| | ............................................................ | |
| 4861 | TTTGCTGGCTGTTGGTTTTGTAAACCTGCCCTTTGAAGCTTAATGTTACCGCTAATGCCT | 4920 |
| | ............................................................ | |
| 4921 | CCTCCACCTACACAGTGTATATAGTCGTGCATTGACCTGAGCTCATTTATGGGCGGTGGA | 4980 |
| | ............................................................ | |
| 4981 | TTTGTAATTAAATCCACATGGAGGCAGTAGTTACATCTGGCAGGAACTTTAAAGAGTCTT | 5040 |
| | ............................................................ | |
| 5041 | CTCCCTGAATAACAGTGAACGCAAAGTGGGAGATGTCACAAAATGTGATATTTATCCAAA | 5100 |
| | ............................................................ | |
| 5101 | ATAAAGAATACGATAAAGTGGCCAGAACAATTTATTTTTGTTATTAATGTAGTGTAGGGG | 5160 |
| | ............................................................ | |
| 5161 | AATTTAATGTCTTATAATTAGCAGCTAATAACTTGCCCATCATTTTGTTGAATTTCTGTG | 5220 |
| | ............................................................ | |
| 5221 | TGAATGATGAAGTTTTACTGGGTCAATGCTCAAATCTTAAGGTGATTAATGAGTATTTGC | 5280 |
| | ............................................................ | |
| 5281 | A | 5281 |
| | . | |

SEQ ID NOs 76 and 78 (Hiat1a mutant allele-17 nt deletion)
LENGTH: 5281 bp and 234 aa
TYPE: cDNA (SEQ ID NO: 76) and Protein (SEQ ID NO: 78)
ORGANISM: Nile tilapia

| | | |
|---|---|---|
| 1 | TTCTGCTTCGCCCTTGTATTAGACAGCCAATCGCTGGACGTCACTCCGCCAGAAGGGGTG | 60 |
| | ............................................................ | |
| 61 | GGTTGACGTAGTACAGGAAGCCAGGCGAGGTGAGGTGGGGAGGAGAGATCACAAAATTGT | 120 |
| | ............................................................ | |
| 121 | TAGCTCGCTGCTAGCTGCCTCCTCCGATTTGCCCGAAGTGCGATGAGCCCAGGAGGCGAA | 180 |
| | ............................................................ | |
| 181 | ATTTGTGGGGTTTTTTGGTTTTGATTGGCGCGACGATGACCCTCTGACCCTAAGAATGGA | 240 |
| | ............................................................ | |
| 241 | CATAAGTTAATGATGACGGGGGAGAAGAAGAAGAAGAAGCGGCTGAACCGCAGCATTCTT | 300 |
| | .........-M--M--T--G--E--K--K--K--K--K--R--L--N--R--S--I--L- | 17 |
| 301 | CTTGCAAAGAAAATTATAATAAAAGATGGAGGAAGTCCTCAGGGAATCGGGGAGCCCAGT | 360 |
| 18 | -L--A--K--K--I--I--I--K--D--G--G--S--P--Q--G--I--G--E--P--S- | 37 |
| 361 | GTTTACCATGCTGTGGTGGTCATCTTCCTGGAGTTTTTTGCATGGGGTCTGCTCACTACC | 420 |
| 38 | -V--Y--H--A--V--V--V--I--F--L--E--F--F--A--W--G--L--L--T--T- | 57 |
| 421 | CCGATGCTCACGGTATTACACCAGACATTCCCCCAACACACATTCCTGATGAATGGGCTC | 480 |
| 58 | -P--M--L--T--V--L--H--Q--T--F--P--Q--H--T--F--L--M--N--G--L- | 77 |
| 481 | ATTCATGGTGTGAAGGGCCTGTTATCATTTCTCAGTGCTCCGCTAATTGGAGCGTTGTCA | 540 |
| 78 | -I--H--G--V--K--G--L--L--S--F--L--S--A--P--L--I--G--A--L--S- | 97 |
| 541 | GACGTATGGGGACGCAAGTCCTTCCTGCTGCTAACGGTCTTCTTCACTTGCGCGCCCATT | 600 |
| 98 | -D--V--W--G--R--K--S--F--L--L--L--T--V--F--F--T--C--A--P--I- | 117 |
| 601 | CCGCTGATGAAGATCAGTCCATGGTGGTACTTTGCAGTCATCTCGATGTCCGGTGTTTTT | 660 |
| 118 | -P--L--M--K--I--S--P--W--W--Y--F--A--V--I--S--M--S--G--V--F- | 137 |
| 661 | GCCGTCACCTTCTCTGTGATCTTTGCCTATGTGGCAGACATCACACAAGAGCATGAGAGG | 720 |
| 138 | -A--V--T--F--S--V--I--F--A--Y--V--A--D--I--T--Q--E--H--E--R- | 157 |
| 721 | AGCACAGCTTATGGTTTGGTATCAGCTACCTTTGCAGCAAGCCTGGTTACCAGCCCAGCC | 780 |
| 158 | -S--T--A--Y--G--L--V--S--A--T--F--A--A--S--L--V--T--S--P--A- | 177 |
| 781 | ATTGGAGCCTACCTGTCTGAGGCTTACAGTGACACCTTGGTTGTGATCCTGGCCACAGCC | 840 |
| 178 | -I--G--A--Y--L--S--E--A--Y--S--D--T--L--V--V--I--L--A--T--A- | 197 |
| 841 | ATCGCACTGCTCGACATCTGCTTCATCCTGGTGGCTGTACCAGAGTCGCTGCCGGAGAAG | 900 |
| 198 | -I--A--L--L--D--I--C--F--I--L--V--A--V--P--E--S--L--P--E--K- | 217 |
| 901 | ATGAGCGCCCATCTCCTGGGAACAGGCAGACCCCTTCGCTTCTCTGCGTAAAGTGGGCCA | 960 |
| 218 | -M--S--A--H--L--L--G--T--G--R--P--L--R--C--V--S--A--*- | 234 |

SEQ ID NOs 79 and 81 (wild-type Smap2)
LENGTH: 4207 bp and 429 aa
TYPE: cDNA (SEQ ID NO: 79) and Protein (SEQ ID NO: 81)
ORGANISM: Nile tilapia

| | | |
|---|---|---|
| 1 | ATGACGGGCAAATCTGTGAAAGACGTTGACAGATACCAGGCTGTCCTCAACTCTTTACTG | 60 |
| 1 | -M--T--G--K--S--V--K--D--V--D--R--Y--Q--A--V--L--N--S--L--L- | 20 |
| 61 | GCGCTGGAGGAGAACAAATACTGCGCTGACTGTGAATCGAAAGGTCCACGATGGGCATCC | 120 |
| 21 | -A--L--E--E--N--K--Y--C--A--D--C--E--S--K--G--P--R--W--A--S- | 40 |
| 121 | TGGAATTTGGGCATCTTCATCTGTATCCGCTGTGCTGGTATCCATCGAAACCTGGGGGTT | 180 |
| 41 | -W--N--L--G--I--F--I--C--I--R--C--A--G--I--H--R--N--L--G--V- | 60 |
| 181 | CACATCTCCAAGGTCAAGTCTGTCAACCTGGATCAGTGGACGCAGGAGCAAGTCCAGTGT | 240 |
| 61 | -H--I--S--K--V--K--S--V--N--L--D--Q--W--T--Q--E--Q--V--Q--C- | 80 |
| 241 | GTTCAAGAGATGGGAAATGCCAAGGCCAAACGGCTCTACGAGGCTTTTTTACCCGAGTGC | 300 |
| 81 | -V--Q--E--M--G--N--A--K--A--K--R--L--Y--E--A--F--L--P--E--C- | 100 |
| 301 | TTCCAGCGTCCCGAGACAGACCAGGCTGCCGAGATCTTCATTAGGGACAAATACGAAAAG | 360 |
| 101 | -F--Q--R--P--E--T--D--Q--A--A--E--I--F--I--R--D--K--Y--E--K- | 120 |
| 361 | AAGAAATACATGGATAAAGTTATTGACATCCAGATGCTCAGGAAAGAAAAGAGTTGTGAC | 420 |
| 121 | -K--K--Y--M--D--K--V--I--D--I--Q--M--L--R--K--E--K--S--C--D- | 140 |
| 421 | AACATCCCAAAGGAGCCAGTTGTATTTGAGAAGATGAAATTGGTAGTTAAAAAGGAGAAC | 480 |
| 141 | -N--I--P--K--E--P--V--V--F--E--K--M--K--L--V--V--K--K--E--N- | 160 |
| 481 | ACTAAGAAAAAAGACGTCAGCCCAAAGACAGATTCCCAGTCTGTCACAGACCTGCTCGGA | 540 |
| 161 | -T--K--K--K--D--V--S--P--K--T--D--S--Q--S--V--T--D--L--L--G- | 180 |
| 541 | CTAGAACTGCTTTTATGTTGCAAGTCTGCACCTAAAAAGCAAATAAACACGTCAGACTCT | 600 |
| 181 | -L--E--L--L--L--C--C--K--S--A--P--K--K--Q--I--N--T--S--D--S- | 200 |
| 601 | GCCCTGGATCTCTTCAGCTCCCTCGCAGCCCCCTCCCCTGCTTCCTCTACAAAAAGCACG | 660 |
| 201 | -A--L--D--L--F--S--S--L--A--A--P--S--P--A--S--S--T--K--S--T- | 220 |
| 661 | GTAGTAGACACCATGCCTCAGAGCAGAGTGACTGCCTCAGTGCCTGAGAATCTGAGCTTG | 720 |
| 221 | -V--V--D--T--M--P--Q--S--R--V--T--A--S--V--P--E--N--L--S--L- | 240 |
| 721 | TTCTTAGGCCCAGCACCCAAAGCAGAGGAGGGCACAGTCAAGAAACTATCCAAGGACTCC | 780 |
| 241 | -F--L--G--P--A--P--K--A--E--E--G--T--V--K--K--L--S--K--D--S- | 260 |
| 781 | ATTCTTTCCCTGTACGCCTCCACTCCCTCGGTACATGCCAGCAGTATGGCCGCACATGGC | 840 |
| 261 | -I--L--S--L--Y--A--S--T--P--S--V--H--A--S--S--M--A--A--H--G- | 280 |
| 841 | TTGTACATGAACCAAATGGGATATCCAACACACCCGTACGGTCCATACCATTCTTTAGCC | 900 |
| 281 | -L--Y--M--N--Q--M--G--Y--P--T--H--P--Y--G--P--Y--H--S--L--A- | 300 |
| 901 | CAGGCAGGGGGAATGGGAGGCACTATGATGACATCACAGATGGCCATGATGGGGCAGCAG | 960 |
| 301 | -Q--A--G--G--M--G--G--T--M--M--T--S--Q--M--A--M--M--G--Q--Q- | 320 |
| 961 | CAGAGCGGGGTGATGGCGGTGCCACAAAACAGCATGATTGGAATTCAGCAGAACTGCATG | 1020 |
| 321 | -Q--S--G--V--M--A--V--P--Q--N--S--M--I--G--I--Q--Q--N--C--M- | 340 |
| 1021 | ATGGGGCAGCAGAATGGCTTAATGGGACAGCAACAAAGTGGGATGATAGGACAGCAGCAG | 1080 |
| 341 | -M--G--Q--Q--N--G--L--M--G--Q--Q--Q--S--G--M--I--G--Q--Q--Q- | 360 |
| 1081 | CAGGTTGGGGGTTTGCCCGCATTACCCCAGCAGCAGGCTTACGGAGTCCAGCAAGCCCAG | 1140 |
| 361 | -Q--V--G--G--L--P--A--L--P--Q--Q--Q--A--Y--G--V--Q--Q--A--Q- | 380 |
| 1141 | CAGCTACAGTGGAACATCAGCCAGATGACTCAGCACATGGCCGGCGTGAATCTTTACAAC | 1200 |
| 381 | -Q--L--Q--W--N--I--S--Q--M--T--Q--H--M--A--G--V--N--L--Y--N- | 400 |
| 1201 | ACCAGCGGTATGATGGGATACAGCGGTCAACAAATGGGAGGTTCAGCAGCTCCAAGTTCG | 1260 |
| 401 | -T--S--G--M--M--G--Y--S--G--Q--Q--M--G--G--S--A--A--P--S--S- | 420 |
| 1261 | GCACACATGACAGCGCACGTGTGGAAATGAGCTTGTCTATCTGAGATTCGATGGAGTGCC | 1320 |
| 421 | -A--H--M--T--A--H--V--W--K--*-.............................. | 429 |
| 1321 | AACGACCCACAAAAGGAGAAGAGAAACGCCGTGGATCAGACTCTCCATTAAACATTTTCT | 1380 |
| | ............................................................ | |
| 1381 | GATGCAAGGGAGGAGGAGGAGGAGAAGAAGAAGAAGAAGGTTTGAGAAACCACTACTACC | 1440 |
| | ............................................................ | |
| 1441 | TCTCTCTCTCCTCTCTGGCCGCGCTTCCTCTTGCCGTCTCATGCATAGCCATGTTCTGCA | 1500 |
| | ............................................................ | |
| 1501 | GATTTCCATGTTTGCCTTCAGGACCTTTTCATATGATGACTAAGACAAGGGGGTTCTGAG | 1560 |
| | ............................................................ | |
| 1561 | GCCACTGGTTAGGACTCCAGAGCTTTCTTTCTGCCTAGCCTTTATGAGAGAGCGCTCGTG | 1620 |
| | ............................................................ | |
| 1621 | TGCAGAAACATTATGAGGGTATCAAGCAGCTGCAGAATTGCACTGTTTCTTATTTAATCA | 1680 |
| | ............................................................ | |
| 1681 | GATGGCACTGGGGTTGGCATTGGGGTTAGCCTAGCTTTAAAAGCTCAAATAGACCGAGAT | 1740 |
| | ............................................................ | |
| 1741 | ATATAATCTGGTAACCTAAATAGGTGGCTCATACTTTAAATTCATTAGCCCTACATTACC | 1800 |
| | ............................................................ | |
| 1801 | AGTATTTACCCAACTGATGGAGCGACATTTAGTGATGATATGTACAGTGGCCCTGAGAGG | 1860 |
| | ............................................................ | |
| 1861 | TCAAACACACTGCAGCCTAATAAAACACCAGCAAAAATGAAAAATGGTGCAAAAGCACAC | 1920 |
| | ............................................................ | |
| 1921 | AAAACATAATGGAAGGTCAATAAAACCCAATGGAAATAGAAAGAAAAACACTGGAGAAGC | 1980 |
| | ............................................................ | |
| 1981 | TAGCAGAAAAAAATCTCACAAAACACAACAGAAATGTTTTTGGCTAAAATGTGACGGCTA | 2040 |
| | ............................................................ | |
| 2041 | ACAGCTAACAGTAAACGGCTAACAGCAACCATGTACCTACAGTGTCCATTGTGTTTTGTC | 2100 |
| | ............................................................ | |
| 2101 | AGAATTTTTTTTTCTATGTCCATTGTATTTTAATCAACTTCTGTGGTGCTTTTGCAAAAT | 2160 |
| | ............................................................ | |
| 2161 | TTTTCTGTTTTGCTGGTGTTTCCTACAGTTGCAGTGCATGTGACCTCTCAGGGCCACCGT | 2220 |
| | ............................................................ | |
| 2221 | AGACATAGCTACATTTTAACAGCAGCCATATTTGCAAAGTGTAGCAACTACAACTTTATT | 2280 |
| | ............................................................ | |
| 2281 | CAGCCAATTTCAAGGTAGAGATTTAGAGCTTTTCAAAAGTATATTTTCACATAAGTGAGA | 2340 |
| | ............................................................ | |
| 2341 | TGAGCTGCTGCTAATTCACTTAATAATCATTAACAAATATAAAAGCTAGGCTAGCCTAAT | 2400 |
| | ............................................................ | |
| 2401 | AGTCCCTTCATGCTGCATGCAGAAGACAAATACACATAACCATTTTTAGCAACATATATC | 2460 |
| | ............................................................ | |
| 2461 | TAGAAATTTCTACTCATTTAACAATATTTAATTCAAGCAACAAAACCTACCTACACAGCC | 2520 |
| | ............................................................ | |
| 2521 | CGTAATATTGATGTCTTCATCTCAATTTCTAGAGGGCTTCTTTTAGAATCTTTAATCTTG | 2580 |
| | ............................................................ | |
| 2581 | ACTTTAAAGTGTCAAAAGTCCAAAACCATATTTTGGGAGACCAAAGATCAACACTAGCTT | 2640 |
| | ............................................................ | |
| 2641 | TACTGTAAGTGGACAGTATTCCTGTATGCTTATTCCTGTTCAACCACTTAACTAGTGATT | 2700 |
| | ............................................................ | |
| 2701 | AATAGAAAAAAAAAACAGCAATTCAGCAGTCCGGCATCACTGTCTTCACTGTGCTGTTCT | 2760 |
| | ............................................................ | |
| 2761 | TTCACCAAGGGTAGGACACTTAAAAAAAAGAAAAAGAAGAAAGAAATCATTTTGCATGCA | 2820 |
| | ............................................................ | |
| 2821 | GTGTCATCAGCGCCCGCACACCTCCAGTTAAGAATCTACCTGGTGCATTAGTGGCCTCAA | 2880 |
| | ............................................................ | |
| 2881 | ATAACGTTGAATGTCTGTAAATAGGAGGTGAACAGAGAAGTGGGAGTAGAGACGGAAAAC | 2940 |
| | ............................................................ | |
| 2941 | TTCAAGGTGAAGGTCAGCCGGGTTTCAGATGCTTCCACTGAATTGCATGAAAAGAATGTG | 3000 |
| | ............................................................ | |
| 3001 | TATCTAGCTCTGATTGTATGTACTGTACTGTATGTTTGTTAAGATTTGCGAATGTGTCTC | 3060 |
| | ............................................................ | |
| 3061 | TCTGAATGTTTCTCCCTCTGACTCAGTCTTTGACAAAGACTGACAAAAAAACTATAAAAA | 3120 |
| | ............................................................ | |
| 3121 | AAAATAGGTAAAACATATGTTCTGAATGTGATCTCGGTTGACTCGTTTGATCGCGCGCAA | 3180 |
| | ............................................................ | |
| 3181 | TTGTTCTTCGGTGTGTTTTTGTTTTTTATATATTCCTTGTCTAGAAACGTACACCTTGTG | 3240 |
| | ............................................................ | |
| 3241 | TCTCTGGAATGTCTGTGCTCGATGGCATCCTGTGGGTTTCCAGTTTTGCTGTAACGGCCT | 3300 |
| | ............................................................ | |
| 3301 | CACCTTTGCGTTGGGGGCAAACAGTGAGCTGTTTTGTTTTTTTTTTCTTTTTGAGAGGGG | 3360 |
| | ............................................................ | |
| 3361 | ATGGGAGTATTTAACAATCTGGCCAAACCACATCGTGAAGCATAAAGCGATTGTAAAACC | 3420 |
| | ............................................................ | |
| 3421 | ACAATCTTTCACGTCTGTTTAAGCTGATGCTTGTACGCTTCTCCCACACAAACCATCTCT | 3480 |
| | ............................................................ | |
| 3481 | GTGCCCCGATTTCTCTTAAAAGTGTTGCTAAATCTGCCTTTTCTGATAAATGCTTATGGA | 3540 |
| | ............................................................ | |
| 3541 | AATGCTGTGTTTCTCTTATTTAATTTTATTTGACACTTGTGTTAAGCTGGTAAGATGCTG | 3600 |
| | ............................................................ | |
| 3601 | CTTTTAATGTGAGTGGCAGCAATATAGGAGGTGCCTATGTGCAGCATATAAGGTCTTATT | 3660 |
| | ............................................................ | |
| 3661 | TCACAACAGTGTGACAGCAGCAGTCACCTTCTCCACTGAGAGCAACATTTATATAAGAGA | 3720 |
| | ............................................................ | |
| 3721 | GAGCACATCCAGCACAGCAACAGCAAATCTGTCAGTCAACAAAAGTTTCTGGAAAGGCAG | 3780 |
| | ............................................................ | |
| 3781 | TGCAAGTCCACCTCTGTGGACGCTCAGGCCTCACCTGAGTTTTTCCATTTGTGATCAGGC | 3840 |
| | ............................................................ | |
| 3841 | TACTTTTTTTTTGGTCCGATATTTTTTCAATGAAACAAAAACGAATAAAGGAATGTAACT | 3900 |
| | ............................................................ | |
| 3901 | TTGTACGTACTTGTCGATCAAGATACTGTATATTTTAATTCTTTATCAAAATATCGCTGT | 3960 |
| | ............................................................ | |
| 3961 | ATATTATGTTTCTTAAACAACATGTTCTGTATATTAGTTTTTCTTTTCCACATGCTTTGC | 4020 |
| | ............................................................ | |
| 4021 | CCCACTTTACACAATTTCAATAAAATTTAACAATGTATATGTGACATATGATAATTGTCC | 4080 |
| | ............................................................ | |
| 4081 | CTGTGAAAACATGCAAATAAATATTGTTTTGGTTAAATTTTATGTTGTTTTGTTTGTTGT | 4140 |
| | ............................................................ | |
| 4141 | GTTCATTGCTGGGTGTCAGGAGTTTTCCTGTTATGCAACTCAGGTCAGAATAAAACGCTC | 4200 |
| | ............................................................ | |
| 4201 | AGACAGG | 4207 |
| | ....... | |

SEQ ID NOs 80 and 82 (Smap2 mutant allele- 17 nt deletion)
LENGTH: 4207 bp and 118 aa
TYPE: cDNA (SEQ ID NO: 80) and Protein (SEQ ID NO: 82)
ORGANISM: Nile tilapia

| | | |
|---|---|---|
| 1 | ATGACGGGCAAATCTGTGAAAGACGTTGACAGATACCAGGCTGTCCTCAACTCTTTACTG | 60 |
| 1 | -M--T--G--K--S--V--K--D--V--D--R--Y--Q--A--V--L--N--S--L--L- | 20 |
| 61 | GCGCTGGAGGAGAACAAATACTGCGCTGACTGTGAATCGAAAGGTCCACGATGGGCATCC | 120 |
| 21 | -A--L--E--E--N--K--Y--C--A--D--C--E--S--K--G--P--R--W--A--S- | 40 |
| 121 | TGGAATTTGGGCATCTTCATCTGTATCCGCTGTGCTGGGGGTTCACATCTCCAAGGTCAA | 180 |
| 41 | -W--N--L--G--I--F--I--C--I--R--C--A--G--G--S--H--L--Q--G--Q- | 60 |
| 181 | GTCTGTCAACCTGGATCAGTGGACGCAGGAGCAAGTCCAGTGTGTTCAAGAGATGGGAAA | 240 |
| 61 | -V--C--Q--P--G--S--V--D--A--G--A--S--P--V--C--S--R--D--G--K- | 80 |
| 241 | TGCCAAGGCCAAACGGCTCTACGAGGCTTTTTTACCCGAGTGCTTCCAGCGTCCCGAGAC | 300 |
| 81 | -C--Q--G--Q--T--A--L--R--G--F--F--T--R--V--L--P--A--S--R--D- | 100 |
| 301 | AGACCAGGCTGCCGAGATCTTCATTAGGGACAAATACGAAAAGAAGAAATACATGGATAA | 360 |
| 101 | -R--P--G--C--R--D--L--H--*- | 118 |

SEQ ID NOs 83 and 85 (wild-type Csnk2a2)
LENGTH: 1053 bp and 350 aa
TYPE: cDNA (SEQ ID NO: 83) and Protein (SEQ ID NO: 85)
ORGANISM: Nile tilapia

| | | |
|---|---|---|
| 1 | ATGCCTGGCCCCACACCGACCATCAGCAAAGCTCGGGTTTACACCGACGTTAATACACAG | 60 |
| 1 | -M--P--G--P--T--P--T--I--S--K--A--R--V--Y--T--D--V--N--T--Q- | 20 |
| 61 | AAGAACAGAGAGTACTGGGACTACGATGCTCATGTGCCAAACTGGAGTAATCAAGACAAC | 120 |
| 21 | -K--N--R--E--Y--W--D--Y--D--A--H--V--P--N--W--S--N--Q--D--N- | 40 |
| 121 | TATCAGCTGGTGCGTAAACTGGGCAGAGGGAAGTACAGTGAAGTGTTTGAGGCCATAAAT | 180 |
| 41 | -Y--Q--L--V--R--K--L--G--R--G--K--Y--S--E--V--F--E--A--I--N- | 60 |
| 181 | GTGACCAATAATGAGAAAGTGGTGGTGAAAATCCTGAAGCCTGTCAAGAAGAAGAAGATC | 240 |
| 61 | -V--T--N--N--E--K--V--V--V--K--I--L--K--P--V--K--K--K--K--I- | 80 |
| 241 | AAACGCGAAATCAAAATTCTTGAAAACTTGCGAGGAGGAACCAACATCATCCGCCTGGTG | 300 |
| 81 | -K--R--E--I--K--I--L--E--N--L--R--G--G--T--N--I--I--R--L--V- | 100 |
| 301 | GACACGGTCAAAGACCCGGTGTCCAGAACACCAGCGCTAGTCTTTGAGTACATCAATAAC | 360 |
| 101 | -D--T--V--K--D--P--V--S--R--T--P--A--L--V--F--E--Y--I--N--N- | 120 |
| 361 | ACAGATTTTAAGGAGCTTTACCAGAAGCTGACAGACTACGATATCCGTTACTACATGTAT | 420 |
| 121 | -T--D--F--K--E--L--Y--Q--K--L--T--D--Y--D--I--R--Y--Y--M--Y- | 140 |
| 421 | GAGCTTCTAAAGGCTCTGGACTTCTGTCACAGTATGGGGATCATGCACAGGGACGTGAAG | 480 |
| 141 | -E--L--L--K--A--L--D--F--C--H--S--M--G--I--M--H--R--D--V--K- | 160 |
| 481 | CCGCACAATGTGATGATTGACCACCAGCTGAGGAAGCTGCGTCTTATAGATTGGGGTTTG | 540 |
| 161 | -P--H--N--V--M--I--D--H--Q--L--R--K--L--R--L--I--D--W--G--L- | 180 |
| 541 | GCTGAATTTTACCATCCCGCTCAGGAATATAATGTCAGGGTGGCCTCGCGCTATTTCAAA | 600 |
| 181 | -A--E--F--Y--H--P--A--Q--E--Y--N--V--R--V--A--S--R--Y--F--K- | 200 |
| 601 | GGCCCCGAGCTGCTAGTGGACTATCAGATGTATGATTACAGTTTGGACATGTGGAGTCTC | 660 |
| 201 | -G--P--E--L--L--V--D--Y--Q--M--Y--D--Y--S--L--D--M--W--S--L- | 220 |
| 661 | GGCTGCATGTTGGCCAGTATGATTTTCCTGAAGGAACCGTTTTTTCATGGCCAGGACAAC | 720 |
| 221 | -G--C--M--L--A--S--M--I--F--L--K--E--P--F--F--H--G--Q--D--N- | 240 |
| 721 | TATGACCAGCTGGTCCGCATCGCTAAGGTTCTCGGCACCGATGAGCTCTTTGGCTACCTG | 780 |
| 241 | -Y--D--Q--L--V--R--I--A--K--V--L--G--T--D--E--L--F--G--Y--L- | 260 |
| 781 | CACAAATATCACATAGAACTGGACACTCGCTTCAAAGACATGCTGGGGCAGCAAACACGG | 840 |
| 261 | -H--K--Y--H--I--E--L--D--T--R--F--K--D--M--L--G--Q--Q--T--R- | 280 |
| 841 | AAACGCTGGGAGCAGTTCATCCAATCAGAGAACCAGCACCTGGTGAGTCCAGAGGCTCTG | 900 |
| 281 | -K--R--W--E--Q--F--I--Q--S--E--N--Q--H--L--V--S--P--E--A--L- | 300 |
| 901 | GACCTGCTGGACAAGCTGCTGCGCTATGACCACCAGCAGAGGCTGACGGCGGCCGAGGCC | 960 |
| 301 | -D--L--L--D--K--L--L--R--Y--D--H--Q--Q--R--L--T--A--A--E--A- | 320 |
| 961 | ATGCAGCACCCGTACTTCTATCCTGTGGTGAAGGAACAAGCAAATGCCAACACAGATGGC | 1020 |
| 321 | -M--Q--H--P--Y--F--Y--P--V--V--K--E--Q--A--N--A--N--T--D--G- | 340 |
| 1021 | TCAAAGGCAATAAGCAGCTCCAATGCAACATGA | 1053 |
| 341 | -S--K--A--I--S--S--S--N--A--T--*- | 350 |

SEQ ID NOs 84 and 86 (Csnk2a2 mutant allele-22 nt deletion)
LENGTH: 1053 bp and 31 aa
TYPE: cDNA (SEQ ID NO: 84) and Protein (SEQ ID NO: 86)
ORGANISM: Nile tilapia

| | | |
|---|---|---|
| 1 | ATGCTCATGTGCCAAACTGGAGTAATCAAGACAACTATCAGCTGGTGCGTAAACTGGGCA | 60 |
| 1 | -M--L--M--C--Q--T--G--V--I--K--T--T--I--S--W--C--V--N--W--A- | 20 |
| 61 | GAGGGAAGTACAGTGAAGTGTTTGAGGCCATAAATGTGACCAATAATGAGAAAGTGGTG | 120 |
| 21 | -E--G--S--T--V--K--C--L--R--P--*- | 31 |

SEQ ID NOs 87 and 89 (wild-type Gone)
LENGTH: 1335 bp and 444 aa
TYPE: cDNA (SEQ ID NO: 87) and Protein (SEQ ID NO: 89)
ORGANISM: Nile tilapia

| | | |
|---|---|---|
| 1 | ATGTCTGCTTCGACTGGATGCTCCCCATCGGGCCAGCACTCGGGCCTTGTCCCCAGTATG | 60 |
| 1 | -M--S--A--S--T--G--C--S--P--S--G--Q--H--S--G--L--V--P--S--M- | 20 |
| 61 | TCCATGTTTCGATGGCTAGAAGTGCTGGAGAAGGAATTTGATAAGGCTTTCGTGGATGTG | 120 |
| 21 | -S--M--F--R--W--L--E--V--L--E--K--E--F--D--K--A--F--V--D--V- | 40 |
| 121 | GATCTGTTGCTTGGAGAAATAGATCCAGATCAAGTGGATATAACGTATGAGGGTCGGCAG | 180 |
| 41 | -D--L--L--L--G--E--I--D--P--D--Q--V--D--I--T--Y--E--G--R--Q- | 60 |
| 181 | AAGATGACCAGCCTCAGCTCCTGTTTCGCTCAGCTCTGTCATAAAACCCAGACTGTCTTC | 240 |
| 61 | -K--M--T--S--L--S--S--C--F--A--Q--L--C--H--K--T--Q--T--V--F- | 80 |
| 241 | CAGCTCAACCATAAACTAGAGGCTCAGCTGGTGGACCTGCGCTCAGAGTTGACCGAAGCT | 300 |
| 81 | -Q--L--N--H--K--L--E--A--Q--L--V--D--L--R--S--E--L--T--E--A- | 100 |
| 301 | AAAGCTGCACGGGTGGTGGCAGAAAGGGAGGTCCACGACTTGCTCCTGCAGCTTCATGCT | 360 |
| 101 | -K--A--A--R--V--V--A--E--R--E--V--H--D--L--L--L--Q--L--H--A- | 120 |
| 361 | CTCCAACTGCAGCTTCATGTCAAGCAAGGCCAAGCTGAGGAGTCAGATACCATCAAAGAT | 420 |
| 121 | -L--Q--L--Q--L--H--V--K--Q--G--Q--A--E--E--S--D--T--I--K--D- | 140 |
| 421 | AAACTGCCTACACCAACCTTAGAAGAGCTGGAACAGGAGCTCGAGGCCAGTAAGAAGGAG | 480 |
| 141 | -K--L--P--T--P--T--L--E--E--L--E--Q--E--L--E--A--S--K--K--E- | 160 |
| 481 | AAATTAGCAGAGGCAAAAATGGAGGCAGAAACCAGACTATATAAGAAAGAAAACGAGGCC | 540 |
| 161 | -K--L--A--E--A--K--M--E--A--E--T--R--L--Y--K--K--E--N--E--A- | 180 |
| 541 | CTTCGCAGGCACATGGCAGTACTGCAGGCCGAAGTCTACGGAGCCAGACTGGCTGCTAAA | 600 |
| 181 | -L--R--R--H--M--A--V--L--Q--A--E--V--Y--G--A--R--L--A--A--K- | 200 |
| 601 | TACTTGGACAAGGAACTGGCTGGCAGGGTGCAGCAGATACAGTTACTGGGTCGTGACATG | 660 |
| 201 | -Y--L--D--K--E--L--A--G--R--V--Q--Q--I--Q--L--L--G--R--D--M- | 220 |
| 661 | AAAGGGCCAGCACATGACAAGCTCTGGAATCAACTGGAGGCAGAAATTCACCTTCACCGC | 720 |
| 221 | -K--G--P--A--H--D--K--L--W--N--Q--L--E--A--E--I--H--L--H--R- | 240 |
| 721 | CATAAAACTGTGATCCGAGCATGTAGAGGTCGAAGTGACCCTAAGAGACCTCTTCCCTCT | 780 |
| 241 | -H--K--T--V--I--R--A--C--R--G--R--S--D--P--K--R--P--L--P--S- | 260 |
| 781 | CCTGTGGGACATGATCCAGACATGCTGAAGAAAACCCAGGGAGTTGGCCCTATCCGAAAG | 840 |
| 261 | -P--V--G--H--D--P--D--M--L--K--K--T--Q--G--V--G--P--I--R--K- | 280 |
| 841 | GTTGTGCTGGTCAAAGAGGATCATGAGGGTCTAGGAATTTCCATTACAGGTGGGAAGGAG | 900 |
| 281 | -V--V--L--V--K--E--D--H--E--G--L--G--I--S--I--T--G--G--K--E- | 300 |
| 901 | CACGGCGTTCCCATTTTAATTTCAGAGATCCATCCCAGTCAGCCCGCAGACAGATGTGGA | 960 |
| 301 | -H--G--V--P--I--L--I--S--E--I--H--P--S--Q--P--A--D--R--C--G- | 320 |
| 961 | GGGCTGCATGTTGGAGATGCCATCCTTGCTGTCAACAGCATCAATTTGCGAGATGCCAAA | 1020 |
| 321 | -G--L--H--V--G--D--A--I--L--A--V--N--S--I--N--L--R--D--A--K- | 340 |
| 1021 | CATAAGGAAGCTGTCACCATTCTCTCTCAGCAGCGAGGACAGATAGAGTTTGAGGTCGTG | 1080 |
| 341 | -H--K--E--A--V--T--I--L--S--Q--Q--R--G--Q--I--E--F--E--V--V- | 360 |
| 1081 | TACGTGGCTCCTGAAGTGGACAGCGATGATGAGAATGTGGAGTACGAGGATGACAGCGGT | 1140 |
| 361 | -Y--V--A--P--E--V--D--S--D--D--E--N--V--E--Y--E--D--D--S--G- | 380 |
| 1141 | CATCGCTACAGACTCTACCTGGATGAACTGGATGACAGCATCACAGCACCACCTAGCAAC | 1200 |
| 381 | -H--R--Y--R--L--Y--L--D--E--L--D--D--S--I--T--A--P--P--S--N- | 400 |
| 1201 | AGTTCAGCATCACTTCAAGCACTGGAGAAGTTGTCACTGAGCAATGGAGCAGAGTCTGGA | 1260 |
| 401 | -S--S--A--S--L--Q--A--L--E--K--L--S--L--S--N--G--A--E--S--G- | 420 |
| 1261 | GATACTGGGATGTCCAGTGAGACACCTTCAGGGGAAACCCCTTCAAAGCCACCAGAAACT | 1320 |
| 421 | -D--T--G--M--S--S--E--T--P--S--G--E--T--P--S--K--P--P--E--T- | 440 |
| 1321 | GACTGCTCTTCCTAG | 1335 |
| 441 | -D--C--S--S--*- | 444 |

SEQ ID NOs 88 and 90 (Gone mutant allele- 8 nt deletion)
LENGTH: 1335 bp and 30 aa
TYPE: cDNA (SEQ ID NO: 88) and Protein (SEQ ID NO: 90)
ORGANISM: Nile tilapia

| | | |
|---|---|---|
| 1 | ATGTCTGCTTCGACTGGATGCTCCCCAGCACTCGGGCCTTGTCCCCAGTATGTCCATGTT | 60 |
| 1 | -M--S--A--S--T--G--C--S--P--A--L--G--P--C--P--Q--Y--V--H--V- | 20 |
| 61 | TCGATGGCTAGAAGTGCTGGAGAAGGAATTTGATAAGGCTTTCGTGGATGTGGATCTGTC | 120 |
| 21 | -S--M--A--R--S--A--G--E--G--I--*- | 30 |

SEQ ID NOs 91 and 94 (wild-type DMRT-1)
LENGTH: 882 bp and 293 aa
TYPE: cDNA (SEQ ID NO: 91) and Protein (SEQ ID NO: 94)
ORGANISM: Nile tilapia

| | | |
|---|---|---|
| 1 | ATGAGCCAGGACAAACAGAGTAAGCAGGTACCGGATTGCAGCGGACCGATGTCCCCGACC | 60 |
| 1 | -M--S--Q--D--K--Q--S--K--Q--V--P--D--C--S--G--P--M--S--P--T- | 20 |
| 61 | AAAGCCCAGAAATCCCCCAGGATGCCCAAGTGCTCTCGCTGTAGAAATCACGGATACGTG | 120 |
| 21 | -K--A--Q--K--S--P--R--M--P--K--C--S--R--C--R--N--H--G--Y--V- | 40 |
| 121 | TCTCCACTGAAGGGACACAAGCGCTTTTGCAACTGGAGGGACTGCCAGTGTCCCAAATGC | 180 |
| 41 | -S--P--L--K--G--H--K--R--F--C--N--W--R--D--C--Q--C--P--K--C- | 60 |
| 181 | AAATTGATCGCGGAGAGGCAGAGAGTCATGGCGGCCCAGGTTGCTCTGAGGAGGCAGCAG | 240 |
| 61 | -K--L--I--A--E--R--Q--R--V--M--A--A--Q--V--A--L--R--R--Q--Q- | 80 |
| 241 | GCCCAAGAAGAAGAGCTTGGGATTTGTAGTCCTGTGTCTCTGTCCGGTTCCGAGATGATG | 300 |
| 81 | -A--Q--E--E--E--L--G--I--C--S--P--V--S--L--S--G--S--E--M--M- | 100 |
| 301 | GTCAAGAATGAAGTTGGAGCAGACTGCCTGTTCTCTGTGGAGGGACGGTCCCCGACACCT | 360 |
| 101 | -V--K--N--E--V--G--A--D--C--L--F--S--V--E--G--R--S--P--T--P- | 120 |
| 361 | ACCAGCCACGCCACCTCTGCTGTCACAGGGACCCGCTCGGCATCGTCCCCCAGCCCATCT | 420 |
| 121 | -T--S--H--A--T--S--A--V--T--G--T--R--S--A--S--S--P--S--P--S- | 140 |
| 421 | GCTGCTGCCAGGGCTCATACCGAGGGACCGTCTGACCTCCTGCTGGAAACCCCCTATTAC | 480 |
| 141 | -A--A--A--R--A--H--T--E--G--P--S--D--L--L--L--E--T--P--Y--Y- | 160 |
| 481 | AATTTCTACCAGCCTTCGCGCTACCCCACCTACTATGGAAACCTTTACAACTACTCGCAG | 540 |
| 161 | -N--F--Y--Q--P--S--R--Y--P--T--Y--Y--G--N--L--Y--N--Y--S--Q- | 180 |
| 541 | TACCAGCAGATGCCTCATGGTGATGGCCGCCTGCCCAGCCACAGCGTGTCGTCTCAGTAC | 600 |
| 181 | -Y--Q--Q--M--P--H--G--D--G--R--L--P--S--H--S--V--S--S--Q--Y- | 200 |
| 601 | CGCATGCACTCCTACTACCCAGCAGCCACCTACCTGACTCAGGGCCTGGGCTCCACCAGC | 660 |
| 201 | -R--M--H--S--Y--Y--P--A--A--T--Y--L--T--Q--G--L--G--S--T--S- | 220 |
| 661 | TGTGTGCCACCCTTCTTTAGCCTGGATGACAACAATAACAGCTGCTCTGAGACCATGGCA | 720 |
| 221 | -C--V--P--P--F--F--S--L--D--D--N--N--N--S--C--S--E--T--M--A- | 240 |
| 721 | GCCTCCTTCTCACCCGGCAGCATCTCCGCTGGTCACGACTCCACCATGGTCTGCCGCTCC | 780 |
| 241 | -A--S--F--S--P--G--S--I--S--A--G--H--D--S--T--M--V--C--R--S- | 260 |
| 781 | ATCAGCTCCCTGGTTAACGGCGACGCCAAGGCTGAATGCGAGGCCAGCAGCCAGGCAGCC | 840 |
| 261 | -I--S--S--L--V--N--G--D--A--K--A--E--C--E--A--S--S--Q--A--A- | 280 |
| 841 | GGCTTCACCGTCGACGCCATCGAAGGCGGCGCCACCAAATAA | 882 |
| 281 | -G--F--T--V--D--A--I--E--G--G--A--T--K--*- | 293 |

SEQ ID NOs 92 and 95 (DMRT-1 mutant allele- 7 nt deletion)
LENGTH: 882 bp and 40 aa
TYPE: cDNA (SEQ ID NO: 92) and Protein (SEQ ID NO: 95)
ORGANISM: Nile tilapia

| | | |
|---|---|---|
| 1 | ATGAGCCAGGACAAACAGAGTAAGCAGGTACCGGATTGCAGCGGACCCCGACCAAAGCCC | 60 |
| 1 | -M--S--Q--D--K--Q--S--K--Q--V--P--D--C--S--G--P--R--P--K--P- | 20 |
| 61 | AGAAATCCCCCAGGATGCCCAAGTGCTCTCGCTGTAGAAATCACGGATACGTGTCTCCAC | 120 |
| 21 | -R--N--P--P--G--C--P--S--A--L--A--V--E--I--T--D--T--C--L--H- | 40 |
| 121 | TGAAGGGACACAAGCGCTTTTGCAACTGGAGGGACTGCCAGTGTCCCAAATGCAAATTGA | 180 |
| 41 | -*- | 40 |

SEQ ID NOs 93 and 96 (DMRT-1 mutant allele- 13 nt deletion)
LENGTH: 882 bp and 38 aa
TYPE: cDNA (SEQ ID NO: 93) and Protein (SEQ ID NO: 96)
ORGANISM: Nile tilapia

| | | |
|---|---|---|
| 1 | ATGAGCCAGGACAAACAGAGTAAGCAGGTACCGGATTGCAGCGGACCAAAGCCCAGAAAT | 60 |
| 1 | -M--S--Q--D--K--Q--S--K--Q--V--P--D--C--S--G--P--K--P--R--N- | 20 |
| 61 | CCCCCAGGATGCCCAAGTGCTCTCGCTGTAGAAATCACGGATACGTGTCTCCACTGAAGG | 120 |
| 21 | -P--P--G--C--P--S--A--L--A--V--E--I--T--D--T--C--L--H--*- | 38 |

SEQ ID NOs 97 and 100 (wild-type GSDF)
LENGTH: 840 bp and 213 aa
TYPE: cDNA (SEQ ID NO: 97) and Protein (SEQ ID NO: 100)
ORGANISM: Nile tilapia

| | | |
|---|---|---|
| 1 | AACAGGGGAAAAGTCTACAGTGTTAACTATGTCAAGGCCACCTTGGGGTACAAGCAGATA | 60 |
| | ............................................................ | |
| 61 | AAAACCGTGGTTCTCAGACCCTGACAAACAATACCTAGGGCAGCATCCCAGTTTTGTCGC | 120 |
| | ............................................................ | |
| 121 | TACTATCTCCTCCTCCGACCAGACGTTCGGGACCAACCGCAGCTTTTGTCTGCAGCCAGT | 180 |
| | ............................................................ | |
| 181 | CTTACGTGTTCATCCACCATGGCCTTTCCATTCATTGTCATGACATTACTTTTGGGCTCT | 240 |
| | ..................-M--A--F--P--F--I--V--M--T--L--L--L--G--S- | 14 |
| 241 | TCCATGATGATGGCATTTGTCTTGGATCCATCCAGGAAAGAACCCGAAGCTGCCGTCTTA | 300 |
| 15 | -S--M--M--M--A--F--V--L--D--P--S--R--K--E--P--E--A--A--V--L- | 34 |
| 301 | GGTGACAGGTGCCAAGGTGAGTCATGGCAGTCCATCAGAAAGAACCTCCTTAGGGTTCTG | 360 |
| 35 | -G--D--R--C--Q--G--E--S--W--Q--S--I--R--K--N--L--L--R--V--L- | 54 |
| 361 | AACTTGCAGACTGAGCCGCAGCTACCTGCCGGTGCACTGGACAGTGTCAGAGAGCAGTGG | 420 |
| 55 | -N--L--Q--T--E--P--Q--L--P--A--G--A--L--D--S--V--R--E--Q--W- | 74 |
| 421 | AACCGAACCTTCAGCATCGTTTCTCACACAGCCAAGCATACTGCAACCCCAGCAGTCCCA | 480 |
| 75 | -N--R--T--F--S--I--V--S--H--T--A--K--H--T--A--T--P--A--V--P- | 94 |
| 481 | GGCTACTCTGCATCAGCTGATAATGGAAACAGTGCGAGCCTGAAGTGTTGTTCCATTGCC | 540 |
| 95 | -G--Y--S--A--S--A--D--N--G--N--S--A--S--L--K--C--C--S--I--A- | 114 |
| 541 | TCAGAGATCTTCATGAAAGATCTGGGCTGGGACAGCTGGGTGATCCACCCGTTGAGTCTT | 600 |
| 115 | -S--E--I--F--M--K--D--L--G--W--D--S--W--V--I--H--P--L--S--L- | 134 |
| 601 | ACCTATGTTCAGTGCGCAACCTGCAACTCTGCCATGACCACTGTTCAATGTCCATCATCC | 660 |
| 135 | -T--Y--V--Q--C--A--T--C--N--S--A--M--T--T--V--Q--C--P--S--S- | 154 |
| 661 | CAAGTAAATGTCCAGGATGCCAACACACAGGACCAGGTGCCATGCTGTCGGCCCACCTCC | 720 |
| 155 | -Q--V--N--V--Q--D--A--N--T--Q--D--Q--V--P--C--C--R--P--T--S- | 174 |
| 721 | CAAGAAGAGGTGCCCATAGTCTATATGGATGGATCCAGCGCCATTGTCATGTCCTCCATG | 780 |
| 175 | -Q--E--E--V--P--I--V--Y--M--D--G--S--S--A--I--V--M--S--S--M- | 194 |
| 781 | CAGCTGACCCGCAGTTGTGGCTGTGAGCTGGGCAACTCTGAGGATCGTGGCAAGGAGTAG | 840 |
| 195 | -Q--L--T--R--S--C--G--C--E--L--G--N--S--E--D--R--G--K--E--*- | 213 |

SEQ ID NOs 98 and 101 (GSDF mutant allele- 5 nt deletion)
LENGTH: 840 bp and 56 aa
TYPE: cDNA (SEQ ID NO: 98) and Protein (SEQ ID NO: 101)
ORGANISM: Nile tilapia

| | | |
|---|---|---|
| 1 | AACAGGGGAAAAGTCTACAGTGTTAACTATGTCAAGGCCACCTTGGGGTACAAGCAGATA | 60 |
| | ............................................................ | |
| 61 | AAAACCGTGGTTCTCAGACCCTGACAAACAATACCTAGGGCAGCATCCCAGTTTTGTCGC | 120 |
| | ............................................................ | |
| 121 | TACTATCTCCTCCTCCGACCAGACGTTCGGGACCAACCGCAGCTTTTGTCTGCAGCCAGT | 180 |
| | ............................................................ | |
| 181 | CTTACGTGTTCATCCACCATGGCCTTTCCATTCATTGTCATGACATTACTTTTGGGCTCT | 240 |
| | ..................-M--A--F--P--F--I--V--M--T--L--L--L--G--S- | 14 |
| 241 | TCCATGATGATGGCATTTGTCTTGGATCCATCCAGGAAAGAACCCGAAGCTGCCGTCTTA | 300 |
| 15 | -S--M--M--M--A--F--V--L--D--P--S--R--K--E--P--E--A--A--V--L- | 34 |
| 301 | GGTGACAGGTGCCAAGGTGAGTCATGGCAGTCCATCAGAAAGAACCTCCGTTCTGAACTT | 360 |
| 35 | -G--D--R--C--Q--G--E--S--W--Q--S--I--R--K--N--L--L--R--S--E- | 54 |
| 361 | GCAGACTGAGCCGCAGCTACCTGCCGGTGCACTGGACAGTGTCAGAGAGCAGTGGAACCG | 420 |
| 55 | -L--A--*- | 56 |

SEQ ID NOs 99 and 102 (GSDF mutant allele- 22 nt deletion)
LENGTH: 840 bp and 46 aa
TYPE: cDNA (SEQ ID NO: 99) and Protein (SEQ ID NO: 102)
ORGANISM: Nile tilapia

| | | |
|---|---|---|
| 1 | AACAGGGGAAAAGTCTACAGTGTTAACTATGTCAAGGCCACCTTGGGGTACAAGCAGATA | 60 |
| | ............................................................ | |
| 61 | AAAACCGTGGTTCTCAGACCCTGACAAACAATACCTAGGGCAGCATCCCAGTTTTGTCGC | 120 |
| | ............................................................ | |
| 121 | TACTATCTCCTCCTCCGACCAGACGTTCGGGACCAACCGCAGCTTTTGTCTGCAGCCAGT | 180 |
| | ............................................................ | |
| 181 | CTTACGTGTTCATCCACCATGGCCTTTCCATTCATTGTCATGACATTACTTTTGGGCTCT | 240 |
| | ..................-M--A--F--P--F--I--V--M--T--L--L--L--G--S- | 14 |
| 241 | TCCATGATGATGGCATTTGTCTTGGATCCATCCAGGAAAGAACCCGAAGCTGCCGTCTTA | 300 |
| 15 | -S--M--M--M--A--F--V--L--D--P--S--R--K--E--P--E--A--A--V--L- | 34 |
| 301 | GGTGACAGGTGCCAAGGTGAGTCATGGCAGTCCATCTGAACTTGCAGACTGAGCCGCAGC | 360 |
| 35 | -G--D--R--C--Q--G--E--S--W--Q--S--I--*- | 46 |

SEQ ID NOs 103 and 105 (wild-type FSHR)
LENGTH: 5853 bp and 689 aa
TYPE: cDNA (SEQ ID NO: 103) and Protein (SEQ ID NO: 105)
ORGANISM: Nile tilapia

| | | |
|---|---|---|
| 1 | GCATTCACTACTGCATGACAGAAAACACCAAAACACCTCACATTTCTCTCTAGCTGACCT | 60 |
| | ............................................................ | |
| 61 | GGCGCCGAACCCTCGAGCGGACAGACAGGCAAAGGCGTTCATATCAAATGTGGAGTGTGG | 120 |
| | ...............................................-M--W--S--V-- | 4 |
| 121 | ACCAGAGACAATATCAGAGTAAAATACACAAAAGAAGACAAACTAGAAAAGTGAAACCAC | 180 |
| 5 | D--Q--R--Q--Y--Q--S--K--I--H--K--R--R--Q--T--R--K--V--K--P-- | 24 |
| 181 | TCTGTGGACCCAGGCAGACTGAAATGATGCTGGTGATGTTTGGAGTCACGGCGTTTCCCT | 240 |
| 25 | L--C--G--P--R--Q--T--E--M--M--L--V--M--F--G--V--T--A--F--P-- | 44 |
| 241 | CCAACATCTCCAACGCCCAGTGCCTGGAAGTTAAGCAGACGCAGATCAGAGAGATTCAGC | 300 |
| 45 | S--N--I--S--N--A--Q--C--L--E--V--K--Q--T--Q--I--R--E--I--Q-- | 64 |
| 301 | AGGGCGCCCTCTCCAGCCTCCAGCATCTAATGGAACTGACCATTTCTGAGAACGACCTGC | 360 |
| 65 | Q--G--A--L--S--S--L--Q--H--L--M--E--L--T--I--S--E--N--D--L-- | 84 |
| 361 | TGGAGAGTATCGGTGCTTTTGCCTTTTCTGGCCTCCCTCACCTCACCAAAATCTTAATAT | 420 |
| 85 | L--E--S--I--G--A--F--A--F--S--G--L--P--H--L--T--K--I--L--I-- | 104 |
| 421 | CTAAAAATGCTGCTCTGAGGAATATCGGGGCTTTTGTTTTCTCCAACCTCCCTGAACTCA | 480 |
| 105 | S--K--N--A--A--L--R--N--I--G--A--F--V--F--S--N--L--P--E--L-- | 124 |
| 481 | GTGAGATAATCATAACAAAATCAAAACACCTGAGTTTCATCCACCCCGATGCATTCAGGA | 540 |
| 125 | S--E--I--I--I--T--K--S--K--H--L--S--F--I--H--P--D--A--F--R-- | 144 |
| 541 | ACATGGCAAGACTACGGTTCTTGACTATCTCCAACACCGGGCTGAGGATTTTTCCAGACT | 600 |
| 145 | N--M--A--R--L--R--F--L--T--I--S--N--T--G--L--R--I--F--P--D-- | 164 |
| 601 | TCTCCAAGATCCATTCCACCGCCTGCTTTCTGCTGGATCTTCAGGACAACAGCCACATAA | 660 |
| 165 | F--S--K--I--H--S--T--A--C--F--L--L--D--L--Q--D--N--S--H--I-- | 184 |
| 661 | AGAGAGTCCCTGCCAATGCCTTCAGAGGCCTCTGCACTCAAACCTTCGCAGAGATACGGC | 720 |
| 185 | K--R--V--P--A--N--A--F--R--G--L--C--T--Q--T--F--A--E--I--R-- | 204 |
| 721 | TCACCAGAAATGGCATCAAGGAGGTGGCAAGTGACGCCTTCAACGGAACAAAGATGCACA | 780 |
| 205 | L--T--R--N--G--I--K--E--V--A--S--D--A--F--N--G--T--K--M--H-- | 224 |
| 781 | GACTGTTCCTAGGAGGCAACCGACAGCTTACTCACATCAGTCCCAATGCCTTTGTGGGTT | 840 |
| 225 | R--L--F--L--G--G--N--R--Q--L--T--H--I--S--P--N--A--F--V--G-- | 244 |
| 841 | CCAGTGAGTTGGTGGTACTAGACGTCTCCGAAACAGCCCTCACCTCTTTGCCAGACTCGA | 900 |
| 245 | S--S--E--L--V--V--L--D--V--S--E--T--A--L--T--S--L--P--D--S-- | 264 |
| 901 | TCCTTGATGGCCTCAAGAGGCTGATTGCCGAGTCAGCCTTCAACCTGAAAGAACTTCCTC | 960 |
| 265 | I--L--D--G--L--K--R--L--I--A--E--S--A--F--N--L--K--E--L--P-- | 284 |
| 961 | CTATTCAGCTCTTTACCAAACTGCACCAGGCAAAGCTGACATACCCATCACACTGCTGCG | 1020 |
| 285 | P--I--Q--L--F--T--K--L--H--Q--A--K--L--T--Y--P--S--H--C--C-- | 304 |
| 1021 | CTTTCCTGAACATGCACAGAAACAGATCGAGATGGCACTCACTGTGTGACAACCCCGAGG | 1080 |
| 305 | A--F--L--N--M--H--R--N--R--S--R--W--H--S--L--C--D--N--P--E-- | 324 |
| 1081 | CTAAAAATAACCTGCACTTCTTCAGGGAATACTGCTCCAACTCCACCAACATCACTTGCA | 1140 |
| 325 | A--K--N--N--L--H--F--F--R--E--Y--C--S--N--S--T--N--I--T--C-- | 344 |
| 1141 | GCCCGGCCCCTGATGACTTTAACCCCTGTGAAGATATCATGTCTGCTACCCCCTTACGCA | 1200 |
| 345 | S--P--A--P--D--D--F--N--P--C--E--D--I--M--S--A--T--P--L--R-- | 364 |
| 1201 | TCCTCATCTGGATCATCTCTGTCCTCGCCCTGCTGGGCAACGCAGTAGTTCTCCTTGTAT | 1260 |
| 365 | I--L--I--W--I--I--S--V--L--A--L--L--G--N--A--V--V--L--L--V-- | 384 |
| 1261 | TGTTAGGCAGCCGCTATAAGCTGACTGTTCCTCGATTCCTCATGTGCCACCTGGCCTTTG | 1320 |
| 385 | L--L--G--S--R--Y--K--L--T--V--P--R--F--L--M--C--H--L--A--F-- | 404 |
| 1321 | CTGACCTCTGCATGGGCATCTACCTGGTAGTCATAGCAACCGTGGATATGCTCACACGTG | 1380 |
| 405 | A--D--L--C--M--G--I--Y--L--V--V--I--A--T--V--D--M--L--T--R-- | 424 |
| 1381 | GACGGTACTACAACTATGCTATAGACTGGCAGATGGGCTTGGGCTGCAATGCTGCAGGCT | 1440 |
| 425 | G--R--Y--Y--N--Y--A--I--D--W--Q--M--G--L--G--C--N--A--A--G-- | 444 |
| 1441 | TCTTCACGGTGTTCGCCAGTGAGCTGTCAGTGTTTACCTTGACAGCAATCACCGTGGAGC | 1500 |
| 445 | F--F--T--V--F--A--S--E--L--S--V--F--T--L--T--A--I--T--V--E-- | 464 |
| 1501 | GCTGGCACACCATCACGCATGCTCTGCGACTTGACCGCAAACTTCGCCTGAGACACGCCT | 1560 |
| 465 | R--W--H--T--I--T--H--A--L--R--L--D--R--K--L--R--L--R--H--A-- | 484 |
| 1561 | GCATCATCATGACAATAGGTTGGATCTTCTCCTTGCTGGCTGCACTGCTGCCCACAGTTG | 1620 |
| 485 | C--I--I--M--T--I--G--W--I--F--S--L--L--A--A--L--L--P--T--V-- | 504 |
| 1621 | GGATCAGCAGCTATGGCAAAGTGAGCATCTGCCTCCCCATGGATGTTGAGTCCCTAGTCT | 1680 |
| 505 | G--I--S--S--Y--G--K--V--S--I--C--L--P--M--D--V--E--S--L--V-- | 524 |
| 1681 | CCCAGTTCTACGTGGTCTGTCTTCTCCTCCTCAACATCTTGGCGTTCTTCTGTGTGTGCG | 1740 |
| 525 | S--Q--F--Y--V--V--C--L--L--L--L--N--I--L--A--F--F--C--V--C-- | 544 |
| 1741 | GCTGCTACCTCAGCATCTACCTCACCTTTCGCAAGCCTTCATCAGCGGCAGCCCACGCCG | 1800 |
| 545 | G--C--Y--L--S--I--Y--L--T--F--R--K--P--S--S--A--A--A--H--A-- | 564 |
| 1801 | ACACCCGTGTGGCTCAACGCATGGCCGTCCTCATCTTCACAGACTTCATCTGCATGGCTC | 1860 |
| 565 | D--T--R--V--A--Q--R--M--A--V--L--I--F--T--D--F--I--C--M--A-- | 584 |
| 1861 | CGATCTCCTTCTTCGCCATCTCAGCTGCCCTCAAGCTCCCTCTCATCACCGTCTCAGACT | 1920 |
| 585 | P--I--S--F--F--A--I--S--A--A--L--K--L--P--L--I--T--V--S--D-- | 604 |
| 1921 | CCAAGCTACTGTTGGTGCTATTCTACCCCATCAACTCGTGCTCCAACCCCTTCTTATATG | 1980 |
| 605 | S--K--L--L--L--V--L--F--Y--P--I--N--S--C--S--N--P--F--L--Y-- | 624 |
| 1981 | CCTTTTTCACCCGTAACTTCAGAAGGGATTTCTTTCTCCTCGCAGCTCGCTTCGGGCTGT | 2040 |
| 625 | A--F--F--T--R--N--F--R--R--D--F--F--L--L--A--A--R--F--G--L-- | 644 |
| 2041 | TTAAGACTCGAGCACAGATTTACCGGACAGAGGGTTCCTCGTGTCAGCAGCCAACATGGA | 2100 |
| 645 | F--K--T--R--A--Q--I--Y--R--T--E--G--S--S--C--Q--Q--P--T--W-- | 664 |
| 2101 | CCTCTCCAAAGAACAGCCGTGTTATCTTGTATTCCTTGGTCAATACGTTAAGTCTAGATG | 2160 |
| 665 | T--S--P--K--N--S--R--V--I--L--Y--S--L--V--N--T--L--S--L--D-- | 684 |
| 2161 | GAAAACAAGAGTGCTGACTTTTACGCACATTTACAGGTACGGACTGTTTGCCTTGATTGC | 2220 |
| 685 | G--K--Q--E--C--*-........................................... | 689 |
| 2221 | ATATTATATCCATACAAACAGGCTGCTAATTCCTTAAAATGATGCCTCAGATCATGTCTT | 2280 |
| | ............................................................ | |
| 2281 | TTGATCACTACCTGGGAAAATTTTTCTATCTACTTAGACTAGAAAGAAAAAAAACACAAA | 2340 |
| | ............................................................ | |
| 2341 | AGGCAACCAAGTGGAAGGCAAAAGAGCTGAGAACTCTTTTTTGACAATTTGACCCAGGAG | 2400 |
| | ............................................................ | |
| 2401 | TCTGCAAAACACAGTGATTGTTAAAATAAACAATGCTCTTGCTCTTGCTTCTGTTTGTGC | 2460 |
| | ............................................................ | |
| 2461 | TCCTAATCTGATGCTGTGTTTTTTGGGCTTGAGCCAGTGAAGGCTTCCACTGAAGACTGC | 2520 |
| | ............................................................ | |
| 2521 | TCTTCAGTCAATAAATAGCATCCAGAGACCCAGCTCTCAACAGAGGTGATGATCCTCTAT | 2580 |
| | ............................................................ | |
| 2581 | ATAAAGATGTTGGTCAGTTCAACAAAGAAGTTGATGCTTGTCTCTGTGCAAGTCTGAGAT | 2640 |
| | ............................................................ | |
| 2641 | CTCTGTTAGGGATGTACATGTACAAGTGGTCAAGATTGGACTTCCAGGCCATGAGACCAG | 2700 |
| | ............................................................ | |
| 2701 | AGGTCTACAAGTCACAAAACCTTTTAAAGCTTTTTATAAAATTATATATATCTATGTCGC | 2760 |
| | ............................................................ | |
| 2761 | CACAATCTGAGCAGTTCAGACACTGATGATTCCAGACTGATCACTGACCCAAGAGAAAGC | 2820 |
| | ............................................................ | |
| 2821 | ATGCATACATGTTCCCACCTGTCTTTTAAGGTTACACATAAATCAACATGTTTCAATCAC | 2880 |
| | ............................................................ | |
| 2881 | AATAGTATCAGTTGACTATTCAGCACAAAGTACACACAGCGTTCAGTGGCATGTCTAAAC | 2940 |
| | ............................................................ | |
| 2941 | CTGGTTACCTGAGCTATGCTCTGCAGCAATCCATGCAAACATGACCACAAAAGAACTAAT | 3000 |
| | ............................................................ | |
| 3001 | TATACACTCACTGGCCACTTTATTAGGTATACTTGTTTGGCTGCTTGGTAATGCAAATAC | 3060 |
| | ............................................................ | |
| 3061 | TTAATGAGCCAATCGCATGGTAGCAGCTCAGTGCATTTAGACATGTAATCTGGGGCATTT | 3120 |
| | ............................................................ | |
| 3121 | TTAAGATTTTTTAAATGTGGTGGCACGGCAGAGACCAAGAACACAGTAGAGGGGGACATT | 3180 |
| | ............................................................ | |
| 3181 | TAAATATTTGATTAGCAAAAAGATCAGAAAACTGACAGAAATTATTGGGCATGATTTTTG | 3240 |
| | ............................................................ | |
| 3241 | GTGTGCAACCTTATGTTTTATTACAAGTTTATTGTGTGAAAAGTGGTGCTGCAGAATGCT | 3300 |
| | ............................................................ | |
| 3301 | CTACATAGAATTTTGTGTTGGACAATTGTTTTGCAACGTGGAAAAAGAAGTATTTAGACT | 3360 |
| | ............................................................ | |
| 3361 | TAACCTAAGTAAAAGTTGTAATTGCACTTAAATAGCTTAATAGTTCACAAGTTATATAAT | 3420 |
| | ............................................................ | |
| 3421 | CAAAATGTATTCAAAGTGCCTAAAGTAAACACACTCTTTATATAGAATGGCCCTTTTTTT | 3480 |
| | ............................................................ | |
| 3481 | CTCGTCTCTTTAATGAGGCAGCTGTTGATGAGTTTGATTCCTGATATATTGTTCAATAGA | 3540 |
| | ............................................................ | |
| 3541 | TTCATTTATAAAAAATACAATTAATGTACAAAATAAGAAGAAGCTAAAATAATTTGGGGT | 3600 |
| | ............................................................ | |
| 3601 | GGGCTAATGCCACTCCAAGCTCCTCCCCCTCCAAACATGCCTCTATGTAGACATAATCAA | 3660 |
| | ............................................................ | |
| 3661 | GACAACTTGCTAAAGTTCAAAATGAGCATCAGAATGGGAAAAAGGTGACTGAAGTGACTT | 3720 |
| | ............................................................ | |
| 3721 | TGAAAGTTTAATTGTTGTTGGTGCCAGATGGTCCCACATGTCGCCACAATCTGCTGGCCG | 3780 |
| | ............................................................ | |
| 3781 | GACTGCAAACTGATAGGAAAGCAACAGTAACTTAAATAACTTCTATACAATCAAGGTGTA | 3840 |
| | ............................................................ | |
| 3841 | CAAGTTACATAAGAAAACTGGGCATCACTTAGTCTGATAACTCTTGATTTCTATTCTGAC | 3900 |
| | ............................................................ | |
| 3901 | ATTCTTATAGTAGGTTCAGAGTTTGATTTAACTGAGCAAACTGAGTCACAAAGCTCAGAT | 3960 |
| | ............................................................ | |
| 3961 | CATCTAAAACTGATTTCTTGAAAATGAAAAGGAGTTCACCACAGTCACCACATTTCAATC | 4020 |
| | ............................................................ | |
| 4021 | CAGCAGAGCACATTTGGGATTTAGTCAAATGGGAGATTGCCATCACAGATGTGCAGCTGA | 4080 |
| | ............................................................ | |
| 4081 | CAAACTTGCAGCACCTGTGTGATGCTATCACATCAATATGGACCACAATCTCTGAGGAAT | 4140 |
| | ............................................................ | |
| 4141 | GTTTCCTCCACTCAATTCCAGTTGAATATATTTTAAAAATTAAGACAGTGTGAAGACAAA | 4200 |
| | ............................................................ | |
| 4201 | GGGGTGTTTAACCTAGCAAAATGTACCCAATAAAGTAGCTAGTGAGTGTAGTTTGACTAA | 4260 |
| | ............................................................ | |
| 4261 | ATCTGGGTCAGACAGCTCTTTTAGATACCCATGGGTTTCTTTTAACTCAAGTGAAGTGCC | 4320 |
| | ............................................................ | |
| 4321 | AGATGGGTGGAGTTCTCAGCAACATAATTTAGAGGTAAAAGAAGAAAAGAATGGAGGGGG | 4380 |
| | ............................................................ | |
| 4381 | GAGAAAACTAATGACTTCATCTACTATGTAACAAACACCATCCGTCTGGCATCCCAAGAT | 4440 |
| | ............................................................ | |
| 4441 | AATCTAACAAACTAAAATGCCTCAGAATGGTTTTTAAGCAGGTTGGATGCTTGGGATTTC | 4500 |
| | ............................................................ | |
| 4501 | AGCATATGCACACTGCAAAAGAAACATATTCATTCAACATTCAGTGCTGTGATTGAATGA | 4560 |
| | ............................................................ | |
| 4561 | TATTCATTAAGAAGAACACTGCAGGGACCTGCTGATTAACAATCTCCTCATACACCCAGT | 4620 |
| | ............................................................ | |
| 4621 | CTGCTGAACCTCTCAATGTCTACAATTTGCCACCAACTCCGTCTATTTTGTAAGCCACAG | 4680 |
| | ............................................................ | |
| 4681 | ACCTGTAATTATCTTTGAAATGTAATTATGTTTACGTTTTCAAACAAACATCCAATTAAG | 4740 |
| | ............................................................ | |
| 4741 | TGTCACTTTTGAATCTGTTTTCCTGAAGAATATTTCAATGTGCTGTTTTTTACACTATTT | 4800 |
| | ............................................................ | |
| 4801 | TATAAAGTGTTTTATTATATCCTCTCAGCTTGAATAGATTTTGTATGATGAATGTGAGCG | 4860 |
| | ............................................................ | |
| 4861 | TTTGAAGAGGCGTGACAAACAGAAAAACTCTCTCACACACACACATATGCAATAATTGAG | 4920 |
| | ............................................................ | |
| 4921 | CTGTCTTTATCTAGCAATGCTGTCCTTCAGAGCATCCAAAGCTTTCAAGGACAAAGTGAC | 4980 |
| | ............................................................ | |
| 4981 | CCTCCCAACCTCTGCTCTGTGCAGCAAAGTGGGTGGGTGGGCGTAGGAGGAGAGGTACGC | 5040 |
| | ............................................................ | |
| 5041 | AGCTGCTCTTTCTGCTTATTACGGGGGGATGGATATGGCAGCTAGATAAGCTGTGTGTGT | 5100 |
| | ............................................................ | |
| 5101 | GCGCGCACACACACACACACACACACACACAATAGCAACCCACACTCTCAAGGCTGCAGC | 5160 |
| | ............................................................ | |
| 5161 | TGCAAGAAGGAATCCAAGACCATCTCATTGATATGGATACACTGCCTCCTACATGCCAAC | 5220 |
| | ............................................................ | |
| 5221 | ATTCAAAGTTAGGGTGCAATTATATACTTTCACCACCAGGTGATGCTACTGGGGCTAGAT | 5280 |
| | ............................................................ | |
| 5281 | TTCTGGTGAGTTTACCTCCATCTGTTTGCACAAAAGTCCAAACAAATTCACCAGTCTCAG | 5340 |
| | ............................................................ | |
| 5341 | TAGATCCTACAAATTTTGCTCGATGTTGTCTTATGAGAAAAATAAATAAATAAATATTTT | 5400 |
| | ............................................................ | |
| 5401 | TTTCCTAAATTTGCTTTTTTTTAAAATAACTTTTTATTTCTACATAATTTTCATAAAAGA | 5460 |
| | ............................................................ | |
| 5461 | TTATATCAATTCCTGCATGAGGATTAATGCTCATCAGACAGTTACCTGTCCCCTACATAC | 5520 |
| | ............................................................ | |
| 5521 | ACTGTATTTCTTCTTCATTTTTATATCATATCATATAGTTTTCCAAGTAAAAGATAAATC | 5580 |
| | ............................................................ | |
| 5581 | ACTCTAATGCATTTGCACTCAAATTTATGTGCACAAAAAAAAGTGAGTGTTGCAATACAG | 5640 |
| | ............................................................ | |
| 5641 | AAAGACATGCCGTTATGCTCTCTGACATCTTCTCTAGACAGCACTGGAGATGGTATAACA | 5700 |
| | ............................................................ | |
| 5701 | AAACACCCTCAGTATAAAGCCTTCAAGTTCATGACTAATCGTTGGCAGCTAAACAATGCC | 5760 |
| | ............................................................ | |
| 5761 | CTCTGGTGGTCGTCGTGCATAATAAATATACAAGTTAAAGTGTTAAAGTTGTATTCCACT | 5820 |
| | ............................................................ | |
| 5821 | CAAAATCTGTAATTTGGTTTGGGGTCAGTGTCC | 5853 |
| | ................................. | |

SEQ ID NOs 104 and 106 (FSHR mutant allele- 5 nt deletion)
LENGTH: 5853 bp and 264 aa
TYPE: cDNA (SEQ ID NO: 104) and Protein (SEQ ID NO: 106)
ORGANISM: Nile tilapia

| | | |
|---|---|---|
| 1 | GCATTCACTACTGCATGACAGAAAACACCAAAACACCTCACATTTCTCTCTAGCTGACCT | 60 |
| | ............................................................ | |
| 61 | GGCGCCGAACCCTCGAGCGGACAGACAGGCAAAGGCGTTCATATCAAATGTGGAGTGTGG | 120 |
| | ...............................................-M--W--S--V-- | 4 |
| 121 | ACCAGAGACAATATCAGAGTAAAATACACAAAAGAAGACAAACTAGAAAAGTGAAACCAC | 180 |
| 5 | D--Q--R--Q--Y--Q--S--K--I--H--K--R--R--Q--T--R--K--V--K--P-- | 24 |
| 181 | TCTGTGGACCCAGGCAGACTGAAATGATGCTGGTGATGTTTGGAGTCACGGCGTTTCCCT | 240 |
| 25 | L--C--G--P--R--Q--T--E--M--M--L--V--M--F--G--V--T--A--F--P-- | 44 |
| 241 | CCAACATCTCCAACGCCCAGTGCCTGGAAGTTAAGCAGACGCAGATCAGAGAGATTCAGC | 300 |
| 45 | S--N--I--S--N--A--Q--C--L--E--V--K--Q--T--Q--I--R--E--I--Q-- | 64 |
| 301 | AGGGCGCCCTCTCCAGCCTCCAGCATCTAATGGAACTGACCATTTCTGAGAACGACCTGC | 360 |
| 65 | Q--G--A--L--S--S--L--Q--H--L--M--E--L--T--I--S--E--N--D--L-- | 84 |
| 361 | TGGAGAGTATCGGTGCTTTTGCCTTTTCTGGCCTCCCTCACCTCACCAAAATCTTAATAT | 420 |
| 85 | L--E--S--I--G--A--F--A--F--S--G--L--P--H--L--T--K--I--L--I-- | 104 |
| 421 | CTAAAAATGCTGCTCTGAGGAATATCGGGGCTTTTGTTTTCTCCAACCTCCCTGAACTCA | 480 |
| 105 | S--K--N--A--A--L--R--N--I--G--A--F--V--F--S--N--L--P--E--L-- | 124 |
| 481 | GTGAGATAATCATAACAAAATCAAAACACCTGAGTTTCATCCACCCCGATGCATTCAGGA | 540 |
| 125 | S--E--I--I--I--T--K--S--K--H--L--S--F--I--H--P--D--A--F--R-- | 144 |
| 541 | ACATGGCAAGACTACGGTTCTTGACTATCTCCAACACCGGGCTGAGGATTTTTCCAGACT | 600 |
| 145 | N--M--A--R--L--R--F--L--T--I--S--N--T--G--L--R--I--F--P--D-- | 164 |
| 601 | TCTCCAAGATCCATTCCACCGCCTGCTTTCTGCTGGATCTTCAGGACAACAGCCACATAA | 660 |
| 165 | F--S--K--I--H--S--T--A--C--F--L--L--D--L--Q--D--N--S--H--I-- | 184 |
| 661 | AGAGAGTCCCTGCCAATGCCTTCAGAGGCCTCTGCACTCAAACCTTCGCAGAGATACGGC | 720 |
| 185 | K--R--V--P--A--N--A--F--R--G--L--C--T--Q--T--F--A--E--I--R-- | 204 |
| 721 | TCACCAGAAATGGCATCAAGGAGGTGGCAAGTGACGCCTTCAACGGAACAAAGATGCACA | 780 |
| 205 | L--T--R--N--G--I--K--E--V--A--S--D--A--F--N--G--T--K--M--H-- | 224 |
| 781 | GACTGTTCCTAGGAGGCAACCGACAGCTTACTCACATCAGTCCCAATGCCTTTGTGGGTT | 840 |
| 225 | R--L--F--L--G--G--N--R--Q--L--T--H--I--S--P--N--A--F--V--G-- | 244 |
| 841 | CCAGTGAGTTGGTGGTACTAGACGTCTCCGAAACAGCCCTCTTTGCCAGACTCGATCCTT | 900 |
| 245 | S--S--E--L--V--V--L--D--V--S--E--T--A--L--F--A--R--L--D--P-- | 264 |
| 901 | GATGGCCTCAAGAGGCTGATTGCCGAGTCAGCCTTCAACCTGAAAGAACTTCCTCCTATT | 960 |
| 265 | *- | 264 |

SEQ ID NOs 107 and 110 (wild-type VtgAa)
LENGTH: 4974 bp and 1657 aa
TYPE: cDNA (SEQ ID NO: 107) and Protein (SEQ ID NO: 110)
ORGANISM: Nile tilapia

| | | |
|---|---|---|
| 1 | ATGAGAGCGCTCGTGCTCGCCCTGATTCTGGCCTTTGTGGCTGGTGATCTTCAACATCAA | 60 |
| 1 | -M--R--A--L--V--L--A--L--I--L--A--F--V--A--G--D--L--Q--H--Q- | 20 |
| 61 | GATCCTGTTTTTGAAGCTGATAAAACCTATGTGTACAAGTATGAGGCGCTGCTCCTGGCG | 120 |
| 21 | -D--P--V--F--E--A--D--K--T--Y--V--Y--K--Y--E--A--L--L--L--A- | 40 |
| 121 | GGCCTGCTCGAGAAAGGTTCAGCGAGAGCTGGACTAAATATCAGCAGCAAAGTTAGCATC | 180 |
| 41 | -G--L--L--E--K--G--S--A--R--A--G--L--N--I--S--S--K--V--S--I- | 60 |
| 181 | AATGCTATAGACCAGAACACATACTTCATTAAGCTTGAGGAACCTGAGCTCCAGGAGTAT | 240 |
| 61 | -N--A--I--D--Q--N--T--Y--F--I--K--L--E--E--P--E--L--Q--E--Y- | 80 |
| 241 | AGTGGAATTTGGCCTGAGGATCCTTTTATCCCAGCAACTGAGCTGACTTCAGCCCTCCAA | 300 |
| 81 | -S--G--I--W--P--E--D--P--F--I--P--A--T--E--L--T--S--A--L--Q- | 100 |
| 301 | GCTGAGCTCACGACTCCCATTAAGTTTGAATATGTCAATGGTGCTGTTGGAAAAGTCTTC | 360 |
| 101 | -A--E--L--T--T--P--I--K--F--E--Y--V--N--G--A--V--G--K--V--F- | 120 |
| 361 | GCCCCTGAAACCGTCTCAACAACAGTGCTTAACATCTACAGAGGTATCCTGAATGTCTTT | 420 |
| 121 | -A--P--E--T--V--S--T--T--V--L--N--I--Y--R--G--I--L--N--V--F- | 140 |
| 421 | CAGCTCAACGTCAAAAAGACACTAAATGTCTACGAGTTGCAGGAGGCTGGAACTCAGGGT | 480 |
| 141 | -Q--L--N--V--K--K--T--L--N--V--Y--E--L--Q--E--A--G--T--Q--G- | 160 |
| 481 | GTGTGCAAGACACTTTACTCCATCACTGAGGACACAGAGGCTGAACGTGTCTATCTGAGA | 540 |
| 161 | -V--C--K--T--L--Y--S--I--T--E--D--T--E--A--E--R--V--Y--L--R- | 180 |
| 541 | AAGACCAGGGACATGAGCCACTGTCAAGAAAGAATAACTAAAGACATGGGGTTAGCATAC | 600 |
| 181 | -K--T--R--D--M--S--H--C--Q--E--R--I--T--K--D--M--G--L--A--Y- | 200 |
| 601 | ACAGAGAAATGTGGAAAGTGCCAGGAGGACACTAAAAACCTGAAAGGAGTTTCATCATAC | 660 |
| 201 | -T--E--K--C--G--K--C--Q--E--D--T--K--N--L--K--G--V--S--S--Y- | 220 |
| 661 | AGTTACATCATGAAACCACTCGATAATGGCATCCAGATCAAGGAGGCATCGGTCCATGAG | 720 |
| 221 | -S--Y--I--M--K--P--L--D--N--G--I--Q--I--K--E--A--S--V--H--E- | 240 |
| 721 | CTGATCCAGTTCTCACCTTTCAGTGAGCAGCATGGAGCCGCCCATATGGAGACCAAGCAA | 780 |
| 241 | -L--I--Q--F--S--P--F--S--E--Q--H--G--A--A--H--M--E--T--K--Q- | 260 |
| 781 | TCCTTGATGCTCCTTGACGTTCGAAGACCCCCTTATGCACCCACTACACCACCACCCCAG | 840 |
| 261 | -S--L--M--L--L--D--V--R--R--P--P--Y--A--P--T--T--P--P--P--Q- | 280 |
| 841 | GCTGAGTATTCACACCGTGGAAATCTCACATATCAGTTCTCCACTGAGCTTCTTCAGTTA | 900 |
| 281 | -A--E--Y--S--H--R--G--N--L--T--Y--Q--F--S--T--E--L--L--Q--L- | 300 |
| 901 | CCCATTCTGCTCCTCAATATCAACGACATAGAGTCTCAGCTCGAGGACACTCTGGTCAAA | 960 |
| 301 | -P--I--L--L--L--N--I--N--D--I--E--S--Q--L--E--D--T--L--V--K- | 320 |
| 961 | CAGGCTGTAGAAAGAGTTCATGAAGATGCACCTCTGGAATTTTTGAAGTTTGTTCAACTC | 1020 |
| 321 | -Q--A--V--E--R--V--H--E--D--A--P--L--E--F--L--K--F--V--Q--L- | 340 |
| 1021 | CTCCGTGCAGCCTCCAATGAAACTCTGGAGAACCTCTGGAGCAAACACTCAGGGATTTCT | 1080 |
| 341 | -L--R--A--A--S--N--E--T--L--E--N--L--W--S--K--H--S--G--I--S- | 360 |
| 1081 | GCCCACAGAAAATGGATCATGGACGCCATCCCTGCTGTGGGAAATCCTGATGCTCTGAGA | 1140 |
| 361 | -A--H--R--K--W--I--M--D--A--I--P--A--V--G--N--P--D--A--L--R- | 380 |
| 1141 | TTTATCAAAGAGAAATACCTAGCAGAAACCATAACTGTGTTTGAAGCCGTTCAGGCTTTG | 1200 |
| 381 | -F--I--K--E--K--Y--L--A--E--T--I--T--V--F--E--A--V--Q--A--L- | 400 |
| 1201 | ATTACTTCATTTCACATGGTGACAGCAACCACTGAGGCCATTGAGGTCATCGAGAGCCTA | 1260 |
| 401 | -I--T--S--F--H--M--V--T--A--T--T--E--A--I--E--V--I--E--S--L- | 420 |
| 1261 | ACAAAGGAAAGCAAAATAGTGAGAAACCCAGTTCTGCGTCAGATTGTATTCCTTGGCTAC | 1320 |
| 421 | -T--K--E--S--K--I--V--R--N--P--V--L--R--Q--I--V--F--L--G--Y- | 440 |
| 1321 | GGTACCATGATTTACAAACACTGCTATGAGAGGACTTCCTGTCCTGCTGAGCTCATACAG | 1380 |
| 441 | -G--T--M--I--Y--K--H--C--Y--E--R--T--S--C--P--A--E--L--I--Q- | 460 |
| 1381 | CCCATTCAAGACCTTCTTGCGCAGGCACTGAAAGATGGAAACACAGAGGACATCATCCTG | 1440 |
| 461 | -P--I--Q--D--L--L--A--Q--A--L--K--D--G--N--T--E--D--I--I--L- | 480 |
| 1441 | TTTGTGAAGGCTTTGGGAAATGCTGCGCATCCTTCTAGCCTCAAGAAAATCACAAAGATG | 1500 |
| 481 | -F--V--K--A--L--G--N--A--A--H--P--S--S--L--K--K--I--T--K--M- | 500 |
| 1501 | CTGCCCCTACATAGTAAATTAGGTTCATCACTGCCAGTGAGAGTTCATGCTGAAGCCATG | 1560 |
| 501 | -L--P--L--H--S--K--L--G--S--S--L--P--V--R--V--H--A--E--A--M- | 520 |
| 1561 | ATGGCCTTGAAGAACATCGCCAAAAAGGAGCCTAAAACGGTCCAGTATTTAGCCTTTCAG | 1620 |
| 521 | -M--A--L--K--N--I--A--K--K--E--P--K--T--V--Q--Y--L--A--F--Q- | 540 |
| 1621 | CTCTACGGGGACAAGACTCTTCATTCAGAGATCCGCATGCTTGCGTGCATGGTGCTCTTT | 1680 |
| 541 | -L--Y--G--D--K--T--L--H--S--E--I--R--M--L--A--C--M--V--L--F- | 560 |
| 1681 | GAGACAAAACCTTCAATGAGTTTGGTGTCAGCTGTTGTTCATATTGTGAAGACAGATACA | 1740 |
| 561 | -E--T--K--P--S--M--S--L--V--S--A--V--V--H--I--V--K--T--D--T- | 580 |
| 1741 | AATTTGCAAGTAGTAAGCTTCACCTATTCCCACATGAAGTCCCTGACTAGGAGCACCAGC | 1800 |
| 581 | -N--L--Q--V--V--S--F--T--Y--S--H--M--K--S--L--T--R--S--T--S- | 600 |
| 1801 | GTTATTTATGCCTCAGTTGCTGCAGCATGCAAAGCTGCCCTGAGAATGTTGGGCCCAAAC | 1860 |
| 601 | -V--I--Y--A--S--V--A--A--A--C--K--A--A--L--R--M--L--G--P--N- | 620 |
| 1861 | CTGGACAAACTGAGCTCACGTTTCAGCAAAGCCATCCATGTCGACGTCTATAGCAGTCCC | 1920 |
| 621 | -L--D--K--L--S--S--R--F--S--K--A--I--H--V--D--V--Y--S--S--P- | 640 |
| 1921 | TTTATGCTTGGTGCTGCTGCGACTGCTTACTACATCAATGATGCTGCCACCATCATGCCC | 1980 |
| 641 | -F--M--L--G--A--A--A--T--A--Y--Y--I--N--D--A--A--T--I--M--P- | 660 |
| 1981 | AAATCTATTACGACTAGGATCAAGGCTTTCTTTGCTGGAGCTGCTGCTGACATTCTGGAG | 2040 |
| 661 | -K--S--I--T--T--R--I--K--A--F--F--A--G--A--A--A--D--I--L--E- | 680 |
| 2041 | GTTGGAGTAAGAACTGAGGGACTACAGGAGGCTTTTCTGAAAAACCCAGCAGTTTTTGAT | 2100 |
| 681 | -V--G--V--R--T--E--G--L--Q--E--A--F--L--K--N--P--A--V--F--D- | 700 |
| 2101 | AGTGCTGACAGGGTCACCAGGATGAAACATGTCATTAAGGCTCTCTCTCACTGGAAGTCT | 2160 |
| 701 | -S--A--D--R--V--T--R--M--K--H--V--I--K--A--L--S--H--W--K--S- | 720 |
| 2161 | GCACCCAACAGCAAATCCCTGACTTCCATCTATGTCAAGTTCTTTGGACAAGAAGTTGCC | 2220 |
| 721 | -A--P--N--S--K--S--L--T--S--I--Y--V--K--F--F--G--Q--E--V--A- | 740 |
| 2221 | TTTGTTGACTTTGACAAAATCTGGTTTGACAACATCTTTAATCTCATCTTTGCCAATAAC | 2280 |
| 741 | -F--V--D--F--D--K--I--W--F--D--N--I--F--N--L--I--F--A--N--N- | 760 |
| 2281 | AATGCTGACACGTTTGGTAGAGATGTTTTCAAGGCTCTGCAGTCTGGTCCTACTTTGCGC | 2340 |
| 761 | -N--A--D--T--F--G--R--D--V--F--K--A--L--Q--S--G--P--T--L--R- | 780 |
| 2341 | TTTGTTAAGCCTCTGCTGGCTAATGAGGTGAGACGTATCATGCCTACTATAGCTGGTTTT | 2400 |
| 781 | -F--V--K--P--L--L--A--N--E--V--R--R--I--M--P--T--I--A--G--F- | 800 |
| 2401 | CCCATGGAGCTCGGTCTGTACACTGCTGCTGTGGCTGCTGTTCCTGGTCAAATCAAAGTC | 2460 |
| 801 | -P--M--E--L--G--L--Y--T--A--A--V--A--A--V--P--G--Q--I--K--V- | 820 |
| 2461 | ACCACGACTCCAGCTCTGCCAGAAGACTTTTATCTCAGATACCTTCTCAAGGCAGATATA | 2520 |
| 821 | -T--T--T--P--A--L--P--E--D--F--Y--L--R--Y--L--L--K--A--D--I- | 840 |
| 2521 | CACATTAGTACCAAGGTCACACCAAGTGTCGCTGTGAACACATTTGCTGTGTTTGGGATA | 2580 |
| 841 | -H--I--S--T--K--V--T--P--S--V--A--V--N--T--F--A--V--F--G--I- | 860 |
| 2581 | AACACTGCCATACTCCAGGCTGTCATGGTATCCAGAGCCAAACTCTACTCCATCACACCA | 2640 |
| 861 | -N--T--A--I--L--Q--A--V--M--V--S--R--A--K--L--Y--S--I--T--P- | 880 |
| 2641 | GCCAAAACTGAAGTCACATTTAACATCAATGAGGGCTACTTGAATTTCACAGCTCTTCCT | 2700 |
| 881 | -A--K--T--E--V--T--F--N--I--N--E--G--Y--L--N--F--T--A--L--P- | 900 |
| 2701 | GTTTCAGTGCCTGAAAACATTACAGCTGTGGAGGTTGAGACTTTTGCTGTGGTAAGAAAT | 2760 |
| 901 | -V--S--V--P--E--N--I--T--A--V--E--V--E--T--F--A--V--V--R--N- | 920 |
| 2761 | CCTGCTTCGGGAGAAAGAATCACTCCTGTGATCCCTGCCAACCCAAGACAGATTCTTATA | 2820 |
| 921 | -P--A--S--G--E--R--I--T--P--V--I--P--A--N--P--R--Q--I--L--I- | 940 |
| 2821 | TCCAGTAATACTTCTTCTGATGCTGTTAGTGAGTCAAGATCCGAAGAGTTCATTTCTCAG | 2880 |
| 941 | -S--S--N--T--S--S--D--A--V--S--E--S--R--S--E--E--F--I--S--Q- | 960 |
| 2881 | CGTCAGAAAGCTGGCATGCACATCAAATCTAAAATGGTGAAGAGTAAGAAGAAGTACTGC | 2940 |
| 961 | -R--Q--K--A--G--M--H--I--K--S--K--M--V--K--S--K--K--K--Y--C- | 980 |
| 2941 | GCTCAGACTGTTAACGCTGGACTCAAGGCCTGTCTCAAGATTGCCACTGCTTACACGGGG | 3000 |
| 981 | -A--Q--T--V--N--A--G--L--K--A--C--L--K--I--A--T--A--Y--T--G- | 1000 |
| 3001 | GATGCTGCAGTGTATAAACTGGCTGGAAAGCACTCCGCTGCTTTTTCTGTCACACCAATT | 3060 |
| 1001 | -D--A--A--V--Y--K--L--A--G--K--H--S--A--A--F--S--V--T--P--I- | 1020 |
| 3061 | GAAGGTGAAGCTGCTGAGAGACTGGAATTAGAGGTTCAACTTGGAAGTAAGGCTGCACAG | 3120 |
| 1021 | -E--G--E--A--A--E--R--L--E--L--E--V--Q--L--G--S--K--A--A--Q- | 1040 |
| 3121 | AAGATCATCAAACACATCACGCTTAGAGAAGAAGAAATCCCAGAGGAAACACCAGTCTTA | 3180 |
| 1041 | -K--I--I--K--H--I--T--L--R--E--E--E--I--P--E--E--T--P--V--L- | 1060 |
| 3181 | ATGAAGCTCCACAAAATCCTGGCCTCTACCCAGAAGAATAGCACCATGTCCTCCTCATCC | 3240 |
| 1061 | -M--K--L--H--K--I--L--A--S--T--Q--K--N--S--T--M--S--S--S--S- | 1080 |
| 3241 | TCCAGTTCCAGGAGCTCTCGCTTTCATGTCAGATCCTCTTCTTCCAATTCCAGCTCTTCA | 3300 |
| 1081 | -S--S--S--R--S--S--R--F--H--V--R--S--S--S--S--N--S--S--S--S- | 1100 |
| 3301 | TCCCATTCTAGCAGGAAGACCATTGATGCAACTGCTCAACAAGTCTTCAGCTTCTCCACC | 3360 |
| 1101 | -S--H--S--S--R--K--T--I--D--A--T--A--Q--Q--V--F--S--F--S--T- | 1120 |
| 3361 | TCTGTCAGTACTTCCAAGTCCAGCTTTGCATCGAGCTTTGCATCACTCTTCAGTCTTAGT | 3420 |
| 1121 | -S--V--S--T--S--K--S--S--F--A--S--S--F--A--S--L--F--S--L--S- | 1140 |
| 3421 | TCAAGCTCTTCTCACTACAGTGCGCACCACAGAAAGCATCCTGCGAGTCGCCACAAACCC | 3480 |
| 1141 | -S--S--S--S--H--Y--S--A--H--H--R--K--H--P--A--S--R--H--K--P- | 1160 |
| 3481 | AAGGAGAAACACAAGCATCCCACCTCTAAAGCCACATCGTCACAGGTTTTCAAAAGCAGA | 3540 |
| 1161 | -K--E--K--H--K--H--P--T--S--K--A--T--S--S--Q--V--F--K--S--R- | 1180 |
| 3541 | AGCAGTGGCTCAAGCTTGGACGCTATCCAACATAAGAAGCGGTTCCTTGACAGTCAAGCT | 3600 |
| 1181 | -S--S--G--S--S--L--D--A--I--Q--H--K--K--R--F--L--D--S--Q--A- | 1200 |
| 3601 | GCTATCTTTGGCATGATCTTCCGTGCTGTTAAAGCTGACACGAAGAAGCAGGGATACCAG | 3660 |
| 1201 | -A--I--F--G--M--I--F--R--A--V--K--A--D--T--K--K--Q--G--Y--Q- | 1220 |
| 3661 | TTCACTGCTTACATGGACAAAACCACCAGCAGACTTCAAATCATTCTAGATGACATTGTT | 3720 |
| 1221 | -F--T--A--Y--M--D--K--T--T--S--R--L--Q--I--I--L--D--D--I--V- | 1240 |
| 3721 | CCTGATAACAACTGGAGGCTCTGTGCTGATGGAGCCGTGTTGAGCATGCACAAAGTCAAA | 3780 |
| 1241 | -P--D--N--N--W--R--L--C--A--D--G--A--V--L--S--M--H--K--V--K- | 1260 |
| 3781 | GCTAAAATGAACTGGGGAGCAGAATGCAACCAATATGACACCACGATTACAACAGAAACT | 3840 |
| 1261 | -A--K--M--N--W--G--A--E--C--N--Q--Y--D--T--T--I--T--T--E--T- | 1280 |
| 3841 | GGTCTTGTCGGTCGAAACCCTGCAGCTCGGCTGAAGGTGGACTGGAATCGGCTACCGTCT | 3900 |
| 1281 | -G--L--V--G--R--N--P--A--A--R--L--K--V--D--W--N--R--L--P--S- | 1300 |
| 3901 | GATCTCAAGCACCATGCAAAGACGATGTATAAGTACATTTCTGCTCACATGCCTGCCGGC | 3960 |
| 1301 | -D--L--K--H--H--A--K--T--M--Y--K--Y--I--S--A--H--M--P--A--G- | 1320 |
| 3961 | TTGATTCAGGAAAAGGACAGAAACAGCGACAAGCAGCTCTCGTTGACTGTGGCTGTAGTA | 4020 |
| 1321 | -L--I--Q--E--K--D--R--N--S--D--K--Q--L--S--L--T--V--A--V--V- | 1340 |
| 4021 | TCTGACAAGATCATCGACCTGATTTGGAAAACACCGAGAAGCACTGTTCATAAGCGGGCT | 4080 |
| 1341 | -S--D--K--I--I--D--L--I--W--K--T--P--R--S--T--V--H--K--R--A- | 1360 |
| 4081 | TTGCATCTTCCCATCACTCTGCCACGTAACGAGATCAAAGATCTTACTTCCTTCAGTGAC | 4140 |
| 1361 | -L--H--L--P--I--T--L--P--R--N--E--I--K--D--L--T--S--F--S--D- | 1380 |
| 4141 | GTCTCTGGAAAAGTCAAGCACTTGTTAGCTGCGGCTGGCGCAGCTGAATGTAGCTTCACC | 4200 |
| 1381 | -V--S--G--K--V--K--H--L--L--A--A--A--G--A--A--E--C--S--F--T- | 1400 |
| 4201 | GACAATACGCTGACCACATTCAACAACAAGAAATTAAAGAACGAGATGCCCTCAAACTGC | 4260 |
| 1401 | -D--N--T--L--T--T--F--N--N--K--K--L--K--N--E--M--P--S--N--C- | 1420 |
| 4261 | TATCAGGTTCTGGCACAGGATGGCACAGACGAGCTGAAATTCATCGTTCTACTGAGGAAG | 4320 |
| 1421 | -Y--Q--V--L--A--Q--D--G--T--D--E--L--K--F--I--V--L--L--R--K- | 1440 |
| 4321 | GATCGCACTGAACAGAAGCAGATCAGTGTGAAAATTGCTCATATAGACATTGACCTCTAT | 4380 |
| 1441 | -D--R--T--E--Q--K--Q--I--S--V--K--I--A--H--I--D--I--D--L--Y- | 1460 |
| 4381 | CAGAGGAGAACCAGTGTGACTGTGAATGTGAATGGGCTGGAAATACCCATGAGCAACCTG | 4440 |
| 1461 | -Q--R--R--T--S--V--T--V--N--V--N--G--L--E--I--P--M--S--N--L- | 1480 |
| 4441 | CCATATCGTTATCCCCAAGCTGACATCCAGATCAAACAAAATGGCGAAGGCATCTCTGTG | 4500 |
| 1481 | -P--Y--R--Y--P--Q--A--D--I--Q--I--K--Q--N--G--E--G--I--S--V- | 1500 |
| 4501 | TATGCAGCTAGCTATGGTCTTCATGAAGTCTACTTTGACAAGAAGTCATGGAAGATTAAA | 4560 |
| 1501 | -Y--A--A--S--Y--G--L--H--E--V--Y--F--D--K--K--S--W--K--I--K- | 1520 |
| 4561 | GTTGTGGACTGGATGAAGGGGAAGACTTGTGGGCTCTGTGGAAAGGCTGACGGGGAGACC | 4620 |
| 1521 | -V--V--D--W--M--K--G--K--T--C--G--L--C--G--K--A--D--G--E--T- | 1540 |
| 4621 | ATGCAGGAGTATCGCACACCCACTGGATGGATAGCCACGACAGCAGTGAGCTTTGCTCAT | 4680 |
| 1541 | -M--Q--E--Y--R--T--P--T--G--W--I--A--T--T--A--V--S--F--A--H- | 1560 |
| 4681 | TCTTGGATTCTGCCAGCTGAGAGCTGCAGAGACGCCACTGAGTGCCGTATGAGGCATGAA | 4740 |
| 1561 | -S--W--I--L--P--A--E--S--C--R--D--A--T--E--C--R--M--R--H--E- | 1580 |
| 4741 | TCTGTGCAGCTGGAGAAACAGGAAAACGTGCAAGCTCAGAACTCCAAGTGCTACTCTGTC | 4800 |
| 1581 | -S--V--Q--L--E--K--Q--E--N--V--Q--A--Q--N--S--K--C--Y--S--V- | 1600 |
| 4801 | GACCCTGTGCTGCGCTGCATGGCTGGGTGCTTCCCTGTGCGCACCACCAACGTCACTGTT | 4860 |
| 1601 | -D--P--V--L--R--C--M--A--G--C--F--P--V--R--T--T--N--V--T--V- | 1620 |
| 4861 | GGCTTCCACTGCCTTCCAGCTGGTTCCAGCCCCTCCAGCATGTATACGAGCGTGGACCTG | 4920 |
| 1621 | -G--F--H--C--L--P--A--G--S--S--P--S--S--M--Y--T--S--V--D--L- | 1640 |
| 4921 | ATGGAAACTACGGAGAGTCACCTCGCCTGCACCTGCACTGCTCAGTGTGCTTAA | 4974 |
| 1641 | -M--E--T--T--E--S--H--L--A--C--T--C--T--A--Q--C--A--*- | 1657 |

SEQ ID NOs 108 and 111 (VtaAa mutant allele- 5 nt deletion)
LENGTH: 4974 bp and 279 aa
TYPE: cDNA (SEQ ID NO: 108) and Protein (SEQ ID NO: 111)
ORGANISM: Nile tilapia

| | | |
|---|---|---|
| 1 | ATGAGAGCGCTCGTGCTCGCCCTGATTCTGGCCTTTGTGGCTGGTGATCTTCAACATCAA | 60 |
| 1 | -M--R--A--L--V--L--A--L--I--L--A--F--V--A--G--D--L--Q--H--Q- | 20 |
| 61 | GATCCTGTTTTTGAAGCTGATAAAACCTATGTGTACAAGTATGAGGCGCTGCTCCTGGCG | 120 |
| 21 | -D--P--V--F--E--A--D--K--T--Y--V--Y--K--Y--E--A--L--L--L--A- | 40 |
| 121 | GGCCTGCTCGAGAAAGGTTCAGCGAGAGCTGGACTAAATATCAGCAGCAAAGTTAGCATC | 180 |
| 41 | -G--L--L--E--K--G--S--A--R--A--G--L--N--I--S--S--K--V--S--I- | 60 |
| 181 | AATGCTATAGACCAGAACACATACTTCATTAAGCTTGAGGAACCTGAGCTCCAGGAGTAT | 240 |
| 61 | -N--A--I--D--Q--N--T--Y--F--I--K--L--E--E--P--E--L--Q--E--Y- | 80 |
| 241 | AGTGGAATTTGGCCTGAGGATCCTTTTATCCCAGCAACTGAGCTGACTTCAGCCCTCCAA | 300 |
| 81 | -S--G--I--W--P--E--D--P--F--I--P--A--T--E--L--T--S--A--L--Q- | 100 |
| 301 | GCTGAGCTCACGACTCCCATTAAGTTTGAATATGTCAATGGTGCTGTTGGAAAAGTCTTC | 360 |
| 101 | -A--E--L--T--T--P--I--K--F--E--Y--V--N--G--A--V--G--K--V--F- | 120 |
| 361 | GCCCCTGAAACCGTCTCAACAACAGTGCTTAACATCTACAGAGGTATCCTGAATGTCTTT | 420 |
| 121 | -A--P--E--T--V--S--T--T--V--L--N--I--Y--R--G--I--L--N--V--F- | 140 |
| 421 | CAGCTCAACGTCAAAAAGACACTAAATGTCTACGAGTTGCAGGAGGCTGGAACTCAGGGT | 480 |
| 141 | -Q--L--N--V--K--K--T--L--N--V--Y--E--L--Q--E--A--G--T--Q--G- | 160 |
| 481 | GTGTGCAAGACACTTTACTCCATCACTGAGGACACAGAGGCTGAACGTGTCTATCTGAGA | 540 |
| 161 | -V--C--K--T--L--Y--S--I--T--E--D--T--E--A--E--R--V--Y--L--R- | 180 |
| 541 | AAGACCAGGGACATGAGCCACTGTCAAGAAAGAATAACTAAAGACATGGGGTTAGCATAC | 600 |
| 181 | -K--T--R--D--M--S--H--C--Q--E--R--I--T--K--D--M--G--L--A--Y- | 200 |
| 601 | ACAGAGAAATGTGGAAAGTGCCAGGAGGACACTAAAAACCTGAAAGGAGTTTCATCATAC | 660 |
| 201 | -T--E--K--C--G--K--C--Q--E--D--T--K--N--L--K--G--V--S--S--Y- | 220 |
| 661 | AGTTACATCATGAAACCACTCGATAATGGCATCCAGATCAAGGAGGCATCGGTCCATGAG | 720 |
| 221 | -S--Y--I--M--K--P--L--D--N--G--I--Q--I--K--E--A--S--V--H--E- | 240 |
| 721 | CTGATCCAGTTCTCACCTTTCAGTGAGCAGCATGGAGCCGCCCATATGGAGACCAAGCAA | 780 |
| 241 | -L--I--Q--F--S--P--F--S--E--Q--H--G--A--A--H--M--E--T--K--Q- | 260 |
| 781 | TCCTTGATGCTCCTTGACGTTCGAAGACCCCCTTATGCACCCACTACACCACCAGGCTGA | 840 |
| 261 | -S--L--M--L--L--D--V--R--R--P--P--Y--A--P--T--T--P--P--G--*- | 279 |

SEQ ID NOs 109 and 112 (VtoAa mutant allele- 25 nt deletion)
LENGTH: 4974 bp and 301 aa
TYPE: cDNA (SEQ ID NO: 109) and Protein (SEQ ID NO: 112)
ORGANISM: Nile tilapia

| | | |
|---|---|---|
| 1 | ATGAGAGCGCTCGTGCTCGCCCTGATTCTGGCCTTTGTGGCTGGTGATCTTCAACATCAA | 60 |
| 1 | -M--R--A--L--V--L--A--L--I--L--A--F--V--A--G--D--L--Q--H--Q- | 20 |
| 61 | GATCCTGTTTTTGAAGCTGATAAAACCTATGTGTACAAGTATGAGGCGCTGCTCCTGGCG | 120 |
| 21 | -D--P--V--F--E--A--D--K--T--Y--V--Y--K--Y--E--A--L--L--L--A- | 40 |
| 121 | GGCCTGCTCGAGAAAGGTTCAGCGAGAGCTGGACTAAATATCAGCAGCAAAGTTAGCATC | 180 |
| 41 | -G--L--L--E--K--G--S--A--R--A--G--L--N--I--S--S--K--V--S--I- | 60 |
| 181 | AATGCTATAGACCAGAACACATACTTCATTAAGCTTGAGGAACCTGAGCTCCAGGAGTAT | 240 |
| 61 | -N--A--I--D--Q--N--T--Y--F--I--K--L--E--E--P--E--L--Q--E--Y- | 80 |
| 241 | AGTGGAATTTGGCCTGAGGATCCTTTTATCCCAGCAACTGAGCTGACTTCAGCCCTCCAA | 300 |
| 81 | -S--G--I--W--P--E--D--P--F--I--P--A--T--E--L--T--S--A--L--Q- | 100 |
| 301 | GCTGAGCTCACGACTCCCATTAAGTTTGAATATGTCAATGGTGCTGTTGGAAAAGTCTTC | 360 |
| 101 | -A--E--L--T--T--P--I--K--F--E--Y--V--N--G--A--V--G--K--V--F- | 120 |
| 361 | GCCCCTGAAACCGTCTCAACAACAGTGCTTAACATCTACAGAGGTATCCTGAATGTCTTT | 420 |
| 121 | -A--P--E--T--V--S--T--T--V--L--N--I--Y--R--G--I--L--N--V--F- | 140 |
| 421 | CAGCTCAACGTCAAAAAGACACTAAATGTCTACGAGTTGCAGGAGGCTGGAACTCAGGGT | 480 |
| 141 | -Q--L--N--V--K--K--T--L--N--V--Y--E--L--Q--E--A--G--T--Q--G- | 160 |
| 481 | GTGTGCAAGACACTTTACTCCATCACTGAGGACACAGAGGCTGAACGTGTCTATCTGAGA | 540 |
| 161 | -V--C--K--T--L--Y--S--I--T--E--D--T--E--A--E--R--V--Y--L--R- | 180 |
| 541 | AAGACCAGGGACATGAGCCACTGTCAAGAAAGAATAACTAAAGACATGGGGTTAGCATAC | 600 |
| 181 | -K--T--R--D--M--S--H--C--Q--E--R--I--T--K--D--M--G--L--A--Y- | 200 |
| 601 | ACAGAGAAATGTGGAAAGTGCCAGGAGGACACTAAAAACCTGAAAGGAGTTTCATCATAC | 660 |
| 201 | -T--E--K--C--G--K--C--Q--E--D--T--K--N--L--K--G--V--S--S--Y- | 220 |
| 661 | AGTTACATCATGAAACCACTCGATAATGGCATCCAGATCAAGGAGGCATCGGTCCATGAG | 720 |
| 221 | -S--Y--I--M--K--P--L--D--N--G--I--Q--I--K--E--A--S--V--H--E- | 240 |
| 721 | CTGATCCAGTTCTCACCTTTCAGTGAGCAGCATGGAGCCGCCCATATGGAGACCAAGCAA | 780 |
| 241 | -L--I--Q--F--S--P--F--S--E--Q--H--G--A--A--H--M--E--T--K--Q- | 260 |
| 781 | TCCTTGATGCTCCTTGACGTTCGAAGACACCCCAGGCTGAGTATTCACACCGTGGAAATC | 840 |
| 261 | -S--L--M--L--L--D--V--R--R--H--P--R--L--S--I--H--T--V--E--I- | 280 |
| 841 | TCACATATCAGTTCTCCACTGAGCTTCTTCAGTTACCCATTCTGCTCCTCAATATCAACG | 900 |
| 281 | -S--H--I--S--S--P--L--S--F--F--S--Y--P--F--C--S--S--I--S--T- | 300 |
| 901 | ACATAGAGTCTCAGCTCGAGGACACTCTGGTCAAACAGGCTGTAGAAAGAGTTCATGAAG | 960 |
| 301 | -T--*- | 301 |

SEQ ID NOs 113 and 115 (wild-type VtgAb)
LENGTH: 5339 bp and 1747 aa
TYPE: cDNA (SEQ ID NO: 113) and Protein (SEQ ID NO: 115)
ORGANISM: Nile tilapia

| | | |
|---|---|---|
| 1 | CGCCATTTAGTTAATGATACATTTGATGGGCAACGTCAGCAAAAAATCTGCTTAAAAAGG | 60 |
| | ............................................................ | |
| 61 | ACGCCTCTGCCTGCAGATCCTCACATCCACCAGCCATGAGGGTGCTTGTACTAGCTCTTG | 120 |
| | ...................................-M--R--V--L--V--L--A--L-- | 8 |
| 121 | CTGTGGCTCTCGCAGTGGGGGACCAGTCCAACTTGGCCCCAGGATTCGCCTCTGTTAAGA | 180 |
| 9 | A--V--A--L--A--V--G--D--Q--S--N--L--A--P--G--F--A--S--V--K-- | 28 |
| 181 | CCTACATGTACAAATATGAAGCGGTTCTTATGGGCGGCCTGCCTGAAGAGGGCCTGGCTC | 240 |
| 29 | T--Y--M--Y--K--Y--E--A--V--L--M--G--G--L--P--E--E--G--L--A-- | 48 |
| 241 | GAGCTGGGGTTAAAATCCGGGGCAAAGTTTTGATCAGTGCAACAAGTGCCAACGACTACA | 300 |
| 49 | R--A--G--V--K--I--R--G--K--V--L--I--S--A--T--S--A--N--D--Y-- | 68 |
| 301 | TTCTGAAGCTTGTAGACCCTCAGTTGCTGGAGTACAGTGGCATCTGGCCCAAAGATCCTT | 360 |
| 69 | I--L--K--L--V--D--P--Q--L--L--E--Y--S--G--I--W--P--K--D--P-- | 88 |
| 361 | TCCATCCAGCCACCAAGCTCACCACAGCCCTGGCTACTCAGCTCTCGACACCGGTCAAGT | 420 |
| 89 | F--H--P--A--T--K--L--T--T--A--L--A--T--Q--L--S--T--P--V--K-- | 108 |
| 421 | TTGAGTATACAAACGGCGTTGTTGGGAGACTGGCTGCACCTCCTGGGGTCTCCACAACAG | 480 |
| 109 | F--E--Y--T--N--G--V--V--G--R--L--A--A--P--P--G--V--S--T--T-- | 128 |
| 481 | TGCTGAATATCTACAGGGGCATCATCAACCTCCTGCAGCTGAATGTAAAGAAGACACAGA | 540 |
| 129 | V--L--N--I--Y--R--G--I--I--N--L--L--Q--L--N--V--K--K--T--Q-- | 148 |
| 541 | ATGTCTACGAGATGCAAGAGTCTGGAGCTCATGGTGTGTGCAAGACCAACTATGTGATCA | 600 |
| 149 | N--V--Y--E--M--Q--E--S--G--A--H--G--V--C--K--T--N--Y--V--I-- | 168 |
| 601 | GGGAGGACGCGAGGGCCGAACGCATTCATCTGACCAAGACCAAGGACCTGAACCACTGCC | 660 |
| 169 | R--E--D--A--R--A--E--R--I--H--L--T--K--T--K--D--L--N--H--C-- | 188 |
| 661 | AGGAGAAAATCATGAAGGCCATCGGCTTGGAACACGTAGAGAAATGCCATGATTGTGAAG | 720 |
| 189 | Q--E--K--I--M--K--A--I--G--L--E--H--V--E--K--C--H--D--C--E-- | 208 |
| 721 | CTAGAGGAAAGAGCCTGAAGGGAACTGCTTCCTATAACTACATCATGAAGCCAGCACCCA | 780 |
| 209 | A--R--G--K--S--L--K--G--T--A--S--Y--N--Y--I--M--K--P--A--P-- | 228 |
| 781 | GTGGTTCTCTGATTATGGAGGCTGTCGCTAGAGAGGTCATCGAGTTTTCACCTTTCAACA | 840 |
| 229 | S--G--S--L--I--M--E--A--V--A--R--E--V--I--E--F--S--P--F--N-- | 248 |
| 841 | TTTTGAATGGCGCTGCTCAGATGGAGTCTAAGCAAATTCTGACCTTCCTGGATATTGAGA | 900 |
| 249 | I--L--N--G--A--A--Q--M--E--S--K--Q--I--L--T--F--L--D--I--E-- | 268 |
| 901 | ACACCCCTGTGGATCATGCCAGATACACCTATGTTCACCGCGGATCCCTGCAGTATGAGC | 960 |
| 269 | N--T--P--V--D--H--A--R--Y--T--Y--V--H--R--G--S--L--Q--Y--E-- | 288 |
| 961 | ATGGCAGCGAGATTCTCCAGACACCCATCCATCTTCTGAGGGTCACCCATGCCGAGGCTC | 1020 |
| 289 | H--G--S--E--I--L--Q--T--P--I--H--L--L--R--V--T--H--A--E--A-- | 308 |
| 1021 | AGATTGTCAGCACTCTGAACCACCTGGTAGCCTCCAACGTGGCCAAGGTCCATGAAGATG | 1080 |
| 309 | Q--I--V--S--T--L--N--H--L--V--A--S--N--V--A--K--V--H--E--D-- | 328 |
| 1081 | CCCCTCTGAAGTTTGTTGAGCTCATCCAGGTGATGCGTGTGGCCAGATTTGAGACTATTG | 1140 |
| 329 | A--P--L--K--F--V--E--L--I--Q--V--M--R--V--A--R--F--E--T--I-- | 348 |
| 1141 | AGTCCCTCTGGGCTCAGTTTAAATCTAGACCTGATCACAGGTACTGGTTACTGAATGCTG | 1200 |
| 349 | E--S--L--W--A--Q--F--K--S--R--P--D--H--R--Y--W--L--L--N--A-- | 368 |
| 1201 | TCCCCCACATTCGCACTCACGCTGCGCTTAAGTTCCTCATTGAGAAGCTCCTTGCTAATG | 1260 |
| 369 | V--P--H--I--R--T--H--A--A--L--K--F--L--I--E--K--L--L--A--N-- | 388 |
| 1261 | AGTTAAGTGAGACTGAAGCTGCTATGGCTCTCTTGGAATGTCTGCACTCTGTGACAGCTG | 1320 |
| 389 | E--L--S--E--T--E--A--A--M--A--L--L--E--C--L--H--S--V--T--A-- | 408 |
| 1321 | ACCAGAAAACCATTGAACTTGTCAGAAGCCTGGCTGAGAACCACAGAGTGAAACGTAACG | 1380 |
| 409 | D--Q--K--T--I--E--L--V--R--S--L--A--E--N--H--R--V--K--R--N-- | 428 |
| 1381 | CTGTGCTCAACGAGATTGTGATGCTGGGCTGGGGCACTGTAATTTCCAGGTTCTGTAAAG | 1440 |
| 429 | A--V--L--N--E--I--V--M--L--G--W--G--T--V--I--S--R--F--C--K-- | 448 |
| 1441 | CGCAGCCATCTTGCTCATCTGATCTTGTGACACCTGTACATAGACAAGTTGCAGAGGCTG | 1500 |
| 449 | A--Q--P--S--C--S--S--D--L--V--T--P--V--H--R--Q--V--A--E--A-- | 468 |
| 1501 | TTGAAACTGGTGACATCGATCAGCTCACTGTCACTCTCAAATGCCTGGATAACGCTGGAC | 1560 |
| 469 | V--E--T--G--D--I--D--Q--L--T--V--T--L--K--C--L--D--N--A--G-- | 488 |
| 1561 | ATCCTGCTAGCCTTAAGACAATCATGAAGTTCCTGCCTGGCTTTGGCAGTGCTGCTGCCC | 1620 |
| 489 | H--P--A--S--L--K--T--I--M--K--F--L--P--G--F--G--S--A--A--A-- | 508 |
| 1621 | GAGTCCCACTCAAAGTTCAGGTTGACGCTGTTCTAGCCCTGAGGAGAATTGCAAAGAGGG | 1680 |
| 509 | R--V--P--L--K--V--Q--V--D--A--V--L--A--L--R--R--I--A--K--R-- | 528 |
| 1681 | AACCCAAGATGGTCCAGGAAATAGCTGCTCAGTTGCTCATGGAAAAGCATCTCCATGCAG | 1740 |
| 529 | E--P--K--M--V--Q--E--I--A--A--Q--L--L--M--E--K--H--L--H--A-- | 548 |
| 1741 | AACTGCGTATGGTTGCTGCCATGGTGCTCTTTGAGACTAAACTCCCCGTGGGTCTAGCAG | 1800 |
| 549 | E--L--R--M--V--A--A--M--V--L--F--E--T--K--L--P--V--G--L--A-- | 568 |
| 1801 | CTAGCATTTCCACAGCCTTGATCAAAGAAAAGAACCTGCAGGTCGTTAGCTTTGTCTACT | 1860 |
| 569 | A--S--I--S--T--A--L--I--K--E--K--N--L--Q--V--V--S--F--V--Y-- | 588 |
| 1861 | CTTACATGAAGGCCATGGCCAAGACCACATCCCCTGACCACGTTTCTGTTGCTGCAGCAT | 1920 |
| 589 | S--Y--M--K--A--M--A--K--T--T--S--P--D--H--V--S--V--A--A--A-- | 608 |
| 1921 | GTAATGTTGCCTTGAGGTTCCTCAACCCCAAATTAGGCAGACTGAACTTCCGCTACAGCC | 1980 |
| 609 | C--N--V--A--L--R--F--L--N--P--K--L--G--R--L--N--F--R--Y--S-- | 628 |
| 1981 | GAGCCTTCCATGTGGATACCTATAACAATGCCTGGATGATGGGTGCTGCCGCCAGTGCCG | 2040 |
| 629 | R--A--F--H--V--D--T--Y--N--N--A--W--M--M--G--A--A--A--S--A-- | 648 |
| 2041 | TCTTAATTAACGACGCTGCAACCGTGTTACCAAGAATGATTATGGCCAAAGCCCGTACTT | 2100 |
| 649 | V--L--I--N--D--A--A--T--V--L--P--R--M--I--M--A--K--A--R--T-- | 668 |
| 2101 | ACATGGCCGGAGCTTATGTTGATGCTTTTGAGGTTGGAGTGAGGACTGAGGGAATCCAGG | 2160 |
| 669 | Y--M--A--G--A--Y--V--D--A--F--E--V--G--V--R--T--E--G--I--Q-- | 688 |
| 2161 | AGGCTCTTTTGAAAAGACGACATGAAAATTCTGAGAATGCAGACAGGATCACCAAGATTA | 2220 |
| 689 | E--A--L--L--K--R--R--H--E--N--S--E--N--A--D--R--I--T--K--I-- | 708 |
| 2221 | AACAAGCCATGAGAGCTCTTTCTGAGTGGAGGGCTAATCCTTCGAGCCAGGCCCTGGCCT | 2280 |
| 709 | K--Q--A--M--R--A--L--S--E--W--R--A--N--P--S--S--Q--A--L--A-- | 728 |
| 2281 | CTATGTATGTGAAGGTCTTCGGACAAGAAATTGCATTTGCCAACATTGACAAATCCAAGG | 2340 |
| 729 | S--M--Y--V--K--V--F--G--Q--E--I--A--F--A--N--I--D--K--S--K-- | 748 |
| 2341 | TTGACCAGCTTATCCAGTTTGCCAGTGGACCTTTGAGAAACGTATTCAGAGATGCTGTGA | 2400 |
| 749 | V--D--Q--L--I--Q--F--A--S--G--P--L--R--N--V--F--R--D--A--V-- | 768 |
| 2401 | ATTCTGTGCTGTCTGGTTATGCAACACATTTTGCTAAACCAATGCTGCTCGGTGAGCTCC | 2460 |
| 769 | N--S--V--L--S--G--Y--A--T--H--F--A--K--P--M--L--L--G--E--L-- | 788 |
| 2461 | GTCTCATCCTTCCCACCACTGTTGGGTTGCCCATGGAGATCAGCCTCATTACATCCGCTG | 2520 |
| 789 | R--L--I--L--P--T--T--V--G--L--P--M--E--I--S--L--I--T--S--A-- | 808 |
| 2521 | TGACTGCTGCATCTGTTGACGTCCAAGCCACTGTGTCACCACCTCTGCCTGTCAACTACC | 2580 |
| 809 | V--T--A--A--S--V--D--V--Q--A--T--V--S--P--P--L--P--V--N--Y-- | 828 |
| 2581 | GAGTTTCCCAGCTTCTGGAGTCCGATATCCAACTGAGGGCTACAGTTGCTCCAAGTCTTG | 2640 |
| 829 | R--V--S--Q--L--L--E--S--D--I--Q--L--R--A--T--V--A--P--S--L-- | 848 |
| 2641 | CCATGCAGACCTATGCATTCATGGGTGTGAACACCGCCTTAATCCAGGCTGCAGTGATGA | 2700 |
| 849 | A--M--Q--T--Y--A--F--M--G--V--N--T--A--L--I--Q--A--A--V--M-- | 868 |
| 2701 | CAAAAGCCAAAGTTTACACAGCTGTTCCTGCACAGATAAAAGCAAGGATTGACATTGTTA | 2760 |
| 869 | T--K--A--K--V--Y--T--A--V--P--A--Q--I--K--A--R--I--D--I--V-- | 888 |
| 2761 | AGGGCAACTTGAAGGTTGAGTTCCTGTCACTCCAGGGCATTAACACAATTGCATCTGCAC | 2820 |
| 889 | K--G--N--L--K--V--E--F--L--S--L--Q--G--I--N--T--I--A--S--A-- | 908 |
| 2821 | ATGCGGAGACGGTTGCCATTGCAAGAAATGTGGAAGACCTCCCAGCCGCAAGAAGCACAC | 2880 |
| 909 | H--A--E--T--V--A--I--A--R--N--V--E--D--L--P--A--A--R--S--T-- | 928 |
| 2881 | CACTGATCTCATCTGAAACTGCATCACAACTTTCAAAGGCCTCTCTCAACTCAAAGATCT | 2940 |
| 929 | P--L--I--S--S--E--T--A--S--Q--L--S--K--A--S--L--N--S--K--I-- | 948 |
| 2941 | CCAGGATGGCATCCTCTGTGACTGGTGGCATGTCTGCGTCATCTGAAATCATTCCTGCTG | 3000 |
| 949 | S--R--M--A--S--S--V--T--G--G--M--S--A--S--S--E--I--I--P--A-- | 968 |
| 3001 | ACCTGCCAAGTAAGATTGGGAGGAAAATGAAACTCCCTAAAACCTACAGGAAGAAAATCC | 3060 |
| 969 | D--L--P--S--K--I--G--R--K--M--K--L--P--K--T--Y--R--K--K--I-- | 988 |
| 3061 | GTGCTTCAAGCAGAATGCTAGGATTCAAGGCCTACGCTGAGATTAAATCTCACAATGCCG | 3120 |
| 989 | R--A--S--S--R--M--L--G--F--K--A--Y--A--E--I--K--S--H--N--A-- | 1008 |
| 3121 | CCTACATCAGAGACTGCCCTCTCTACGCTCTGATCGGAAAGCATGCTGCTTCTGTTAGGA | 3180 |
| 1009 | A--Y--I--R--D--C--P--L--Y--A--L--I--G--K--H--A--A--S--V--R-- | 1028 |
| 3181 | TTGCTCCAGCTTCTGGACCAGTCATTGAGAAGATTGAAGTTGAGATTCAGGTCGGAGATA | 3240 |
| 1029 | I--A--P--A--S--G--P--V--I--E--K--I--E--V--E--I--Q--V--G--D-- | 1048 |
| 3241 | AAGCAGCAGAAAATATGATTAAAGCGATTGACATGAGCGAAGAGGAGGAAGCTCTTGAGG | 3300 |
| 1049 | K--A--A--E--N--M--I--K--A--I--D--M--S--E--E--E--E--A--L--E-- | 1068 |
| 3301 | ATAAGAATGTCCTCTTGAAAATCAAGAAAATACTGGCACCTGGTCTCAAGAACACCACAT | 3360 |
| 1069 | D--K--N--V--L--L--K--I--K--K--I--L--A--P--G--L--K--N--T--T-- | 1088 |
| 3361 | CATCTTCCTCCAGCTCCTCCAGCTCCTCTTCATCCAGCTCTAGCTCCAACAAGTCTTCTT | 3420 |
| 1089 | S--S--S--S--S--S--S--S--S--S--S--S--S--S--S--S--N--K--S--S-- | 1108 |
| 3421 | CATCCAGTTCCCGCTCCAGCAGCTCCCAGTCATCCAGCTCTCGTTCCCATAGGTCTCGCT | 3480 |
| 1109 | S--S--S--S--R--S--S--S--S--Q--S--S--S--S--R--S--H--R--S--R-- | 1128 |
| 3481 | CCAGAAAGTCCCAGTCTAGCAGCTCTCAGTCAAGCCGCTCTCCCTCAAGCTCTTCCTCCT | 3540 |
| 1129 | S--R--K--S--Q--S--S--S--S--Q--S--S--R--S--P--S--S--S--S--S-- | 1148 |
| 3541 | CTTCCTCCTCTTCATCATCCAGATCTTCTTCCAGGTCATCTTCCAGATCATCTTCCAGAT | 3600 |
| 1149 | S--S--S--S--S--S--S--R--S--S--S--R--S--S--S--R--S--S--S--R-- | 1168 |
| 3601 | CTTCTTCTAGGTCCTCCTCTCGCTCCAGAACTAAGATGGCTGACATTGTTGCTCCTATTA | 3660 |
| 1169 | S--S--S--R--S--S--S--R--S--R--T--K--M--A--D--I--V--A--P--I-- | 1188 |
| 3661 | TCACGACGTCCACCAGAGTGAGCAGTTCCTCCAGTCGATCAGCCTCTAACAGCTCCTCCA | 3720 |
| 1189 | I--T--T--S--T--R--V--S--S--S--S--S--R--S--A--S--N--S--S--S-- | 1208 |
| 3721 | GCAGTGCTTCATACTTGCTCAGCTCATCTAAGTCATCAAGCTCTAGATCCTCTCGGCGCA | 3780 |
| 1209 | S--S--A--S--Y--L--L--S--S--S--K--S--S--S--S--R--S--S--R--R-- | 1228 |
| 3781 | GTGCTCAGTCTAAGCAACAACTGCTTGCCTTGAAGTTCAGAAAGAACCACGTCCACAGGC | 3840 |
| 1229 | S--A--Q--S--K--Q--Q--L--L--A--L--K--F--R--K--N--H--V--H--R-- | 1248 |
| 3841 | ATGCCATCTCCACACAGCGCGGCAGCAGTCACAGCAGTGCCCGCAGCTTCGATTCCATCT | 3900 |
| 1249 | H--A--I--S--T--Q--R--G--S--S--H--S--S--A--R--S--F--D--S--I-- | 1268 |
| 3901 | ACAATAAGGCCAAGTACCTCGCTAACACACTCACTCCTGCCATGTCCATTGCAATCCGTG | 3960 |
| 1269 | Y--N--K--A--K--Y--L--A--N--T--L--T--P--A--M--S--I--A--I--R-- | 1288 |
| 3961 | CCGTGAGAGTCGACCACAAGGTCCAGGGATACCAGCTAGCAGCTTACCTGGACAAACAGA | 4020 |
| 1289 | A--V--R--V--D--H--K--V--Q--G--Y--Q--L--A--A--Y--L--D--K--Q-- | 1308 |
| 4021 | CCAATAGACTGCAGCTGATTTTTGCCAGAGTCGCTGAGAAGGACAACTGGAGAATCTGTG | 4080 |
| 1309 | T--N--R--L--Q--L--I--F--A--R--V--A--E--K--D--N--W--R--I--C-- | 1328 |
| 4081 | CCGACATTGTGCAGCTGAGTTCGCACAAGATGATGGCCAAGATTGCCTGGGGTGCTGAAT | 4140 |
| 1329 | A--D--I--V--Q--L--S--S--H--K--M--M--A--K--I--A--W--G--A--E-- | 1348 |
| 4141 | GCAAGCAATACTCCACCATGATTGTAGCTGAAACTGGTCTTTTGGGTCATGAGCCCGCAG | 4200 |
| 1349 | C--K--Q--Y--S--T--M--I--V--A--E--T--G--L--L--G--H--E--P--A-- | 1368 |
| 4201 | CCCGCTTGAAGCTGACCTGGGACAAACTGCCAGGAAGCATAAAGCACTACGCAAAGAGGG | 4260 |
| 1369 | A--R--L--K--L--T--W--D--K--L--P--G--S--I--K--H--Y--A--K--R-- | 1388 |
| 4261 | CGTTGAAATCCATTGTCCCTATTGCTCAAGAATATGGAGTAAACTACGCAAAGGCCAAGA | 4320 |
| 1389 | A--L--K--S--I--V--P--I--A--Q--E--Y--G--V--N--Y--A--K--A--K-- | 1408 |
| 4321 | ATCCTCGTAATCAAATCAAACTGACTGTAGCTGTTGCTACTGAGACAAGCATGAATATTG | 4380 |
| 1409 | N--P--R--N--Q--I--K--L--T--V--A--V--A--T--E--T--S--M--N--I-- | 1428 |
| 4381 | TGCTGAACACACCAAAGGCAATCATTTACAAGCGTGGGGTGTGTCTACCTGTTGCTTTAC | 4440 |
| 1429 | V--L--N--T--P--K--A--I--I--Y--K--R--G--V--C--L--P--V--A--L-- | 1448 |
| 4441 | CAATTGGAAACACTGCTGCCGAGCTGCAAGCGACCCGGGACAACTGGGCTGACAAGATGT | 4500 |
| 1449 | P--I--G--N--T--A--A--E--L--Q--A--T--R--D--N--W--A--D--K--M-- | 1468 |
| 4501 | CCTATTTGGTTACCAAAGCTAACGCAGTTGAATGCTCCCTCATCAACAACACACTGACCA | 4560 |
| 1469 | S--Y--L--V--T--K--A--N--A--V--E--C--S--L--I--N--N--T--L--T-- | 1488 |
| 4561 | CATTCAACAACAGGAAAGCTAGAGATGAGCTGCCACACTCGTGCTACCAGGTCTTGGCTC | 4620 |
| 1489 | T--F--N--N--R--K--A--R--D--E--L--P--H--S--C--Y--Q--V--L--A-- | 1508 |
| 4621 | AGGATTGCACACCAGAACTCAAATTCATGGTTCTGCTGAAGAAAGACCAAATACAGGATC | 4680 |
| 1509 | Q--D--C--T--P--E--L--K--F--M--V--L--L--K--K--D--Q--I--Q--D-- | 1528 |
| 4681 | AGAAGCAGATCAATGTTAAGATTTCAGACATCGATGTGGACATGTATCGGAAGAACAACG | 4740 |
| 1529 | Q--K--Q--I--N--V--K--I--S--D--I--D--V--D--M--Y--R--K--N--N-- | 1548 |
| 4741 | CCATTGCGGTGATGGTTAACGGAGTTGAAATCCCTAACAGCAACCTGCCATACCTGCATC | 4800 |
| 1549 | A--I--A--V--M--V--N--G--V--E--I--P--N--S--N--L--P--Y--L--H-- | 1568 |
| 4801 | CATCAGGTAACATACATATAAGACAGTCAAATGAAGGCATTACTCTCAATGCACCCAGCC | 4860 |
| 1569 | P--S--G--N--I--H--I--R--Q--S--N--E--G--I--T--L--N--A--P--S-- | 1588 |
| 4861 | ATGGTCTTCAGGAGGTCTTCCTTGGCTTCAACGAGCTGAGGGTTAAAGTTGCAGACTGGA | 4920 |
| 1589 | H--G--L--Q--E--V--F--L--G--F--N--E--L--R--V--K--V--A--D--W-- | 1608 |
| 4921 | TGAAAGGAAAGACTTGTGGTGCCTGTGGAACGGCAAGCGGAAATGTCGGAGACGAGTACC | 4980 |
| 1609 | M--K--G--K--T--C--G--A--C--G--T--A--S--G--N--V--G--D--E--Y-- | 1628 |
| 4981 | GCACACCCAGTGAACAGGTGACCAAGGATGCCATCAGCTACGCCCACTCCTGGGTTCTGT | 5040 |
| 1629 | R--T--P--S--E--Q--V--T--K--D--A--I--S--Y--A--H--S--W--V--L-- | 1648 |
| 5041 | CTTCAAACACCTGCCGTGATCCCTCCGAGTGTTCCATCAAGCAGGAATCTGTGAAGCTGG | 5100 |
| 1649 | S--S--N--T--C--R--D--P--S--E--C--S--I--K--Q--E--S--V--K--L-- | 1668 |
| 5101 | AGAAGCGGGTGATCTTTGAAGGTGTGGAGTCCAAATGCTACTCTGTTGAGCCCGTGCTGC | 5160 |
| 1669 | E--K--R--V--I--F--E--G--V--E--S--K--C--Y--S--V--E--P--V--L-- | 1688 |
| 5161 | AGTGCCTGCCCGGCTGTATCCCAGTGAGAACCACTACCGTCAACGTTGGCTTTCACTGCC | 5220 |
| 1689 | Q--C--L--P--G--C--I--P--V--R--T--T--T--V--N--V--G--F--H--C-- | 1708 |
| 5221 | TGCCCAGTGACACAACTGTGGACCGTTCTGGTCTGAGCAGCTTCTTTGAGAAGAGCATCG | 5280 |
| 1709 | L--P--S--D--T--T--V--D--R--S--G--L--S--S--F--F--E--K--S--I-- | 1728 |
| 5281 | ACCTGAGGGATACTGCAGAAGCCCACCTGGCCTGTCGCTGCACTCCTCAGTGTGCTTAA | 5339 |
| 1729 | D--L--R--D--T--A--E--A--H--L--A--C--R--C--T--P--Q--C--A--*- | 1747 |

SEQ ID NOs 114 and 116 (VtgAb mutant allele- 8 nt deletion)
LENGTH: 5339 bp and 202 aa
TYPE: cDNA (SEQ ID NO: 114) and Protein (SEQ ID NO: 116)
ORGANISM: Nile tilapia

| | | |
|---|---|---|
| 1 | CGCCATTTAGTTAATGATACATTTGATGGGCAACGTCAGCAAAAAATCTGCTTAAAAAGG | 60 |
| | ............................................................ | |
| 61 | ACGCCTCTGCCTGCAGATCCTCACATCCACCAGCCATGAGGGTGCTTGTACTAGCTCTTG | 120 |
| | ...................................-M--R--V--L--V--L--A--L-- | 8 |
| 121 | CTGTGGCTCTCGCAGTGGGGGACCAGTCCAACTTGGCCCCAGGATTCGCCTCTGTTAAGA | 180 |
| 9 | A--V--A--L--A--V--G--D--Q--S--N--L--A--P--G--F--A--S--V--K-- | 28 |
| 181 | CCTACATGTACAAATATGAAGCGGTTCTTATGGGCGGCCTGCCTGAAGAGGGCCTGGCTC | 240 |
| 29 | T--Y--M--Y--K--Y--E--A--V--L--M--G--G--L--P--E--E--G--L--A-- | 48 |
| 241 | GAGCTGGGGTTAAAATCCGGGGCAAAGTTTTGATCAGTGCAACAAGTGCCAACGACTACA | 300 |
| 49 | R--A--G--V--K--I--R--G--K--V--L--I--S--A--T--S--A--N--D--Y-- | 68 |
| 301 | TTCTGAAGCTTGTAGACCCTCAGTTGCTGGAGTACAGTGGCATCTGGCCCAAAGATCCTT | 360 |
| 69 | I--L--K--L--V--D--P--Q--L--L--E--Y--S--G--I--W--P--K--D--P-- | 88 |
| 361 | TCCATCCAGCCACCAAGCTCACCACAGCCCTGGCTACTCAGCTCTCGACACCGGTCAAGT | 420 |
| 89 | F--H--P--A--T--K--L--T--T--A--L--A--T--Q--L--S--T--P--V--K-- | 108 |
| 421 | TTGAGTATACAAACGGCGTTGTTGGGAGACTGGCTGCACCTCCTGGGGTCTCCACAACAG | 480 |
| 109 | F--E--Y--T--N--G--V--V--G--R--L--A--A--P--P--G--V--S--T--T-- | 128 |
| 481 | TGCTGAATATCTACAGGGGCATCATCAACCTCCTGCAGCTGAATGTAAAGAAGACACAGA | 540 |
| 129 | V--L--N--I--Y--R--G--I--I--N--L--L--Q--L--N--V--K--K--T--Q-- | 148 |
| 541 | ATGTCTACGAGATGCAAGAGTCTGGAGCTCATGGTGTGTGCAAGACCAACTATGTGATCA | 600 |
| 149 | N--V--Y--E--M--Q--E--S--G--A--H--G--V--C--K--T--N--Y--V--I-- | 168 |
| 601 | GGGAGGGCCGAACGCATTCATCTGACCAAGACCAAGGACCTGAACCACTGCCAGGAGAAA | 660 |
| 169 | R--E--G--R--T--H--S--S--D--Q--D--Q--G--P--E--P--L--P--G--E-- | 188 |
| 661 | ATCATGAAGGCCATCGGCTTGGAACACGTAGAGAAATGCCATGATTGTGAAGCTAGAGGA | 720 |
| 189 | N--H--E--G--H--R--L--G--T--R--R--E--M--P--*- | 202 |

SEQ ID NO 117
LENGTH: 18
TYPE: DNA
ORGANISM: Artificial Sequence
OTHER INFORMATION: Description of Artificial Sequence: 5′ tailed primer extension sequence (FAM)
SEQUENCE: 1
TGTAAAACGACGGCCAGT
SEQ ID NO 118
LENGTH: 18
TYPE: DNA
ORGANISM: Artificial Sequence
OTHER INFORMATION: Description of Artificial Sequence: 5′ tailed primer extension sequence (NED)
SEQUENCE: 3
TAGGAGTGCAGCAAGCAT

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

1. (canceled)

2. A method of generating a sterile sex-determined fish, crustacean, or mollusk, comprising the step of:
(a) breeding (i) a fertile homozygous mutated female fish, crustacean, or mollusk having at least a first mutation and a second mutation with (ii) a fertile homozygous mutated male fish, crustacean, or mollusk having at least the first mutation and the second mutation to produce the sterile sex-determined fish, crustacean, or mollusk,
wherein the first mutation disrupts one or more genes that specify sexual differentiation,
wherein the second mutation disrupts one or more genes that specify gamete function, and
wherein the fertility of the fertile homozygous female fish, crustacean, or mollusk and the fertile homozygous mutated male fish, crustacean, or mollusk has been rescued, or
(b) breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation; and
selecting a progenitor that is homozygous by genotypic selection, the homozygous mutated progenitor being the sterile sex-determined fish, crustacean, or mollusk,
wherein the first mutation disrupts one or more genes that specify sexual differentiation, and
wherein the second mutation disrupts one or more genes that specify gamete function.

3. The method of claim 2, wherein the fertility rescue comprises germline stem cell transplantation, and optionally sex steroid alteration, for example, an alteration of estrogen, or an alteration of an aromatase inhibitor.

4-5. (canceled)

6. The method of claim 3, wherein the germine stem cell transplantation comprises the steps of:
obtaining a germline stem cell from a sterile homozygous male fish, crustacean, or mollusk having at least the first mutation and the second mutation or a germline stem cell from a sterile homozygous female fish, crustacean, or mollusk having at least the first mutation and the second mutation; and
transplanting the germline stem cell into a germ cell-less recipient male fish, crustacean, or mollusk, or into a germ cell-less recipient female fish, crustacean, or mollusk, or
obtaining a spermatogonial stem cell from a sterile homozygous male fish, crustacean, or mollusk having at least the first mutation and the second mutation or a oogonial stem cell from a sterile homozygous female fish, crustacean, or mollusk having at least the first mutation and the second mutation; and
transplanting the spermatogonial stem cell into a testis of a germ cell-less fertile male fish, crustacean, or mollusk or the oogonial stem cell into an ovary of a germ cell-less fertile female fish, crustacean, or mollusk.

7. The method of claim 6, wherein the germ cell-less recipient male fish, crustacean, or mollusk and the germ cell-less recipient female fish, crustacean, or mollusk are homozygous for a null mutation of the dnd, Elavl2, vasa, nanos3, or piwi-like gene.

8. The method of claim 6, wherein the germ cell-less recipient male fish, crustacean, or mollusk and the germ cell-less recipient female fish crustacean, or mollusk are created using ploidy manipulation, hybridization, or exposure to high levels of sex hormones.

9-11. (canceled)

12. The method of claim 6, wherein the germ cell-less fertile male fish, crustacean, or mollusk and the germ cell-less fertile female fish, crustacean, or mollusk are homozygous for the mutation of the dnd, Elavl2, vasa, nanos3, or piwi-like gene.

13. The method of claim 6, wherein the germ cell-less male fish, crustacean, or mollusk and the germ cell-less recipient female fish crustacean, or mollusk are created using ploidy manipulation, hybridization, or exposure to high levels of sex hormones.

14.-15. (canceled)

16. The method of claim 2, wherein the sterile sex-determined sterile fish, crustacean, or mollusk is a sterile male fish, crustacean, or mollusk.

17. The method of claim 2, wherein the first mutation comprises a mutation in one or more genes that modulates the synthesis of androgen and/or estrogen.

18. The method of claim 17, wherein the first mutation comprises a mutation in one or more genes that modulate the expression of: (a) aromatase Cyp19a1a, for example one or more genes selected from the group consisting of cyp19a1a, FoxL2, and an ortholog thereof; (b) Cyp17, for example, cyp17l or an ortholog thereof; or (c) a combination thereof.

19-20. (canceled)

21. The method of claim 2, wherein the second mutation comprises a mutation in one or more genes that modulate spermiogenesis.

22. The method of claim 21, wherein the second mutation comprises a mutation in one or more genes that cause globozoospermia, for example one or more genes that cause sperm with round-headed, round nucleus, disorganized midpiece, partially coiled tails, or a combination thereof, such as Gopc, Hiat1, Tjp1a, Smap2, Csnk2a2, and an ortholog thereof.

23-24. (canceled)

25. The method of claim 2, wherein the sterile sex-determined sterile fish, crustacean, or mollusk is a sterile female fish, crustacean, or mollusk.

26. The method of claim 25, wherein the first mutation comprises a mutation in one or more genes that modulate the expression of an aromatase Cyp19a1a inhibitor, for example, one or more genes selected from the group consisting of Gsdf, dmrt1, Amh, Amhr, and an ortholog thereof.

27. (canceled)

28. The method of claim 25, wherein the second mutation comprises a mutation in one or more genes that modulate:
(a) oogenesis, for example, one or more genes that modulate the synthesis of estrogen, such as FSHR or an ortholog thereof;
(b) folliculogenesis, for example, (i) one or more genes that modulate the expression of vitellogenins, such as vtgs or an ortholog thereof; or (ii) a mutation in a gene encoding or regulating: Vitellogenin; Estrogen receptor1; Cytochromo p450, family 1, subfamily a; zona pellucida glycoprotein; Choriogenin H; Peroxisome proliferator-activated receptor; Steroidogenic acute regulatory protein, or an ortholog thereof; or
(c) a combination thereof.

29-33. (canceled)

34. A method of generating a sterile sex-determined fish, crustacean, or mollusk, comprising the step of:
breeding (i) a fertile female fish, crustacean, or mollusk having a one or more homozygous mutations with (ii) a fertile male fish, crustacean, or mollusk having one or more homozygous mutations to produce the sterile sex-determined fish, crustacean, or mollusk,
wherein the one or more mutations directly or indirectly disrupts spermiogenesis, and/or directly disrupts vitellogenesis, and
wherein the fertility of the fertile female fish, crustacean, or mollusk and the fertile male fish, crustacean, or mollusk have been rescued.

35. The method of claim 34, wherein the one or more mutations that directly or indirectly disrupts spermiogenesis is a mutation in Gopc, Hiat1, Tjp1a, Smap2, Csnk2a2, or an ortholog thereof.

36. The method of claim 34, wherein the one or more mutations that directly disrupts vitellogenesis is a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof.

37. (canceled)

38. The method of claim 34, wherein the fertility rescue comprises germline stem cell transplantation, and optionally sex steriod alteration, for example, an alteration of estrogen, or an alteration of an aromatase inhibitor.

39-40. (canceled)

41. The method of claim 38, wherein the germline stem cell transplantation comprises the steps of:
obtaining a germline stem cell from a sterile homozygous male fish, crustacean, or mollusk having at least the homozygous mutation or a germline stem cell from a sterile homozygous female fish, crustacean, or mollusk having at least the homozygous mutation; and
transplanting the germline stem cell into a germ cell-less recipient male fish, crustacean, or mollusk, or into a germ cell-less recipient female fish, crustacean, or mollusk.

42. The method of claim 41, wherein the germ cell-less recipient male fish, crustacean, or mollusk and the germ cell-less recipient female fish, crustacean, or mollusk are homozygous for a null mutation of the dnd, Elavl2, vasa, nanos3, or piwi-like gene.

43. The method of claim 41, wherein the germ cell-less recipient male fish, crustacean, or mollusk and the germ cell-less recipient female fish crustacean, or mollusk are created using ploidy manipulation, hybridization, or exposure to high levels of sex hormones.

44-45. (canceled)

46. The method of claim 34, wherein the fertile female fish, crustacean, or mollusk and the fertile male fish, crustacean, or mollusk have an additional homozygous mutation that specifies sexual differentiation.

47. The method of claim 46, wherein the mutation that specifies sexual differentiation modulates the expression of: (a) aromatase Cyp19a1a; (b) Cyp17, for example, a mutation in cyp17l or an ortholog thereof; (c) an inhibitor to aromatase Cyp19a1a, for example, a mutation in Gsdf, dmrt1, Amh, Amhr, or an ortholog thereof; or (d) a combination thereof.

48-49. (canceled)

50. The method of claim 34, wherein the breeding step comprises hybridization or hormonal manipulation and breeding strategies, to specify sexual differentiation.

51. (canceled)

52. A fertile homozygous mutated fish, crustacean, or mollusk for producing a sterile sex-determined fish, crustacean, or mollusk, the fertile homozygous mutated fish, crustacean, or mollusk having at least a first mutation and a second mutation, wherein the first mutation disrupts one or more genes that specify sexual differentiation, wherein the second mutation disrupts one or more genes that specify gamete function, and wherein the fertility of the fertile homozygous mutated fish, crustacean, or mollusk has been rescued.

53. The fertile homozygous mutated fish, crustacean, or mollusk of claim 52, wherein the fertility rescue comprises germline stem cell transplantation, and optionally sex steroid alteration, for example, an alteration of estrogen, or an alteration of an aromatase inhibitor.

54-55. (canceled)

56. The fertile homozygous mutated fish, crustacean, or mollusk of claim 53, wherein the germline stem cell transplantation comprises the steps of:
obtaining a germline stem cell from a sterile homozygous male fish, crustacean, or mollusk having at least the first mutation and the second mutation or a germline stem cell from a sterile homozygous female fish, crustacean, or mollusk having at least the first mutation and the second mutation; and
transplanting the germline stem cell into a germ cell-less recipient male fish, crustacean, or mollusk, or into a germ cell-less recipient female fish, crustacean, or mollusks, or
obtaining a spermatogonial stem cell from a sterile homozygous male fish, crustacean, or mollusk having at least the first mutation and the second mutation or a oogonial stem cell from a sterile homozygous female fish, crustacean, or mollusk having at least the first mutation and the second mutation; and
transplanting the spermatogonial stem cell into a testis of a germ cell-less fertile male fish, crustacean, or mollusk or the oogonial stem cell into an ovary of a germ cell-less fertile female fish, crustacean, or mollusk.

57. The fertile homozygous mutated fish, crustacean, or mollusk of claim 56, wherein the germ cell-less recipient male fish, crustacean, or mollusk and the germ cell-less recipient female fish, crustacean, or mollusk are homozygous for a null mutation of the dnd, Elavl2, vasa, nanos3, or piwi-like gene.

58. The fertile homozygous mutated fish, crustacean, or mollusk of claim 56, wherein the germ cell-less recipient male fish, crustacean, or mollusk and the germ cell-less recipient female fish crustacean, or mollusk are created using ploidy manipulation, hybridization, or exposure to high levels of sex hormones.

59-61. (canceled)

62. The fertile homozygous mutated fish, crustacean, or mollusk of claim 56, wherein the germ cell-less fertile male fish, crustacean, or mollusk and the germ cell-less fertile female fish, crustacean, or mollusk are homozygous for the mutation of the dnd, Elavl2, vasa, nanos3, or piwi-like gene.

63. The fertile homozygous mutated fish, crustacean, or mollusk of claim 56, wherein the germ cell-less recipient male fish, crustacean, or mollusk and the germ cell-less recipient female fish crustacean, or mollusk are created using ploidy manipulation, hybridization, or exposure to high levels of sex hormones.

64-65. (canceled)

66. The fertile homozygous mutated fish, crustacean, or mollusk of claim 52, wherein the sterile sex-determined sterile fish, crustacean, or mollusk is a sterile male fish, crustacean, or mollusk.

67. The fertile homozygous mutated fish, crustacean, or mollusk of claim 52, wherein the first mutation comprises a mutation in one or more genes that modulates the synthesis of androgen and/or estrogen.

68. The fertile homozygous mutated fish, crustacean, or mollusk of claim 67, wherein the first mutation comprises a mutation in one or more genes that modulate the expression of: (a) aromatase Cyp19a1a, for example, one or more genes selected from the group consisting of cyp19a1a, FoxL2, and an ortholog thereof; (b) Cyp17, for example, cyp17l or an ortholog thereof; or (c) a combination thereof.

69-70. (canceled)

71. The fertile homozygous mutated fish, crustacean, or mollusk of claim 52, wherein the second mutation comprises a mutation in one or more genes that modulate spermiogenesis.

72. The fertile homozygous mutated fish, crustacean, or mollusk of claim 71, wherein the second mutation comprises a mutation in one or more genes that cause globozoospermia, for example, one or more genes that cause sperm with round-headed, round nucleus, disorganized midpiece, partially coiled tails, or a combination thereof, such as Gopc, Hiat1, Tjp1a, Smap2, Csnk2a2, and an ortholog thereof.

73-74. (canceled)

75. The fertile homozygous mutated fish, crustacean, or mollusk of claim 52, wherein the sterile sex-determined sterile fish, crustacean, or mollusk is a sterile female fish, crustacean, or mollusk.

76. The fertile homozygous mutated fish, crustacean, or mollusk of claim 75, wherein the first mutation comprises a mutation in one or more genes that modulate the expression of an aromatase Cyp19a1a inhibitor, for example, one or more genes selected from the group consisting of Gsdf, dmrt1, Amh, Amhr, and an ortholog thereof.

77. (canceled)

78. The fertile homozygous mutated fish, crustacean, or mollusk of claim 75, wherein the second mutation comprises a mutation in one or more genes that modulate:
(a) oogenesis, for example, one or more genes that modulate the synthesis of estrogen, such as FSHR or an ortholog thereof;
(b) folliculogenesis, for example, (i) one or more genes that modulate the expression of vitellogenins, such as vtgs or an ortholog thereof; or (ii) a mutation in a gene encoding or regulating: Vitellogenin; Estrogen receptor1; Cytochromo p450, family 1, subfamily a; zona pellucida glycoprotein; Choriogenin H; Peroxisome proliferator-activated receptor; Steroidogenic acute regulatory protein, or an ortholog thereof; or
(c) a combination.

79-83. (canceled)

84. A fertile fish, crustacean, or mollusk having a homozygous mutation for producing a sterile sex-determined fish, crustacean, or mollusk, wherein the mutation directly or indirectly disrupts spermiogenesis, and/or directly disrupts vitellogenesis, and wherein the fertility of the fertile fish, crustacean, or mollusk has been rescued.

85. The fertile fish, crustacean, or mollusk of claim 84, wherein the mutation that directly or indirectly disrupts spermiogenesis is a mutation in Gopc, Hiat1, Tjp1a, Smap2, Csnk2a2, or an ortholog thereof.

86. The fertile fish, crustacean, or mollusk of claim 84, wherein the mutation that directly disrupts vitellogenesis is a mutation in a gene encoding or regulating: Vitellogenin; Estrogen receptor1; Cytochrome p450, family 1, subfamily a; zona pellucida glycoprotein; Choriogenin H; Peroxisome proliferator-activated receptor; Steroidogenic acute regulatory protein, or an ortholog thereof.

87. The fertile fish, crustacean, or mollusk of claim 84, wherein the fertile fish, crustacean, or mollusk has a plurality of homozygous mutations that, in combination: directly or indirectly disrupt spermiogenesis; directly disrupt vitellogenesis; or both.

88. The fertile fish, crustacean, or mollusk of claim 84, wherein the fertility rescue comprises germline stem cell transplantation, and optionally sex steroid alteration, for example, an alteration of estrogen, or an alteration of an aromatase inhibitor.

89-90. (canceled)

91. The fertile fish, crustacean, or mollusk of claim 88, wherein the germline stem cell transplantation comprises the steps of:
obtaining a germline stem cell from a sterile homozygous male fish, crustacean, or mollusk having at least the homozygous mutation or a germline stem cell from a sterile homozygous female fish, crustacean, or mollusk having at least the homozygous mutation; and
transplanting the germline stem cell into a germ cell-less recipient male fish, crustacean, or mollusk, or into a germ cell-less recipient female fish, crustacean, or mollusk.

92. The fertile fish, crustacean, or mollusk of claim 91, wherein the germ cell-less recipient male fish, crustacean, or mollusk and the germ cell-less recipient female fish, crustacean, or mollusk are homozygous for a null mutation of the dnd, Elavl2, vasa, nanos3, or piwi-like gene.

93. The fertile fish, crustacean, or mollusk of claim 91, wherein the germ cell-less recipient male fish, crustacean, or mollusk and the germ cell-less recipient female fish crustacean, or mollusk are created using ploidy manipulation, hybridization, or exposure to high levels of sex hormones.

94-95. (canceled)

96. The fertile fish, crustacean, or mollusk of claim 84, wherein the fertile fish, crustacean, or mollusk has an additional homozygous mutation that specifies sexual differentiation.

97. The fertile fish, crustacean, or mollusk of claim 96, wherein the mutation that specifies sexual differentiation modulates the expression of: (a) aromatase Cyp19a1a, for example, one or more genes selected from the group consisting of cyp19a1a, FoxL2, and an ortholog thereof; (b) Cyp17, for example, Gsdf, dmrt1, Amh, Amhr, or an ortholog thereof; (c) an inhibitor to aromatase Cyp19a1a; or (d) a combination thereof.

98-99. (canceled)

100. The fertile fish, crustacean, or mollusk of claim 84, wherein producing a sterile sex-determined fish, crustacean, or mollusk comprises a breeding step comprising hybridization or hormonal manipulation and breeding strategies, to specify sexual differentiation.

101. (canceled)

102. A method of making a fertile homozygous mutated fish, crustacean, or mollusk that generates a sterile sex-determined fish, crustacean, or mollusk, comprising the steps of:
breeding (i) a fertile hemizygous mutated female fish, crustacean, or mollusk having at least a first mutation and a second mutation with (ii) a fertile hemizygous mutated male fish, crustacean, or mollusk having at least the first mutation and the second mutation;
selecting a progenitor that is homozygous by genotypic selection; and
rescuing the fertility of the homozygous progenitor,
wherein the first mutation disrupts one or more genes that specify sexual differentiation, and
wherein the second mutation disrupts one or more genes that specify gamete function.


SEQUENCE LISTING (118 sequences)

SEQ ID NO: 1 - DNA, 38 bp, Artificial Sequence
  Description of Artificial Sequence: Synthetic Forward tailed Primer (FAM)
tgtaaaacgacggccagtttgaagttgctacataaaag

SEQ ID NO: 2 - DNA, 22 bp, Artificial Sequence
  Description of Artificial Sequence: Synthetic primer
tggttgatgacaatcacactgt

SEQ ID NO: 3 - DNA, 41 bp, Artificial Sequence
  Description of Artificial Sequence: Synthetic Forward tailed Primer (FAM)
taggagtgcagcaagcattgttctacatcatcacccttctc

SEQ ID NO: 4 - DNA, 23 bp, Artificial Sequence
  Description of Artificial Sequence: Synthetic primer
agcagacagacgagcagtatcag

SEQ ID NO: 5 - DNA, 41 bp, Artificial Sequence
  Description of Artificial Sequence: Synthetic Forward tailed Primer (FAM)
tgtaaaacgacggccagttgatggagagcttcatctacgaa

SEQ ID NO: 6 - DNA, 20 bp, Artificial Sequence
  Description of Artificial Sequence: Synthetic primer
gttccaggttaaattgattg

SEQ ID NO: 7 - DNA, 41 bp, Artificial Sequence
  Description of Artificial Sequence: Synthetic Forward tailed Primer (NED)
taggagtgcagcaagcatgcgtgatttgctgacctttttac

SEQ ID NO: 8 - DNA, 21 bp, Artificial Sequence
  Description of Artificial Sequence: Synthetic primer
acacttaccctgagaatctgg

SEQ ID NO: 9 - DNA, 41 bp, Artificial Sequence
  Description of Artificial Sequence: Synthetic Forward tailed Primer (FAM)
tgtaaaacgacggccagtgaaaaaggatggtgagggatgac

SEQ ID NO: 10 - DNA, 23 bp, Artificial Sequence
  Description of Artificial Sequence: Synthetic primer
gagtgtgtctaccacacggaaaa

SEQ ID NO: 11 - DNA, 41 bp, Artificial Sequence
  Description of Artificial Sequence: Synthetic Forward tailed Primer (FAM)
tgtaaaacgacggccagtgtatttagaaggcggtgaaggtc

SEQ ID NO: 12 - DNA, 23 bp, Artificial Sequence
  Description of Artificial Sequence: Synthetic primer
cagtttggcacatgagcatcgta

SEQ ID NO: 13 - DNA, 37 bp, Artificial Sequence
  Description of Artificial Sequence: Synthetic Forward tailed Primer (FAM)
taggagtgcagcaagcatatgctcatgtgccaaactg

SEQ ID NO: 14 - DNA, 23 bp, Artificial Sequence
  Description of Artificial Sequence: Synthetic primer
ccttcaggattttcaccaccact

SEQ ID NO: 15 - DNA, 41 bp, Artificial Sequence
  Description of Artificial Sequence: Synthetic Forward tailed Primer (FAM)
tgtaaaacgacggccagttactgacacatccagcagcgtct

SEQ ID NO: 16 - DNA, 23 bp, Artificial Sequence
  Description of Artificial Sequence: Synthetic primer
cagcactgagccgtcagtattct

SEQ ID NO: 17 - DNA, 37 bp, Artificial Sequence
  Description of Artificial Sequence: Synthetic Forward tailed Primer (NED)
taggagtgcagcaagcattggagcctacctgtctgag

SEQ ID NO: 18 - DNA, 20 bp, Artificial Sequence
  Description of Artificial Sequence: Synthetic primer
tactcacagcgaaggggtct

SEQ ID NO: 19 - DNA, 38 bp, Artificial Sequence
  Description of Artificial Sequence: Synthetic Forward tailed Primer (NED)
taggagtgcagcaagcatgctcctctgcgaagactctc

SEQ ID NO: 20 - DNA, 23 bp, Artificial Sequence
  Description of Artificial Sequence: Synthetic primer
aagacctccgacctggacttgct

SEQ ID NO: 21 - DNA, 41 bp, Artificial Sequence
  Description of Artificial Sequence: Synthetic Forward tailed Primer (FAM)
tgtaaaacgacggccagtagaggagggcacagtcaagaaac

SEQ ID NO: 22 - DNA, 22 bp, Artificial Sequence
  Description of Artificial Sequence: Synthetic primer
ttggatatcccatttggttcat

SEQ ID NO: 23 - DNA, 40 bp, Artificial Sequence
  Description of Artificial Sequence: Synthetic Forward tailed Primer (NED)
taggagtgcagcaagcattttaacggtgttggcagagatt

SEQ ID NO: 24 - DNA, 22 bp, Artificial Sequence
  Description of Artificial Sequence: Synthetic primer
agatccacatccacgaaagcct

SEQ ID NO: 25 - DNA, 37 bp, Artificial Sequence
  Description of Artificial Sequence: Synthetic Forward tailed Primer (FAM)
tgtaaaacgacggccagttgcccctttaaaccaccta

SEQ ID NO: 26 - DNA, 23 bp, Artificial Sequence
  Description of Artificial Sequence: Synthetic primer
ctcagcttggccttgcttgacat

SEQ ID NO: 27 - DNA, 39 bp, Artificial Sequence
  Description of Artificial Sequence: Synthetic Forward tailed Primer (NED)
taggagtgcagcaagcatttgccaggacccatgagccag

SEQ ID NO: 28 - DNA, 23 bp, Artificial Sequence
  Description of Artificial Sequence: Synthetic primer
agacacgtatccgtgatttctac

SEQ ID NO: 29 - DNA, 41 bp, Artificial Sequence
  Description of Artificial Sequence: Synthetic Forward tailed Primer (FAM)
tgtaaaacgacggccagtctcttcatcctctgtgtctcatc

SEQ ID NO: 30 - DNA, 22 bp, Artificial Sequence
  Description of Artificial Sequence: Synthetic primer
gggtttccagcaggaggtcaga

SEQ ID NO: 31 - DNA, 39 bp, Artificial Sequence
  Description of Artificial Sequence: Synthetic Forward tailed Primer (NED)
taggagtgcagcaagcatttatgttcaggtgccaaggtg

SEQ ID NO: 32 - DNA, 23 bp, Artificial Sequence
  Description of Artificial Sequence: Synthetic primer
tggctgtgtgagaaacgatgctg

SEQ ID NO: 33 - DNA, 35 bp, Artificial Sequence
  Description of Artificial Sequence: Synthetic Forward tailed Primer (FAM)
tgtaaaacgacggccagtagatctgggctgggaca

SEQ ID NO: 34 - DNA, 23 bp, Artificial Sequence
  Description of Artificial Sequence: Synthetic primer
tgttaactatacctgtgtgttgg

SEQ ID NO: 35 - DNA, 38 bp, Artificial Sequence
  Description of Artificial Sequence: Synthetic Forward tailed Primer (NED)
taggagtgcagcaagcatttttctccgcttgcttctgc

SEQ ID NO: 36 - DNA, 23 bp, Artificial Sequence
  Description of Artificial Sequence: Synthetic primer
aaagagctgaataggaggaagtt

SEQ ID NO: 37 - DNA, 39 bp, Artificial Sequence
  Description of Artificial Sequence: Synthetic Forward tailed Primer (FAM)
tgtaaaacgacggccagtcatcttggcgttcttctgtgt

SEQ ID NO: 38 - DNA, 21 bp, Artificial Sequence
  Description of Artificial Sequence: Synthetic primer
cttgagggcagctgagatggc

SEQ ID NO: 39 - DNA, 40 bp, Artificial Sequence
  Description of Artificial Sequence: Synthetic Forward tailed Primer (NED)
taggagtgcagcaagcatgcaatccttgatgctccttgac

SEQ ID NO: 40 - DNA, 22 bp, Artificial Sequence
  Description of Artificial Sequence: Synthetic primer
ctgagactctatgtcgttgata

SEQ ID NO: 41 - DNA, 41 bp, Artificial Sequence
  Description of Artificial Sequence: Synthetic Forward tailed Primer (FAM)
tgtaaaacgacggccagtagaagatcatcaaacacatcacg

SEQ ID NO: 42 - DNA, 23 bp, Artificial Sequence
  Description of Artificial Sequence: Synthetic primer
gacttgttgagcagttgcatcaa

SEQ ID NO: 43 - DNA, 38 bp, Artificial Sequence
  Description of Artificial Sequence: Synthetic Forward tailed Primer (NED)
taggagtgcagcaagcatttttgtgatctagtctggag

SEQ ID NO: 44 - DNA, 22 bp, Artificial Sequence
  Description of Artificial Sequence: Synthetic primer
gctcttacagcttcacaatcat

SEQ ID NO: 45 - DNA, 41 bp, Artificial Sequence; identical to SEQ ID NO: 41
  Description of Artificial Sequence: Synthetic Forward tailed Primer (FAM)

SEQ ID NO: 46 - DNA, 23 bp, Artificial Sequence
  Description of Artificial Sequence: Synthetic primer
gacttgttgagcagttgcatcaa

SEQ ID NO: 47 - DNA, 22 bp, Artificial Sequence
  Description of Artificial Sequence: Synthetic primer
gaaccaaacccctctgtcactg

SEQ ID NO: 48 - DNA, 22 bp, Artificial Sequence
  Description of Artificial Sequence: Synthetic primer
gtaattcactccgcaggctcag

SEQ ID NO: 49 - DNA, 17 bp, Artificial Sequence
  Description of Artificial Sequence: Synthetic primer
ggcgatgaatcctgtag

SEQ ID NO: 50 - DNA, 22 bp, Artificial Sequence
  Description of Artificial Sequence: Synthetic primer
atggcatttgaggtcacagaga

SEQ ID NO: 51 - DNA, 19 bp, Artificial Sequence
  Description of Artificial Sequence: Synthetic primer
gttcaagaagggagagagt

SEQ ID NO: 52 - DNA, 18 bp, Artificial Sequence
  Description of Artificial Sequence: Synthetic primer
aaaaattcccacatcgtt

SEQ ID NO: 53 - DNA, 20 bp, Artificial Sequence
  Description of Artificial Sequence: Synthetic primer
tgctttggcttcagtgtatc

SEQ ID NO: 54 - DNA, 19 bp, Artificial Sequence
  Description of Artificial Sequence: Synthetic primer
aatgcgttcgaatgtagaa

SEQ ID NO: 55 - DNA, 23 bp, Artificial Sequence
  Description of Artificial Sequence: Synthetic primer
catctgcttcatcctggtggctg

SEQ ID NO: 56 - DNA, 23 bp, Artificial Sequence
  Description of Artificial Sequence: Synthetic primer
aatttgggcatcttcatctgtat

SEQ ID NO: 57 - DNA, 22 bp, Artificial Sequence
  Description of Artificial Sequence: Synthetic primer
gacagacttgaccttggagatg

SEQ ID NO: 58 - DNA, 21 bp, Artificial Sequence
  Description of Artificial Sequence: Synthetic primer
atgtctgcttcgactggatgc

SEQ ID NO: 59 - DNA, 23 bp, Artificial Sequence
  Description of Artificial Sequence: Synthetic primer
gccatcgaaacatggacatactg

SEQ ID NO: 60 - DNA, 1563 bp, Oreochromis niloticus
  CDS (1)..(1563)
gaaccaaacccctctgtcactgatatggcttggtttttgtgtctgtgcgtgttcatggcggtgggcctcactttgttagcactgcagttcaagttcaggatgtctgcacatggttctggggagccgccacacctccctgcactaccactgattggcagcctgctgagcctgcggagtgaattaccaccgcatgtgcttttcaaagaactgcaggtaaaatacggacatacatactcgctgatgatgggctcccacagtgtgattgtcatcaaccagcatgtgcacgccaaagaagtcttgctcaagaagggaaagacgtttgcaggaagacctagaactgtaaccacagatattctgactagagatgggaaggacattgcatttggagactacagtgctacgtggaagttccacaggaagatagtccatggagccctgtgcatgtttggagaaggttctgcctctattgagaagaccatttgtgcagaggcccagtctctgtgctccgtgctgtctgaggcagcagatgtgggactggccctggatcttgctcctgagctgactcgcgctgtcaccaacgttatctgttctctctgcttcaactcgtcctactgccgaggcgactcagagtttgagacaatgctgcagtacagccagggcattgtggacactgtggctaaagacagcctggtagacattttcccctggcttcagatctttcctaatgcggacctacgtctcctaaaacattgtgtttccatcagagacaaacttctacagaggaaatttgatgaacacaaggtgaattacaatgatcacgtgcagagagacttgatagacgctctgctaagagccaagcgcagtgcggagaacaacaacacatcagagataagtgcagagtctgtgggcctgagtgatgaccacattctcatgacagtgggagacatctttggcgctggcgtggaaaccactaccactgtgctcaaatgggccataacgtacctcattcatcacccagaggtgcaaagacgtatccaggatgagctggacaggacggtgggtgacagccgctctcctaaactcaccgacagaggcagtctgccttatctggaggccaccattagggaagtattgcggattcgccccgtggcaccactactcatcccccatgtggctctctgtgacaccagcattggagatttcacagtgagaaaaggaactcgagtcattatcaacctgtgggctctgcaccatgatgagaaggagtggaagaacccagagcggtttgaccctggccggttcttgaaaagtgaaggcacaggactgacaatcccatcacccagctacctgccctttggtgctgggctgagagtatgtttaggtgaggccttggccaagatggagctctttctcttcctgtcctggatcctgcagcgcttcactctgtctgtcccaccaggccacagtctgcccagtctggagggaaagtttggagtggtcctgcagacagccaagtacaaggtgaatgccacaatcagaccagactgggcaagacataagtgc

SEQ ID NO: 61 - DNA, 180 bp, Oreochromis niloticus
  CDS (1)..(132)
gaaccaaacccctctgtcactgatatggcttggtttttgtgtctgtgcggtgggcctcactttgttagcactgcagttcaagttcaggatgtctgcacatggttctggggagccacctccctgcactaccactgattggcagcctgctgagcctgcggagtgaattaccaccgcatgtgc

SEQ ID NO: 62 - protein, 521 aa, Oreochromis niloticus
EPNPSVTDMAWFLCLCVFMAVGLTLLALQFKFRMSAHGSGEPPHLPALPLIGSLLSLRSELPPHVLFKELQVKYGHTYSLMMGSHSVIVINQHVHAKEVLLKKGKTFAGRPRTVTTDILTRDGKDIAFGDYSATWKFHRKIVHGALCMFGEGSASIEKTICAEAQSLCSVLSEAADVGLALDLAPELTRAVTNVICSLCFNSSYCRGDSEFETMLQYSQGIVDTVAKDSLVDIFPWLQIFPNADLRLLKHCVSIRDKLLQRKFDEHKVNYNDHVQRDLIDALLRAKRSAENNNTSEISAESVGLSDDHILMTVGDIFGAGVETTTTVLKWAITYLIHHPEVQRRIQDELDRTVGDSRSPKLTDRGSLPYLEATIREVLRIRPVAPLLIPHVALCDTSIGDFTVRKGTRVIINLWALHHDEKEWKNPERFDPGRFLKSEGTGLTIPSPSYLPFGAGLRVCLGEALAKMELFLFLSWILQRFTLSVPPGHSLPSLEGKFGVVLQTAKYKVNATIRPDWARHKC

SEQ ID NO: 63 - protein, 44 aa, Oreochromis niloticus
EPNPSVTDMAWFLCLCGGPHFVSTAVQVQDVCTWFWGATSLHYH

SEQ ID NO: 64 - skipped

SEQ ID NO: 65 - DNA, 1707 bp, Oreochromis niloticus
  CDS (4)..(1536)
gcgatgaatcctgtaggcttagacgccgtggtggcagatctctctgtgacctcaaatgccatccaatcgcatgggatatcaatggcaaccagaacgctgatactgctcgtctgtctgctgttggttgcctggagtcacacggacaagaaaattgtgccaggtccttctttctgtttgggtttgggcccacttctgtcatatctgagatttatctggactggcataggcacagccagcaactactacaataacaagtatggagacattgttagagtctggatcaacggagaagagacgctcatactaagcagatcttcagcagtgcaccatgtgctgaagaacggaaactatacttcacgttttgggagcatccagggactcagctgcctcggcatgaacgagagaggcatcatattcaacaacaacgtaactctgtggaaaaagatacgcacctattttgctaaagctctgacaggcccaaatttgcagcagacggcggatgtttgcgtctcctccatacaggctcacctggaccacctggacagcctgggacacgttgatgtcctcaatttgctgcgctgcaccgtgctggacatctctaaccgactcttcctggacgtacctctcaatgagaaagagctgatgctgaagattcaaaagtattttcacacatggcaggatgtgcttatcaaacctgacatctacttcaagttcggctggattcaccacaggcacaagacagcaacccaggagttacaagatgccattaaacgtcttgtagatcaaaagaggaaaaatatggagcaggcagatacgctggacaacatcaacttcacggcagagctcatatttgcacaaaaccacggtgagctgtctgctgagaatgtgacgcagtgcgtgctggagatggtgattgcagctccggacactctgtccctcagtctcttcttcatgcttctgctcctcaaacaaaacccgcacgtggagccgcagctgctacaggagatagacgctgttgtgggtgagagacagcttcagaaccaggatcttcacaagctgcaggtgatggagagcttcatctacgaatgcttgcgcttccacccagtggtggacttcaccatgcgtcgagccctgtctgatgacatcatagaaggctacaggatctcgaagggcacaaacatcattctgaacacaggccgaatgcaccgcaccgagtttttcctcaaagccaatcaatttaacctggaacactttgaaaacaatgttcctcggcgctactttcagccgttcggttcaggccctcgcgcatgcattggcaagcacatcgccatggtgatgatgaaatccattttggtgacactgctgtctcagtactctgtttgtactcacgagggcccgatcctggactgcctcccacaaaccaacaacctttcccagcagcctgtagagcaccagcaggcggagactgaacatctccacatgaggttcttacccaggcagagaagcagctgtcaaaccctccgagacccgaacctttagctgtacctgcacttttgtatacttaatttgtataatcttataacgacacacagctagcctttatattttgatagacgcaaagattgtatttgtactcaaactgtatgcatgatgtgaaatgtacatttaaacctgctaacactgaaataaatgtaagttattgtgtca

SEQ ID NO: 66 - DNA, 60 bp, Oreochromis niloticus
  CDS (4)..(39)
gcgatgaatcctgtaggcttagactggcagatctctctgtgacctcaaatgccatccaat

SEQ ID NO: 67 - DNA, 60 bp, Oreochromis niloticus
  CDS (4)..(36)
gcgatgaatcctgtaggctggtggcagatctctctgtgacctcaaatgccatccaatcgc

SEQ ID NO: 68 - protein, 511 aa, Oreochromis niloticus
MNPVGLDAVVADLSVTSNAIQSHGISMATRTLILLVCLLLVAWSHTDKKIVPGPSFCLGLGPLLSYLRFIWTGIGTASNYYNNKYGDIVRVWINGEETLILSRSSAVHHVLKNGNYTSRFGSIQGLSCLGMNERGIIFNNNVTLWKKIRTYFAKALTGPNLQQTADVCVSSIQAHLDHLDSLGHVDVLNLLRCTVLDISNRLFLDVPLNEKELMLKIQKYFHTWQDVLIKPDIYFKFGWIHHRHKTATQELQDAIKRLVDQKRKNMEQADTLDNINFTAELIFAQNHGELSAENVTQCVLEMVIAAPDTLSLSLFFMLLLLKQNPHVEPQLLQEIDAVVGERQLQNQDLHKLQVMESFIYECLRFHPVVDFTMRRALSDDIIEGYRISKGTNIILNTGRMHRTEFFLKANQFNLEHFENNVPRRYFQPFGSGPRACIGKHIAMVMMKSILVTLLSQYSVCTHEGPILDCLPQTNNLSQQPVEHQQAETEHLHMRFLPRQRSSCQTLRDPNL

SEQ ID NO: 69 - protein, 12 aa, Oreochromis niloticus
MNPVGLDWQISL

SEQ ID NO: 70 - protein, 11 aa, Oreochromis niloticus
MNPVGWWQISL

SEQ ID NO: 71 - DNA, 6674 bp, Oreochromis niloticus
  CDS (1)..(4956)
aaagaggaaaacaatgcatcatataactttataagtaagagtgcggcgatggaggaaaccgtcatatgggaacagcacacagttacccttcacagggccccaggatttgggtttggcattgccatctcgggtgggcgagacaaccctcatttccagagtggtgaaacatctattgtaatatcggatgtgctgaaaggaggtcctgcagagggtctgctacaagaaaatgatcgagtagtaatggtcaatgcagtctctatggacaatgtagagcatgcctatgctgttcaacaacttcgaaagagtggcaaaaatgcaaagataactattcgcagaaaaaggaaagtacaaatcccagcgtcacggcacggggacagggagacgatgtctgagcacgaggaggaggacagcgatgaggctgatgcttacgatcaccgcagtggacgtggtggacaaaacgatcgggagcgtagcagcagtgggaggcgggatcacagtgcctcacaggagaggagcatctcaccacgctccgatcgccgatcacaagcctcttctgctccacccaggccctccaaggtcactcttgtcaagtcccgcaaaaacgaagaatatggactgcggctggccagccatatctttgtgaaggacatctctccagagagccttgcagccagagatggaaacattcaggagggagatgttgtacttaagattaacggcacagttacagagaacctatcactgacagatgccaagaagctgattgagaggtcaaagggcaagctgaagatggtagtgcagagagacgagcgggccacgctgctcaatattcctgaccttgacgacagcatcccatcagccaataactcagacagagatgacatttcagagatacattcactgacatccgatcattccaatcgatcccatgggagaggaagtcaatcccattcgcctgacagggttgaaacatccgagcatctccgccactcaccgcggcagatcagcaatggcagtaatggatttctctgggaaagagctgaggaattaatcaaacaggaatgggtggtgaaacaggaatgttattttgcctgtgcccatactataaaatgtgtaataaccgtgacagtttgggcaaaaaaaccccaaaacagtaacatgccagaaccaaagccagtttatgcacagcctggtcagcctgacgtggacctgcctgtcagcccatctgatgcccctgtacccagtgctgcacatgatgacagcattctcagaccaagtatgaagctggtcaagttcaagaagggagagagtgtcggtctgaggttagcaggcggaaacgatgtgggaatttttgtggcaggagttttggaagacagccccgcagccaaggaggggctggaagagggagaccagattctcagggtgaacaacgtggactttgctaacatcatccgggaagaggctgtgctttttctgctcgatcttccaaaaggagatgacgttactattctggctcagaagaaaaaggatgtgtatcgaaggatagtggaatcagacgtgggtgactccttctacattcgaacgcattttgaatatgaaaaagagtcaccgtatgggctgagctttaacaagggagaggttttccgtgtggtagacacactctataatggcaaattaggctcctggctcgctatccgtatcggcaagaaccaccaggaagtggaaagaggcataatccccaacaagaatagagccgagcagctatccagtgtgcagtacacccttcctaaaacgcctgggggcgacagagctgacttctggaggttcagagggctgcggagttccaagaggaatttgcggaaaagcagggaggacctgtcggcccagcctgttcagaccaagttccctgcctatgagagggtggtgctgagggaagctgggttcctgaggcctgtggttatctttgggccgattgcagacgtggcccgagagaaactggccagggaggtgcccgaagtgtttgagctagccaagagtgaacccagggatgcaggaacagaccagaagagctctggcatcatccgcctgcacaccattaagcagatcattgatcgagacaagcatgcagtgctggatataaccccgaatgcagtggaccgactgaactacgctcagtggtatccaattgtggtgtttctcaacccggacaccaagcagggcatcaagaacatgaggacacggctctgccccgagtctaggaagagcgcgagaaagctttatgatcgagccctcaagttaagaaagaacaaccaccacctcttcaccacaaccattaacttgaacaacatgaacgatggttggtttggagcactgaaagaaatcatccatcagcagcagaaccagctggtgtgggtttcagagggcaaggctgatggagttggcgacgatgacctggacatccacgacgaccgcctttcctacctgtcggcgccaggcagtgagtattccatgtacagcaccgacagccgccacacctccgattacgaggacacggacacagagggaggagcctacaccgaccaggagctggatgaaacgctgaacgatgacgtgggtccacccacggagcctgccatcacgcggtcctctgagcctgtccgtgaggacccgcctgtcatccaagagccccctggctatgtcagctacccgcacacagtgcagccggaccccctgaaccgcatcgacccggctggtttcaaggcaccagcgccgcagcagatgtttcagaaggatccgtacagcacagacaacataggcagaggtggtcacggcatgaagcctgtgacgtacaaccctcagcaggggtatcaccccgacgagcagccatacagagattacgatcacccacccagccggtatgacatcagcagcagtggtgtcggcggtggctaccaggagccaaagtaccgtaactatgagagctatgagaacagcgtgcctcactacgaccagcaaccgtggaacccctacaaccagccgttctccactgccaacacccaggcctacgatccccgtcctccttacggtgagggccccgactctcattacacccctcccctgcgctacgacgagccgccacctcagcagggatttgacggacggcctcgctacggcaaaccgacagtttcagcacctgtccgttacgatgatcttccgcctccccctcagccgtctgaattgcactatgacccaaattctcacctgagcacatacccctcagctgcccgctcaccagaacccgctgcccagcgacccgcctataaccagggaccagcatcgcagcagaaaggttacaaacctcagcagtacgatcctgctcctgtgaactctgaatccagccccagccttcataaagtcgagacgccctcaccttcacctgctgatgttccaaaagctgcacctgcaagagatgagcagcaggaggaggatccagccatgcggcctcagtcagtactgacgagggtcaaaatgtttgagaacaaacgctctgtgtccatggaccgagccagagatgccggggattcatttgggaataaggcagccgatttgcccttgaaagctggtggagtaatccctaaagcaaattctctgagcaacctggatcaagagaagacctttagcagagggccagagcctcagaagcctcagtccaagggatccgatgacatcgtgcgctccaaccattatgaccctgatgaggatgaggactactacaggaaacagttgtcttactttgacagactgcagactggctccaataaaccccaaccacaagcacagtccagccacagcttccccagccattatacacattttggatattcaagtgtctttcttttcttttccttaatgatggactctgtggagaaaccaagcccactggagaaaaaatatgaaccagttccccaagtgacaccagctgtgccaccggccacgctgcccaagccctcacctgatggtaaaattgactgtagtcaggatttttatctcatctctttgactgatgtgcgttgctcttccacagccaaacctcctgctcgagaggacacggtccagaccaactttcttcctcacaagagcttccctgagaagtctccagtcaatggcaccagtgaacagcctccaaagacggtcactagcaccgggggtttgcccacatccacctacaaccgctttgcgcccaagccctacacctcctctgccaagcctttttcgcgcaagttcgacagtcctaaattcaaccacaacctcctgtccaatgacaagcctgagagtgctcccaagggacggagctcgagtccggtaaagcctcaggtacccccacagccccagaacgcagaccaagacagtggcctggacactttcacacgcacaacggacccccgatccaaataccagcagaacaacgtaaacgccgtgcccaaggccatccctgtgagccccagtgccctagaggatgatgaagatgaagacgaaggtcacactgtggtggcaacagctcgtggcatcttcaactctaacggtggcgttctgagctccatcgagacaggtgtcagcatcattatcccacagggtgccatccccgacggcgtggagcaagagatttacttcaaggtctgtcgagacaacagcatcctgccgccactcgacaaggagaaaggagagactctgctcagccctctggtgatgtgtggacctcacggcctaaagttcctgaagcctgtggagctacgcttacctcactgtgcgtcaatgacccctgatggttggtcttttgctctaaaatcctccgactcctcgtcgggtgatccaaaaagctggcagaacaagtctctcaccggagaccccaactacctggtgggagccaactgtgtctctgtgctcattgaccacttttaaagaagaagcagcaggtgtgatgttactgaatgtggaagaatggcggatgaaatgaagacgatggaaacgcacgcacgcaaacacacacatataccactacacacacacacacactgacagacgcactccaagcaaaccaacacacagcatagagtatgaagaagacccagacagtgctggacgaaggagagacaccaatgatcgttacgagctgttctttaaactcaatttcaaagttttgatgtaaaatgatgcatgcccaacgtcactgacgattgacacttatatataaagcaatgtttaatgtaatttttcttttttctttttttacaaaagtatagatggatgtatggcttttgaggcagcatacatgcttgaaaaatctgtgtcaatgtatttatgctatatatgcctacagtatatatagaagaatagagaagaaattggactcgaattcgatcgccagtcaacatcttgttgttttttcagttcaggggactggattttttgtttgtttgtttgtttgtttttttcccttccacattgaaggaatcttactgaaggtttgatacagttggtttaaggaggtggcaagacatgagctggacatgaacccaagcagcagcaacagcacacttttagagacgttcttcctacacttctcactttgttcttccttttcttaccttttgtagcttcctctcttactgagcaccacctctctccttccagcctgagggagatctatgcatgttctttactcaggtccagtagcctcctcggttccttcctcacatctacttaatatctttcctttctctgtgcactctttgcactcacacaaataagcagtgatgccttatctgcagattattcacttttcattaagaaaaaaaagtaagttatgataaattatggtataatgtcatttgttttgccatttttttgtgaaccctctgtataaataaacttgggtttagcacacgcagaaacagtcgataaaagataacaaaggtatgctcttcttttatctgctatgcattgcttaaaaacaaaaaaccatcagagaagaagtggctgtaaataaagctagcatattgccttgtttcttttttgttgtaaatgaactttttgtatgtctttcttttttgtataaaacttagagaaaacatgttttaaaaaagcagaaggaagtgaaagtggttatctttgtattatgggcataccttcaagcctttgaattgtaacctaacaataatacatcacaccttttctaccgatatgttgccgccgctattttaccgtctcaaaaaggtcgtctttttttatttttatttctatttttattactttgtccacgtagggttaaggaaagtatttgcggctcagatgcatttaaaacatcttcatttggaagggtgtgctctcaaagtgtccctctcactgatttctgatactggatgctatattgtgtgcccttaaatgtatttttgtactaatagacaatatattacgtatggcacaccagtactgttttacttttagatataacacggctttattggatattagctcttcacttgtggctgacttttttttttttcccctctgcaacacaattttaagtataccactgtattaataaataaaatcatttttaaattaaagagtgtgtaaggattttttatttttttttaccctacagggttttgtat

SEQ ID NO: 72 - DNA, 1320 bp, Oreochromis niloticus
  CDS (1)..(1317)
aaagaggaaaacaatgcatcatataactttataagtaagagtgcggcgatggaggaaaccgtcatatgggaacagcacacagttacccttcacagggccccaggatttgggtttggcattgccatctcgggtgggcgagacaaccctcatttccagagtggtgaaacatctattgtaatatcggatgtgctgaaaggaggtcctgcagagggtctgctacaagaaaatgatcgagtagtaatggtcaatgcagtctctatggacaatgtagagcatgcctatgctgttcaacaacttcgaaagagtggcaaaaatgcaaagataactattcgcagaaaaaggaaagtacaaatcccagcgtcacggcacggggacagggagacgatgtctgagcacgaggaggaggacagcgatgaggctgatgcttacgatcaccgcagtggacgtggtggacaaaacgatcgggagcgtagcagcagtgggaggcgggatcacagtgcctcacaggagaggagcatctcaccacgctccgatcgccgatcacaagcctcttctgctccacccaggccctccaaggtcactcttgtcaagtcccgcaaaaacgaagaatatggactgcggctggccagccatatctttgtgaaggacatctctccagagagccttgcagccagagatggaaacattcaggagggagatgttgtacttaagattaacggcacagttacagagaacctatcactgacagatgccaagaagctgattgagaggtcaaagggcaagctgaagatggtagtgcagagagacgagcgggccacgctgctcaatattcctgaccttgacgacagcatcccatcagccaataactcagacagagatgacatttcagagatacattcactgacatccgatcattccaatcgatcccatgggagaggaagtcaatcccattcgcctgacagggttgaaacatccgagcatctccgccactcaccgcggcagatcagcaatggcagtaatggatttctctgggaaagagctgaggaattaatcaaacaggaatgggtggtgaaacaggaatgttattttgcctgtgcccatactataaaatgtgtaataaccgtgacagtttgggcaaaaaaaccccaaaacagtaacatgccagaaccaaagccagtttatgcacagcctggtcagcctgacgtggacctgcctgtcagcccatctgatgcccctgtacccagtgctgcacatgatgacagcattctcagaccaagtatgaagctggtcaagttcaagaagggagagagtgtcggttag

SEQ ID NO: 73 - protein, 1652 aa, Oreochromis niloticus
KEENNASYNFISKSAAMEETVIWEQHTVTLHRAPGFGFGIAISGGRDNPHFQSGETSIVISDVLKGGPAEGLLQENDRVVMVNAVSMDNVEHAYAVQQLRKSGKNAKITIRRKRKVQIPASRHGDRETMSEHEEEDSDEADAYDHRSGRGGQNDRERSSSGRRDHSASQERSISPRSDRRSQASSAPPRPSKVTLVKSRKNEEYGLRLASHIFVKDISPESLAARDGNIQEGDVVLKINGTVTENLSLTDAKKLIERSKGKLKMVVQRDERATLLNIPDLDDSIPSANNSDRDDISEIHSLTSDHSNRSHGRGSQSHSPDRVETSEHLRHSPRQISNGSNGFLWERAEELIKQEWVVKQECYFACAHTIKCVITVTVWAKKPQNSNMPEPKPVYAQPGQPDVDLPVSPSDAPVPSAAHDDSILRPSMKLVKFKKGESVGLRLAGGNDVGIFVAGVLEDSPAAKEGLEEGDQILRVNNVDFANIIREEAVLFLLDLPKGDDVTILAQKKKDVYRRIVESDVGDSFYIRTHFEYEKESPYGLSFNKGEVFRVVDTLYNGKLGSWLAIRIGKNHQEVERGIIPNKNRAEQLSSVQYTLPKTPGGDRADFWRFRGLRSSKRNLRKSREDLSAQPVQTKFPAYERVVLREAGFLRPVVIFGPIADVAREKLAREVPEVFELAKSEPRDAGTDQKSSGIIRLHTIKQIIDRDKHAVLDITPNAVDRLNYAQWYPIVVFLNPDTKQGIKNMRTRLCPESRKSARKLYDRALKLRKNNHHLFTTTINLNNMNDGWFGALKEIIHQQQNQLVWVSEGKADGVGDDDLDIHDDRLSYLSAPGSEYSMYSTDSRHTSDYEDTDTEGGAYTDQELDETLNDDVGPPTEPAITRSSEPVREDPPVIQEPPGYVSYPHTVQPDPLNRIDPAGFKAPAPQQMFQKDPYSTDNIGRGGHGMKPVTYNPQQGYHPDEQPYRDYDHPPSRYDISSSGVGGGYQEPKYRNYESYENSVPHYDQQPWNPYNQPFSTANTQAYDPRPPYGEGPDSHYTPPLRYDEPPPQQGFDGRPRYGKPTVSAPVRYDDLPPPPQPSELHYDPNSHLSTYPSAARSPEPAAQRPAYNQGPASQQKGYKPQQYDPAPVNSESSPSLHKVETPSPSPADVPKAAPARDEQQEEDPAMRPQSVLTRVKMFENKRSVSMDRARDAGDSFGNKAADLPLKAGGVIPKANSLSNLDQEKTFSRGPEPQKPQSKGSDDIVRSNHYDPDEDEDYYRKQLSYFDRLQTGSNKPQPQAQSSHSFPSHYTHFGYSSVFLFFSLMMDSVEKPSPLEKKYEPVPQVTPAVPPATLPKPSPDGKIDCSQDFYLISLTDVRCSSTAKPPAREDTVQTNFLPHKSFPEKSPVNGTSEQPPKTVTSTGGLPTSTYNRFAPKPYTSSAKPFSRKFDSPKFNHNLLSNDKPESAPKGRSSSPVKPQVPPQPQNADQDSGLDTFTRTTDPRSKYQQNNVNAVPKAIPVSPSALEDDEDEDEGHTVVATARGIFNSNGGVLSSIETGVSIIIPQGAIPDGVEQEIYFKVCRDNSILPPLDKEKGETLLSPLVMCGPHGLKFLKPVELRLPHCASMTPDGWSFALKSSDSSSGDPKSWQNKSLTGDPNYLVGANCVSVLIDHF

SEQ ID NO: 74 - protein, 439 aa, Oreochromis niloticus
KEENNASYNFISKSAAMEETVIWEQHTVTLHRAPGFGFGIAISGGRDNPHFQSGETSIVISDVLKGGPAEGLLQENDRVVMVNAVSMDNVEHAYAVQQLRKSGKNAKITIRRKRKVQIPASRHGDRETMSEHEEEDSDEADAYDHRSGRGGQNDRERSSSGRRDHSASQERSISPRSDRRSQASSAPPRPSKVTLVKSRKNEEYGLRLASHIFVKDISPESLAARDGNIQEGDVVLKINGTVTENLSLTDAKKLIERSKGKLKMVVQRDERATLLNIPDLDDSIPSANNSDRDDISEIHSLTSDHSNRSHGRGSQSHSPDRVETSEHLRHSPRQISNGSNGFLWERAEELIKQEWVVKQECYFACAHTIKCVITVTVWAKKPQNSNMPEPKPVYAQPGQPDVDLPVSPSDAPVPSAAHDDSILRPSMKLVKFKKGESVG

SEQ ID NO: 75 - DNA, 5281 bp, Oreochromis niloticus
  CDS (250)..(1722)
ttctgcttcgcccttgtattagacagccaatcgctggacgtcactccgccagaaggggtgggttgacgtagtacaggaagccaggcgaggtgaggtggggaggagagatcacaaaattgttagctcgctgctagctgcctcctccgatttgcccgaagtgcgatgagcccaggaggcgaaatttgtggggttttttggttttgattggcgcgacgatgaccctctgaccctaagaatggacataagttaatgatgacgggggagaagaagaagaagaagcggctgaaccgcagcattcttcttgcaaagaaaattataataaaagatggaggaagtcctcagggaatcggggagcccagtgtttaccatgctgtggtggtcatcttcctggagttttttgcatggggtctgctcactaccccgatgctcacggtattacaccagacattcccccaacacacattcctgatgaatgggctcattcatggtgtgaagggcctgttatcatttctcagtgctccgctaattggagcgttgtcagacgtatggggacgcaagtccttcctgctgctaacggtcttcttcacttgcgcgcccattccgctgatgaagatcagtccatggtggtactttgcagtcatctcgatgtccggtgtttttgccgtcaccttctctgtgatctttgcctatgtggcagacatcacacaagagcatgagaggagcacagcttatggtttggtatcagctacctttgcagcaagcctggttaccagcccagccattggagcctacctgtctgaggcttacagtgacaccttggttgtgatcctggccacagccatcgcactgctcgacatctgcttcatcctggtggctgtaccagagtcgctgccggagaagatgaggccagcgtcatggggagcgcccatctcctgggaacaggcagaccccttcgcttctctgcgtaaagtgggccaggactctacggtgctcctcatctgtatcacagtgttcctctcctacctccctgaggccggccagtactccagcttcttcctctatctcagacaggtcataggcttctcctcagagacagtggctgccttcatcgctgttgtgggaatcctctcaatattagctcagacggtcgtgttggggatcctgatgcgctctataggaaataagaacacaatcctgctcggcctcggctttcagatcctccagctggcctggtatggctttggatctcagccctggatgatgtgggcagctggagccgttgctgccatgtccagcatcacgttccccgccatcagcgccattgtgtcccgtaatgcggatcctgaccagcaaggtgttgttcagggcatgatcactggaattcgaggcctctgtaacggtttgggtcctgctctttacggcttcgtcttctacttattccacgtggagctgaccgacacggacggctctgagaaaggtgccaaagggaacatggccaaccccactgacgagagtgccatcatcccaggtcctcccttcctctttggtgcatgctcagtgctgctgtctctgctggtggcgctgttcatcccggagcacactgggcccggtatgaggcccggctcctacaagaagcacagcaacggggcacagagtcactcccacagcccgcaaggcagcggggcagagggcaaggagccgctgctggaggacagcagcgtataacctcagctcagggggggcagactccctcgctccacctcaaaatgccctgcacacatggacagatacacataatttatcacaaggacacacacgcacctcaggcacacgtcacactcgagtgccgcaaagagatgtttgtctgttttgctgtccacagcacaaagttgggcgctccttccttagcaacccttttctttataatagctgggttattgtgaggactttctaaagaccctgtgtgaagaaagtgtgtcgagcatcatcagggctgcagtggaagaccgtgtatgtgtgtgtgtgtgtgtgtgtgtgtgtgtgtgtgtggctgagctgagctgagctggactccaatctttggtttgtctgaagttgtaacagtggagcacacaacagcttgtccccctcctggcgcgaaacaggactgaagtgactttggtttaatgtgcgagtggggatatatctctgatacgttactaaatacctgtgtgactcttgattattcctctttagttagccaagtggcaccttcgtttgtcagaggagagcgtgacgaacgccctctcacatgctaatacttctgttctgatgcttgtctttatgactacagctctgtttaggcgtccaagaaggaaacatagttcttcctctgtgtggacaacaggggagcgcagcagctgttaaacctgtgaaaggagcctgcaaaccagtattggagaggcgctgcctaattgcagtcagggttggcaaccagttcagatacaaaaagctttgttaggaccaggttttgttcaaatatcaaacttcttacagagagatgactagaagagaccactttattagctcaaaatggtttttcaatgtttacttgccattctctagattagtagtacagtttgggttgtatatttttctctgttcaaactgaaggctagttgtgcttcaagtttttattcaagaaacaaatgttgccttgaagtgacttaagatatatatggagacattacgtaacctgtatgaagaccgaggtctgagaaggctctgtaatcttgcgctattgctcccatcggagccgttacacactttttattcctttgtattcatgcccttcctgttactttgtttcctgacatttatcaccatcaagttgaggcttacagagacacggttttatttttaaaaagcctctggaccatttggagctggagcattgctatcaggatgtcggtgtctgcactgactgtttgagttgatatcattaggttcagcagaatatcagccatgctgctgcagtagtaaatacaaaggttaatcagtgtggcgtaaagtggtggataagaattataactgtgtcttgtagtccctgacatttaagctaacatgcgtacactcaaagaggcaggccacacttctcccaatgcctaacatgaagcacctcacggacgtgtctggcaacttgtgtagaagctctgcagatgccagcctgcgccacctaagaggcagaaacaaatagcagtagtggagtagatggctggaaatgttcatgttatcctcaaacagtgaagcaaagtaaaaatctggaggttgtgtcaatgtggagagtattgcgaaatctgcaatgatcccagatttcattagtttaaaaaaaagagaaaataagaagaagaagaaaatccacttaaaagtgtaaatcctgaatttttattatcgttcagatctgcagatgtctctgggtttttctgcaggtctgaactgctgctgccacgtttatttttattttccccggtcaacaggtggcgcagtctgtacctggcatgcctgtaaggtgctcgtgtggtttttgttttctttttttcagtcatgtggatcagcgatactgcgttcccttcattcacatactatgtcgccacctttccacattgtaactttgatctgtgaatgcctctcgtagctaacaactggtttcatgctgtttaacatctgtatgaactgaaacatacgtcacgtatttagtgccatatcttcttgatttgcttttttcttttgtactgtgtgtgtgaatgtacacttgtgtgatttgagtgtttttgttgttctttttattttctcttgtcttaatttctttgactgaagatttaagttttaatgctatttttttaatagctttttaaaacttcagtcatttttttaggattaattgtcaaaattggatggtaaattatcaaatgtccatctgtcccctttgttatgttgtttgtttttgatttcagcctcggtcttcatttaataacaagcatttcaccatggtttgttaagctcataattttttcccagatttctctgaatgtttccaatgaaactgaacatgttgaccacacagtaccctcaatctttaggttttttttgttttgtcttttaagaggggatgttactacacaggaggccattattcccgtttttttttttttgtttgttttttttaaatcatgtaattgaacaacagaaaatcggatcctggtaagattctgcaccagccccccaccaccaccacccacgtgcacacctacagcctccaagcagacgactgtaaatgtacaaaaatcacctgtacctagagaaaaatgtatatatttattcctcaaggagatggccacctcttggtcaatttggttgtatggtgcaattattattataattattatatatttctccagaattacctgctagccactcctgtttttagtacaatgtggtttgtggcctgaactcccctctctgtgtgcctaaaattagccaagaaatgagtatggcaacctaagtaagtaaaatggtggttattaatgtaaatatgggaaactaatgataaactatttattaaaggtttattgtacaatgaaacgtttcgggttgcctctgtggtttctgggtgggtaacacaggtgaaatcatgttactgtagcagtgagtgagcatctgagcagcgataatcatttggtcgttgcatttacggcgatgatcctatagttaatggctgctaaatcccagtaagtctcactataaactggtagcattcctgttgggctttacttgctgttatattactgcacccccattttttttttaatgtaatgctctgactttgctggctgttggttttgtaaacctgccctttgaagcttaatgttaccgctaatgcctcctccacctacacagtgtatatagtcgtgcattgacctgagctcatttatgggcggtggatttgtaattaaatccacatggaggcagtagttacatctggcaggaactttaaagagtcttctccctgaataacagtgaacgcaaagtgggagatgtcacaaaatgtgatatttatccaaaataaagaatacgataaagtggccagaacaatttatttttgttattaatgtagtgtaggggaatttaatgtcttataattagcagctaataacttgcccatcattttgttgaatttctgtgtgaatgatgaagttttactgggtcaatgctcaaatcttaaggtgattaatgagtatttgca

SEQ ID NO: 76 - DNA, 960 bp, Oreochromis niloticus
  CDS (250)..(948)
ttctgcttcgcccttgtattagacagccaatcgctggacgtcactccgccagaaggggtgggttgacgtagtacaggaagccaggcgaggtgaggtggggaggagagatcacaaaattgttagctcgctgctagctgcctcctccgatttgcccgaagtgcgatgagcccaggaggcgaaatttgtggggttttttggttttgattggcgcgacgatgaccctctgaccctaagaatggacataagttaatgatgacgggggagaagaagaagaagaagcggctgaaccgcagcattcttcttgcaaagaaaattataataaaagatggaggaagtcctcagggaatcggggagcccagtgtttaccatgctgtggtggtcatcttcctggagttttttgcatggggtctgctcactaccccgatgctcacggtattacaccagacattcccccaacacacattcctgatgaatgggctcattcatggtgtgaagggcctgttatcatttctcagtgctccgctaattggagcgttgtcagacgtatggggacgcaagtccttcctgctgctaacggtcttcttcacttgcgcgcccattccgctgatgaagatcagtccatggtggtactttgcagtcatctcgatgtccggtgtttttgccgtcaccttctctgtgatctttgcctatgtggcagacatcacacaagagcatgagaggagcacagcttatggtttggtatcagctacctttgcagcaagcctggttaccagcccagccattggagcctacctgtctgaggcttacagtgacaccttggttgtgatcctggccacagccatcgcactgctcgacatctgcttcatcctggtggctgtaccagagtcgctgccggagaagatgagcgcccatctcctgggaacaggcagaccccttcgcttctctgcgtaaagtgggcca

SEQ ID NO: 77 - protein, 491 aa, Artificial Sequence
  Description of Artificial Sequence: Synthetic Oreochromis niloticus
MMTGEKKKKKRLNRSILLAKKIIIKDGGSPQGIGEPSVYHAVVVIFLEFFAWGLLTTPMLTVLHQTFPQHTFLMNGLIHGVKGLLSFLSAPLIGALSDVWGRKSFLLLTVFFTCAPIPLMKISPWWYFAVISMSGVFAVTFSVIFAYVADITQEHERSTAYGLVSATFAASLVTSPAIGAYLSEAYSDTLVVILATAIALLDICFILVAVPESLPEKMRPASWGAPISWEQADPFASLRKVGQDSTVLLICITVFLSYLPEAGQYSSFFLYLRQVIGFSSETVAAFIAVVGILSILAQTVVLGILMRSIGNKNTILLGLGFQILQLAWYGFGSQPWMMWAAGAVAAMSSITFPAISAIVSRNADPDQQGVVQGMITGIRGLCNGLGPALYGFVFYLFHVELTDTDGSEKGAKGNMANPTDESAIIPGPPFLFGACSVLLSLLVALFIPEHTGPGMRPGSYKKHSNGAQSHSHSPQGSGAEGKEPLLEDSSV

SEQ ID NO: 78 - protein, 233 aa, Oreochromis niloticus
MMTGEKKKKKRLNRSILLAKKIIIKDGGSPQGIGEPSVYHAVVVIFLEFFAWGLLTTPMLTVLHQTFPQHTFLMNGLIHGVKGLLSFLSAPLIGALSDVWGRKSFLLLTVFFTCAPIPLMKISPWWYFAVISMSGVFAVTFSVIFAYVADITQEHERSTAYGLVSATFAASLVTSPAIGAYLSEAYSDTLVVILATAIALLDICFILVAVPESLPEKMSAHLLGTGRPLRFSA

SEQ ID NO: 79 - DNA, 4207 bp, Oreochromis niloticus
  CDS (1)..(1287)
atgacgggcaaatctgtgaaagacgttgacagataccaggctgtcctcaactctttactggcgctggaggagaacaaatactgcgctgactgtgaatcgaaaggtccacgatgggcatcctggaatttgggcatcttcatctgtatccgctgtgctggtatccatcgaaacctgggggttcacatctccaaggtcaagtctgtcaacctggatcagtggacgcaggagcaagtccagtgtgttcaagagatgggaaatgccaaggccaaacggctctacgaggcttttttacccgagtgcttccagcgtcccgagacagaccaggctgccgagatcttcattagggacaaatacgaaaagaagaaatacatggataaagttattgacatccagatgctcaggaaagaaaagagttgtgacaacatcccaaaggagccagttgtatttgagaagatgaaattggtagttaaaaaggagaacactaagaaaaaagacgtcagcccaaagacagattcccagtctgtcacagacctgctcggactagaactgcttttatgttgcaagtctgcacctaaaaagcaaataaacacgtcagactctgccctggatctcttcagctccctcgcagccccctcccctgcttcctctacaaaaagcacggtagtagacaccatgcctcagagcagagtgactgcctcagtgcctgagaatctgagcttgttcttaggcccagcacccaaagcagaggagggcacagtcaagaaactatccaaggactccattctttccctgtacgcctccactccctcggtacatgccagcagtatggccgcacatggcttgtacatgaaccaaatgggatatccaacacacccgtacggtccataccattctttagcccaggcagggggaatgggaggcactatgatgacatcacagatggccatgatggggcagcagcagagcggggtgatggcggtgccacaaaacagcatgattggaattcagcagaactgcatgatggggcagcagaatggcttaatgggacagcaacaaagtgggatgataggacagcagcagcaggttgggggtttgcccgcattaccccagcagcaggcttacggagtccagcaagcccagcagctacagtggaacatcagccagatgactcagcacatggccggcgtgaatctttacaacaccagcggtatgatgggatacagcggtcaacaaatgggaggttcagcagctccaagttcggcacacatgacagcgcacgtgtggaaatgagcttgtctatctgagattcgatggagtgccaacgacccacaaaaggagaagagaaacgccgtggatcagactctccattaaacattttctgatgcaagggaggaggaggaggagaagaagaagaagaaggtttgagaaaccactactacctctctctctcctctctggccgcgcttcctcttgccgtctcatgcatagccatgttctgcagatttccatgtttgccttcaggaccttttcatatgatgactaagacaagggggttctgaggccactggttaggactccagagctttctttctgcctagcctttatgagagagcgctcgtgtgcagaaacattatgagggtatcaagcagctgcagaattgcactgtttcttatttaatcagatggcactggggttggcattggggttagcctagctttaaaagctcaaatagaccgagatatataatctggtaacctaaataggtggctcatactttaaattcattagccctacattaccagtatttacccaactgatggagcgacatttagtgatgatatgtacagtggccctgagaggtcaaacacactgcagcctaataaaacaccagcaaaaatgaaaaatggtgcaaaagcacacaaaacataatggaaggtcaataaaacccaatggaaatagaaagaaaaacactggagaagctagcagaaaaaaatctcacaaaacacaacagaaatgtttttggctaaaatgtgacggctaacagctaacagtaaacggctaacagcaaccatgtacctacagtgtccattgtgttttgtcagaatttttttttctatgtccattgtattttaatcaacttctgtggtgcttttgcaaaatttttctgttttgctggtgtttcctacagttgcagtgcatgtgacctctcagggccaccgtagacatagctacattttaacagcagccatatttgcaaagtgtagcaactacaactttattcagccaatttcaaggtagagatttagagcttttcaaaagtatattttcacataagtgagatgagctgctgctaattcacttaataatcattaacaaatataaaagctaggctagcctaatagtcccttcatgctgcatgcagaagacaaatacacataaccatttttagcaacatatatctagaaatttctactcatttaacaatatttaattcaagcaacaaaacctacctacacagcccgtaatattgatgtcttcatctcaatttctagagggcttcttttagaatctttaatcttgactttaaagtgtcaaaagtccaaaaccatattttgggagaccaaagatcaacactagctttactgtaagtggacagtattcctgtatgcttattcctgttcaaccacttaactagtgattaatagaaaaaaaaaacagcaattcagcagtccggcatcactgtcttcactgtgctgttctttcaccaagggtaggacacttaaaaaaaagaaaaagaagaaagaaatcattttgcatgcagtgtcatcagcgcccgcacacctccagttaagaatctacctggtgcattagtggcctcaaataacgttgaatgtctgtaaataggaggtgaacagagaagtgggagtagagacggaaaacttcaaggtgaaggtcagccgggtttcagatgcttccactgaattgcatgaaaagaatgtgtatctagctctgattgtatgtactgtactgtatgtttgttaagatttgcgaatgtgtctctctgaatgtttctccctctgactcagtctttgacaaagactgacaaaaaaactataaaaaaaaataggtaaaacatatgttctgaatgtgatctcggttgactcgtttgatcgcgcgcaattgttcttcggtgtgtttttgttttttatatattccttgtctagaaacgtacaccttgtgtctctggaatgtctgtgctcgatggcatcctgtgggtttccagttttgctgtaacggcctcacctttgcgttgggggcaaacagtgagctgttttgttttttttttctttttgagaggggatgggagtatttaacaatctggccaaaccacatcgtgaagcataaagcgattgtaaaaccacaatctttcacgtctgtttaagctgatgcttgtacgcttctcccacacaaaccatctctgtgccccgatttctcttaaaagtgttgctaaatctgccttttctgataaatgcttatggaaatgctgtgtttctcttatttaattttatttgacacttgtgttaagctggtaagatgctgcttttaatgtgagtggcagcaatataggaggtgcctatgtgcagcatataaggtcttatttcacaacagtgtgacagcagcagtcaccttctccactgagagcaacatttatataagagagagcacatccagcacagcaacagcaaatctgtcagtcaacaaaagtttctggaaaggcagtgcaagtccacctctgtggacgctcaggcctcacctgagtttttccatttgtgatcaggctactttttttttggtccgatattttttcaatgaaacaaaaacgaataaaggaatgtaactttgtacgtacttgtcgatcaagatactgtatattttaattctttatcaaaatatcgctgtatattatgtttcttaaacaacatgttctgtatattagtttttcttttccacatgctttgccccactttacacaatttcaataaaatttaacaatgtatatgtgacatatgataattgtccctgtgaaaacatgcaaataaatattgttttggttaaattttatgttgttttgtttgttgtgttcattgctgggtgtcaggagttttcctgttatgcaactcaggtcagaataaaacgctcagacagg

SEQ ID NO: 80 - DNA, 360 bp, Oreochromis niloticus
  CDS (1)..(324)
atgacgggcaaatctgtgaaagacgttgacagataccaggctgtcctcaactctttactggcgctggaggagaacaaatactgcgctgactgtgaatcgaaaggtccacgatgggcatcctggaatttgggcatcttcatctgtatccgctgtgctgggggttcacatctccaaggtcaagtctgtcaacctggatcagtggacgcaggagcaagtccagtgtgttcaagagatgggaaatgccaaggccaaacggctctacgaggcttttttacccgagtgcttccagcgtcccgagacagaccaggctgccgagatcttcattagggacaaatacgaaaagaagaaatacatggataa

SEQ ID NO: 81 - protein, 429 aa, Oreochromis niloticus
MTGKSVKDVDRYQAVLNSLLALEENKYCADCESKGPRWASWNLGIFICIRCAGIHRNLGVHISKVKSVNLDQWTQEQVQCVQEMGNAKAKRLYEAFLPECFQRPETDQAAEIFIRDKYEKKKYMDKVIDIQMLRKEKSCDNIPKEPVVFEKMKLVVKKENTKKKDVSPKTDSQSVTDLLGLELLLCCKSAPKKQINTSDSALDLFSSLAAPSPASSTKSTVVDTMPQSRVTASVPENLSLFLGPAPKAEEGTVKKLSKDSILSLYASTPSVHASSMAAHGLYMNQMGYPTHPYGPYHSLAQAGGMGGTMMTSQMAMMGQQQSGVMAVPQNSMIGIQQNCMMGQQNGLMGQQQSGMIGQQQQVGGLPALPQQQAYGVQQAQQLQWNISQMTQHMAGVNLYNTSGMMGYSGQQMGGSAAPSSAHMTAHVWK

SEQ ID NO: 82 - protein, 108 aa, Oreochromis niloticus
MTGKSVKDVDRYQAVLNSLLALEENKYCADCESKGPRWASWNLGIFICIRCAGGSHLQGQVCQPGSVDAGASPVCSRDGKCQGQTALRGFFTRVLPASRDRPGCRDLH

SEQ ID NO: 83 - DNA, 1053 bp, Oreochromis niloticus
  CDS (1)..(1050)
atgcctggccccacaccgaccatcagcaaagctcgggtttacaccgacgttaatacacagaagaacagagagtactgggactacgatgctcatgtgccaaactggagtaatcaagacaactatcagctggtgcgtaaactgggcagagggaagtacagtgaagtgtttgaggccataaatgtgaccaataatgagaaagtggtggtgaaaatcctgaagcctgtcaagaagaagaagatcaaacgcgaaatcaaaattcttgaaaacttgcgaggaggaaccaacatcatccgcctggtggacacggtcaaagacccggtgtccagaacaccagcgctagtctttgagtacatcaataacacagattttaaggagctttaccagaagctgacagactacgatatccgttactacatgtatgagcttctaaaggctctggacttctgtcacagtatggggatcatgcacagggacgtgaagccgcacaatgtgatgattgaccaccagctgaggaagctgcgtcttatagattggggtttggctgaattttaccatcccgctcaggaatataatgtcagggtggcctcgcgctatttcaaaggccccgagctgctagtggactatcagatgtatgattacagtttggacatgtggagtctcggctgcatgttggccagtatgattttcctgaaggaaccgttttttcatggccaggacaactatgaccagctggtccgcatcgctaaggttctcggcaccgatgagctctttggctacctgcacaaatatcacatagaactggacactcgcttcaaagacatgctggggcagcaaacacggaaacgctgggagcagttcatccaatcagagaaccagcacctggtgagtccagaggctctggacctgctggacaagctgctgcgctatgaccaccagcagaggctgacggcggccgaggccatgcagcacccgtacttctatcctgtggtgaaggaacaagcaaatgccaacacagatggctcaaaggcaataagcagctccaatgcaacatga

SEQ ID NO: 84 - DNA, 119 bp, Oreochromis niloticus
  CDS (1)..(90)
atgctcatgtgccaaactggagtaatcaagacaactatcagctggtgcgtaaactgggcagagggaagtacagtgaagtgtttgaggccataaatgtgaccaataatgagaaagtggtg

SEQ ID NO: 85 - protein, 350 aa, Oreochromis niloticus
MPGPTPTISKARVYTDVNTQKNREYWDYDAHVPNWSNQDNYQLVRKLGRGKYSEVFEAINVTNNEKVVVKILKPVKKKKIKREIKILENLRGGTNIIRLVDTVKDPVSRTPALVFEYINNTDFKELYQKLTDYDIRYYMYELLKALDFCHSMGIMHRDVKPHNVMIDHQLRKLRLIDWGLAEFYHPAQEYNVRVASRYFKGPELLVDYQMYDYSLDMWSLGCMLASMIFLKEPFFHGQDNYDQLVRIAKVLGTDELFGYLHKYHIELDTRFKDMLGQQTRKRWEQFIQSENQHLVSPEALDLLDKLLRYDHQQRLTAAEAMQHPYFYPVVKEQANANTDGSKAISSSNAT

SEQ ID NO: 86 - protein, 30 aa, Oreochromis niloticus
MLMCQTGVIKTTISWCVNWAEGSTVKCLRP

SEQ ID NO: 87 - DNA, 1335 bp, Oreochromis niloticus
  CDS (1)..(1332)
atgtctgcttcgactggatgctccccatcgggccagcactcgggccttgtccccagtatgtccatgtttcgatggctagaagtgctggagaaggaatttgataaggctttcgtggatgtggatctgttgcttggagaaatagatccagatcaagtggatataacgtatgagggtcggcagaagatgaccagcctcagctcctgtttcgctcagctctgtcataaaacccagactgtcttccagctcaaccataaactagaggctcagctggtggacctgcgctcagagttgaccgaagctaaagctgcacgggtggtggcagaaagggaggtccacgacttgctcctgcagcttcatgctctccaactgcagcttcatgtcaagcaaggccaagctgaggagtcagataccatcaaagataaactgcctacaccaaccttagaagagctggaacaggagctcgaggccagtaagaaggagaaattagcagaggcaaaaatggaggcagaaaccagactatataagaaagaaaacgaggcccttcgcaggcacatggcagtactgcaggccgaagtctacggagccagactggctgctaaatacttggacaaggaactggctggcagggtgcagcagatacagttactgggtcgtgacatgaaagggccagcacatgacaagctctggaatcaactggaggcagaaattcaccttcaccgccataaaactgtgatccgagcatgtagaggtcgaagtgaccctaagagacctcttccctctcctgtgggacatgatccagacatgctgaagaaaacccagggagttggccctatccgaaaggttgtgctggtcaaagaggatcatgagggtctaggaatttccattacaggtgggaaggagcacggcgttcccattttaatttcagagatccatcccagtcagcccgcagacagatgtggagggctgcatgttggagatgccatccttgctgtcaacagcatcaatttgcgagatgccaaacataaggaagctgtcaccattctctctcagcagcgaggacagatagagtttgaggtcgtgtacgtggctcctgaagtggacagcgatgatgagaatgtggagtacgaggatgacagcggtcatcgctacagactctacctggatgaactggatgacagcatcacagcaccacctagcaacagttcagcatcacttcaagcactggagaagttgtcactgagcaatggagcagagtctggagatactgggatgtccagtgagacaccttcaggggaaaccccttcaaagccaccagaaactgactgctcttcctag

SEQ ID NO: 88 - DNA, 120 bp, Oreochromis niloticus
  CDS (1)..(90)
atgtctgcttcgactggatgctccccagcactcgggccttgtccccagtatgtccatgtttcgatggctagaagtgctggagaaggaatttgataaggctttcgtggatgtggatctgtc

SEQ ID NO: 89 - protein, 444 aa, Oreochromis niloticus
MSASTGCSPSGQHSGLVPSMSMFRWLEVLEKEFDKAFVDVDLLLGEIDPDQVDITYEGRQKMTSLSSCFAQLCHKTQTVFQLNHKLEAQLVDLRSELTEAKAARVVAEREVHDLLLQLHALQLQLHVKQGQAEESDTIKDKLPTPTLEELEQELEASKKEKLAEAKMEAETRLYKKENEALRRHMAVLQAEVYGARLAAKYLDKELAGRVQQIQLLGRDMKGPAHDKLWNQLEAEIHLHRHKTVIRACRGRSDPKRPLPSPVGHDPDMLKKTQGVGPIRKVVLVKEDHEGLGISITGGKEHGVPILISEIHPSQPADRCGGLHVGDAILAVNSINLRDAKHKEAVTILSQQRGQIEFEVVYVAPEVDSDDENVEYEDDSGHRYRLYLDELDDSITAPPSNSSASLQALEKLSLSNGAESGDTGMSSETPSGETPSKPPETDCSS

SEQ ID NO: 90 - protein, 30 aa, Oreochromis niloticus
MSASTGCSPALGPCPQYVHVSMARSAGEGI

SEQ ID NO: 91 - DNA, 882 bp, Oreochromis niloticus
  CDS (1)..(879)
atgagccaggacaaacagagtaagcaggtaccggattgcagcggaccgatgtccccgaccaaagcccagaaatcccccaggatgcccaagtgctctcgctgtagaaatcacggatacgtgtctccactgaagggacacaagcgcttttgcaactggagggactgccagtgtcccaaatgcaaattgatcgcggagaggcagagagtcatggcggcccaggttgctctgaggaggcagcaggcccaagaagaagagcttgggatttgtagtcctgtgtctctgtccggttccgagatgatggtcaagaatgaagttggagcagactgcctgttctctgtggagggacggtccccgacacctaccagccacgccacctctgctgtcacagggacccgctcggcatcgtcccccagcccatctgctgctgccagggctcataccgagggaccgtctgacctcctgctggaaaccccctattacaatttctaccagccttcgcgctaccccacctactatggaaacctttacaactactcgcagtaccagcagatgcctcatggtgatggccgcctgcccagccacagcgtgtcgtctcagtaccgcatgcactcctactacccagcagccacctacctgactcagggcctgggctccaccagctgtgtgccacccttctttagcctggatgacaacaataacagctgctctgagaccatggcagcctccttctcacccggcagcatctccgctggtcacgactccaccatggtctgccgctccatcagctccctggttaacggcgacgccaaggctgaatgcgaggccagcagccaggcagccggcttcaccgtcgacgccatcgaaggcggcgccaccaaataa

SEQ ID NO: 92 - DNA, 180 bp, Oreochromis niloticus
  CDS (1)..(120)
atgagccaggacaaacagagtaagcaggtaccggattgcagcggaccccgaccaaagcccagaaatcccccaggatgcccaagtgctctcgctgtagaaatcacggatacgtgtctccactgaagggacacaagcgcttttgcaactggagggactgccagtgtcccaaatgcaaattga

SEQ ID NO: 93 - DNA, 120 bp, Oreochromis niloticus
  CDS (1)..(114)
atgagccaggacaaacagagtaagcaggtaccggattgcagcggaccaaagcccagaaatcccccaggatgcccaagtgctctcgctgtagaaatcacggatacgtgtctccactgaagg

SEQ ID NO: 94 - protein, 293 aa, Oreochromis niloticus
MSQDKQSKQVPDCSGPMSPTKAQKSPRMPKCSRCRNHGYVSPLKGHKRFCNWRDCQCPKCKLIAERQRVMAAQVALRRQQAQEEELGICSPVSLSGSEMMVKNEVGADCLFSVEGRSPTPTSHATSAVTGTRSASSPSPSAAARAHTEGPSDLLLETPYYNFYQPSRYPTYYGNLYNYSQYQQMPHGDGRLPSHSVSSQYRMHSYYPAATYLTQGLGSTSCVPPFFSLDDNNNSCSETMAASFSPGSISAGHDSTMVCRSISSLVNGDAKAECEASSQAAGFTVDAIEGGATK

SEQ ID NO: 95 - protein, 40 aa, Oreochromis niloticus
MSQDKQSKQVPDCSGPRPKPRNPPGCPSALAVEITDTCLH

SEQ ID NO: 96 - protein, 38 aa, Oreochromis niloticus
MSQDKQSKQVPDCSGPKPRNPPGCPSALAVEITDTCLH

SEQ ID NO: 97 - DNA, 840 bp, Artificial Sequence
  Description of Artificial Sequence: Synthetic Oreochromis niloticus
  CDS (199)..(837)
aacaggggaaaagtctacagtgttaactatgtcaaggccaccttggggtacaagcagataaaaaccgtggttctcagaccctgacaaacaatacctagggcagcatcccagttttgtcgctactatctcctcctccgaccagacgttcgggaccaaccgcagcttttgtctgcagccagtcttacgtgttcatccaccatggcctttccattcattgtcatgacattacttttgggctcttccatgatgatggcatttgtcttggatccatccaggaaagaacccgaagctgccgtcttaggtgacaggtgccaaggtgagtcatggcagtccatcagaaagaacctccttagggttctgaacttgcagactgagccgcagctacctgccggtgcactggacagtgtcagagagcagtggaaccgaaccttcagcatcgtttctcacacagccaagcatactgcaaccccagcagtcccaggctactctgcatcagctgataatggaaacagtgcgagcctgaagtgttgttccattgcctcagagatcttcatgaaagatctgggctgggacagctgggtgatccacccgttgagtcttacctatgttcagtgcgcaacctgcaactctgccatgaccactgttcaatgtccatcatcccaagtaaatgtccaggatgccaacacacaggaccaggtgccatgctgtcggcccacctcccaagaagaggtgcccatagtctatatggatggatccagcgccattgtcatgtcctccatgcagctgacccgcagttgtggctgtgagctgggcaactctgaggatcgtggcaaggagtag

SEQ ID NO: 98 - DNA, 420 bp, Oreochromis niloticus
  CDS (199)..(366)
aacaggggaaaagtctacagtgttaactatgtcaaggccaccttggggtacaagcagataaaaaccgtggttctcagaccctgacaaacaatacctagggcagcatcccagttttgtcgctactatctcctcctccgaccagacgttcgggaccaaccgcagcttttgtctgcagccagtcttacgtgttcatccaccatggcctttccattcattgtcatgacattacttttgggctcttccatgatgatggcatttgtcttggatccatccaggaaagaacccgaagctgccgtcttaggtgacaggtgccaaggtgagtcatggcagtccatcagaaagaacctccgttctgaacttgcagactgagccgcagctacctgccggtgcactggacagtgtcagagagcagtggaaccg

SEQ ID NO: 99 - DNA, 360 bp, Artificial Sequence
  Description of Artificial Sequence: Synthetic Oreochromis niloticus
  CDS (199)..(336)
aacaggggaaaagtctacagtgttaactatgtcaaggccaccttggggtacaagcagataaaaaccgtggttctcagaccctgacaaacaatacctagggcagcatcccagttttgtcgctactatctcctcctccgaccagacgttcgggaccaaccgcagcttttgtctgcagccagtcttacgtgttcatccaccatggcctttccattcattgtcatgacattacttttgggctcttccatgatgatggcatttgtcttggatccatccaggaaagaacccgaagctgccgtcttaggtgacaggtgccaaggtgagtcatggcagtccatctgaacttgcagactgagccgcagc

SEQ ID NO: 100 - protein, 213 aa, Artificial Sequence
  Description of Artificial Sequence: Synthetic Oreochromis niloticus
MAFPFIVMTLLLGSSMMMAFVLDPSRKEPEAAVLGDRCQGESWQSIRKNLLRVLNLQTEPQLPAGALDSVREQWNRTFSIVSHTAKHTATPAVPGYSASADNGNSASLKCCSIASEIFMKDLGWDSWVIHPLSLTYVQCATCNSAMTTVQCPSSQVNVQDANTQDQVPCCRPTSQEEVPIVYMDGSSAIVMSSMQLTRSCGCELGNSEDRGKE

SEQ ID NO: 101 - protein, 55 aa, Oreochromis niloticus
MAFPFIVMTLLLGSSMMMAFVLDPSRKEPEAAVLGDRCQGESWQSIRKNLRSELA

SEQ ID NO: 102 - protein, 46 aa, Artificial Sequence
  Description of Artificial Sequence: Synthetic Oreochromis niloticus
MAFPFIVMTLLLGSSMMMAFVLDPSRKEPEAAVLGDRCQGESWQSI

SEQ ID NO: 103 - DNA, 5853 bp, Oreochromis niloticus
  CDS (108)..(2174)
gcattcactactgcatgacagaaaacaccaaaacacctcacatttctctctagctgacctggcgccgaaccctcgagcggacagacaggcaaaggcgttcatatcaaatgtggagtgtggaccagagacaatatcagagtaaaatacacaaaagaagacaaactagaaaagtgaaaccactctgtggacccaggcagactgaaatgatgctggtgatgtttggagtcacggcgtttccctccaacatctccaacgcccagtgcctggaagttaagcagacgcagatcagagagattcagcagggcgccctctccagcctccagcatctaatggaactgaccatttctgagaacgacctgctggagagtatcggtgcttttgccttttctggcctccctcacctcaccaaaatcttaatatctaaaaatgctgctctgaggaatatcggggcttttgttttctccaacctccctgaactcagtgagataatcataacaaaatcaaaacacctgagtttcatccaccccgatgcattcaggaacatggcaagactacggttcttgactatctccaacaccgggctgaggatttttccagacttctccaagatccattccaccgcctgctttctgctggatcttcaggacaacagccacataaagagagtccctgccaatgccttcagaggcctctgcactcaaaccttcgcagagatacggctcaccagaaatggcatcaaggaggtggcaagtgacgccttcaacggaacaaagatgcacagactgttcctaggaggcaaccgacagcttactcacatcagtcccaatgcctttgtgggttccagtgagttggtggtactagacgtctccgaaacagccctcacctctttgccagactcgatccttgatggcctcaagaggctgattgccgagtcagccttcaacctgaaagaacttcctcctattcagctctttaccaaactgcaccaggcaaagctgacatacccatcacactgctgcgctttcctgaacatgcacagaaacagatcgagatggcactcactgtgtgacaaccccgaggctaaaaataacctgcacttcttcagggaatactgctccaactccaccaacatcacttgcagcccggcccctgatgactttaacccctgtgaagatatcatgtctgctacccccttacgcatcctcatctggatcatctctgtcctcgccctgctgggcaacgcagtagttctccttgtattgttaggcagccgctataagctgactgttcctcgattcctcatgtgccacctggcctttgctgacctctgcatgggcatctacctggtagtcatagcaaccgtggatatgctcacacgtggacggtactacaactatgctatagactggcagatgggcttgggctgcaatgctgcaggcttcttcacggtgttcgccagtgagctgtcagtgtttaccttgacagcaatcaccgtggagcgctggcacaccatcacgcatgctctgcgacttgaccgcaaacttcgcctgagacacgcctgcatcatcatgacaataggttggatcttctccttgctggctgcactgctgcccacagttgggatcagcagctatggcaaagtgagcatctgcctccccatggatgttgagtccctagtctcccagttctacgtggtctgtcttctcctcctcaacatcttggcgttcttctgtgtgtgcggctgctacctcagcatctacctcacctttcgcaagccttcatcagcggcagcccacgccgacacccgtgtggctcaacgcatggccgtcctcatcttcacagacttcatctgcatggctccgatctccttcttcgccatctcagctgccctcaagctccctctcatcaccgtctcagactccaagctactgttggtgctattctaccccatcaactcgtgctccaaccccttcttatatgcctttttcacccgtaacttcagaagggatttctttctcctcgcagctcgcttcgggctgtttaagactcgagcacagatttaccggacagagggttcctcgtgtcagcagccaacatggacctctccaaagaacagccgtgttatcttgtattccttggtcaatacgttaagtctagatggaaaacaagagtgctgacttttacgcacatttacaggtacggactgtttgccttgattgcatattatatccatacaaacaggctgctaattccttaaaatgatgcctcagatcatgtcttttgatcactacctgggaaaatttttctatctacttagactagaaagaaaaaaaacacaaaaggcaaccaagtggaaggcaaaagagctgagaactcttttttgacaatttgacccaggagtctgcaaaacacagtgattgttaaaataaacaatgctcttgctcttgcttctgtttgtgctcctaatctgatgctgtgttttttgggcttgagccagtgaaggcttccactgaagactgctcttcagtcaataaatagcatccagagacccagctctcaacagaggtgatgatcctctatataaagatgttggtcagttcaacaaagaagttgatgcttgtctctgtgcaagtctgagatctctgttagggatgtacatgtacaagtggtcaagattggacttccaggccatgagaccagaggtctacaagtcacaaaaccttttaaagctttttataaaattatatatatctatgtcgccacaatctgagcagttcagacactgatgattccagactgatcactgacccaagagaaagcatgcatacatgttcccacctgtcttttaaggttacacataaatcaacatgtttcaatcacaatagtatcagttgactattcagcacaaagtacacacagcgttcagtggcatgtctaaacctggttacctgagctatgctctgcagcaatccatgcaaacatgaccacaaaagaactaattatacactcactggccactttattaggtatacttgtttggctgcttggtaatgcaaatacttaatgagccaatcgcatggtagcagctcagtgcatttagacatgtaatctggggcatttttaagattttttaaatgtggtggcacggcagagaccaagaacacagtagagggggacatttaaatatttgattagcaaaaagatcagaaaactgacagaaattattgggcatgatttttggtgtgcaaccttatgttttattacaagtttattgtgtgaaaagtggtgctgcagaatgctctacatagaattttgtgttggacaattgttttgcaacgtggaaaaagaagtatttagacttaacctaagtaaaagttgtaattgcacttaaatagcttaatagttcacaagttatataatcaaaatgtattcaaagtgcctaaagtaaacacactctttatatagaatggccctttttttctcgtctctttaatgaggcagctgttgatgagtttgattcctgatatattgttcaatagattcatttataaaaaatacaattaatgtacaaaataagaagaagctaaaataatttggggtgggctaatgccactccaagctcctccccctccaaacatgcctctatgtagacataatcaagacaacttgctaaagttcaaaatgagcatcagaatgggaaaaaggtgactgaagtgactttgaaagtttaattgttgttggtgccagatggtcccacatgtcgccacaatctgctggccggactgcaaactgataggaaagcaacagtaacttaaataacttctatacaatcaaggtgtacaagttacataagaaaactgggcatcacttagtctgataactcttgatttctattctgacattcttatagtaggttcagagtttgatttaactgagcaaactgagtcacaaagctcagatcatctaaaactgatttcttgaaaatgaaaaggagttcaccacagtcaccacatttcaatccagcagagcacatttgggatttagtcaaatgggagattgccatcacagatgtgcagctgacaaacttgcagcacctgtgtgatgctatcacatcaatatggaccacaatctctgaggaatgtttcctccactcaattccagttgaatatattttaaaaattaagacagtgtgaagacaaaggggtgtttaacctagcaaaatgtacccaataaagtagctagtgagtgtagtttgactaaatctgggtcagacagctcttttagatacccatgggtttcttttaactcaagtgaagtgccagatgggtggagttctcagcaacataatttagaggtaaaagaagaaaagaatggaggggggagaaaactaatgacttcatctactatgtaacaaacaccatccgtctggcatcccaagataatctaacaaactaaaatgcctcagaatggtttttaagcaggttggatgcttgggatttcagcatatgcacactgcaaaagaaacatattcattcaacattcagtgctgtgattgaatgatattcattaagaagaacactgcagggacctgctgattaacaatctcctcatacacccagtctgctgaacctctcaatgtctacaatttgccaccaactccgtctattttgtaagccacagacctgtaattatctttgaaatgtaattatgtttacgttttcaaacaaacatccaattaagtgtcacttttgaatctgttttcctgaagaatatttcaatgtgctgttttttacactattttataaagtgttttattatatcctctcagcttgaatagattttgtatgatgaatgtgagcgtttgaagaggcgtgacaaacagaaaaactctctcacacacacacatatgcaataattgagctgtctttatctagcaatgctgtccttcagagcatccaaagctttcaaggacaaagtgaccctcccaacctctgctctgtgcagcaaagtgggtgggtgggcgtaggaggagaggtacgcagctgctctttctgcttattacggggggatggatatggcagctagataagctgtgtgtgtgcgcgcacacacacacacacacacacacacaatagcaacccacactctcaaggctgcagctgcaagaaggaatccaagaccatctcattgatatggatacactgcctcctacatgccaacattcaaagttagggtgcaattatatactttcaccaccaggtgatgctactggggctagatttctggtgagtttacctccatctgtttgcacaaaagtccaaacaaattcaccagtctcagtagatcctacaaattttgctcgatgttgtcttatgagaaaaataaataaataaatatttttttcctaaatttgcttttttttaaaataactttttatttctacataattttcataaaagattatatcaattcctgcatgaggattaatgctcatcagacagttacctgtcccctacatacactgtatttcttcttcatttttatatcatatcatatagttttccaagtaaaagataaatcactctaatgcatttgcactcaaatttatgtgcacaaaaaaaagtgagtgttgcaatacagaaagacatgccgttatgctctctgacatcttctctagacagcactggagatggtataacaaaacaccctcagtataaagccttcaagttcatgactaatcgttggcagctaaacaatgccctctggtggtcgtcgtgcataataaatatacaagttaaagtgttaaagttgtattccactcaaaatctgtaatttggtttggggtcagtgtcc

SEQ ID NO: 104 - DNA, 960 bp, Oreochromis niloticus
  CDS (108)..(899)
gcattcactactgcatgacagaaaacaccaaaacacctcacatttctctctagctgacctggcgccgaaccctcgagcggacagacaggcaaaggcgttcatatcaaatgtggagtgtggaccagagacaatatcagagtaaaatacacaaaagaagacaaactagaaaagtgaaaccactctgtggacccaggcagactgaaatgatgctggtgatgtttggagtcacggcgtttccctccaacatctccaacgcccagtgcctggaagttaagcagacgcagatcagagagattcagcagggcgccctctccagcctccagcatctaatggaactgaccatttctgagaacgacctgctggagagtatcggtgcttttgccttttctggcctccctcacctcaccaaaatcttaatatctaaaaatgctgctctgaggaatatcggggcttttgttttctccaacctccctgaactcagtgagataatcataacaaaatcaaaacacctgagtttcatccaccccgatgcattcaggaacatggcaagactacggttcttgactatctccaacaccgggctgaggatttttccagacttctccaagatccattccaccgcctgctttctgctggatcttcaggacaacagccacataaagagagtccctgccaatgccttcagaggcctctgcactcaaaccttcgcagagatacggctcaccagaaatggcatcaaggaggtggcaagtgacgccttcaacggaacaaagatgcacagactgttcctaggaggcaaccgacagcttactcacatcagtcccaatgcctttgtgggttccagtgagttggtggtactagacgtctccgaaacagccctctttgccagactcgatccttgatggcctcaagaggctgattgccgagtcagccttcaacctgaaagaacttcctcctatt

SEQ ID NO: 105 - protein, 689 aa, Oreochromis niloticus
MWSVDQRQYQSKIHKRRQTRKVKPLCGPRQTEMMLVMFGVTAFPSNISNAQCLEVKQTQIREIQQGALSSLQHLMELTISENDLLESIGAFAFSGLPHLTKILISKNAALRNIGAFVFSNLPELSEIIITKSKHLSFIHPDAFRNMARLRFLTISNTGLRIFPDFSKIHSTACFLLDLQDNSHIKRVPANAFRGLCTQTFAEIRLTRNGIKEVASDAFNGTKMHRLFLGGNRQLTHISPNAFVGSSELVVLDVSETALTSLPDSILDGLKRLIAESAFNLKELPPIQLFTKLHQAKLTYPSHCCAFLNMHRNRSRWHSLCDNPEAKNNLHFFREYCSNSTNITCSPAPDDFNPCEDIMSATPLRILIWIISVLALLGNAVVLLVLLGSRYKLTVPRFLMCHLAFADLCMGIYLVVIATVDMLTRGRYYNYAIDWQMGLGCNAAGFFTVFASELSVFTLTAITVERWHTITHALRLDRKLRLRHACIIMTIGWIFSLLAALLPTVGISSYGKVSICLPMDVESLVSQFYVVCLLLLNILAFFCVCGCYLSIYLTFRKPSSAAAHADTRVAQRMAVLIFTDFICMAPISFFAISAALKLPLITVSDSKLLLVLFYPINSCSNPFLYAFFTRNFRRDFFLLAARFGLFKTRAQIYRTEGSSCQQPTWTSPKNSRVILYSLVNTLSLDGKQEC

SEQ ID NO: 106 - protein, 264 aa, Oreochromis niloticus
MWSVDQRQYQSKIHKRRQTRKVKPLCGPRQTEMMLVMFGVTAFPSNISNAQCLEVKQTQIREIQQGALSSLQHLMELTISENDLLESIGAFAFSGLPHLTKILISKNAALRNIGAFVFSNLPELSEIIITKSKHLSFIHPDAFRNMARLRFLTISNTGLRIFPDFSKIHSTACFLLDLQDNSHIKRVPANAFRGLCTQTFAEIRLTRNGIKEVASDAFNGTKMHRLFLGGNRQLTHISPNAFVGSSELVVLDVSETALFARLDP

SEQ ID NO: 107 - DNA, 4974 bp, Oreochromis niloticus
  CDS (1)..(4971)
atgagagcgctcgtgctcgccctgattctggcctttgtggctggtgatcttcaacatcaagatcctgtttttgaagctgataaaacctatgtgtacaagtatgaggcgctgctcctggcgggcctgctcgagaaaggttcagcgagagctggactaaatatcagcagcaaagttagcatcaatgctatagaccagaacacatacttcattaagcttgaggaacctgagctccaggagtatagtggaatttggcctgaggatccttttatcccagcaactgagctgacttcagccctccaagctgagctcacgactcccattaagtttgaatatgtcaatggtgctgttggaaaagtcttcgcccctgaaaccgtctcaacaacagtgcttaacatctacagaggtatcctgaatgtctttcagctcaacgtcaaaaagacactaaatgtctacgagttgcaggaggctggaactcagggtgtgtgcaagacactttactccatcactgaggacacagaggctgaacgtgtctatctgagaaagaccagggacatgagccactgtcaagaaagaataactaaagacatggggttagcatacacagagaaatgtggaaagtgccaggaggacactaaaaacctgaaaggagtttcatcatacagttacatcatgaaaccactcgataatggcatccagatcaaggaggcatcggtccatgagctgatccagttctcacctttcagtgagcagcatggagccgcccatatggagaccaagcaatccttgatgctccttgacgttcgaagacccccttatgcacccactacaccaccaccccaggctgagtattcacaccgtggaaatctcacatatcagttctccactgagcttcttcagttacccattctgctcctcaatatcaacgacatagagtctcagctcgaggacactctggtcaaacaggctgtagaaagagttcatgaagatgcacctctggaatttttgaagtttgttcaactcctccgtgcagcctccaatgaaactctggagaacctctggagcaaacactcagggatttctgcccacagaaaatggatcatggacgccatccctgctgtgggaaatcctgatgctctgagatttatcaaagagaaatacctagcagaaaccataactgtgtttgaagccgttcaggctttgattacttcatttcacatggtgacagcaaccactgaggccattgaggtcatcgagagcctaacaaaggaaagcaaaatagtgagaaacccagttctgcgtcagattgtattccttggctacggtaccatgatttacaaacactgctatgagaggacttcctgtcctgctgagctcatacagcccattcaagaccttcttgcgcaggcactgaaagatggaaacacagaggacatcatcctgtttgtgaaggctttgggaaatgctgcgcatccttctagcctcaagaaaatcacaaagatgctgcccctacatagtaaattaggttcatcactgccagtgagagttcatgctgaagccatgatggccttgaagaacatcgccaaaaaggagcctaaaacggtccagtatttagcctttcagctctacggggacaagactcttcattcagagatccgcatgcttgcgtgcatggtgctctttgagacaaaaccttcaatgagtttggtgtcagctgttgttcatattgtgaagacagatacaaatttgcaagtagtaagcttcacctattcccacatgaagtccctgactaggagcaccagcgttatttatgcctcagttgctgcagcatgcaaagctgccctgagaatgttgggcccaaacctggacaaactgagctcacgtttcagcaaagccatccatgtcgacgtctatagcagtccctttatgcttggtgctgctgcgactgcttactacatcaatgatgctgccaccatcatgcccaaatctattacgactaggatcaaggctttctttgctggagctgctgctgacattctggaggttggagtaagaactgagggactacaggaggcttttctgaaaaacccagcagtttttgatagtgctgacagggtcaccaggatgaaacatgtcattaaggctctctctcactggaagtctgcacccaacagcaaatccctgacttccatctatgtcaagttctttggacaagaagttgcctttgttgactttgacaaaatctggtttgacaacatctttaatctcatctttgccaataacaatgctgacacgtttggtagagatgttttcaaggctctgcagtctggtcctactttgcgctttgttaagcctctgctggctaatgaggtgagacgtatcatgcctactatagctggttttcccatggagctcggtctgtacactgctgctgtggctgctgttcctggtcaaatcaaagtcaccacgactccagctctgccagaagacttttatctcagataccttctcaaggcagatatacacattagtaccaaggtcacaccaagtgtcgctgtgaacacatttgctgtgtttgggataaacactgccatactccaggctgtcatggtatccagagccaaactctactccatcacaccagccaaaactgaagtcacatttaacatcaatgagggctacttgaatttcacagctcttcctgtttcagtgcctgaaaacattacagctgtggaggttgagacttttgctgtggtaagaaatcctgcttcgggagaaagaatcactcctgtgatccctgccaacccaagacagattcttatatccagtaatacttcttctgatgctgttagtgagtcaagatccgaagagttcatttctcagcgtcagaaagctggcatgcacatcaaatctaaaatggtgaagagtaagaagaagtactgcgctcagactgttaacgctggactcaaggcctgtctcaagattgccactgcttacacgggggatgctgcagtgtataaactggctggaaagcactccgctgctttttctgtcacaccaattgaaggtgaagctgctgagagactggaattagaggttcaacttggaagtaaggctgcacagaagatcatcaaacacatcacgcttagagaagaagaaatcccagaggaaacaccagtcttaatgaagctccacaaaatcctggcctctacccagaagaatagcaccatgtcctcctcatcctccagttccaggagctctcgctttcatgtcagatcctcttcttccaattccagctcttcatcccattctagcaggaagaccattgatgcaactgctcaacaagtcttcagcttctccacctctgtcagtacttccaagtccagctttgcatcgagctttgcatcactcttcagtcttagttcaagctcttctcactacagtgcgcaccacagaaagcatcctgcgagtcgccacaaacccaaggagaaacacaagcatcccacctctaaagccacatcgtcacaggttttcaaaagcagaagcagtggctcaagcttggacgctatccaacataagaagcggttccttgacagtcaagctgctatctttggcatgatcttccgtgctgttaaagctgacacgaagaagcagggataccagttcactgcttacatggacaaaaccaccagcagacttcaaatcattctagatgacattgttcctgataacaactggaggctctgtgctgatggagccgtgttgagcatgcacaaagtcaaagctaaaatgaactggggagcagaatgcaaccaatatgacaccacgattacaacagaaactggtcttgtcggtcgaaaccctgcagctcggctgaaggtggactggaatcggctaccgtctgatctcaagcaccatgcaaagacgatgtataagtacatttctgctcacatgcctgccggcttgattcaggaaaaggacagaaacagcgacaagcagctctcgttgactgtggctgtagtatctgacaagatcatcgacctgatttggaaaacaccgagaagcactgttcataagcgggctttgcatcttcccatcactctgccacgtaacgagatcaaagatcttacttccttcagtgacgtctctggaaaagtcaagcacttgttagctgcggctggcgcagctgaatgtagcttcaccgacaatacgctgaccacattcaacaacaagaaattaaagaacgagatgccctcaaactgctatcaggttctggcacaggatggcacagacgagctgaaattcatcgttctactgaggaaggatcgcactgaacagaagcagatcagtgtgaaaattgctcatatagacattgacctctatcagaggagaaccagtgtgactgtgaatgtgaatgggctggaaatacccatgagcaacctgccatatcgttatccccaagctgacatccagatcaaacaaaatggcgaaggcatctctgtgtatgcagctagctatggtcttcatgaagtctactttgacaagaagtcatggaagattaaagttgtggactggatgaaggggaagacttgtgggctctgtggaaaggctgacggggagaccatgcaggagtatcgcacacccactggatggatagccacgacagcagtgagctttgctcattcttggattctgccagctgagagctgcagagacgccactgagtgccgtatgaggcatgaatctgtgcagctggagaaacaggaaaacgtgcaagctcagaactccaagtgctactctgtcgaccctgtgctgcgctgcatggctgggtgcttccctgtgcgcaccaccaacgtcactgttggcttccactgccttccagctggttccagcccctccagcatgtatacgagcgtggacctgatggaaactacggagagtcacctcgcctgcacctgcactgctcagtgtgcttaa

SEQ ID NO: 108 - DNA, 840 bp, Oreochromis niloticus
  CDS (1)..(837)
atgagagcgctcgtgctcgccctgattctggcctttgtggctggtgatcttcaacatcaagatcctgtttttgaagctgataaaacctatgtgtacaagtatgaggcgctgctcctggcgggcctgctcgagaaaggttcagcgagagctggactaaatatcagcagcaaagttagcatcaatgctatagaccagaacacatacttcattaagcttgaggaacctgagctccaggagtatagtggaatttggcctgaggatccttttatcccagcaactgagctgacttcagccctccaagctgagctcacgactcccattaagtttgaatatgtcaatggtgctgttggaaaagtcttcgcccctgaaaccgtctcaacaacagtgcttaacatctacagaggtatcctgaatgtctttcagctcaacgtcaaaaagacactaaatgtctacgagttgcaggaggctggaactcagggtgtgtgcaagacactttactccatcactgaggacacagaggctgaacgtgtctatctgagaaagaccagggacatgagccactgtcaagaaagaataactaaagacatggggttagcatacacagagaaatgtggaaagtgccaggaggacactaaaaacctgaaaggagtttcatcatacagttacatcatgaaaccactcgataatggcatccagatcaaggaggcatcggtccatgagctgatccagttctcacctttcagtgagcagcatggagccgcccatatggagaccaagcaatccttgatgctccttgacgttcgaagacccccttatgcacccactacaccaccaggctga

SEQ ID NO: 109 - DNA, 960 bp, Oreochromis niloticus
  CDS (1)..(903)
atgagagcgctcgtgctcgccctgattctggcctttgtggctggtgatcttcaacatcaagatcctgtttttgaagctgataaaacctatgtgtacaagtatgaggcgctgctcctggcgggcctgctcgagaaaggttcagcgagagctggactaaatatcagcagcaaagttagcatcaatgctatagaccagaacacatacttcattaagcttgaggaacctgagctccaggagtatagtggaatttggcctgaggatccttttatcccagcaactgagctgacttcagccctccaagctgagctcacgactcccattaagtttgaatatgtcaatggtgctgttggaaaagtcttcgcccctgaaaccgtctcaacaacagtgcttaacatctacagaggtatcctgaatgtctttcagctcaacgtcaaaaagacactaaatgtctacgagttgcaggaggctggaactcagggtgtgtgcaagacactttactccatcactgaggacacagaggctgaacgtgtctatctgagaaagaccagggacatgagccactgtcaagaaagaataactaaagacatggggttagcatacacagagaaatgtggaaagtgccaggaggacactaaaaacctgaaaggagtttcatcatacagttacatcatgaaaccactcgataatggcatccagatcaaggaggcatcggtccatgagctgatccagttctcacctttcagtgagcagcatggagccgcccatatggagaccaagcaatccttgatgctccttgacgttcgaagacaccccaggctgagtattcacaccgtggaaatctcacatatcagttctccactgagcttcttcagttacccattctgctcctcaatatcaacgacatagagtctcagctcgaggacactctggtcaaacaggctgtagaaagagttcatgaag

SEQ ID NO: 110 - protein, 1657 aa, Oreochromis niloticus
MRALVLALILAFVAGDLQHQDPVFEADKTYVYKYEALLLAGLLEKGSARAGLNISSKVSINAIDQNTYFIKLEEPELQEYSGIWPEDPFIPATELTSALQAELTTPIKFEYVNGAVGKVFAPETVSTTVLNIYRGILNVFQLNVKKTLNVYELQEAGTQGVCKTLYSITEDTEAERVYLRKTRDMSHCQERITKDMGLAYTEKCGKCQEDTKNLKGVSSYSYIMKPLDNGIQIKEASVHELIQFSPFSEQHGAAHMETKQSLMLLDVRRPPYAPTTPPPQAEYSHRGNLTYQFSTELLQLPILLLNINDIESQLEDTLVKQAVERVHEDAPLEFLKFVQLLRAASNETLENLWSKHSGISAHRKWIMDAIPAVGNPDALRFIKEKYLAETITVFEAVQALITSFHMVTATTEAIEVIESLTKESKIVRNPVLRQIVFLGYGTMIYKHCYERTSCPAELIQPIQDLLAQALKDGNTEDIILFVKALGNAAHPSSLKKITKMLPLHSKLGSSLPVRVHAEAMMALKNIAKKEPKTVQYLAFQLYGDKTLHSEIRMLACMVLFETKPSMSLVSAVVHIVKTDTNLQVVSFTYSHMKSLTRSTSVIYASVAAACKAALRMLGPNLDKLSSRFSKAIHVDVYSSPFMLGAAATAYYINDAATIMPKSITTRIKAFFAGAAADILEVGVRTEGLQEAFLKNPAVFDSADRVTRMKHVIKALSHWKSAPNSKSLTSIYVKFFGQEVAFVDFDKIWFDNIFNLIFANNNADTFGRDVFKALQSGPTLRFVKPLLANEVRRIMPTIAGFPMELGLYTAAVAAVPGQIKVTTTPALPEDFYLRYLLKADIHISTKVTPSVAVNTFAVFGINTAILQAVMVSRAKLYSITPAKTEVTFNINEGYLNFTALPVSVPENITAVEVETFAVVRNPASGERITPVIPANPRQILISSNTSSDAVSESRSEEFISQRQKAGMHIKSKMVKSKKKYCAQTVNAGLKACLKIATAYTGDAAVYKLAGKHSAAFSVTPIEGEAAERLELEVQLGSKAAQKIIKHITLREEEIPEETPVLMKLHKILASTQKNSTMSSSSSSSRSSRFHVRSSSSNSSSSSHSSRKTIDATAQQVFSFSTSVSTSKSSFASSFASLFSLSSSSSHYSAHHRKHPASRHKPKEKHKHPTSKATSSQVFKSRSSGSSLDAIQHKKRFLDSQAAIFGMIFRAVKADTKKQGYQFTAYMDKTTSRLQIILDDIVPDNNWRLCADGAVLSMHKVKAKMNWGAECNQYDTTITTETGLVGRNPAARLKVDWNRLPSDLKHHAKTMYKYISAHMPAGLIQEKDRNSDKQLSLTVAVVSDKIIDLIWKTPRSTVHKRALHLPITLPRNEIKDLTSFSDVSGKVKHLLAAAGAAECSFTDNTLTTFNNKKLKNEMPSNCYQVLAQDGTDELKFIVLLRKDRTEQKQISVKIAHIDIDLYQRRTSVTVNVNGLEIPMSNLPYRYPQADIQIKQNGEGISVYAASYGLHEVYFDKKSWKIKVVDWMKGKTCGLCGKADGETMQEYRTPTGWIATTAVSFAHSWILPAESCRDATECRMRHESVQLEKQENVQAQNSKCYSVDPVLRCMAGCFPVRTTNVTVGFHCLPAGSSPSSMYTSVDLMETTESHLACTCTAQCA

SEQ ID NO: 111 - protein, 279 aa, Oreochromis niloticus
MRALVLALILAFVAGDLQHQDPVFEADKTYVYKYEALLLAGLLEKGSARAGLNISSKVSINAIDQNTYFIKLEEPELQEYSGIWPEDPFIPATELTSALQAELTTPIKFEYVNGAVGKVFAPETVSTTVLNIYRGILNVFQLNVKKTLNVYELQEAGTQGVCKTLYSITEDTEAERVYLRKTRDMSHCQERITKDMGLAYTEKCGKCQEDTKNLKGVSSYSYIMKPLDNGIQIKEASVHELIQFSPFSEQHGAAHMETKQSLMLLDVRRPPYAPTTPPG

SEQ ID NO: 112 - protein, 301 aa, Oreochromis niloticus
MRALVLALILAFVAGDLQHQDPVFEADKTYVYKYEALLLAGLLEKGSARAGLNISSKVSINAIDQNTYFIKLEEPELQEYSGIWPEDPFIPATELTSALQAELTTPIKFEYVNGAVGKVFAPETVSTTVLNIYRGILNVFQLNVKKTLNVYELQEAGTQGVCKTLYSITEDTEAERVYLRKTRDMSHCQERITKDMGLAYTEKCGKCQEDTKNLKGVSSYSYIMKPLDNGIQIKEASVHELIQFSPFSEQHGAAHMETKQSLMLLDVRRHPRLSIHTVEISHISSPLSFFSYPFCSSISTT

SEQ ID NO: 113 - DNA, 5339 bp, Oreochromis niloticus
  CDS (96)..(5336)
cgccatttagttaatgatacatttgatgggcaacgtcagcaaaaaatctgcttaaaaaggacgcctctgcctgcagatcctcacatccaccagccatgagggtgcttgtactagctcttgctgtggctctcgcagtgggggaccagtccaacttggccccaggattcgcctctgttaagacctacatgtacaaatatgaagcggttcttatgggcggcctgcctgaagagggcctggctcgagctggggttaaaatccggggcaaagttttgatcagtgcaacaagtgccaacgactacattctgaagcttgtagaccctcagttgctggagtacagtggcatctggcccaaagatcctttccatccagccaccaagctcaccacagccctggctactcagctctcgacaccggtcaagtttgagtatacaaacggcgttgttgggagactggctgcacctcctggggtctccacaacagtgctgaatatctacaggggcatcatcaacctcctgcagctgaatgtaaagaagacacagaatgtctacgagatgcaagagtctggagctcatggtgtgtgcaagaccaactatgtgatcagggaggacgcgagggccgaacgcattcatctgaccaagaccaaggacctgaaccactgccaggagaaaatcatgaaggccatcggcttggaacacgtagagaaatgccatgattgtgaagctagaggaaagagcctgaagggaactgcttcctataactacatcatgaagccagcacccagtggttctctgattatggaggctgtcgctagagaggtcatcgagttttcacctttcaacattttgaatggcgctgctcagatggagtctaagcaaattctgaccttcctggatattgagaacacccctgtggatcatgccagatacacctatgttcaccgcggatccctgcagtatgagcatggcagcgagattctccagacacccatccatcttctgagggtcacccatgccgaggctcagattgtcagcactctgaaccacctggtagcctccaacgtggccaaggtccatgaagatgcccctctgaagtttgttgagctcatccaggtgatgcgtgtggccagatttgagactattgagtccctctgggctcagtttaaatctagacctgatcacaggtactggttactgaatgctgtcccccacattcgcactcacgctgcgcttaagttcctcattgagaagctccttgctaatgagttaagtgagactgaagctgctatggctctcttggaatgtctgcactctgtgacagctgaccagaaaaccattgaacttgtcagaagcctggctgagaaccacagagtgaaacgtaacgctgtgctcaacgagattgtgatgctgggctggggcactgtaatttccaggttctgtaaagcgcagccatcttgctcatctgatcttgtgacacctgtacatagacaagttgcagaggctgttgaaactggtgacatcgatcagctcactgtcactctcaaatgcctggataacgctggacatcctgctagccttaagacaatcatgaagttcctgcctggctttggcagtgctgctgcccgagtcccactcaaagttcaggttgacgctgttctagccctgaggagaattgcaaagagggaacccaagatggtccaggaaatagctgctcagttgctcatggaaaagcatctccatgcagaactgcgtatggttgctgccatggtgctctttgagactaaactccccgtgggtctagcagctagcatttccacagccttgatcaaagaaaagaacctgcaggtcgttagctttgtctactcttacatgaaggccatggccaagaccacatcccctgaccacgtttctgttgctgcagcatgtaatgttgccttgaggttcctcaaccccaaattaggcagactgaacttccgctacagccgagccttccatgtggatacctataacaatgcctggatgatgggtgctgccgccagtgccgtcttaattaacgacgctgcaaccgtgttaccaagaatgattatggccaaagcccgtacttacatggccggagcttatgttgatgcttttgaggttggagtgaggactgagggaatccaggaggctcttttgaaaagacgacatgaaaattctgagaatgcagacaggatcaccaagattaaacaagccatgagagctctttctgagtggagggctaatccttcgagccaggccctggcctctatgtatgtgaaggtcttcggacaagaaattgcatttgccaacattgacaaatccaaggttgaccagcttatccagtttgccagtggacctttgagaaacgtattcagagatgctgtgaattctgtgctgtctggttatgcaacacattttgctaaaccaatgctgctcggtgagctccgtctcatccttcccaccactgttgggttgcccatggagatcagcctcattacatccgctgtgactgctgcatctgttgacgtccaagccactgtgtcaccacctctgcctgtcaactaccgagtttcccagcttctggagtccgatatccaactgagggctacagttgctccaagtcttgccatgcagacctatgcattcatgggtgtgaacaccgccttaatccaggctgcagtgatgacaaaagccaaagtttacacagctgttcctgcacagataaaagcaaggattgacattgttaagggcaacttgaaggttgagttcctgtcactccagggcattaacacaattgcatctgcacatgcggagacggttgccattgcaagaaatgtggaagacctcccagccgcaagaagcacaccactgatctcatctgaaactgcatcacaactttcaaaggcctctctcaactcaaagatctccaggatggcatcctctgtgactggtggcatgtctgcgtcatctgaaatcattcctgctgacctgccaagtaagattgggaggaaaatgaaactccctaaaacctacaggaagaaaatccgtgcttcaagcagaatgctaggattcaaggcctacgctgagattaaatctcacaatgccgcctacatcagagactgccctctctacgctctgatcggaaagcatgctgcttctgttaggattgctccagcttctggaccagtcattgagaagattgaagttgagattcaggtcggagataaagcagcagaaaatatgattaaagcgattgacatgagcgaagaggaggaagctcttgaggataagaatgtcctcttgaaaatcaagaaaatactggcacctggtctcaagaacaccacatcatcttcctccagctcctccagctcctcttcatccagctctagctccaacaagtcttcttcatccagttcccgctccagcagctcccagtcatccagctctcgttcccataggtctcgctccagaaagtcccagtctagcagctctcagtcaagccgctctccctcaagctcttcctcctcttcctcctcttcatcatccagatcttcttccaggtcatcttccagatcatcttccagatcttcttctaggtcctcctctcgctccagaactaagatggctgacattgttgctcctattatcacgacgtccaccagagtgagcagttcctccagtcgatcagcctctaacagctcctccagcagtgcttcatacttgctcagctcatctaagtcatcaagctctagatcctctcggcgcagtgctcagtctaagcaacaactgcttgccttgaagttcagaaagaaccacgtccacaggcatgccatctccacacagcgcggcagcagtcacagcagtgcccgcagcttcgattccatctacaataaggccaagtacctcgctaacacactcactcctgccatgtccattgcaatccgtgccgtgagagtcgaccacaaggtccagggataccagctagcagcttacctggacaaacagaccaatagactgcagctgatttttgccagagtcgctgagaaggacaactggagaatctgtgccgacattgtgcagctgagttcgcacaagatgatggccaagattgcctggggtgctgaatgcaagcaatactccaccatgattgtagctgaaactggtcttttgggtcatgagcccgcagcccgcttgaagctgacctgggacaaactgccaggaagcataaagcactacgcaaagagggcgttgaaatccattgtccctattgctcaagaatatggagtaaactacgcaaaggccaagaatcctcgtaatcaaatcaaactgactgtagctgttgctactgagacaagcatgaatattgtgctgaacacaccaaaggcaatcatttacaagcgtggggtgtgtctacctgttgctttaccaattggaaacactgctgccgagctgcaagcgacccgggacaactgggctgacaagatgtcctatttggttaccaaagctaacgcagttgaatgctccctcatcaacaacacactgaccacattcaacaacaggaaagctagagatgagctgccacactcgtgctaccaggtcttggctcaggattgcacaccagaactcaaattcatggttctgctgaagaaagaccaaatacaggatcagaagcagatcaatgttaagatttcagacatcgatgtggacatgtatcggaagaacaacgccattgcggtgatggttaacggagttgaaatccctaacagcaacctgccatacctgcatccatcaggtaacatacatataagacagtcaaatgaaggcattactctcaatgcacccagccatggtcttcaggaggtcttccttggcttcaacgagctgagggttaaagttgcagactggatgaaaggaaagacttgtggtgcctgtggaacggcaagcggaaatgtcggagacgagtaccgcacacccagtgaacaggtgaccaaggatgccatcagctacgcccactcctgggttctgtcttcaaacacctgccgtgatccctccgagtgttccatcaagcaggaatctgtgaagctggagaagcgggtgatctttgaaggtgtggagtccaaatgctactctgttgagcccgtgctgcagtgcctgcccggctgtatcccagtgagaaccactaccgtcaacgttggctttcactgcctgcccagtgacacaactgtggaccgttctggtctgagcagcttctttgagaagagcatcgacctgagggatactgcagaagcccacctggcctgtcgctgcactcctcagtgtgcttaa

SEQ ID NO: 114 - DNA, 720 bp, Oreochromis niloticus
  CDS (96)..(701)
cgccatttagttaatgatacatttgatgggcaacgtcagcaaaaaatctgcttaaaaaggacgcctctgcctgcagatcctcacatccaccagccatgagggtgcttgtactagctcttgctgtggctctcgcagtgggggaccagtccaacttggccccaggattcgcctctgttaagacctacatgtacaaatatgaagcggttcttatgggcggcctgcctgaagagggcctggctcgagctggggttaaaatccggggcaaagttttgatcagtgcaacaagtgccaacgactacattctgaagcttgtagaccctcagttgctggagtacagtggcatctggcccaaagatcctttccatccagccaccaagctcaccacagccctggctactcagctctcgacaccggtcaagtttgagtatacaaacggcgttgttgggagactggctgcacctcctggggtctccacaacagtgctgaatatctacaggggcatcatcaacctcctgcagctgaatgtaaagaagacacagaatgtctacgagatgcaagagtctggagctcatggtgtgtgcaagaccaactatgtgatcagggagggccgaacgcattcatctgaccaagaccaaggacctgaaccactgccaggagaaaatcatgaaggccatcggcttggaacacgtagagaaatgccatgattgtgaagctagagga

SEQ ID NO: 115 - protein, 1747 aa, Oreochromis niloticus
MRVLVLALAVALAVGDQSNLAPGFASVKTYMYKYEAVLMGGLPEEGLARAGVKIRGKVLISATSANDYILKLVDPQLLEYSGIWPKDPFHPATKLTTALATQLSTPVKFEYTNGVVGRLAAPPGVSTTVLNIYRGIINLLQLNVKKTQNVYEMQESGAHGVCKTNYVIREDARAERIHLTKTKDLNHCQEKIMKAIGLEHVEKCHDCEARGKSLKGTASYNYIMKPAPSGSLIMEAVAREVIEFSPFNILNGAAQMESKQILTFLDIENTPVDHARYTYVHRGSLQYEHGSEILQTPIHLLRVTHAEAQIVSTLNHLVASNVAKVHEDAPLKFVELIQVMRVARFETIESLWAQFKSRPDHRYWLLNAVPHIRTHAALKFLIEKLLANELSETEAAMALLECLHSVTADQKTIELVRSLAENHRVKRNAVLNEIVMLGWGTVISRFCKAQPSCSSDLVTPVHRQVAEAVETGDIDQLTVTLKCLDNAGHPASLKTIMKFLPGFGSAAARVPLKVQVDAVLALRRIAKREPKMVQEIAAQLLMEKHLHAELRMVAAMVLFETKLPVGLAASISTALIKEKNLQVVSFVYSYMKAMAKTTSPDHVSVAAACNVALRFLNPKLGRLNFRYSRAFHVDTYNNAWMMGAAASAVLINDAATVLPRMIMAKARTYMAGAYVDAFEVGVRTEGIQEALLKRRHENSENADRITKIKQAMRALSEWRANPSSQALASMYVKVFGQEIAFANIDKSKVDQLIQFASGPLRNVFRDAVNSVLSGYATHFAKPMLLGELRLILPTTVGLPMEISLITSAVTAASVDVQATVSPPLPVNYRVSQLLESDIQLRATVAPSLAMQTYAFMGVNTALIQAAVMTKAKVYTAVPAQIKARIDIVKGNLKVEFLSLQGINTIASAHAETVAIARNVEDLPAARSTPLISSETASQLSKASLNSKISRMASSVTGGMSASSEIIPADLPSKIGRKMKLPKTYRKKIRASSRMLGFKAYAEIKSHNAAYIRDCPLYALIGKHAASVRIAPASGPVIEKIEVEIQVGDKAAENMIKAIDMSEEEEALEDKNVLLKIKKILAPGLKNTTSSSSSSSSSSSSSSSSNKSSSSSSRSSSSQSSSSRSHRSRSRKSQSSSSQSSRSPSSSSSSSSSSSSRSSSRSSSRSSSRSSSRSSSRSRTKMADIVAPIITTSTRVSSSSSRSASNSSSSSASYLLSSSKSSSSRSSRRSAQSKQQLLALKFRKNHVHRHAISTQRGSSHSSARSFDSIYNKAKYLANTLTPAMSIAIRAVRVDHKVQGYQLAAYLDKQTNRLQLIFARVAEKDNWRICADIVQLSSHKMMAKIAWGAECKQYSTMIVAETGLLGHEPAARLKLTWDKLPGSIKHYAKRALKSIVPIAQEYGVNYAKAKNPRNQIKLTVAVATETSMNIVLNTPKAIIYKRGVCLPVALPIGNTAAELQATRDNWADKMSYLVTKANAVECSLINNTLTTFNNRKARDELPHSCYQVLAQDCTPELKFMVLLKKDQIQDQKQINVKISDIDVDMYRKNNAIAVMVNGVEIPNSNLPYLHPSGNIHIRQSNEGITLNAPSHGLQEVFLGFNELRVKVADWMKGKTCGACGTASGNVGDEYRTPSEQVTKDAISYAHSWVLSSNTCRDPSECSIKQESVKLEKRVIFEGVESKCYSVEPVLQCLPGCIPVRTTTVNVGFHCLPSDTTVDRSGLSSFFEKSIDLRDTAEAHLACRCTPQCA

SEQ ID NO: 116 - protein, 202 aa, Oreochromis niloticus
MRVLVLALAVALAVGDQSNLAPGFASVKTYMYKYEAVLMGGLPEEGLARAGVKIRGKVLISATSANDYILKLVDPQLLEYSGIWPKDPFHPATKLTTALATQLSTPVKFEYTNGVVGRLAAPPGVSTTVLNIYRGIINLLQLNVKKTQNVYEMQESGAHGVCKTNYVIREGRTHSSDQDQGPEPLPGENHEGHRLGTRREMP

SEQ ID NO: 117 - DNA, 18 bp, Artificial Sequence
  Description of Artificial Sequence: Synthetic primer
tgtaaaacgacggccagt

SEQ ID NO: 118 - DNA, 18 bp, Artificial Sequence
  Description of Artificial Sequence: Synthetic primer
taggagtgcagcaagcat